(19) United States
(12) Patent Application Publication
(10) Pub. No.: US 2006/0154954 A1
(43) Pub. Date: Jul. 13, 2006

(54) Combinations and use of selected pharmaceutically active compounds

(75) Inventors: Wilm Buhr, KONSTANZ (DE); Wolfgang Kromer, Konstanz (DE); Peter jan Zimmermann, Radolfzell (DE); Andreas Palmer, Konstanz (DE); Christof Brehm, Konstanz (DE); Thomas Klein, Konstanz (DE); Joerg Senn-Bilfinger, Konstanz (DE); Wolfgang-Alexander Simon, Konstanz (DE); Stefan Postius, Konstanz (DE)

Correspondence Address: NATH & ASSOCIATES PLLC, 112 South West Street, Alexandria, VA, 22314, US

(73) Assignee: ALTANA PHARMA AG, KONSTANZ (DE)

(21) Appl. No.: 10/545,031

(22) PCT Filed: Feb. 16, 2004
(86) PCT No.: PCT/EP04/50138

§ 371 Date: Nov. 10, 2005

(30) Foreign Application Priority Data
Feb. 17, 2003 (EP) 03003530.7

(51) Int. Cl. A61K 31/4745

(52) U.S. Cl. 514/292

(57) ABSTRACT

The present invention relates to new combinations and new uses of certain selected tricyclic imidazo [1,2-a]pyridine compounds in the prevention or treatment of medicament caused gastrointestinal diseases.

### FIELD OF APPLICATION OF THE INVENTION

The invention relates to now use of certain selected tricyclic imidazo[-1,2-a]pyridine compounds in the prevention or treatment of medicament caused gastrointestinal diseases and/or medicament associated gastrointestinal disorders, to new use of said compounds in combination therapy, and to new combinations comprising said selected tricyclic imidazo[1,2-a]pyridine compounds

### KNOWN TECHNICAL BACKGROUND

Tricyclic imidazo[1,2-a]pyridine compounds are known from prior art as reversible proton pump inhibitors and acid pump antagonists.

The use of tricyclic imidazo[1,2-a]pyridine compounds in the prevention or treatment of medicament caused gastrointestinal diseases is known from a variety of prior art documents such as, for example, the international applications WO 984270, WO 0017200, WO 0026217, WO 0063211, WO 0172756, WO 0172755, WO 0172757, WO 0234749, WO 03014120, WO 03014123, WO 03016310 and WO 03091253.

Abovementioned international applications describe tricyclic imidazo[1,2-a]pyridine compounds which are said to exhibit a marked inhibition of gastric acid secretion and an excellent gastric and intestinal protective action. In this connection, abovementioned international applications teach also the utilizability of these compounds particularly in the prevention or treatment of gastrointestinal inflammatory diseases and lesions which are caused by medicaments. In particular, abovementioned international applications disclose in a specific way the utilizability of tricyclic imidazo[1,2-pyridine compounds in the prevention or treatment of stomach ulcers, duodenal ulcers or medicament related functional gastropathy, which are caused by certain antiinflammatories and antirheumatics.

The international application WO 02069968 describes the use of generically disclosed and stereochemically undefined tricyclic imidazo[1,2-a]pyridine compounds, which are substituted in 3-position with a hydroxy-1-4C-alkyl radical on the imidazo ring, in the prevention of gastric ulcer induced by certain medicaments international application WO 02/069968 also claims combinations comprising said medicaments and generically disclosed and stereochemically undefined tricyclic imidazo[1,2-a]pyridine compounds, which are said to be useful in the prevention of medicament induced gastric ulcer.

J. J. Kaminski et al., J. Med. Chem. 1985, 28, 876-892, describe the cytoprotective properties of certain imidazopyridines.

There is still a severe need in the art of having drugs with good tolerance on the gastrointestinal system.

### DESCRIPTION OF THE INVENTION

Surprisingly and unanticipatedly, it has now been found that certain purposively selected, specifically disclosed and stereochemically predominantly well-defined tricyclic imidazo[1,2-a]pyridine compounds, which are described in greater detail below, have, as a first aspect (aspect 1) of the present invention, advantageous gastro-protective action against certain medicaments (such as, for example, those medicaments mentioned below in the description of this invention, especially antiinflammatories and antirheumatics, and/or, in particular, those medicaments which cause erosive changes and/or lesions in the gastrointestinal system) and/or are well useful and effective in prevention or treatment of gastrointestinal disorders associated with certain medicaments indicated below and/or are particularly useful and effective in prevention or treatment of gastrointestinal diseases caused by certain medicaments selected from the group consisting of NSAIDs (non-steroidal antiinflammatory drugs), COX-2 (cyclooxygenase 2) inhibitors, NO-NSAIDs (nitric oxide releasing NSAID), bisphosphonates and corticosteroids, in particular caused by certain medicaments selected from the group consisting of NSAIDs, COX-2 inhibitors, NO-NSAIDs and bisphosphonates; and/or can be used, as a second aspect (aspect 2) of the present invention, in combination therapy of diseases and/or disorders which can be treated, ameliorated or prevented with said certain medicaments mentioned above in aspect 1, particularly with those medicaments selected from the group consisting of NSAIDs (non-steroidal antiinflammatory drugs), COX-2 (cyclooxygenase 2) inhibitors, NO-NSAIDs (nitric oxide releasing NSAID), bisphosphonates and corticosteroids, whereby said combination therapy is characterized by improved gastrointestinal safety and tolerance compared to monotherapy.

Unexpectedly it has been found in this context, that the gastrointestinal safety and tolerability of a combination or composition comprising (a) at least one tricyclic imidazo[1,2-a]pyridine compound as defined herein, and (b) an agent selected from the group consisting of NSAIDs, COX-2 inhibitors, NO-NSAIDs, bisphosphonates and corticostereoids is greater than that can be achieved with said agent (b) alone, i.e. greater than the gastrointestinal safety and tolerability of a monotherapy using only said agent (b) unpartnered with said tricyclic imidazo[1,2-a]pyridine compound (a).

Within the scope of this invention, the term “selected, specifically disclosed and stereochemically well defined tricyclic imidazo[1,2-a]pyridine compounds” refers in a first embodiment (embodiment a) of the present invention to those tricyclic imidazo[1,2-a]pyridine compounds, which are selected from a group consisting of those tricyclic imidazo[1,2-a]pyridine compounds which are specifically disclosed and/or individualized and/or claimed in the following patent applications and patents:

WO 9842707, WO 0017200, WO 0026217, WO 0063211, WO 0172756, WO 0172755, WO 0172757, WO 0234749, WO 03014120, WO 03014123, WO 03016310 and WO 03091253, and which are not substituted by a hydroxy-1-4C-alkyl radical bonded on the imidazo ring;

and/or to those compounds which are mentioned expressis verbis in the list A below;

and to the salts, solvates and solvates of the salts of these compounds.

List A consists of the following compounds:
(7S,8R,9R)-2,3-dimethyl-7,8-dihydroxy-9-phenyl-7,8,9,10-tetrahydroimidazo[1,2-h][1,7]naphthyridine
(7S,8R,9R)-7,8-isopropylidenedioxy-2,3-dimethyl-9-phenyl-7,8,9,10-tetrahydroimidazo[1,2-h][1,7]naphthyridine,
7,8-dihydroxy-9-phenoxy-2,3-dimethyl-7H-8,9-dihydropyrano[2,3-c]imidazo[1,2-a]pyridine,
(7R,8R,9R)-2,3-dimethyl-8-hydroxy-7-methoxy-9-phenyl-7,8,9,10-tetrahydroimidazo[1,2-h][1,7]naphthyridine,
(7S,8S,9S)-2,3-dimethyl-8-hydroxy-7-methoxy-9-phenyl-7,8,9,10-tetrahydroimidazo[1,2-h][1,7]naphthyridine,
(7S,8S,9S)-2,3-dimethyl-8-hydroxy-7-methoxy-9-phenyl-7,8,9,10-tetrahydroimidazo[1,2-h][1,7]naphthyridine,
(7S,8S,9S)-2,3-dimethyl-8-hydroxy-7-methoxy-9-phenyl-7,8,9,10-tetrahydroimidazo[1,2-h][1,7]naphthyridine,
(7S,8S,9S)-2,3-dimethyl-8-hydroxy-7-methoxy-9-phenyl-7,8,9,10-tetrahydroimidazo[1,2-h][1,7]naphthyridine,
(7S,8S,9S)-2,3-dimethyl-8-hydroxy-7-methoxy-9-phenyl-7,8,9,10-tetrahydroimidazo[1,2-h][1,7]naphthyridine,
(7R,8R,9R)-2,3-dimethyl-8-hydroxy-7-(2-methoxyethoxy)-9-phenyl-7,8,9,10-tetrahydroimidazo-[1,2-h][1,7]naphthyridine,
(7R,8R,9R)-2,3-dimethyl-8-hydroxy-7-(2-methoxyethoxy)-9-phenyl-7,8,9,10-tetrahydroimidazo-[1,2-h][1,7]naphthyridine,
(7R,8R,9R)-2,3-dimethyl-8-hydroxy-7-(2-methoxyethoxy)-9-phenyl-7,8,9,10-tetrahydroimidazo-[1,2-h][1,7]naphthyridine,
(7R,8S,9S)-2,3-dimethyl-8-hydroxy-7-(2-methoxyethoxy)-9-phenyl-7,8,9,10-tetrahydroimidazo[1,2-h][1,7]naphthyridine,
(7S,8R,9R)-2,3-dimethyl-8-hydroxy-9-phenyl-7-(2-methoxyethoxy)-9-phenyl-7,8,9,10-tetrahydroimidazo[1,2-h][1,7]-naphthyridine,
(7R,8R,9R)-2,3-dimethyl-7,8-dimethoxy-9-phenyl-7,8,9,10-tetrahydroimidazo[1,2-h][1,7]naphthyridine,
(7R,8R,9R)-2,3-dimethyl-8-hydroxy-7-(2-methylthioethyloxy)-9-phenyl-7,8,9,10-tetrahydroimidazo-[1,2-h][1,7]naphthylpyridine,
(7S,8R,9R)-2,3-dimethyl-8-hydroxy-7-(2-methylthioethyloxy)-9-phenyl-7,8,9,10-tetrahydroimidazo-[1,2-h][1,7]naphthalene,
(7S,8R,9R)-2,3-dimethyl-8-hydroxy-7-(2-methylsulphinylethoxy)-9-phenyl-7,8,9,10-tetrahydroimidazo-[1,2-h][1,7]naphthyridine,
(7R,8R,9R)-2,3-dimethyl-8-hydroxy-7-(ethylthio)-9-phenyl-7,6,9,10-tetrahydroimidazo[1,2-h][1,7]naphthyridine,
(7S,8R,9R)-2,3-dimethyl-8-hydroxy-7-(ethylthio)-9-phenyl-7,8,9,10-tetrahydroimidazo[1,2-h][1,7]naphthyridine,
(7R,8R,9R)-2,3-dimethyl-8-hydroxy-7-(2,2,2-trifluoroethoxy)-9-phenyl-7,8,9,10-tetrahydroimidazo-[1,2-h][1,7]naphthyridine,
(7S,8R,9R)-2,3-dimethyl-8-hydroxy-7-(2,2,2-trifluoroethoxy)-9-phenyl-7,8,9,10-tetrahydroimidazo-[1,2-h][1,7]naphthyridine,
(7S,8R,9R)-8-acetoxy-7-(2-methoxyethoxy)-2,3-dimethyl-9-phenyl-7,8,9,10-tetrahydroimidazo[1,2-h]-[1,7]naphthyridine,
(7R,8R,9R)-8-acetoxy-7-(2-methoxyethoxy)-2,3-dimethyl-9-phenyl-7,8,9,10-tetrahydroimidazo[1,2-h][1,7]naphthyridine,
(7R,8R,9R)-8-acetoxy-7-ethoxy-2,3-dimethyl-9-phenyl-7,8,9,10-tetrahydroimidazo[1,2-h][1,7]naphthyridine,
(7R,8R,9R)-7-(2-methoxyethoxy)-2,3-dimethyl-9-phenyl-8-propionyloxy-7,8,9,10-tetrahydroimidazo-[1,2-h][1,7]naphthyridine,
(7R,8R,9R)-8-benzoyloxy-7-(2-methoxyethoxy)-2,3-dimethyl-9-phenyl-7,8,9,10-tetrahydroimidazo-[1,2-h][1,7]naphthyridine,
(7R,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,
(7S,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,
(7R,8R,9R)-8-benzoyloxy-7-methoxy-2,3-dimethyl-9-phenyl-7,8,9,10-tetrahydroimidazo[1,2-h][1,7]-naphthyridine,
(7R,8R,9R)-7-(2-methoxyethoxy)-2,3-dimethyl-8-(4-nitrobenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(3-nitrobenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(3-nitrobenzoyloxy)-9-phenyl-7,8,9,10-tetrahydroimidazo[1,2-h][1,7]naphthyridine,
(7S,8R,9R)-7-methoxy-2,3-dimethyl-8-(3-nitrobenzoyloxy)-9-phenyl-7,8,9,10-tetrahydroimidazo-[1,2-h][1,7]naphthyridine,
(7R,8R,9R)-7-methoxy-2,3-dimethyl-8-(3-nitrobenzoyloxy)-9-phenyl-7,8,9,10-tetrahydroimidazo-[1,2-h][1,7]naphthyridine,
(7R,8R,9R)-7-(2-methoxyethoxy)-2,3-dimethyl-8-(4-methoxybenzoyloxy)-9-phenyl-7,8,9,10-tetrahydroimidazo[1,2-h][1,7]napthyridine,
(7R,8R,9R)-7-(2-methoxyethoxy)-2,3-dimethyl-8-(4-methoxybenzoyloxy)-9-phenyl-7,8,9,10-tetrahydroimidazo[1,2-h][1,7]napthyridine,
(7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,
(7S,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,
(7S,8R,9R)-7-(2-methoxyethoxy)-8-(N,N-diethylaminocarbonyloxy)-2,3-dimethyl-9-phenyl-7,8,9,10-tetrahydroimidazo[1,2-h][1,7]naphthyridine,
(7R,8R,9R)-7-(2-methoxyethoxy)-8-N,N-diethylaminocarbonyloxy)-2,3-dimethyl-9-phenyl-7,8,9,10-tetrahydroimidazo[1,2-h][1,7]napthyridine,
(7R,8R,9R)-8-ethylaminocarbonyloxy-7-(2-methoxyethoxy)-2,3-dimethyl-9-phenyl-7,8,9,10-tetrahydroimidazo[1,2-h][1,7]napthyridine,
(7R,8R,9R)-8-benzoyloxy-2,3-dimethyl-7-(2-methoxyethoxy)-9-phenyl-7H-8,9-dihydropyrano[2,3-c]-imidazo[1,2-a]pyridine,
(7S,8R,9R)-9-benzoyloxy-2,3-dimethyl-7-(2-methoxyethoxy)-9-phenyl-7H-8,9-dihydropyrano[2,3-c]-imidazo[1,2-a]pyridine,
(7R,8R,9R)-8-[4-(methoxycarbonyl)-benzoyloxy]-2,3-dimethyl-7-(2-methoxyethoxy)-9-phenyl-7H-8,9-dihydropyrano[2,3-c]imidazo[1,2-a]pyridine,
(7R,8R,9R)-8-[4-(methoxycarbonyl)-benzoyloxy]-2,3-dimethyl-7-(2-methoxyethoxy)-9-phenyl-7H-8,9-dihydropyrano[2,3-c]imidazo[1,2-a]pyridine,
(7S,8R,9R)-2,3-dimethyl-7-methoxy-8-methoxyacetyloxy-9-phenyl-7,8,9,10-tetrahydroimidazo[1,2-h][1,7]naphthyridine,
(7R,8R,9R)-8-(N,N-diethylaminocarbonyloxy)-2,3-dimethyl-7-methoxy-9-phenyl-7,8,9,10-tetrahydroimidazo[1,2-h][1,7]naphthyridine,
(7R,8R,9R)-8-(N,N-diethylaminocarbonyloxy)-2,3-dimethyl-7-methoxy-9-phenyl-7,8,9,10-tetrahydroimidazo[1,2-h][1,7]naphthyridine,
(7R,8R,9R)-7-methoxy-8-methoxycarbonyloxy-2,3-dimethyl-9-phenyl-7,8,9,10-tetrahydroimidazo-[1,2-h][1,7]naphthyridine,
(7S,8R,9R)-7-methoxy-8-methoxycarbonyloxy-2,3-dimethyl-9-phenyl-7,8,9,10-tetrahydroimidazo-[1,2-h][1,7]naphthyridine,
(7R,8R,9R)-2,3-dimethyl-8-formyloxy-7-methoxy-9-phenyl-7,8,9,10-tetrahydroimidazo[1,2-h][1,7]naphthyridine,
(7S,8R,9R)-2,3-dimethyl-8-formyloxy-7-methoxy-9-phenyl-7,8,9,10-tetrahydroimidazo[1,2-h][1,7]naphthyridine,
(7R,8R,9R)-8-benzoyloxy-2,3-dimethyl-7-methoxy-9-phenyl-7,8,9,10-tetrahydroimidazo[1,2-h][1,7]-naphthyridine,
(7R,8S,9R)-2,3-trimethyl-7,8-dihydroxy-9-phenyl-7,8,9,10-tetrahydroimidazo[1,2-h][1,7]naphthyridine,
(7S,8S,9R)-2,3-dimethyl-8-benzyl-7,8-dihydroxy-9-phenyl-7,8,9,10-tetrahydroimidazo[1,2-h][1,7]-naphthyridine,
(7R,8S,9R)-2,3,8-trimethyl-7,8,0,0-isopropylidene-9-phenyl-7,8,9,10-tetrahydroimidazo[1,2-h][1,7]-napthyridine,
(7S,8S,9R)-2,3,8-trimethyl-7-(2-methoxyethoxy)-8-hydroxy-9-phenyl-7,8,9,10-tetrahydroimidazo-[1,2-h][1,7]naphthyridine,
(7S,8S,9R)-2,3,8-trimethyl-7-methoxy-8-hydroxy-9-phenyl-7,8,9,10-tetrahydroimidazo[1,2-h][1,7]-naphthyridine,
(7R,8R,9R)-2,3,7-trimethyl-7,8-dihydroxy-9-phenyl-7,8,9,10-tetrahydroimidazo[1,2-h][1,7]naphthyridine,
(7R,8R,9R)-2,3,7-trimethyl-7,8-[1,3]dioxolo-9-phenyl-7,8,9,10-tetrahydroimidazo[1,2-h][1,7]naphthyridine,
(8S,9R)-2,3-dimethyl-8-hydroxy-7-methylidene-9-phenyl-7,8,9,10-tetrahydroimidazo[1,2-h][1,7]-naphthyridine,
(7S,8R,9R)-2,3,7-trimethyl-7,8-dihydroxy-9-phenyl-7H-8,9-dihydropyrano[2,3-c]imidazo[1,2-a]pyridine,
(7R,8R,9R)-2,3,7-trimethyl-7,8-dihydroxy-9-phenyl-7H-8,9-dihydropyrano[2,3-c]imidazo[1,2-a]pyridine,
(7S,8R,9R)-2,3-dimethyl-7,8-dihydroxy-7,9-diphenyl-7H-8,9-dihydropyrano[2,3-c]imidazo[1,2-a]pyridine,
(7S,8R,9R)-2,3-dimethyl-7-(2′,2′-dimethylvinyl)-7,8-dihydroxy-9-phenyl-7H-8,9-dihydropyrano[2,3-c]-imidazo[1,2-a]pyridine,
(7R,8R,9R)-2,3-dimethyl-7,8-O-isopropylidene-9-phenyl-7-vinyl-7H-8,9-dihydropyrano[2,3-c]imidazo-[1,2-a]pyridine,
(7R,8R,9R)-2,3-dimethyl-8-hydroxy-7-(2-methoxyethoxy)-9-phenyl-7H-8,9-dihydropyrano[2,3-c]-imidazo[1,2-a]pyridine,
(7S,8R,9R)-2,3-dimethyl-8-hydroxy-7-(2-methoxyethoxy)-9-phenyl-7H-8,9-dihydropyrano[2,3-c]-imidazo[1,2-a]pyridine,
(7R,8R,9R)-2,3-dimethyl-8-hydroxy-7-ethoxy-9-phenyl-7H-8,9-dihydropyrano[2,3-c]imidazo[1,2-a]pyridine,
(7S,8R,9R)-2,3-dimethyl-8-hydroxy-7-ethoxy-9-phenyl-7H-8,9-dihydropyrano[2,3-c]imidazo[1,2-a]-pyridine,
(7R,8R,9R)-2,3-dimethyl-8-hydroxy-7-(2-methoxypropoxy)-9-phenyl-7H-8,9-dihydropyrano[2,3-c]imidazo[1,2-a]pyridine,
(7S,8R,9R)-2,3-dimethyl-8-hydroxy-7-(2-methoxypropoxy)-9-phenyl-7H-8,9-dihydropyrano[2,3-c]imidazo[1,2-a]pyridine,
(7R,8R,9R)-2,3-dimethyl-8-hydroxy-7-(2-propoxy)-9-phenyl-7H-8,9-dihydropyrano[2,3-c]imidazo-[1,2-a]pyridine,
(7S,8R,9R)-2,3-dimethyl-8-hydroxy-7-(2-propoxy)-9-phenyl-7H-8,9-dihydropyrano[2,3-c]imidazo-[1,2-a]pyridine,
(7R,8R,9R)-2,3-dimethyl-8-hydroxy-7-butoxy-9-phenyl-7H-8,9-dihydropyrano[2,3-c]imidazo[1,2-a]-pyridine,
(7R,8R,9R)-2,3-dimethyl-8-hydroxy-7-butoxy-9-phenyl-7H-8,9-dihydropyrano[2,3-c]imidazo[1,2-a]-pyridine,
(7R,8R,9R)-7,8-dihydroxy-6-methoxymethyl-2,3-dimethyl-9-phenyl-7,8,9,10-tetrahydroimidazo-[1,2-h][1,7]naphthyridine,
(7R,8R,9R)-7,8-dihydroxy-6-methoxymethyl-2,3-dimethyl-9-phenyl-9-phenyl-7,8,9,10-tetrahydroimidazo[1,2-h]-[1,7]naphthyridine,
(7S,8R,9R)-8-hydroxy-7-methoxy-6-methoxymethyl-2,3-dimethyl-9-phenyl-7,8,9,10-tetrahydroimidazo[1,2-h][1,7]naphthyridine,
(7R,8R,9R)-8-hydroxy-7-methoxy-6-methoxymethyl-2,3-dimethyl-9-phenyl-7,8,9,10-tetrahydroimidazo[1,2-h][1,7]naphthyridine,
(7R,8R,9R)-8-hydroxy-7-(2-methoxyethoxy)-6-methoxymethyl-2,3-dimethyl-9-phenyl-7,8,9,10-tetrahydroimidazo[1,2-h][1,7]naphthyridine,
(7S,8R,9R)-8-hydroxy-7-(2-methoxyethoxy)-6-methoxymethyl-2,3-dimethyl-9-phenyl-7,8,9,10-tetrahydroimidazo[1,2-h][1,7]naphthyridine,
(7R,8R,9R)-8-hydroxy-7-ethoxy-6-methoxymethyl-2,3-dimethyl-9-phenyl-7,8,9,10-tetrahydro-imidazo-[1,2-h][1,7]naphthyridine,
(7S,8R,9R)-8-hydroxy-7-ethoxy-6-methoxymethyl-2,3-dimethyl-8-phenyl-7,8,9,10-tetrahydro-imidazo-[1,2-h][1,7]napthyridine,
7,8-dihydroxy-2,3-dimethyl-9-(3-thienyl)-7,8,9,10-tetrahydroimidazo[1,2-h][1,7]naphthyridine,
7-hydroxy-2,3-dimethyl-9-(3-thienyl)-7,8,9,10-tetrahydroimidazo[1,2-h][1,7]naphthyridine,
9-(3-furyl)-7-hydroxy-2,3-dimethyl-7,8,9,10-tetrahydroimidazo[1,2-h][1,7]naphthyridine,
(7R,8R,9R)-8-hydroxy-7-[2-(2-methoxyethoxy)ethoxy]-2,3-dimethyl-9-phenyl-7,8,9,10-tetrahydroimidazo[1,2-h][1,7]naphthyridine,
(7R,8R,9R)-8-hydroxy-7-[2-(2-methoxyethoxy)ethoxy]-2,3-dimethyl-9-phenyl-7,8,9,10-tetrahydroimidazo[1,2-h][1,7]naphthyridine,
(7R,8R,9R)-7,8-dihydroxy-2-methyl-9-phenyl-7,8,9,10-tetrahydroimidazo[1,2-h][1,7]naphthyridine,
(7S,8R,9R)-8-hydroxy-2-methyl-7-(2-methoxyethoxy)-9-phenyl-7,8,9,10-tetrahydroimidazo[1,2-h]-[1,7]napthyridine,
(7R,8R,9R)-8-hydroxy-2-methyl-7-(2-methoxyethoxy)-9-phenyl-7,8,9,10-tetrahydroimidazo[1,2-h]-[1,7]naphthyridine,
(7R,8R,9R)-3-bromo-8-hydroxy-7-(2-methoxyethoxy)-2-methyl-9-phenyl-7,8,9,10-tetrahydroimidazo-[1,2-h][1,7]naphthyridine,
(7R,8R,9R)-3-chloro-8-hydroxy-7-(2-methoxyethoxy)-2-methyl-9-phenyl-7,8,9,10-tetrahydroimidazo-[1,2-h][1,7]naphthyridine,
(7R,8R,9R)-3-bromo-7-hydroxy-8-(2-methoxyethoxy)-2-methyl-9-phenyl-7,8,9,10-tetrahydroimidazo-[1,2-h][1,7]naphthyridine,
(7R,8R,9R)-3-chloro-8-hydroxy-7-(2-methoxyethoxy)-2-methyl-9-phenyl-7H-8,9-dihydro-pyrano-[2,3-c]imidazo[1,2-a]pyridine,
(7R,8R,9R)-8-hydroxy-7-(2-methoxyethoxy)-2-phenyl-7H-8,9-dihydro-pyrano[2,3-c]imidazo-[1,2-a]pyridine,
(7R,8R,9R)-7,8-dihydroxy-2-methyl-9-phenyl-7H-8,9-dihydropyrano[2,3-c]imidazo[1,2-a]pyridine,
(7S,8R,9R)-7,8-dihydroxy-2-methyl-9-phenyl-7,8,9,10-tetrahydroimidazo[1,2-h][1,7]naphthyridine,
(7R,8R,9R)-8-hydroxy-7-methoxy-2-methyl-9-phenyl-7,8,9,10-tetrahydroimidazo[1,2-h][1,7]naphthyridine,
(7S,8R,9R)-8-hydroxy-7-methoxy-2-methyl-9-phenyl-7,8,9,10-tetrahydroimidazo[1,2-h][1,7]naphthyridine,
(7R,8R,9R)-2,3-dimethyl-8-hydroxy-7-(2-hydroxyethoxy)-9-phenyl-7,8,9,10-tetrahydroimidazo[1,2-h][1,7]naphthyridine,
(7R,8R,9R)-3,9-diphenyl-8-hydroxy-7-(2-methoxyethoxy)-2-methyl-7,8,9,10-tetrahydroimidazo[1,2-h][1,7]napthyridine,
(7R,8R,9R)-7,8-dihydroxy-2-methoxymethyl-3-methyl-9-phenyl-7,8,9,10-tetrahydroimidazo[1,2-h][1,7]naphthyridine,
(7S,8R,9R)-8-hydroxy-7-(2-methoxyethoxy)-2-methoxymethyl-3-methyl-9-phenyl-7,8,9,10-tetrahydroimidazo[1,2-h][1,7]napthyridine,
(7R,8R,9R)-8-hydroxy-7-(2-methoxyethoxy)-2-methoxymethyl-3-methyl-9-phenyl-7,8,9,10-tetrahydroimidazo[1,2-h][1,7]naphthyridine,
(7S,8R,9R)-7-ethoxy-8-hydroxy-2-methoxymethyl-3-methyl-9-phenyl-7,8,9,10-tetrahydroimidazo-[1,2-h][1,7]napthyridine,
(7R,8R,9R)-7-ethoxy-8-hydroxy-2-methoxymethyl-3-methyl-9-phenyl-7,8,9,10-tetrahydroimidazo-[1,2-h][1,7]naphthyridine,
(7R,8S,9R)-10-acetyl-8-hydroxy-2,3-dimethyl-7-(4-morpholino)-7,8,9,10-tetrahydroimidazo[1,2-h][1,7]naphthyridine,
(7R,8S,9R)-8-hydroxy-2,3-dimethyl-7-(4-morpholino)-7,8,9,10-tetrahydroimidazo[1,2-h][1,7]napthyridine,
(7R,8S,9R)-8-hydroxy-2,3-dimethyl-7-methylamino-7,8,9,10-tetrahydroimidazo[1,2-h]-[1,7]naphthyridine,
(7R,8S,9R)-10-acetyl-8-hydroxy-2,3-dimethyl-7-(1-pyrrolidino)-7,8,9,10-tetrahydroimidazo[1,2-h][1,7]naphthyridine,
(7R,8S,9R)-8-hydroxy-2,3-dimethyl-7-(1-pyrrolidino)-7,8,9,10-tetrahydroimidazo-[1,2-h][1,7]naphthyridine,
(7R,8S,9R)-8-hydroxy-2,3-dimethyl-7-(1-pyrrolidino)-7,8,9,10-tetrahydroimidazo-[1,2-h][1,7]naphthyridine,
(7R,8S,9R)-10-acetyl-7-benzylamino-8-hydroxy-2,3-dimethyl-7,8,9,10-tetrahydro-imidazo[1,2-h][1,7]naphthyridine,
(7R,8S,9R)-7-benzylamino-8-hydroxy-2,3-dimethyl-7,8,9,10-tetrahydroimidazo[1,2-h][1,7]-naphthyridine,
(7R,8S,9R)-10-acetyl-8-hydroxy-8-(2-methoxyethylamino)-2,3-dimethyl-7,8,9,10-tetrahydroimidazo[1,2][1,7]napthyridine,
(7R,8S,9R)-8-hydroxy-7-(2-methoxyethylamino)-2,3-dimethyl-7,8,9,10-tetrahydroimidazo[1,2-h][1,7]naphthyridine,
(7R,8S,9R)-10-acetyl-7-(dimethylamino)-8-hydroxy-2,3-dimethyl-7,8,9,10-tetrahydro-imidazo[1,2-h][1,7]naphthyridine,
(7R,8S,9R)-8-hydroxy-7-(dimethylamino)-2,3-dimethyl-7,8,9,10-tetrahydroimidazo[1,2-h][1,7]naphthyridine,
(7S,8S,9R)-8-hydroxy-2,3,7-trimethyl-7,8,9,10-tetrahydroimidazo[1,2-h][1,7]naphthyridine,
(7S,8S,9R)-7-cyanomethyl-8-hydroxy-2,3-dimethyl-7,8,9,10-tetrahydroimidazo[1,2-h][1,7]naphthyridine,
(7S,8S,9R)-8-hydroxy-2,3-dimethyl-7-propyl-7,8,9,10-tetrahydroimidazo[1,2-h][1,7]naphthyridine,
(7R,8S,9R)-8-hydroxy-2,3-dimethyl-7-(3-methoxypropyl)-7,8,9,10-tetrahydroimidazo-[1,2-h][1,7]napthyridine,
2,3-dimethyl-9-phenyl-7H-8,9-dihydro-pyrano-[2,3-c]-N-(diethyl)imidazo[1,2-a]pyridine-6-carboxamide, ethyl 2,3-dimethyl-9-phenyl-7H-8,9-dihydro-pyrano[2,3-c]-imidazo[1,2-a]pyridine-6-carboxylate,
2,3-dimethyl-9-phenyl-7H-8,9-dihydro-pyrano[2,3-c]-imidazo[1,2-a]pyridine-6-(N,N-dimethyl)-carbamide,
(7R,8R,9R)-2,3-dimethyl-7-(2-methoxyethoxy)-9-phenyl-8-(5-nitrooxy-valeryloxy)-7,8,9,10-tetrahydroimidazo[1,2-h][1,7]naphthyridine,
(7R,8R,9R)-2,3-dimethyl-7-(2-methoxyethoxy)-9-phenyl-8-(4-nitrooxy-butyryloxy)-7,8,9,10-tetrahydroimidazo[1,2-h][1,7]naphthyridine,
(7R,8R,9R)-2,3-dimethyl-7-(2-methoxyethoxy)-9-phenyl-8-(5-nitro-oxy-valeryloxy)-7H-8,9-dihydro-pyrano[2,3-c]imidazo[1,2-a]pyridine,
(7R,8R,9R)-2,3-dimethyl-7-(2-methoxyethoxy)-9-phenyl-8-(6-nitro-oxy-2-oxa-capryloxy)-7,8,9,10-tetrahydro-imidazo[1,2-h]1,7]naphthyridine and
(7R,8R,9R)-2,3-dimethyl-7-(2-methoxyethoxy)-9-phenyl-8-(4-nitro-oxymethyl-benzyloxy)-7,8,9,10-tetrahydro-imidazo[1,2-h][1,7]naphthyridine.

Within the scope of this invention, the term “selected, specifically disclosed and stereochemically well defined tricyclic imidazo[1,2-a]pyridine compounds” refers in a second embodiment (embodiment b) of the present invention

either to those tricyclic imidazo[1,2-a]pyridine compounds, which are selected from a group consisting of those tricyclic imidazo[1,2-a]pyridine compounds which are specifically disclosed and/or individualized and/or claimed in the following patent applications and patents:

WO 9842707, WO 0017200, WO 0026217, WO 0063211, WO 0172756, WO 0172756, WO 0172757 and WO 0234749, and which are not substituted by a hydroxy-1-4C-alkyl radical bonded on the imidazo ring;

and/or to those compounds which are mentioned expressis verbis in the list B below;

and to the salts, solvates and solvates of the salts of these compounds.

List B consists of the following stereochemically uniform compounds:
(7S,8R,9R)-2,3-dimethyl-7,8-dihydroxy-9-phenyl-7,8,9,10-tetrahydroimidazo[1,2-h][1,2]naphthyridine,
(7S,8R,9R)-7,8-isopropylidenedioxy-2,3-dimethyl-9-phenyl-7,8,9,10-tetrahydroimidazo[1,2-h][1,7-naphthyridine,
7,8-dihydroxy-9-phenyl-2,3-dimethyl-7H-8,9-dihydropyrano[2,3-c]imidazo[1,2-e]pyridine,
(7R,8R,9R)-2,3-dimethyl-8-hydroxy-7-methoxy-9-phenyl-7,8,9,10-tetrahydroimidazo[1,2-h][1,7]naphthyridine,
(7S,8S,9S)-2,3-dimethyl-8-hydroxy-7-methoxy-9-phenyl-7,8,9,10-tetrahydroimidazo[1,2-h][1,7]naphthyridine,
(7S,8R,9R)-2,3-dimethyl-8-hydroxy-7-methoxy-9-phenyl-7,8,9,10-tetrahydroimidazo[1,2-h][1,7]naphthyridine,
(7R,8S,9S)-2,3-dimethyl-8-hydroxy-7-methoxy-9-phenyl-7,8,9,10-tetrahydroimidazo[1,2-h][1,7]naphthyridine,
(7R,8R,9R)-2,3-dimethyl-7-ethoxy-8-hydroxy-9-phenyl-7,8,9,10-tetrahydroimidazo[1,2-h][1,7]naphthyridine,
(7S,8R,9R)-2,3-dimethyl-7-ethoxy-8-hydroxy-9-phenyl-7,8,9,10-tetrahydroimidazo[1,2-h][1,7]naphthyridine,
(7R,8R,9R)-2,3-dimethyl-8-hydroxy-7-(2-methoxyethoxy)-9-phenyl-7,8,9,10-tetrahydroimidazo[1,2-h]-[1,7]naphthyridine,
(7S,8S,9S)-2,3-dimethyl-8-hydroxy-7-(2-methoxyethoxy)-9-phenyl-7,8,9,10-tetrahydroimidazo[1,2-h]-[1,7]naphthyridine,
(7S,8R,9R)-2,3-dimethyl-8-hydroxy-7-(2-methoxyethoxy)-9-phenyl-7,8,9,10-tetrahydroimidazo[1,2-h]-[1,7]naphthyridine,
(7R,8S,9S)-2,3-dimethyl-8-hydroxy-7-(2-methoxyethoxy)-9-phenyl-7,8,9,10-tetrahydroimidazo[1,2-h]-[1,7]naphthyridine,
(7S,8R,9R)-2,3-dimethyl-8-hydroxy-9-phenyl-7-(2-propoxy)-7,8,9,10-tetrahydroimidazo[1,2-h][1,7]-naphthyridine,
(7R,8R,9R)-2,3-dimethyl-7,8-dimethoxy-9-phenyl-7,8,9,10-tetrahydroimidazo[1,2-h][1,7]naphthyridine,
(7R,8R,9R)-2,3-dimethyl-8-hydroxy-7-(2-methylthioethyloxy)-9-phenyl-7,8,9,10-tetrahydroimidazo-[1,2-h][1,7]naphthyridine,
(7S,8R,9R)-2,3-dimethyl-8-hydroxy-8-(2-methylthioethyloxy)-9-phenyl-7,8,9,10-tetrahydroimidazo[1,2-h][1,7]naphthyridine,
(7R,8R,9R)-2,3-dimethyl-8-hydroxy-7-(2-methylsulphinylethoxy)-9-phenyl-7,8,9,10-tetrahydroimidazo-[1,2-h][1,7]naphthyridine,
(7S,8R,9R)-2,3-dimethyl-8-hydroxy-7-(2-methylsulphinylethoxy)-9-phenyl-7,8,9,10-tetrahydroimidazo-[1,2-h][1,7]naphthyridine,
(7R,8R,9R)-2,3-dimethyl-8-hydroxy-(ethylthio)-9-phenyl-7,8,9,10-tetrahydroimidazo[1,2-h][1,7]naphthyridine,
(7S,8R,9R)-2,3-dimethyl-8-hydroxy-7-(ethylthio)-9-phenyl-7,8,9,10-tetrahydroimidazo[1,2-g][1,7]naphthyridine,
(7R,8R,9R)-2,3-dimethyl-8-hydroxy-7-(2,2,2-trifluoroethoxy)-9-phenyl-7,8,9,10-tetrahydroimidazo-[1,2-h][1,7]naphthyridine
(7S,8R,9R)-2,3-dimethyl-8-hydroxy-7-(2,2,2-trifluoroethoxy)-9-phenyl-7,8,9,10-tetrahydroimidazo-[1,2-h][1,7]naphthyridine,
(7S,8R,9R)-8-acetoxy-7-(2-methoxyethoxy)-2,3-dimethyl-9-phenyl-7,8,9,10-tetrahydroimidazo[1,2-h]-[1,7]naphthyridine,
(7R,8R,9R)-8-acetoxy-7-(2-methoxyethoxy)-2,3-dimethyl-9-phenyl-7,8,9,10-tetrahydroimidazo[1,2-h]-[1,7]naphthyridine,
(7R,8R,9R)-8-acetoxy-7-methoxy-2,3-dimethyl-9-phenyl-7,8,9,10-tetrahydroimidazo[1,2-h][1,7]naphthyridine,
(7R,8R,9R)-8-acetoxy-7-ethoxy-2,3-dimethyl-9-phenyl-7,8,9,10-tetrahydroimidazo[1,2-h][1,7]naphthyridine,
(7R,8R,9R)-7-(2-methoxyethoxy)-2,3-dimethyl-9-phenyl-8-propionyloxy-7,8,9,10-tetrahydroimidazo-[1,2-h][1,7]naphthyridine,
(7R,8R,9R)-8-benzoyloxy-7-(2-methoxyethoxy)-2,3-dimethyl-9-phenyl-7,8,9,10-tetrahydroimidazo-[1,2-h][1,7]naphthyridine,
(7S,8R,9R)-8-benzoyloxy-7-(2-methoxyethoxy)-2,3-dimethyl-9-phenyl-7,8,9,10-tetrahydroimidazo-[1,2-h][1,7]naphthyridine,
(7R,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,
(7S,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,
(7R,8R,9R)-8-benzoyloxy-7-methoxy-2,3-dimethyl-9-phenyl-7,8,9,10-tetrahydroimidazo[1,2-h][1,7]-naphthyridine,
(7S,8R,9R)-8-benzoyloxy-7-methoxy-2,3-dimethyl-9-phenyl-7,8,9,10-tetrahydroimidazo[1,2-h][1,7]-naphthyridine,
(7R,8R,9R)-7-(2-methoxyethoxy)-2,3-dimethyl-8-(4-nitrobenzoyloxy)-9-phenyl-7,8,9,10-tetrahydroimidazo[1,2-h][1,7]naphthyridine,
(7S,8R,9R)-7-(2-methoxyethoxy)-2,3-dimethyl-8-(4-nitrobenzoyloxy)-9-phenyl-7,8,9,10-tetrahydroimidazo[1,2-h][1,7]naphthyridine,
(7S,8R,9R)-7-(2-methoxyethoxy)-2,3-dimethyl-8-(3-nitrobenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(3-nitrobenzoyloxy)-9-phenyl-7,8,9,10-tetrahydroimidazo[1,2-h][1,7]naphthyridine,
(7S,8R,9R)-7-methoxy-2,3-dimethyl-8-(3-nitrobenzoyloxy)-9-phenyl-7,8,9,10-tetrahydroimidazo-[1,2-h][1,7]naphthyridine,
(7R,8R,9R)-7-methoxy-2,3-dimethyl-8-(3-nitrobenzyloxy)-9-phenyl-7,8,9,10-tetrahydroimidazo-[1,2-h][1,7]naphthyridine,
(7S,8R,9R)-7-(2-methoxyethoxy)-2,3-dimethyl-8-(4-methoxybenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(4-methoxybenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,
(7S,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,
(7S,8R,9R)-7-(2-methoxyethoxy)-8-(N,N-diethylaminocarbonyloxy)-2,3-dimethyl-9-phenyl-7,8,9,10-tetrahydroimidazo[1,2-h][1,7]naphthyridine,
(7R,8R,9R)-7-(2-methoxyethoxy)-9-(N,N-diethylaminocarbonyloxy)-2,3-dimethyl-9-phenyl-7,8,9,10-tetrahydroimidazo[1,2-h][1,7]naphthyridine,
(7R,8R,9R)-8-ethylaminocarbonyloxy-7-(2-methoxyethoxy)-2,3-dimethyl-9-phenyl-7,8,9,10-tetrahydroimidazo[1,2-h][1,7]naphthyridine,
(7R,8R,9R)-8-benzoyloxy-2,3-dimethyl-7-(2-methoxyethoxy)-9-phenyl-7H-8,9-dihydropyrano[2,3-c]-imidazo[1,2-a]pyridine,
(7S,8R,9R)-8-benzoyloxy-2,3-dimethyl-7-(2-methoxyethoxy)-9-phenyl-7H-8,9-dihydropyrano[2,3-c]-imidazo[1,2-a]pyridine,
(7R,8R,9R)-8-[4-(methoxycarbonyl)-benzoyloxy]-2,3-dimethyl-7-(2-methoxyethoxy)-9-phenyl-7H-8,9-dihydropyrano[2,3-c]imidazo[1,2-a]pyridine,
(7R,8R,9R)-8-[4-(methoxycarbonyl)-benzoyloxy]-2,3-dimethyl-7-(2-methoxyethoxy)-9-phenyl-7H-8,9-dihydropyrano[2,3-c]imidazo[1,2-a]pyridine,
(7S,8R,9R)-2,3-dimethyl-7-methoxy-8-methoxyacetyloxy-9-phenyl-7,8,9,10-tetrahydroimidazo[1,2-h]-[1,7]naphthyridine,
(7R,8R,9R)-8-(N,N-diethylaminocarbonyloxy)-2,3-dimethyl-7-methoxy-9-phenyl-7,8,9,10-tetrahydroimidazo[1,2-h][1,7]naphthyridine,
(7R,8R,9R)-8-(N,N-diethylaminocarbonyloxy)-2,3-dimethyl-7-methoxy-9-phenyl-7,8,9,10-tetrahydroimidazo[1,2-h][1,7]naphthyridine,
(7R,8R,9R)-7-methoxy-8-methoxycarbonyloxy-2,3-dimethyl-9-phenyl-7,8,9,10-tetrahydroimidazo-[1,2-h][1,7]naphthyridine,
(7S,8R,9R)-7-methoxy-8-methoxycarbonyloxy-2,3-dimethyl-9-phenyl-7,8,9,10-tetrahydroimidazo-[1,2-h][1,7]naphthyridine,
(7R,8R,9R)-2,3-dimethyl-8-formyloxy-7-methoxy-9-phenyl-7,8,9,10-tetrahydroimidazo[1,2-h][1,7]naphthyridine,
(7R,8R,9R)-2,3-dimethyl-8-formyloxy-7-methoxy-9-phenyl-7,8,9,10-tetrahydroimidazo[1,2-h][1,7]naphthyridine,
(7R,8R,9R)-8-benzoyloxy-2,3-dimethyl-7-methoxy-9-phenyl-7,8,9,10-tetrahydroimidazo[1,2-h][1,7]-naphthyridine,
(7R,8S,9R)-2,3,8-trimethyl-7,8-dihydroxy-9-phenyl-7,8,9,10-tetrahydroimidazo[1,2-h][1,7]naphthyridine,
(7S,8S,9R)-2,3-dimethyl-8-benzyl-7,8-dihydroxy-9-phenyl-7,8,9,10-tetrahydroimidazo[1,2-h][1,7]-naphthyridine,
(7R,8S,9R)-2,3,8-trimethyl-7,8,9,10-isopropylidene-9-phenyl-7,8,9,10-tetrahydroimidazo[1,2-h][1,7]-naphthyridine,
(7S,8S,9R)-2,3,8-trimethyl-8-(2-methoxyethoxy)-8-hydroxy-9-phenyl-7,8,9,10-tetrahydroimidazo-[1,2-h][1,7]naphthyridine,
(7S,8S,9R)-2,3,8-trimethyl-7-methoxy-8-hydroxy-9-phenyl-7,8,9,10-tetrahydroimidazo[1,2-h][1,7]-naphthyridine,
(7R,8R,9R)-2,3,7-trimethyl)-7,8-dihydroxy-9-phenyl-7,8,9,10-tetrahydroimidazo[1,2-h][1,7]naphthyridine,
(7R,8R,9R)-2,3,7-trimethyl-7,8-[1,3]dioxolo-9-phenyl-7,8,9,10-tetrahydroimidazo[1,2-h][1,7]naphthyridine,
(8S,9R)-2,3-dimethyl-8-hydroxy-7-methylidene-9-phenyl-7,8,9,10-tetrahydroimidazo[1,2-h][1,7]-naphthyridine,
(7S,8R,9R)-2,3,7-trimethyl-7,8-dihydroxy-9-phenyl-7H-8,9-dihydropyrano[2,3-c]imidazo[1,2-a]pyridine,
(7R,8R,9R)-2,3,7-trimethyl-7,8-dihydroxy-9-phenyl-7H-8,9-dihydropyrano[2,3-c]imidazo[1,2-a]pyridine,
(7S,8R,9R)-2,3-dimethyl-7,8-dihydroxy-7,9-diphenyl-7H-8,9-dihydropyrano[2,3-c]imidazo[1,2-a]pyridine,
(7S,8R,9R)-2,3-dimethyl-7-(2′,2′-dimethylvinyl)-7,8-dihydroxy-9-phenyl-7H-8,9-dihydropyrano[2,3-c]-imidazo[1,2-a]pyridine,
(7R,8R,9R)-2,3-dimethyl-7,8-O-isopropylidene-9-phenyl-7-vinyl-7H-8,9-dihydropyrano[2,3-c]imidazo-[1,2-a]pyridine,
(7R,8R,9R)-2,3-dimethyl-8-hydroxy-7-(2-methoxyethoxy)-9-phenyl-7H-8,9-dihydropyrano[2,3-c]-imidazo[1,2-a]pyridine,
(7R,8R,9R)-2,3-dimethyl-8-hydroxy-7-(2-methoxyethoxy)-9-phenyl-7H-8,9-dihydropyrano[2,3-c]-imidazo[1,2-a]pyridine,
(7R,8R,9R)-2,3-dimethyl-8-hydroxy-7-ethoxy-9-phenyl-7H-8,9-dihydropyrano[2,3-c]imidazo[1,2-a]-pyridine,
(7R,8R,9R)-2,3-dimethyl-8-hydroxy-7-ethoxy-9-phenyl-7H-8,9-dihydropyrano[2,3-c]imidazo[1,2-a]-pyridine,
(7R,8R,9R)-2,3-dimethyl-8-hydroxy-7-(2-methoxypropoxy)-9-phenyl-7H-8,9-dihydropyrano[2,3-c]imidazo[1,2-a]pyridine,
(7R,8R,9R)-2,3-dimethyl-8-hydroxy-7-(2-methoxypropoxy)-9-phenyl-7H-8,9-dihydropyrano[2,3-c]imidazo[1,2-a]pyridine,
(7R,8R,9R)-2,3-dimethyl-8-hydroxy-7-(2-propoxy)-9-phenyl-7H-8,9-dihydropyrano[2,3-c]imidazo-[1,2-a]pyridine,
(7R,8R,9R)-2,3-dimethyl-8-hydroxy-7-(2-propoxy)-9-phenyl-7H-8,9-dihydropyrano[2,3-c]imidazo-[1,2-a]pyridine,
(7R,8R,9R)-2,3-dimethyl-8-hydroxy-7-butoxy-9-phenyl-7H-8,9-dihydropyrano[2,3-c]imidazo[1,2-a]-pyridine,
(7R,8R,9R)-2,3-dimethyl-8-hydroxy-7-butoxy-9-phenyl-7H-8,9-dihydropyrano[2,3-c]imidazo[1,2-a]-pyridine,
(7S,8R,9R)-7,8-dihydroxy-6-methoxymethyl-2,3-dimethyl-9-phenyl-7,8,9,10-tetrahydroimidazo-[1,2-h][1,7]naphthyridine,
(7R,8R,9R)-7,8-dihydroxy-6-methoxymethyl-2,3-dimethyl-9-phenyl-7,8,9,10-tetrahydroimidazo[1,2-h]-[1,7]naphthyridine,
(7S,8R,9R)-8-hydroxy-7-methoxy-6-methoxymethyl-2,3-dimethyl-9-phenyl-7,8,9,10-tetrahydroimidazo[1,2-h][1,7]naphthyridine,
(7R,8R,9R)-8-hydroxy-7-methoxy-6-methoxymethyl-2,3-dimethyl-9-phenyl-7,8,9,10-tetrahydroimidazo[1,2-h][1,7]naphthyridine,
(7R,8R,9R)-8-hydroxy-7-(2-methoxyethoxy)-6-methoxymethyl-2,3-dimethyl-9-phenyl-7,8,9,10-tetrahydroimidazo[1,2-h][1,7]naphthyridine,
(7S,8R,9R)-8-hydroxy-7-(2-methoxyethoxy)-6-methoxymethyl-2,3-dimethyl-9-phenyl-7,8,9,10-tetrahydroimidazo[1,2-h][1,7]naphthyridine,
(7R,8R,9R)-8-hydroxy-7-ethoxy-6-methoxymethyl-2,3-dimethyl-9-phenyl-7,8,9,10-tetrahydro-imidazo-[1,2-h][1,7]naphthyridine,
(7S,8R,9R)-8-hydroxy-7-ethoxy-6-methoxymethyl-2,3-dimethyl-9-phenyl-7,8,9,10-tetrahydro-imidazo-[1,2-h][1,7]naphthyridine,
7,8-dihydroxy-2,3-dimethyl-9-(3-thienyl)-7,8,9,10-tetrahydroimidazo[1,2-h][1,7]naphthyridine,
7-hydroxy-2,3-dimethyl-9-(3-thienyl)-7,8,9,10-tetrahydroimidazo[1,2-h][1,7]naphthyridine,
9-(3-furyl)-7-hydroxy-2,3-dimethyl-7,8,9,10-tetrahydroimidazo[1,2-h][1,7]naphthyridine,
(7R,8R,9R)-8-hydroxy-7-[2-(2-methoxyethoxy)ethoxy]-2,3-dimethyl-9-phenyl-7,8,9,10-tetrahydroimidazo[1,2-h][1,7]naphthyridine,
(7S,8R,9R)-8-hydroxy-7-[2-(2-methoxyethoxy)ethoxy]-2,3-dimethyl-9-phenyl-7,8,9,10-tetrahydroimidazo[1,2-h][1,7]naphthyridine,
(7R,8R,9R)-7,8-dihydroxy-2-methyl-9-phenyl-7,8,9,10-tetrahydroimidazo[1,2-h][1,7]naphthyridine,
(7S,8R,9R)-8-hydroxy-2-methyl-7-(2-methoxyethoxy)-9-phenyl-7,8,9,10-tetrahydroimidazo[1,2-h]-[1,7]naphthyridine,
(7R,8R,9R)-8-hydroxy-2-methyl-7-(2-methoxyethoxy)-9-phenyl-7,8,9,10-tetrahydroimidazo[1,2-h]-[1,7]naphthyridine,
(7R,8R,9R)-3-bromo-8-hydroxy-7-(2-methoxyethoxy)-2-methyl-9-phenyl-7,8,9,10-tetrahydroimidazo-[1,2-h][1,7]naphthyridine,
(7R,8R,9R)-3-chloro-8-hydroxy-7-(2-methoxyethoxy)-2-methyl-9-phenyl-7,8,9,10-tetrahydroimidazo-[1,2-h][1,7]naphthyridine,
(7R,8R,9R)-3-bromo-7-hydroxy-8-(2-methoxyethoxy)-2-methyl-9-phenyl-7,8,9,10-tetrahydroimidazo-[1,2-h][1,7]naphthyridine,
(7R,8R,9R)-3-chloro-8-hydroxy-7-(2-methoxyethoxy)-2-methyl-9-phenyl-7H-8,9-dihydro-pyrano-[2,3-c]imidazo[1,2-a]pyridine,
(7R,8R,9R)-8-hydroxy-7-(2-methoxyethoxy)-2-methyl-9-phenyl-7H-8,9-dihydro-pyrano[2,3-c]imidazo-[1,2-a]pyridine,
(7R,8R,9R)-7,8-dihydroxy-2-methyl-9-phenyl-7H-8,9-dihydropyrano[2,3-c]imidazo[1,2-a]pyridine,
(7S,8R,9R)-7,8-dihydro-2-methyl-9-phenyl-7,8,9,10-tetrahydroimidazo[1,2-h][1,7]naphthyridine,
(7R,8R,9R)-8-hydroxy-7-methoxy-2-methyl-9-phenyl-7,8,9,10-tetrahydroimidazo[1,2-h][1,7]naphthyridine,
(7S,8R,9R)-8-hydroxy-7-methoxy-2-methyl-9-phenyl-7,8,9,10-tetrahydroimidazo[1,2-h][1,7]naphthyridine,
(7R,8R,9R)-2,3-dimethyl-8-hydroxy-7-(2-hydroxyethoxy)-9-phenyl-7,8,9,10-tetrahydroimidazo[1,2-h]-[1,7]naphthyridine,
(7R,8R,9R)-3,9-diphenyl-8-hydroxy-7-(2-methoxyethoxy)-2-methyl-7,8,9,10-tetrahydroimidazo[1,2-h]-[1,7]naphthyridine
(7R,8R,9R)-7,8-dihydroxy-2-methoxymethyl-3-methyl-9-phenyl-7,8,9,10-tetrahydroimidazo[1,2-h]-[1,7]naphthyridine,
(7S,8R,9R)-8-hydroxy-7-(2-methoxyethoxy)-2-methoxymethyl-3-methyl-9-phenyl-7,8,9,10-tetrahydroimidazo[1,2-h][1,7]naphthyridine,
(7R,8R,9R)-8-hydroxy-7-(2-methoxyethoxy)-2-methoxymethyl-3-methyl-9-phenyl-7,8,9,10-tetrahydroimidazo[1,2-h][1,7]naphthyridine,
(7S,8R,9R)-7-ethoxy-8-hydroxy-2-methoxymethyl-3-methyl-9-phenyl-7,8,9,10-tetrahydroimidazo-[1,2-h][1,7]naphthyridine and
(7R,8R,9R)-7-ethoxy-8-hydroxy-2-methoxymethyl-3-methyl-9-phenyl-7,8,9,10-tetrahydroimidazo-[1,2-h][1,7]naphthyridine.

Suitable salts in the scope of this invention are especially all acid addition salts. Particular mention may be made of the pharmacologically tolerable salts of the inorganic or organic adds customarily used in pharmacy. Those suitable are water-soluble and water-insoluble acid addition salts with adds such as, for example, hydrochloric acid, hydrobromic acid, phosphoric acid, nitric add, sulfuric acid, acetic acid, citric acid, D-gluconic add, benzoic acid, 2-(4-hydroxybenzoyl)benzoic acid, butyric acid, sulfosalicylic acid, maleic acid, lauric acid, malic acid, fumaric acid, succinic acid, oxalic acid, tartaric acid, embonic acid, stearic acid, toluenesulfonic ad, methanesulfonic add or 3-hydroxy-2-naphthoic acid, where the acids are employed in salt preparation—depending on whether a mono- or polybasic acid is concerned and depending on which salt is desired—in an equimolar quantitative ratio or one differing therefrom.

On the other hand, salts with bases are—depending on substitution—also suitable. As examples of salts with bases are mentioned the lithium, sodium, potassium, calcium, aluminium, magnesium, titanium, ammonium, meglumine or guanidinium salts, here, too, the bases being employed in salt preparation in an equimolar quantitative ratio or one differing therefrom.

According to the knowledge of the person skilled in the art the tricyclic imidazo[1,2-a]pyridine compounds of the invention as well as their salts may contain, e.g. when isolated in crystalline form, varying amounts of solvents. Within the scope of the invention the term “selected tricyclic imidazo[1,2-a]pyridine compounds”includes therefore all solvates and in particular all hydrates of said selected tricyclic imidazo[1,2-a]pyridine compounds as well as all solvates and in particular all hydrates of the salts of said selected tricyclic imidazo[1,2-a]pyridine compounds.

Within the scope of this invention the terms “medicament caused gastrointestinal diseases” and “gastrointestinal diseases caused by certain medicaments” refer to gastrointestinal diseases which are induced and/or caused by certain medicaments selected from the group consisting of NSAIDs (non-steroidal antiinflammatory drugs), COX-2 (cycloxygenase 2) inhibitors, NO-NSAIDs (nitric oxide releasing NSAID), bisphosphonates and corticosteroids, whereby NSAIDs, COX-2 inhibitors, NO NSAIDs and bisphosphonates are particularly worthy to be mentioned; NSAIDs, COX-2 inhibitors and NO-NSAIDs are to be emphasized, NSAIDs and COX-2 inhibitors are mom to be emphasized, and NSAIDs are particularly to be emphasized.

Exemplary NSAIDs within the meaning of the present invention are, in an embodiment (embodiment 1) according to the present invention, glycolic acid [o-(2,6-dichloroanilino)phenyl]acetate(ester) [INN: ACECLOFENAC]; 1-(4-chlorobenzoyl)-5-methoxy-2-methyl-1H-indole-3-acetic acid carboxymethyl ester [INN: ACEMETACIN; 2-(acetyloxy)benzoic acid [ACETYLSALICYLIC ACID], 2-methoxyphenyl-alpha-methyl-4-(isobutyl)phenylacetate [Research Code: AF-2259), (4-allyloxy-3-chlorophenyl)acetic acid [INN: ALCLOFENAC], p-[(2-methylallyl)amino]hydratropic acid [INN: ALMINOPROFEN], 2-amino-3-benzoylphenylacetic acid [INN: AMFENAC], (plus/minus)-4-(1-hydroxyethoxy)-2-methyl-N-2-pyridyl-2H-1,2-benzothiazine-3-carboxamide ethylcarbonate (ester), 1,1-dioxide [INN: AMPIROXICAM], 2-methoxyphenyl-1-methyl-5-(p-methylbenzoyl)pyrrol-2-acetamido-acetate [INN: AMTOLMETINGUACIL], (plus/minus)-2,3-dihydro-5-(4-methoxybenzoyl)-1H-pyrrolizine-1-carboxylic acid [INN: ANIROLAC], 2-[4-(alpha, alpha, alpha-trifluoro-m-tolyl)-1-piperazinyl]ethyl-N-(7-trifluoromethyl-4-quinolyl)anthranilate [INN: ANTRAFENINE], 5-(dimethylamino)-9-methyl-2-propyl-1H-pyrazolo[1,2-a][1,2,4]benzo-triazine-1,3(2H)-dione [INN: AZAPROPAZONE], 4-acetamidophenyl salicylate acetate [INN: BENORILATE], 2-(8-methyl-10,11-dihydro-11-oxodibenz[b,f]axepin-2-yl)propionic acid [INN: BERMOPROFEN], 2-](1-benzyl-1H-indazol-3-yl)methoxy]-2-methylpropionic acid [INN: BINDARIT], [2-amino-3-(p-bromo-benzoyl)phenyl]acetic acid [INN: BROMFENAC], 3-(3-chloro-4-cyclohexylbenzoyl)propionic acid [INN: BUCLOXIC ACID], 5-butyl-1-cyclohexylbarbituric acid [INN: BUCOLOM], 4-butoxy-N-hydroxy-benzeneacetamide [INN: BUFEXAMAC], butylmalonic acid mono-(1,2-diphenylhydrazide) [INN: BUMADIZONE], alpha-ethyl-4-(2-methylpropyl)benzenacetic acid [INN: BUTIBUFEN], 2-(4-bi-phenylyl)butyric acid, trans-4-phenylcyclohexylamine salt (1:1) [INN: BUTIXIRATE], 2-(acetyloxy)-benzoic acid, calcium salt, compound with urea (1:1) [INN: CARBASALATE CALCIUM], (plus/minus)-6-chloro-alpha-methylcarbazole-2-acetic acid [INN: CARPROFEN], 1-cinnamoyl-5-methoxy-2-methylindole-3-acetic acid [INN: CINMETACIN], N-(2-pyridyl)-2-methyl-4-cinnamoyloxy-2H-1,2-benzothiazine-3-carboxamide-1,1-dioxide [INN: CINNOXICAM], 6-chloro-5-cyclohexyl-1-indan-carboxylic acid [INN: CLIDANAC], 2-[4-(p-chlorophenyl)benzyloxy]-2-methylpropionic acid [INN: CLOBUZARIT], 5-methoxy-2-methyl-3-indolylacetohydroxamic acid [INN: DEBOXAMET], (S)-(+)-p-isobutylhydratropic acid [INN: DEXIBUPROFEN], (+)-(S)-m-benzoylhydratropic acid [INN: DEXKETOPROFEN], 2-[(2,6-dichlorophenyl)amino]benzeneacetic acid [INN: DICLOFENAC], 2′,4′-Difluoro-4-hydroxy-3-biphenylcarboxylic acid [INN: DIFLUNISAL], 4-(2,6-dichloroanilino)-3-thiopheneacetic acid [INN: ELTENAC]N-beta-phenethyl-anthranilic acid [INN: ENFENAMIC ACID]salicylic acid acetate, ester with beta-hydroxy p-acetophenetidide [INN: ETERSALATE], 1,8-diethyl-1,3,4,9-tetrahydropyrano[3,4-b]indole-1-acetic acid [INN: ETODOLAC], 2-[[3-(trifluoromethyl)phenyl]amino]benzoic acid 2-(2-hydroxyethoxy)-ethyl ester [INN: ETOFENAMATE], p-chlorobenzoic acid, ester with 4-butyl-4-(hydroxymethyl)-1,2-diphenyl-3,5-pyrazolidinedione [INN: FECLOBUZONE], 4-biphenylacetic acid [INN: FELBINAC], 3-(4-biphenylcarbonyl)propionic acid [INN: FENBUFEN], [o-(2,4-chlorophenoxy)phenyl]acetic acid [INN: FENCLOFENAC], (plus/minus)-m-phenoxyhydratropic acid [INN: FENOPROFEN], 4-(p-chlorophenyl)-2-phenyl-5-thiazoleacetic acid [INN: FENTIAZAC], (plus/minus)-alpha-[[(2-hydroxy-1,1-dimethylethyl)amino]methyl]-benzyl alcohol [INN: FEPRADINOL], 4-(2′,4′-difluorophenylyl)-4-oxo-2-methylbutanoic acid [INN: FLOBUFEN], 2,3-dihydroxypropyl N-[8-(trifluoromethyl)-4-quinolinylanthranilate [INN: FLOCTAFENINE], N-(alpha, alpha, alpha-trifluoro-m-tolyl)anthranilic acid [INN: FLOCTAFENINE], N-fluorophenyl)alpha-methyl-5-benzoxazoleacetic acid [INN: FLUNOXAPROFEN], 2-fluoro-alpha-methyl-4-biphenylacetic acid [INN: FLURBIPROFEN], (plus/minus)-2-(2-fluoro-4-biphenylyl)propionic add 1 (acetoxy)ethyl ester [INN: FLURBIPROFEN AXETIL], 2-ethyl-2,3-dihydro-5-benzofluoroacetic acid [INN: FUROFENAC], 2-[4-(2′-furoyl)phenyl]propionic acid [INN: FURPROFEN], 2-[2-[1-(p-chlorobenzoyl)-5-methoxy-2-methylindol-3-yl]acetamido]-2-deoxy-D-glucose [INN: GLUCAMETACIN], 2-(2-fluorobiphenyl-4-yl)propionic acid 4-nitrooxybutylester [Research Code: HCT-1026], (p-isobutylphenyl)acetic acid [INN: IBUFENAC], alpha-p-isobutylphenylpropionic acid [INN: IBUPROFEN], methyl 4-(3-thienyl)phenyl-alpha-methylacetate [Research Code: IDPH-8261], (plus/minus)-2-[p-(1-oxo-2-isoindolinyl)phenyl]butyric acid [INN: INDOBUFEN], 1-(4-chlorobenzoyl)-5-methoxy-2-methyl-1H-indole-3-acetic acid [INN: INDOMETACIN], 1-(4-chlorobenzoyl)-5-methoxy-2-methyl-2-methyl-1H-indole-3-acetic acid, 3,7,11-trimethyl-2,6,10-dodecatrienyl ester [INN: INDOMETACIN FARNESIL], p-(1-oxo-2-isoindolinyl)hydratropic acid [INN: INDOPROFEN], 2-(10-methoxy-4H-benzo[4,5]cyclohepta[1,2-b]thiophen-4-yldene)acetic acid [Research Code, IX-207-887], m-benzoylhydratropic acid [INN: KETOPROFEN], (DL)-5-benzoyl-3H-1,2-dihydropyrrolo[1,2-a]pyrrole-1-carboxylic acid [INN: KETOROLAC], 2,3-dihydro-5-hydroxy-6-[2-(hydroxymethyl)cinnamyl]benzofuran [Research Code: L-651896], N-(2-carboxyphenyl)-4-chloroanthranilic acid [INN: LOBENZARIT], 3-(p-chlorophenyl)-1-phenylpyrazole-4-acetic acid [INN: LONAZOLAC], 6-chloro-4-hydroxy-2-methyl-N-2-pyridyl-2H-thieno[2,3-e]-1,2-thiazine-3-carboxamide 1,1-dioxide [INN: LORNOXICAM], 2-[4-(2-oxocyclopentan-1-ylmethyl)phenyl]propionate [INN: LOXOPROFEN], 2(R)-[4-(3-methyl-2-thienyl)phenyl]propionic acid [Research Code: M-5010], N-2,3-xylxyl)anthranilic acid [INN: MEFENAMIC ACID], 4-hydroxy-2-methyl-N-(5-methyl-2-thiazolyl)-2H-1,2-benzothiazine-3-carboxamide 1,1-dioxide [INN: MELOXICAM], 5-aminosalicylic acid [INN: MESALAZINE], (2,2-dimethyl-6-(4-chlorophenyl)-7-phenyl-2,3-dihydro-1H-pyrrolizin-5-yl add [Research Code: ML-3000], 3,4-bis(4-methoxyphenyl)-5-isoxazoleacetic acid [INN: MOFEZOLAC], 4-(6-methoxy-2-naphthyl)-2-butanone [INN: NABUMETONE], (plus)-6-methoxy-alpha-methyl-2-naphthalineacetic acid [INN: NAPROXEN], 2-{3-(trifluoromethyl)anilino]nicotinic acid [INN: NIFLUMIC ACID], 5,5′-azodisalicylic acid [INN: OLSALAZINE], 4,5-diphenyl-2-oxazolepropionic acid [INN: OXAPROZIN], alpha-methyl-4-[(2-oxocyclohexylidene)methyl]benzene acetic acid [INN: PELUBIPROFEN], 4-butyl-1,2-diphenyl-3,5-pyrazolidinedione [INN: PHENYLBUTAZONE], 2-(p-isobutylphenyl)propionic acid 2-pyridyl-methyl ester [INN: PIMEPROFEN], 4-(p-chlorophenyl)-1-(p-trifluoropyrazole-3-acetic acid [INN: PIRAZOLAC], 4-hydroxy-2-methyl-N-2-pyridyl-2H-1,2-benzothiadiazin-3-carboxamide 1,1-dioxide [INN: PIROXICAM], 3-chloro-4-(3-pyrrolin-1-yl)hydratropic acid [INN: PIRPROFEN], 2-[5H-(1)benzopyrano]2,3-b]pyridin-7-yl]propionic acid [INN: PRANOPROFEN], 2,6-di-tert-butyl-4-(2′-thenoyl)phenol [INN: PRIFELONE], alpha-cyano-1-methyl-beta-oxopyrrole-2-propionanilide [INN: PRINOMIDE], 3-[4-(2-hydroxyethyl)-1-piperazinyl-propyl-D,L-4-benzamido-N,N-dipropylglutaramat 1-(p-chlorobenzoyl)-5-methoxy-2-methylindole-3-acetate (ester) [INN: PROGLUMETACIN], 7-methyl-1-(1-methylethyl)-4-phenyl-2(1H)quinazolinone [INN: PROQUAZONE], 7-methoxy-alpha, 10-dimethylphenonthiazine-2-acetic acid [INN: PROTIZINIC ACID], 2-[[2-(p-chlorophenyl)-4-methyl-5-oxazolyl]methoxy]-2-methylpropionic acid [INN: ROMAZARIT], o-hydroxybenzamide [SALICYLAMIDE], 2-hydroxybenzoic acid SALICYLIC ACID], N-acetyl-L-acetyl-L-cysteine salicylate (ester) acetate (ester) [INN: SALMISTEINE], N-acetyl-L-cysteine salicylate (ester) [INN: SALNACEDIN], 2-hydroxybenzoic acid 2-carboxyphenyl ester [INN: SALSALATE], 4-[1-(2-fluorobiphenyl-4-yl)ethyl]N-methylthiazole-2-amine [Research Code: SM-8849], (Z)-5-fluoro-2-methyl-1-[p-(methylsulfinyl)benzylidene]indene-3-acetic acid [INN: SULINDAC], p-2-thenoylhydratropic acid [INN: SURPROFEN]2-(4-(3-methyl-2-butenyl)phenyl)propionic acid [Research Code: TA-60], phthalidyl 2-(alpha, alpha, alpha-trifluoro-m-toluidino)nicotinate [INN: TALNIFLUMATE], (Z)-5-chloro-3-(2-thenoyl)-2-oxoindole-1-carboxamide [INN: TENIDAP], 2-thiophenecarboxylic acid, ester with salicyclic acid [INN: TENOSAL], 4-hydroxy-2-methyl-N-2-pyridyl-2H-thieno[2,3-e]-1,2-thiazine-3-carboxamide [INN: TENOXICAM], 5-(4-chlorophenyl)-N-hydroxy-1-(4-methoxyphenyl)-N-methyl-1H-pyrazole-3-propionamide [INN: TEPOXALIN], alpha-(5-benzoyl-2-thienyl)propionic acid [INN: TIAPROFENIC ACID], 5-chloro-3-[4-(2-hydroxyethyl)-1-piperazinyl]carbonylmethyl-2-benzo-thiazolin one [INN: TIARAMIDE], 2-(2-methyl-5H-[1]benzopyrano[2,3-b]pyridin-7-yl)-propionic acid N,N-dimethylcarbamoylmethyl ester [INN: TILNOPROFEN ARBAMEL], 1-Cyclohexyl-2-(2-methyl-4-quinolinyl)-3-(2-thiazolyl)guanidine [INN: TIMEGADINE], 2-amino-6-benzyl-4,5,6,7-tetrahydrothieno[2,3-c]pyridine [INN: TINORIDINE], N-(3-chloro-o-tolyl)anthranilic acid [INN: TOLFENAMIC ACID], 1-methyl-5-(4-methylbenzoyl)-1H-pyrrole-2-acetic acid [INN: TOLMETIN], hydroxybis[alpha-methyl-4-(2-methylpropyl)benzene acetate-O]aluminium [Research Code: U-18573-G], N-(3-trifluoromethylphenyl)-anthranilic acid n-butyl ester [INN: UFENAMATE], 2-[4-[3-(hydroxyimino)cyclohexyl]phenyl]propionic acid [INN: XIMOPROFEN], 2-(10,11-dihydro-10-oxo-dibenz[b,f]thiepin-2-yl-propionic acid [INN: ZALTOPROFEN]and 2-[4-(2-thiazolyloxy)phenyl]-propionic acid [INN: ZOLIPROFEN], as well as the pharmaceutically acceptable derivatives of these compounds.

Exemplary NSAIDs according to embodiment 1 which are to be emphasized are: ACETYLSALICYLIC ACID, DICLOFENAC DIFLUNISAL, ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID and TOLMETIN, as well as the pharmaceutically acceptable derivatives of these compounds.

In an alternative embodiment, exemplary NSAIDs according to embodiment 1 which are to be emphasized are: DICLOFENAC, DIFLUNISAL, ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID and TOLMETIN, as well as the pharmaceutically acceptable derivatives of these compounds.

Exemplary NSAIDs according to embodiment 1, which are selected from the above-defined group of exemplary NSAIDs, more to be emphasized are 2-(acetyloxy)benzoic acid [ACETYLSALICYLIC ACID], 2-[(2,6-dichlorophenyl)amino]benzeneacetic acid [INN: DICLOFENAC], alpha-p-isobutylphenylpropionic acid [INN: IBUPROFEN], 1-(4-chlorobenzoyl)-5-methoxy-2-methyl-1H-indole-3-acetic acid [INN: INDOMETACIN], (plus)-6-methoxy-alpha-methyl-2-naphthalineacetic acid [INN: NAPROXEN] and 4-hydroxy-2-methyl-N-2-pyridyl-2H-1,2-benzothiadiazin-3-carboxamide 1,1-dioxide [INN: PIROXICAM], as well as the pharmaceutically acceptable derivatives of these compounds.

In an alternative embodiment, exemplary NSAIDs according to embodiment 1, which are selected from the above-defined group of exemplary NSAIDs, also more to be emphasized are 2-[(2,6-dichlorophenyl)amino]benzeneacetic acid [INN: DICLOFENAC], alpha-p-isobutylphenylpropionic acid [INN: IBUPROFEN], 1-(4-chlorobenzoyl)-5-methoxy-2-methyl-1H-indole-3-acetic acid [INN: INDOMETACIN], (plus)-6-methoxy-alpha-methyl-2-naphthalineacetic acid [INN: NAPROXEN] and 4-hydroxy-2-methyl-N-2-pyridyl-2H-1,2-benzothiadiazin-3-carboxamide 1,1-dioxide [INN: PIROXICAM], as well as the pharmaceutically acceptable derivatives of these compounds.

Exemplary NSAIDs according to embodiment 1 in particular to be emphasized are 2-[(2,6-dichlorophenyl)amino]benzeneacetic acid [INN: DICLOFENAC] and 2-(acetyloxy)benzoic acid [ACETYLSALICYLIC ACID], as well as the pharmaceutically acceptable derivatives of these compounds.

An in more particular to be emphasized exemplary NSAID according to embodiment 1 is 2-[(2,6-dichlorophenyl)amino]benzeneacetic acid [INN: DICLOFENAC] or a pharmaceutically acceptable derivative thereof.

Examples of NO-NSAID to be used in the present invention include, but are not limited to, those disclosed, particularly these individualized or disclosed as examples, in WO 96/32948, WO 96/35416, WO 96/38136, WO 96/39409, WO 00/50037, U.S. Pat. No. 8,057,347, WO 94/04484, WO 94/12463, WO 95/09831, WO 95/30641, WO 97/31854, WO 99/44595, WO 99/45004 and WO 01/45703, as well as the pharmaceutically acceptable derivatives of these compounds.

As exemplary COX-2 Inhibitors within the scope of this invention can be mentioned in one embodiment (embodiment 2) according to the present invention, without being restricted to: 5-chloro-6′-methyl-3-[4-(methylsulfonyl)phenyl]-2,3′-bipyridine [INN: ETORICOXIB], 4-[5-(4-methylphenyl)-3-trifluoromethyl)-1H-pyrazol-1-yl]benzene-sulfonamide [INN: CELECOXIB], 4-[p-(methylsulfonyl)phenyl]-3-phenyl-2(5H)-furanone [INN: ROFECOXIB], N-[[p-(5-methyl-3-phenyl-4-isoxazolyl)phenyl]sulfonyl]propion-amide [INN: PARECOXIB], p-(5-methyl-3-phenyl-4-isoxazolyl)benzenesulfonamide [INN: VALDECOXIB], 2-[2-(2-chloro-6-fluorophenylamino)-5-methylphenyl]acetic acid [INN: LUMIRACOXIB], 4-(4-cyclohexyl-2-methyloxazol-5-yl)-2-fluorobenzene-sulfonamide [INN: TILMACOXIB], 4-[4-chloro-5-(3-fluoro-4-methoxyphenyl)-1H-imidazol-1-yl]benzene-sulfonamide [INN: CIMICOXIB], 4′-nitro-2′-phenoxy-methanesulfonanilide [INN: NIMESULIDE], 6-(2,4-difluorophenoxy)-5-methylsulfonylamino-1-indanone [INN: FLOSULIDE], 5-bromo-2-(4-fluoro-phenyl-3-(4-methanesulfonyl-phenyl)-thiophene [DUP-697], acetyl-2-(2,4-difluorophenoxy)methanesulfonanilide [FK-3311], N-[2-(cyclohexyloxy)-4-nitro-phenyl]methanesulfonamide [NS-398], 5-methanesulfonamido-6-(2,4-difluorophenylthio)-1-indanone [L-745337], 8-acetyl-3-(4-fluorophenyl)-2-[4-(methanesulfonyl)phenyl]imidazo[1,2-a]-pyridine [GR-253035], 4-[5-(4-chlorophenyl)-3-trifluoromethyl)pyrazol-1-yl]benzenesulfon-amide [SC-58236], 4-(2,3-dihydro-2-oxo-3-phenyl-4-oxazolyl)-benzenesulfonamide [LAS-33815], CS-502, 2-(3,4-difluorophenyl)-4-(3-hydroxy-3-methylbutoxy)-5-[4-(methyl-sulfonyl)phenyl]-3(2H)-pyridazinone [ABT-963], or GW-406381, or those COX-2 inhibitors disclosed in the applications WO 02096427, WO 098886 or WO 02095885, which are all incorporated by reference into the specification of the present invention in their entirety for all purposes,

as well as the pharmaceutically acceptable derivatives of these compounds.

COX-2 Inhibitors according to embodiment 2 of this invention which are to be emphasized include, but are not limited to, 5-chloro-6′-methyl-3-[4-(methylsulfonyl)phenyl]-2,3′-bipyridine [INN: ETORICOXIB], 4-[5-(4-methylphenyl)-3-(trifluoromethyl)-1H-pyrazol-1-yl]benzene-sulfonamide [INN: CELECOXIM], [p-(methylsulfonyl)phenyl]-3-phenyl-2(5H)-furnace [INN: ROFECOXIB], N-[[p-(5-methyl-3-phenyl-4-isoxazolyl)phenyl]sulfonyl]propion-amide [INN: PARECOXIB], p-(5-methyl-3-phenyl-4-isoxazolyl)benzenesulfonamide [INN: VALDECOXIB], 2-[2-(2-chloro-6-fluorophenylamino)-5-methylphenyl]acetic acid [INN: LUMIRACOXIB], 4-(4-cyclohexyl-2-methyloxazol-5-yl)-2-fluorobenzene-sulfonamide [INN: TILMACOXIB], and 4-[4-chloro-5-(3-fluoro-4-methoxyphenyl)-1H-imidazol-1-yl]benzene-sulfonamide [INN: CIMICOXIB], as well as the pharmaceutically acceptable derivatives of these compounds.

As exemplary COX-2 inhibitors within the scope of this invention can be also mentioned in another embodiment (embodiment 2′) according to the present invention, without being restricted to: CELEBREX (CELECOXIB) or VIOXX (ROFECOXIB), as well as the pharmaceutically acceptable derivatives of these compounds.

As examples of bisphosphonates within the meaning of this invention can be mentioned in one embodiment (embodiment 3) according to the present invention, without being restricted to, ALENDRONIC ACID, RISEDRONIC ACID, TILUDRONIC ACID, IBANDRONIC ACID. ZOLEDRONIC ACID, CLODRONIC ACID, INCADRONIC ACID, OLPADRONIC ACID, MINODRONIC ACID, PAMIDRONIC ACID and ETIDRONIC ACID, as well as the pharmaceutically acceptable derivatives of these compounds.

Examples of bisphosphonates to be used in the present invention include also in another embodiment (embodiment 3′) according to the present invention, but are not limited to, ALENDRONATE. RISEDRONATE, TILUDRONATE, IBANDRONATE, ZOLEDRONATE and ETIDRONATE, as well as the pharmaceutically acceptable derivatives of these compounds.

Examples of corticosteroids which may be useful in the present invention are known to the person skilled in the art. Especially those can be mentioned which are given in high doses for a prolonged period of time and/or those which are given to patients with increased susceptibility for gas intestinal diseases or disorders.

As examples of corticosteroids within the meaning of this invention can be mentioned in one embodiment (embodiment 4) according to the present invention, without being restricted to, HYDROCORTISONE. PREDNISONE, PREDNISOLONE, METHYLPREDNISOLONE, TRIAMCINOLONE ACETONIDE, AMCINONIDE, CLOBETASONE, CLOBETASOL, DEFLAZACORT, DESONIDE, CLOPREDNOL, DEXAMETHASONE, DIFLORASONE, DIFLUCORTOLONE, DIFLUPREDNATE, FLUDROXYCORTIDE, FLUDROCORTISONE, FLUMETASONE, TIXOCORTOL PIVALATE, FLUOCORTIN BUTYL, CLOCORTOLONE, FLUOCINOLONE ACETONIDE, FLUOCORTOLONE, FLUOROMETHOLONE, FLUPREDNIDENE, FLUPREDNISOLONE, BETAMETHASONE, HALCINONIDE, BUDESONIDE, HALOMETASONE, RIMEXOLONE, PARAMETHASONE, PREDNYLIDEN, LOTEPREDNOL ETABONATE, PREDNICARBATE, as well as the pharmaceutically acceptable derivatives of these compounds.

Examples of preferred corticosteroids to be used in the present invention include also in another embodiment (embodiment 4′) according to the present invention, but are not limited to, BETAMETHASONE, HYDROCORTISONE, METHYLPREDNISOLONE, PREDNISOLONE, PREDNISONE, TRIAMCINOLONE ACETONIDE, DEXAMETHASONE, DESONIDE, FLUMETASONE, TIXOCORTOL PIVALATE, FLUDROCORTISONE, DEFLAZACORT, BUDESONIDE, LOTEPREDNOL ETABONATE, FLUOCORTOLONE, as well as the pharmaceutically acceptable derivatives of these corn.

A more preferred corticosteroid to be used in the present invention is BETAMETHASONE, DEXAMETHASONE, FLUOCORTOLONE, METHYLPREDNISOLONE, PREDNISOLONE, PREDNISONE, HYDROCORTISONE, BUDESONIDE, or TRIAMCINOLONE ACETONIDE, as well as the pharmaceutically acceptable derivatives of these compounds.

In the context of the present invention, the term “pharmaceutically acceptable derivative” means a pharmaceutically acceptable salt, ester or solvate (e.g. hydrate) or a pharmaceutically acceptable solvate of such salt or ester.

Within the scope of this invention the term “gastrointestinal diseases” in particular in the context of “medicament caused gastrointestinal diseases” or “gastrointestinal diseases caused by certain medicaments” refers to those gastrointestinal diseases, which are known to the art-skilled person on the base of his/her expert knowledge, to be caused by certain medicaments (particularly those medicaments mentioned above) such as, for example, art-known gastrointestinal inflammatory diseases and lesions, particularly gastric ulcer (i.e. ulcer of the gastrointestinal system such as, for example, stomach ulcer or duodenal ulcer), heartburn, gastrointestinal bleeding or medicament related functional gastropathy, whereby gastric ulcer is particularly to be emphasized.

In the meaning of this invention, the teams “medicament associated gastrointestinal disorders” and “gastrointestinal disorders associated with certain medicaments” refer to gastrointestinal disorders known to the person skilled in the art (such as e.g. Indigestion, mild forms of heartburn, stomach irritation or pain) which are associated with certain medicaments such as, for example, those mentioned above, as well as e.g. chloroquine, theophylline, dihydralazine, salazosulfapyridine, thiazides, iodine-containing contrast media, gold preparations or antibiotics (e.g. tetracyclines, sulfonamides or cotrimoxazol).

In this connection, it is to be understood for the skilled person that the abovementioned gastrointestinal diseases or disorders am caused or associated mainly with the active agents or ingredients of the abovementioned medicaments.

As it is known for the skilled person, the risk of medicament caused gastrointestinal diseases or medicament associated gastrointestinal disorders can vary for each single patient or patient subgroup depending for example, inter alia, from the nature of the medicament given, the dose administered, the duration of medication, the co-medication (e.g. with further gastro-toxic drugs), the age of the patient, the history of prior ulceration or further gastrointestinal diseases, serious systemic co-morbidities or the individual susceptibility of the patient.

In the scope of this invention those medicaments are in particular to be mentioned to be administered cotherapeutically together with said tricyclic imidazo[1,2-a]pyridine compounds, whose use in monotherapy (i.e. the use unpartnered with said tricyclic imidazo[1,2-a]pyridine compounds) is associated with a non-acceptable risk (particularly with a severe or high risk) for inducing said gastrointestinal disorders or, particularly, gastrointestinal diseases in a patient; and/or whose gastrointestinal safety or therapeutic index can be improved; and/or whose therapeutic use can be broadened employing said tricyclic imidazo[1,2-a]pyridine compounds cotherapeutically therewith.

Selected tricyclic imidazo[1,2-a]pyridine compounds according to embodiment a of this invention to be emphasized are those tricyclic imidazo[1,2-a]pyridine compounds, which are selected from a group comprising those tricyclic imidazo[1,2-a]pyridine compounds which are specifically disclosed and/or individualized and/or claimed in the following patent applications and patents: WO 9842707, WO 0017200, WO 0028217, WO 0063211, WO 0172756, WO 0172755, WO 0172757 WO 0234749, WO 03014120, WO 03014123, WO 03016310 and WO 03091253, and which we substituted by at least one methyl radical bonded on the imidazo ring in the position 2 or 3, and which are not substituted by a hydroxy-1-4C-alkyl radical bonded on the imidazo ring;

and/or

those tricyclic imidazo[1,2-a]pyridine compounds, which are mentioned expressis verbis in the abovementioned list A;

and the salts, solvates and solvates of the salts of these compounds.

Preferred selected tricyclic imidazo[1,2-a]pyridine compounds according to embodiment a of this invention are those compounds which are mentioned expressis verbis in the above-mentioned list A, and the salts, solvates and solvates of the salts of these compounds.

Suitable tricyclic imidazo[1,2-a]pyridine compounds according to embodiment a of this invention include in particular, but are not limited to, those tricyclic imidazo[1,2-a]pyridine compounds mentioned expressis verbis by way of example in the following examples, and the salts, solvates and solvates of the salts of these compounds.

A suitable tricyclic imidazo[1,2-a]pyridine compound according to embodiment a of this invention in particular to be emphasized is (7R,8R,9R)-8-hydroxy-7-(2-methoxyethoxy)-2,3-dimethyl-9-phenyl-7,8,9,10-tetrahydro-imidazo[1,2-h][1,7]naphthyridine [INN: Soraprazan] or a salt, solvate or solvate of a salt of this compound.

In particular preferred selected tricyclic imidazo[1,2-e]pyridine compounds according to embodiment a of this invention are compounds selected from the group consisting of those tricyclic imidazo[1,2-a]pyridine compounds mentioned expressis verbis in the following list C, and the salts, solvates and solvates of the salts of these compounds.

List C consists of the following specific compounds
1. (7R,8R,9R)-8-hydroxy-7-(2-methoxyethoxy)-2,3-dimethyl-9-phenyl-7,8,9,10-tetrahydroimidazo[1,2-h][1,7]naphthyridine,
2. (7R,8R,9R)-2,3-dimethyl-8-hydroxy-7-methoxy-9-phenyl-7,8,9,10-tetrahydroimidazo[1,2-h][1,7]naphthyridine,
3. (7S,8R,9R)-2,3-dimethyl-8-hydroxy-7-methoxy-9-phenyl-7,8,9,10-tetrahydroimidazo[1,2-h][1,7]naphthyridine,
4. (7R,8R,9R)-2,3-dimethyl-7-ethoxy-8-hydroxy-9-phenyl-7,8,9,10-tetrahydroimidazo[1,2-h][1,7]naphthyridine,
5. (7S,8R,9R)-2,3-dimethyl-7-ethoxy-8-hydroxy-9-phenyl-7,8,9,10-tetrahydroimidazo[1,2-h][1,7]naphthyridine,
6. (7R,8R,9R)-2,3-dimethyl-8-hydroxy-7-(2-methoxyethoxy)-9-phenyl-7,8,9,10-tetrahydroimidazo[1,2-h][1,7]naphthyridine,
7. (7R,8R,9R)-8-acetoxy-7-(2-methoxyethoxy)-2,3-dimethyl-9-phenyl-7,8,9,10-tetrahydroimidazo[1,2-h][1,7]naphthyridine,
8. (7R,8R,9R)-8-benzyloxy-7-(2-methoxyethoxy)-2,3-dimethyl-9-phenyl-7,8,9,10-tetrahydroimidazo[1,2-h][1,7]naphthyridine,
9. (7R,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,
10. (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,
11. (7R,8S,9R)-2,3,8-trimethyl-7,8-dihydroxy-9-phenyl-7,8,9,10-tetrahydroimidazo[1,2-h][1,7]naphthyridine,
12. (7R,8R,9R)-2,3,7-trimethyl-7,8-dihydroxy-9-phenyl-7,8,9,10-tetrahydroimidazo[1,2-h][1,7]naphthyridine,
13. (7R,8R,9R)-2,3-dimethyl-8-hydroxy-7-(2-methoxyethoxy)-9-phenyl-7H-8,9-dihydropyrano[2,3-c]imidazo[1,2-a]pyridine,
14. (7R,8R,9R)-2,3-dimethyl-8-hydroxy-7-ethoxy-9-phenyl-7H-8,9-dihydropyrano[2,3-c]imidazo-[1,2-a]pyridine,
15. (7R,8R,9R)-8-hydroxy-2-methyl-7-(2-methoxyethoxy)-9-phenyl-7,8,9,10-tetrahydroimidazo[1,2-h][1,7]naphthyridine,
16. (7R,8R,9R)-3-chloro-8-hydroxy-7-(2-methoxyethoxy)-2-methyl-9-phenyl-7,8,9,10-tetrahydroimidazo[1,2-h][1.7]naphthyridine, and
17. (7R,8R,9R)-3-chloro-8-hydroxy-7-(2-methoxyethoxy)-2-methyl-9-phenyl-7H-8,9-dihydro-pyrano[2,3-c]imidazo[1,2-a]pyridine.

According to the present invention it is to be stated that any or all of the tricyclic imidazo[1,2-a]pyridine compounds mentioned expressis verbis in list C, as week as the salts, solvates and solvates of the salts thereof, are useful within this invention and are suitable to be used in the combination therapy, combinations or compositions according to this invention together with NSAIDs, COX-2 inhibitors, NO-NSAIDs, bisphosphonates or corticosteroids as described herein.

In more detail, it is to be stated within the scope of this invention, that each single individual tricyclic imidazo[1,2-a]pyridine compound mentioned expressis verbis in list C as compound 1 to 17 as well as a salt, solvate or solvate ala salt thereof can be individually paired, each in independent specific special embodiments according to the present invention, with respective NSAID, COX-2 inhibitors, NO-NSAIDs, bisphosphonates or corticosteroids in combinations or compositions according to this invention or for use in combination therapies as described herein.

The compounds mentioned in A or C, or list B as well as the salts, solvates and solvate of the salts thereof and their preparation are described in greater details in the applications mentioned in embodiment a or b, respectively.

Selected tricyclic imidazo[1,2-a]pyridine compounds according to embodiment b of this invention to be emphasized are either

those tricyclic imidazo[1,2-a]pyridine compounds, which are selected from a group comprising those tricyclic imidazo[1,2-a]pyridine compounds which are specifically disclosed and/or individualized and/or claimed in the following patent applications and patents:

WO 9847707, WO 0017200, WO 0026217, WO 0063211, WO 0172756. WO 0172755, WO 0172757 and WO 0234749, and which are substituted by two methyl radicals bonded on the imidazo ring in the positions 2 and 3;

and/or

those tricyclic o[1,2-a]pyridine compounds, which are mentioned expresses verbis in the abovementioned list B;

and the salts, solvates and solvates of the salts of these compounds.

Preferred selected tricyclic imidazo[1,2-a]pyridine compounds according to embodiment b of this invention are those compounds which are mentioned expresses verbis in the abovementioned list B, and the salts, solvates and solvates of these salts of these compounds.

Particularly preferred selected tricyclic imidazo[1,2-a]pyridine compounds according to embodiment a and b of this invention are
(7R,8R,9R)-8-hydroxy-7-(2-methoxyethoxy)-2,3-dimethyl-9-phenyl-7,8,9,10-tetrahydro-imidazo[1,2-h]-[1,7]naphthyridine [INN: Soraprazan],
   and the salts, solvates and solvates of the salts of this compound.

A special embodiment of the selected tricyclic imidazo[1,2-a]pyridine compounds mentioned in this invention refers to 7,8,9,10-tetrahydro-imidazo[1,2-][1,7]naphthyridine derivatives and to the salts, solvates and solvates of the salts thereof.

Another special embodiment of the selected tricyclic imidazo[1,2-a]pyridine compounds mentioned in this invention refers to 7H-8,9-dihydro-pyrano[2,3-]imidazo[1,2-a]pyridine derivatives and to the salts, solvates and solvates of the salts thereof.

Another special embodiment of the present invention relates to NSAIDs used in the combinations or compositions according to this invention.

Another special embodiment of the present invention relates to COX-2 inhibitors used in the combinations or compositions according to this invention.

Another special embodiment of the present invention relates to NO-NSAIDs used in the combinations or compositions according to this invention.

Another special embodiment of the present invention relates to bisphosphonates used in the combinations or compositions according to this invention.

Another special embodiment of the present invention relates to corticosteroids used in the combinations or compositions according to this invention.

Any or all of the listed combination partners as defined herein can be suitable to be used in the combination therapy or in the combinations or compositions according to the present invention.

In a further aspect, this invention relates to the use of said selected tricyclic imidazo[1,2-a]pyridine compounds in the prevention or treatment of medicament caused gastrointestinal diseases and/or medicament associated gastrointestinal disorders.

A further aspect of the present invention is the use of said selected tricyclic imidazo[1,2-a]pyridine compounds in the prevention and/or treatment of medicament induced gastric or intestinal ulcer.

A further aspect of the present invention is the use of said selected bicyclic imidazo[1,2-a]pyridine compounds in the manufacture of pharmaceutical compositions for the prevention and/or treatment of medicament caused gastrointestinal diseases, particularly medicament induced gastric or intestinal ulcer.

A further aspect of the present invention is the use of said selected tricyclic imidazo[1,2-a]pyridine compounds in the manufacture of pharmaceutical compositions for the prevention of medicament associated gastrointestinal disorders.

A further aspect of the present invention is the use of said selected tricyclic imidazo[1,2-a]pyridine compounds in the manufacture of pharmaceutical compositions for the well-tolerated treatment and/or prevention of inflammatory diseases and/or inflammation associated disorders.

A further aspect of the present invention is the use of said selected tricyclic imidazo[1,2-a]pyridine compounds in the manufacture of pharmaceutical compositions for treating and/or preventing of non-gastrointestinal inflammatory diseases and/or inflammation associated disorders.

A further aspect of the present invention is the use of said selected tricycle imidazo[1,2-a]pyridine compounds in the manufacture of pharmaceutical compositions for treating and/or preventing of gastrointestinal or, particularly, non-gastrointestinal inflammatory diseases and/or inflammation associated disorders, and for reducing the risk of medicament associated gastrointestinal disorders or, particularly for reducing medicament caused gastrointestinal diseases, particularly medicament induced gastric or intestinal ulcer.

A further aspect of the present invention is the use of said selected tricyclic imidazo[1,2-a]pyridine compounds in the manufacture of pharmaceutical compositions comprising an antiinflammatory, antirheumatic or antipain (analgetic) ingredient which is selected from a group consisting of NSAIDs, COX-2 inhibitors, NO-NSAIDs, bisphosphonates and corticosteroids for use in combination therapy, e.g. for use in the treatment and/or prevention of diseases or disorders conventionally treated, amellorated or prevented monotherapeutically with said antiinflammatory, antirheumatic or analgetic ingredient, particularly those diseases or disorders mentioned in the specification of this invention.

A further aspect of the present invention is the use of said selected tricyclic imidazo[1,2-a]pyridine compounds in the manufacture of pharmaceutical compositions comprising an active ingredient which is selected from a group consisting of NSAIDs, COX-2 inhibitors, NO-NSAIDs, bisphosphonates and corticosteroids for treating and/or preventing of diseases or disorders which can be treated, amellorated or prevented by said active ingredient, particularly those diseases or disorders mentioned in the specification of this invention, and for reducing the risk of medicament associated gastrointestinal disorders or, particularly, for reducing medicament caused gastrointestinal diseases, particularly those mentioned in this invention.

A further aspect of the present invention is the simultaneous, separate or sequential coadministration of a NSAID, a COX-2 inhibitor, a NO-NSAID, a bisphosphonate or a corticosteroid, particularly a NSAID, a COX-2 inhibitor, a NO-NSAID or a bisphosphonate, in more particular a NSAID, a COX-2 inhibitor or a bisphosphonate, in still more particular a NSAID or a COX-2 inhibitor, preferably a NSAID, with one or more of said selected tricyclic imidazo[1,2-a]pyridine compounds to prevent medicament caused gastrointestinal diseases, particularly medicament induced gastric ulcer.

A further aspect of the present invention is the simultaneous, separate or sequential coadministration of a NSAID, a COX-2 inhibitor, a NO-NSAID, a bisphosphonate or a corticosteroid with one or more of said selected tricyclic imidazo[1,2-a]pyridine compounds to treat, ameliorate or prevent diseases or disorders which can be treated, ameliorated or prevented by this NSAID, COX-2 inhibitor, NO-NSAID, bisphosphonate or corticosteroid.

A further aspect of the present invention is a method for prevention and/or treatment of medicament caused gastrointestinal diseases, particularly medicament induced gastric ulcer, comprising administering a therapeutically effective amount of one or more of said selected tricyclic imidazo[1,2-a]pyridine compounds simultaneously, separately or sequentially with at least one NSAID, COX-2 inhibitor, NO-NSAID, bisphosphonate or corticosteroid (or, in an alternative embodiment, at least one NSAID, COX-2 inhibitor, NO-NSAID or bisphosphonate) to a mammal.

A further aspect of the present invention is a method for prevention and/or treatment of medicament associated gastrointestinal disorders comprising administering a therapeutically effective amount of one or more of said selected tricyclic imidazo[1,2-a]pyridine compounds simultaneously, separately or sequentially with said medicament to a human in need thereof.

A further aspect of the present invention is a method for treatment or prevention of inflammatory diseases and/or inflammation associated disorders comprising administering a therapeutically effective amount of one or more of said selected tricyclic imidazo[1,2-a]pyridine compounds simultaneously, separately or sequentially with at least one NSAID, COX-2 inhibitor, NO-NSAID, bisphosphonate or corticosteroid (or, in an alternative embodiment at least one NSAID, COX-2 inhibitor, NO-NSAID or bisphosphonate) to a mammal.

A further aspect of the present invention is a method for amelioration the gastrointestinal tolerance of the therapy of inflammatory diseases and/or inflammation associated disorders comprising administering a therapeutically effective amount of one or more of said selected tricyclic imidazo[1,2-a]pyridine compounds simultaneously, separately or sequentially with at least one NSAID, COX-2 inhibitor, NO-NSAID, bisphosphonate or corticosteroid (or, in an alternative embodiment, at least one NSAID, COX-2 inhibitor, NO-NSAID or bisphosphonate) to a mammal.

A further aspect of the present invention is a method for treating, ameliorating or preventing of diseases or disorders, which can be treated, ameliorated or prevented by an agent selected from a group consisting of NSAIDs, COX-2 inhibitors, NO-NSAIDs, bisphosphonates and corticosteroids, and reducing the risk of gastrointestinal diseases caused by said agent or reducing the risk of gastrointestinal disorders associated with said agent, in a human patent in need of such treatment, amelioration or prevention end at risk of gastrointestinal diseases caused by said agent or gastrointestinal disorders associated with said agent comprising administering to said patient an agent selected from the group consisting of NSAIDs, COX-2 inhibitors, NO-NSAIDs, bisphosphonates and corticosteroids in an amount effective to treat, to ameliorate or to prevent diseases or disorders, which can be treated, ameliorated or prevented by said agent selected from a group consisting of NSAIDs, COX-2 inhibitors, NO-NSAIDs, bisphosphonates and corticosteroids, simultaneously, separately or sequentially with at least one of said selected tricyclic imidazo[1,2-a]pyridine compounds in an amount effective to reduce the risk of gastrointestinal diseases caused by said agent or gastrointestinal disorders associated with said agent.

A further aspect of the present invention is a method for treating, ameliorating or preventing of diseases or disorders, which can be treated, ameliorated or prevented by an agent selected from a group consisting of NSAIDs, COX-2 inhibitors, NO-NSAIDs, bisphosphonates and corticosteroids, and reducing the risk of gastrointestinal diseases or disorders caused by or associated with said agent in a patient in need thereof comprising administering to said patient a combination or a composition according to this invention.

A further aspect of the present invention is a method for preventing of gastrointestinal diseases caused by a medicament selected from the group consisting of NSAIDs, COX-2 inhibitors, NO-NSAIDs, bisphosphonates and corticosteroids, and for treating with said medicament inflammatory, rheumatic or pain diseases comprising administering simultaneously, separately or sequentially a tri cyclic imidazo[1,2-a]pyridine compound mentioned in this invention together with said medicament to a patient in need thereof.

A further aspect of the prevent invention is a preferably orally applicable pharmaceutical composition for simultaneous administration comprising, in admixture, a first active ingredient, which is selected from a group consisting of NSAIDs. COX-2 inhibitors, NO-NSAIDs, bisphosphonates and corticosteroids (or, in an alternative embodiment, selected from a group consisting of NSAIDs, COX-2 inhibitors, NO-NSAIDs and bisphosphonates), and a second active ingredient, which is at least one of said selected tricyclic imidazo[1,2-a]pyridine compounds, to prevent medicament caused gastrointestinal diseases, particularly medicament induced gastric ulcer, in a mammal.

A further aspect of the present invention is a pharmaceutical composition comprising a first active ingredient which is selected from a group consisting of NSAIDs, COX-2 inhibitors, NO-NSAIDs, bisphosphonates or corticostereoids, and a second active ingredient which is at least one of said selected tricyclic imidazo[1,2-a]pyridine compounds, to prevent and/or treat medicament caused gastrointestinal diseases, particularly medicament induced gastric ulcer, in a mammal.

A further aspect of the present invention is a pharmaceutical composition comprising a first active ingredient, which is selected from a group consisting of NSAIDs, COX-2 inhibitors. NO-NSAIDs, bisphosphonates or corticostereoids, and a second active ingredient which is at least one of said selected tricyclic imidazo[1,2-e]pyridine compounds, to prevent and/or treat medicament associated gastrointestinal disorders, e.g. those mentioned herein, in a mammal, including human.

A further aspect of the present invention is a pharmaceutical composition comprising a first active ingredient, which is selected from a group consisting of chloroquine, theophylline, dihydralazine, salazosulfapyridine, thiazides, iodine-containing contrast media, gold preparations and antibiotics (e.g. tetracyclines, sulfonamides or cotrimoxazol), and a second active ingredient, which is at least one of said selected tricyclic imidazo[1,2-a]pyridine compounds, to prevent medicament associated gastrointestinal disorders in a human.

A further aspect of the present invention is a pharmaceutical composition for simultaneous administration comprising a NSAID, a COX-2 inhibitor, a NO-NSAID, a bisphosphonate or a corticosteroid (or, in an alternative embodiment, a NSAID, a COX-2 inhibitor, a NO-NSAID or a bisphosphonate) together with at least one of said selected tricyclic imidazo[1,2-a]pyridine compounds.

A further aspect of the present invention is a composition comprising a first active ingredient, which is a a NSAID or a COX-2 inhibitor or a NO-NSAID or a bisphosphonate or a corticosteroid (or, in an alternative embodiment, a NSAID, a COX-2 inhibitor, a NO-NSAID or a bisphosphonate), and a second active ingredient, which is at least one of said selected tricyclic imidazo[1,2-a]pyridine compounds for simultaneous, sequential or separate use in therapy in any order.

A further aspect of the present invention is a preferably orally applicable pharmaceutical composition in unit dosage form comprising a NSAID or a COX-2 inhibitor or a NO-NSAID or a bisphosphonate or a corticosteroid (or, in an alternative embodiment a NSAID, a COX-2 inhibitor, a NO-NSAID or a bisphosphonate) together with at least one of said selected tricyclic imidazo[1,2-a]pyridine compounds for use in therapy, e.g. to prevent medicament induced gastric ulcer in a mammal.

A further aspect of the present invention is a pharmaceutical composition comprising a NSAID or a COX-2 inhibitor or a NO-NSAID or a bisphosphonate or a corticosteroid (or, in an alternative embodiment, a NSAID, a COX-2 inhibitor, a NO-NSAID or a bisphosphonate) together with at least one of said selected tricyclic imidazo[1,2-a]pyridine compounds, where the NSAID, the COX-2 inhibitor, the NO-NSAID, the bisphosphonate or the corticosteroid and said selected tricyclic imidazo[1,2-a]pyridine compound(s) are administered in a single dosage form, such that the NSAID, the COX-2 inhibitor, the NO-NSAID, the bisphosphonate or the corticosteroid and said selected tricyclic imidazo[1,2-a]pyridine compound(s) are physically separated from each other.

A further aspect of the present invention is a composition comprising a first active ingredient selected from the group consisting of NSAIDs, COX-2 inhibitors, NO-NSAIDs, bisphosphonates and corticosteroids, and a second active ingredient selected from the group consisting of those tricyclic imidazo [1,2-a]pyridine compounds mentioned in this invention, together with a pharmaceutically acceptable carrier or diluent.

A further aspect of this invention is a pharmaceutical composition comprising:
(a) a pharmaceutically effective amount of at least one tricyclic imidazo[1,2-a]pyridine compound selected from abovementioned list A or list B or a pharmaceutically effective salt thereof, and
(b) a pharmaceutically effective amount of at least one NSAID, COX-2 inhibitor, NO-NSAID, bisphosphonate or corticosteroid (or, in an alternative embodiment, at least one NSAID, COX-2 inhibitor, NO-NSAID or bisphosphonate);

A further aspect of this invention is a pharmaceutical composition comprising:
(a) a pharmaceutically effective amount of at least one tricyclic imidazo[1,2-a]pyridine compound selected from abovementioned list A or list B or a pharmaceutically effective salt thereof; and
(b) a pharmaceutically effective amount of at least one NSAID, COX-2 inhibitor, NO-NSAID, bisphosphonate or corticosteroid (or, in an alternative embodiment at least one NSAID, COX-2 inhibitor, NO-NSAID or bisphosphonate);
wherein component (a) and component (b) are maintained in the same delivery vehicle.

A further aspect of this invention is a pharmaceutical composition comprising:
(a) a pharmaceutically effective amount of at least one tricyclic imidazo[1,2-pyridine compound selected from abovementioned List A or list B or a pharmaceutically effective salt thereof; and
(b) a pharmaceutically effective amount of at least one NSAID, COX-2 inhibitor, NO-NSAID, bisphosphonate or corticosteroid (or, in an alternative embodiment, at least one NSAID. COX-2 inhibitor. NO-NSAID or bisphosphonate);
wherein component (a) and component (b) are maintained in different delivery vehicles.

A further aspect of the present invention is a preferably orally applicable pharmaceutical formulation comprising a first active ingredient, which is selected from a group consisting of NSAIDs, COX-2 inhibitors, NO-NSAIDs, bisphosphonates and corticosteroids (or, in an alternative embodiment, selected from a group consisting of NSAIDs, COX-2 inhibitors, NO-NSAIDs and bisphosphonates); a second active ingredient which is at least one of said selected bicyclic imidazo[1,2-a]pyridine compounds; and a pharmaceutically acceptable carrier, diluent, adjuvant, auxiliary or excipient for use in therapy, e.g. to prevent medicament induced gastric ulcer in a mammal.

A further aspect of the present invention is a first pharmaceutical formulation comprising at least one of said selected bicyclic imidazo[1,2-a]pyridine compounds and a pharmaceutically acceptable carrier or diluent; and a second pharmaceutical formulation comprising a NSAID or a COX-2 inhibitor or a NO-NSAID or a bisphosphonate or a corticosteroid (or, in an alternative embodiment, a NSAID or a COX-2 inhibitor or a NO-NSAID or a bisphosphonate) and a pharmaceutically acceptable carrier or diluent.

A further aspect of the present invention is a combination comprising a NSAID, a COX-2 inhibitor, a NO-NSAID, a bisphosphonate or a corticosteroid (or, in an alternative embodiment, a NSAID, a COX-2 inhibitor, a NO-NSAID or a bisphosphonate) and at least one of said selected tricyclic imidazo[1,2-a]pyridine compounds for simultaneous, sequential or separate use in therapy, e.g. to prevent medicament induced gastric ulcer in a mammal.

A further aspect of the present invention is a combination comprising a medicament selected from a group consisting of chloroquine, theophylline, dihydralazine, salazosulfapyridine, thiazides, iodine-containing contrast media, gold preparations and antibiotics (e.g. tetracyclines, sulfonamides or cotrimoxazol), and at least one of said selected bicyclic imidazo[1,2-a]pyridine compounds, for simultaneous sequential or separate use in therapy, e.g. to prevent medicament associated gastrointestinal disorders in a human.

A further aspect of the present invention is a combination, particularly a pharmaceutical combination, such as, for example, a combined preparation, e.g. a kit of parts, or a composition, particularly a pharmaceutical composition, comprising a first active ingredient which is an agent selected from a group consisting of NSAIDs, COX-2 inhibitors, NO-NSAIDs, bisphosphonates and corticosteroids, and a second active ingredient which is an agent selected from those tricyclic imidazo[1,2-pyridine compounds mentioned in the present invention and, optionally, at least one pharmaceutically acceptable carrier for simultaneous, sequential, separate or chronologically staggered use in therapy, and/or for use as combined unit dosage forms or as separate unit dosage forms in therapy, and/or for use as fixed or non-fixed combination in therapy, and/or for use as admixture in therapy, e.g. to treat, to ameliorate or to prevent in a mammal, including human, diseases or disorders, which can be treated, ameliorated or prevented by said first active ingredient, and, in combination therewith, to reduce, to treat, to ameliorate or to prevent in a mammal, including human, gastrointestinal diseases caused by said first active ingredient or to reduce, to treat, to ameliorate or to prevent gastrointestinal disorders associated with said first active ingredient.

A further aspect of the present invention relates to combining separate pharmaceutical compositions in kit form.

A further aspect of the present invention is a pharmaceutical product comprising, in combination, a preparation of a first active ingredient, which is at least one of said selected tricyclic imidazo-[1,2-]pyridine compounds, and a preparation of a second active ingredient, which is selected from a group consisting of NSAIDs. COX-2 inhibitors, NO-NSAIDs, bisphosphonates and corticosteroids (or, in an alternative embodiment, selected from a group consisting of NSAIDs, COX-2 inhibitors, NO-NSAIDs and bisphosphonates), for simultaneous, sequential or separate use in therapy, e.g. to prevent medicament induced gastric ulcer in a mammal.

A further aspect of the present invention is a commercial package comprising as active ingredients a NSAID or a COX-2 inhibitor or a NO-NSAID or a bisphosphonate or a corticosteroid (or, in an alternative embodiment, a NSAID or a COX-2 inhibitor or a NO-NSAID or a bisphosphonate) as well as at least one of said selected tricyclic imidazo[1,2-a]pyridine compounds together with instructions for simultaneous, sequential or separate use in therapy.

A further aspect of the present invention is a commercial package comprising at least one of said selected tricyclic imidazol-1,2-a]pyridine compounds as active ingredient together with instructions for simultaneous, sequential or separate use with a NSAID or a COX-2 inhibitor or a NO-NSAID or a bisphosphonate or a corticosteroid (or, in an alternative embodiment, with a NSAID or a COX-2 inhibitor or a NO-NSAID or a bisphosphonate).

A further aspect of the present invention is a commercial package comprising a NSAID or a COX-2 inhibitor or a NO-NSAID or a bisphosphonate or a corticosteroid (or, in an alternative embodiment, a NSAID or a COX-2 inhibitor or a NO-NSAID or a bisphosphonate) as active ingredient together with instructions for simultaneous, sequential or separate use with at least one of said selected tricyclic imidazo[1,2-a]pyridine compounds.

A further aspect of the present invention is a commercial package comprising a medicament selected from the group consisting of chloroquine, theophylline, dihydralazine, salazosulfapyridine, thiazides, iodine-containing contrast media, gold preparations and antibiotics (e.g. tetracyclines, sulfonamides or cotrimoxazol) together with instructions for simultaneous, sequential or separate use with at least one of said selected tricyclic imidazo[1,2-a]pyridine compounds.

A further aspect of the present invention is a pharmaceutical product, such as, for example, a commercial package, comprising

a combination or composition according to this invention, such as, for example,

a combination, such as, for example, a combined preparation, e.g. a kit of parts, or a pharmaceutical composition, comprising a first active ingredient which is an agent selected from a group consisting of NSAIDs, COX-2 inhibitors NO-NSAIDs, bisphosphonates and corticosteroids, and a second active ingredient which is an agent selected from those tricyclic imidazo[1,2-a]pyridine compounds mentioned in the present invention and, optionally, at least one pharmaceutically acceptable carrier for simultaneous, sequential, separate or chronologically staggered use in therapy, and/or for use as combined unit dosage forms or for use as separate unit dosage forms in therapy, and/or for use as fixed or non-fixed combination in therapy, and/or for use as admixture in therapy;

together with standard packaging material, and

together with instructions for simultaneous, sequential, separate or chronologically staggered use in therapy,

e.g. to treat, to ameliorate or to prevent in a mammal, including human, diseases or disorders, which can be treated, ameliorated or prevented by said first active ingredient and, in combination therewith, to treat, to ameliorate or to prevent in a mammal, including human, gastrointestinal diseases caused by said first active ingredient or to treat, to ameliorate or to prevent gastrointestinal disorders associated with said first active ingredient.

A further aspect of the present invention is a kit comprising at least one dosage unit of a NSAID or a COX-2 inhibitor or a NO-NSAID or a bisphosphonate or a corticosteroid (or, in an alternative embodiment, of a NSAID or a COX-2 inhibitor or a NO-NSAID or a bisphosphonate) as well as at least one dosage unit of at least one of said selected tricyclic imidazo[1,2-a]pyridine compounds for simultaneous, sequential or separate use in therapy. Optionally, abovementioned kit can be provided with instructions for use.

A further aspect of the present invention is a kit comprising a preparation of a first active ingredient, which is at least on a of said selected tricyclic imidazo[1,2-a]pyridine compounds, a preparation of a second active ingredient, which is selected from a group consisting of NSAIDs, COX-2 inhibitors, NO-NSAIDs, bisphosphonates and corticosteroids (or, in an alternative embodiment, selected from the group consisting of NSAIDs, COX-2 inhibitors, NO-NSAIDs and bisphosphonates), and instructions for simultaneous, sequential or separate administration of the preparations to a patient in need thereof.

A further aspect of the present invention is the use of a pharmaceutical composition, pharmaceutical product, formulation, preparation, combination, commercial package or kit according to the invention in the manufacture of a medicament for the treatment of an inflammatory disease and/or inflammation associated disorder.

A further aspect of the present invention is the use of the pharmaceutical compositions, formulations, preparations, combined preparations, combinations or kits according to this invention in the manufacture of a pharmaceutical product, such as e.g. a commercial package, for the treatment or prevention of diseases or disorders which can be conventionally treated by NSAIDs, COX-2 inhibitors, NO-NSAIDs, bisphosphonates or corticosteroids, e.g. inflammatory diseases or inflammation associated disorders.

A further aspect of the present invention in the use of the pharmaceutical compositions, formulations, preparations, combined preparations, combinations or kits according to this invention in the manufacture of a pharmaceutical product, such as e.g. a commercial package, for the treatment or prevention of gastrointestinal diseases or disorders caused by or associated with NSAIDs, COX-2 inhibitors NO-NSAIDs, bisphosphonates or corticosteroids.

A further aspect of the present invention in the use of the pharmaceutical compositions, formulations, preparations, combined preparations, combinations or kits, particularly pharmaceutical compositions and kits, according to this invention in the manufacture of a medicament or a pharmaceutical product for treating or preventing of diseases or disorders which can be treated by agents selected from NSAIDs, COX-2 inhibitors, NO-NSAIDs, bisphosphonates and corticosteroids, and for treating or preventing of gastrointestinal diseases or disorders caused by or associated with the therapeutic use of said agents.

A further aspect of the present invention is a pharmaceutical composition comprising, in admixture, a first active ingredient, which is at least one of said selected tricyclic imidazo[1,2-a]pyridine compounds, and a second active ingredient, which is selected from a group consisting of NSAIDs, COX-2 inhibitors, NO-NSAIDs, bisphosphonates and corticosteroids (or, in an alternative embodiment, selected from the group consisting of NSAIDs, COX-2 inhibitors, NO-NSAIDs and bisphosphonates).

A further aspect of the present invention is a pharmaceutical product comprising, in combination, a preparation of a first active ingredient, which is at least one of said selected tricyclic imidazo-[1,2-a]pyridine compounds, and a preparation of a second active ingredient, which is a NSAID or a COX-2 inhibitor or a NO-NSAID or a bisphosphonate or a corticosteroid (or, in an alternative embodiment, a NSAID or a COX-2 inhibitor or a NO-NSAID or a bisphosphonate), for simultaneous, sequential or separate use in therapy, e.g. to prevent medicament induced gastric ulcer in a mammal.

A preferred aspect of embodiment a of the present invention is a pharmaceutical composition comprising a first active ingredient which is a compound selected from the list A mentioned above, or the salt, solvate or solvate of the salt of this compound; and

a second active ingredient which is

a NSAID, a COX-2 inhibitor, a NO-NSAID, a bisphosphonate or a corticosteroid, or, in particular,

a NSAID, a COX-2 inhibitor or a bisphosphonate,

or more detailed, in a first embodimental subaspect according to this invention,

a NSAID, such as e.g. one of those NSAIDs mentioned exemplarily in embodiment 1 above, particularly one of those NSAIDs mentioned thereby, in further specified embodimental subgroups according to this invention, as to be emphasized, as preferred as particularly preferred or as in more particular preferred,

or, in a second embodimental subaspect according to this invention,

a COX-2 inhibitor, such as e.g. one of those COX-2 inhibitors mentioned exemplarily in embodiment 2 or 2′ above, particularly one of those COX-2 inhibitors mentioned thereby as to be emphasized, or, in a third embodimental subaspect according to this invention,

a bisphosphonate, such as e.g. one of those bisphosphonates mentioned exemplarily in embodiment 3 or 3′ above,

or in a fourth embodimental subaspect according to this invention,

a corticosteroid, such as e.g. one of those corticosteroids mentioned exemplarily in embodiment 4 or 4′ above, particularly one of those corticosteroids mentioned thereby as particularly preferred,

or, in a more specified embodimental subaspect according to this invention,

an agent selected from the group consisting of

ACETYLSALICYLIC ACID, DICLOFENAC, DIFLUNISAL, ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID, TOLMETIN, ETORICOXIB, CELECOXIB, ROFECOXIB, PARECOXIB, VALDECOXIB, LUMIRACOXIB, TILMACOXIB, CIMICOXIB, NIMESULIDE, FLOSULIDE, DUP-67, FK-3311, NS-398, L-745337, GR-253035, SC-58236, LAS-33815, CS-502, ABT-983, GW-406381, and

ALENDRONIC ACID, RISEDRONIC ACID, TILUDRONIC ACID, IBANDRONIC ACID, ZOLEDRONIC ACID, CLODRONIC ACID, INCADRONIC ACID, OLPADRONIC ACID, MINODRONIC ACID, PAMIDRONIC ACID, ETIDRONIC ACID, and BETAMETHASONE, DEXAMETHASONE, FLUOCORTOLONE, METHYLPREDNISOLONE, PREDNISOLONE, PREDNISONE, HYDROCORTISONE, BUDESONIDE and TRIAMCINOLONE ACETONIDE,

a pharmaceutically acceptable derivative thereof,

or, in a particular specified embodimental subaspect according to this invention, an agent selected from the group consisting of

ACETYLSALICYLIC ACID, DICLOFENAC, DIFLUNISAL, ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID, TOLMETIN, ETORICOXIB, CELECOXIB, ROFECOXIB, PARECOXIB, VALDECOXIB, LUMIRACOXIB, TILMACOXIB, CIMICOXIB, ALENDRONIC ACID, RISEDRONIC ACID, TILUDRONIC ACID, IBANDRONIC ACID, ZOLEDRONIC ACID, CLODRONIC ACID, INCADRONIC ACID, OLPADRONIC ACID, MINODRONIC ACID, PAMIDRONIC ACID and ETIDRONIC ACID,

or a pharmaceutically acceptable derivative thereof,

for simultaneous, sequential or separate use in therapy.

A preferred aspect to be more specifically mentioned of embodiment a of the present invention is a pharmaceutical composition comprising a first active ingredient which is a compound selected from the list A mentioned above,

or the salt, solvate or solvate of the salt of this compound; and

a second active ingredient which is

a NSAID, such as e.g. one of those NSAIDs mentioned exemplarily above, or, in a more detailed subaspects according to this invention,

a NSAID selected from the group consisting of

ACETYLSALICYLIC ACID, DICLOFENAC, DIFLUNISAL, ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID and TOLMETIN,

or a pharmaceutically acceptable derivative thereof

for simultaneous, sequential or separate use in therapy.

Yet a preferred aspect to be more specifically mentioned of embodiment a of the present invention is a pharmaceutical composition comprising a first active ingredient which is a compound selected from the list A mentioned above,

or the salt, solvate or solvate of the salt of this compound; and

a second active ingredient which is

a COX-2 inhibitor, such as e.g. one of those COX-2 inhibitors mentioned exemplarily above, or, in a more detailed subaspect according to this invention,

a COX-2 Inhibitor selected from the group consisting of

ETORICOXIB, CELECOXIB, ROFECOXIB, PARECOXIB, VALDECOXIB, LUMIRACOXIB, TILMACOXIB, CIMICOXIB, NIMESULIDE, FLOSULIDE, DUP-697, FK-3311, NS-398, L-745337, GR-253035, SC-58236, LAS-33815, CS-502, ABT-963, GW-406381 and those COX-2 inhibitors disclosed in WO 02096427, WO 02096886 or WO 02096885.

or a pharmaceutically acceptable derivative thereof,

or, in a particular detailed subaspect according to this invention,

a COX-2 inhibitor selected from the group consisting of

ETORICOXIB, CELECOXIB, ROFECOXIB, PARECOXIB, VALDECOXIB, LUMIRACOXIB, TILMACOXIB and CIMICOXIB,

or a pharmaceutically acceptable derivative thereof,

for simultaneous, sequential or separate use in therapy.

Yet a preferred aspect to be more specifically mentioned of embodiment a of the present invention is a pharmaceutical composition comprising a first active ingredient which is a compound selected from the list A mentioned above,

or the salt, solvate or solvate of the salt of this compound; and

a second active ingredient which is

a bisphosphonate, such as e.g. one of those bisphosphonates mentioned exemplarily above, or, in a more detailed subaspect according to this invention,

a bisphosphonate selected from the group consisting of

ALENDRONIC ACID, RISEDRONIC ACID, TILUDRONIC ACID, IBANDRONIC ACID, ZOLEDRONIC ACID, CLODRONIC ACID, INCADRONIC ACID, OLPADRONIC ACID, MINODRONIC ACID, PAMIDRONIC ACID and ETIDRONIC ACID,

or a pharmaceutically acceptable derivative thereof

for simultaneous, sequential or separate use in therapy.

Yet a preferred aspect to be more specifically mentioned of embodiment a of the present invention is a pharmaceutical composition comprising a first active ingredient which is a compound selected from the list A mentioned above,

or the salt, solvate or solvate of the salt of this compound; and

a second active ingredient which is

a corticosteroid, such as e.g. one of those corticosteroids mentioned exemplarily above,

or, in a more detailed subaspect according to this invention,

a corticosteroid selected from the group consisting of those corticosteroid mentioned in embodiment 4 or a pharmaceutically acceptable derivative thereof,

or, in a particular detailed subaspect according to this invention,

a corticosteroid selected from the group consisting of those corticosteroids mentioned in embodiment 4′ or a pharmaceutically acceptable derivative thereof,

or, in a more particular detailed subaspect according to this invention,

a corticosteroid selected from the group consisting of BETAMETHASONE, DEXAMETHASONE, FLUOCORTOLONE, METHYLPREDNISOLONE, PREDNISOLONE, PREDNISONE, HYDROCORTISONE, BUDESONIDE and TRIAMCINOLONE ACETONIDE,

or a pharmaceutically acceptable derivative thereof

for simultaneous, sequential or separate use in therapy.

A preferred aspect more worthy to be mentioned of embodiment a of the present invention is a pharmaceutical composition comprising a first active ingredient which is at least one compound selected from list C,

or a salt solvate or solvate of a salt of this compound; and

a second active ingredient selected from the group consisting of NSAIDs, COX-2 inhibitors, NO-NSAIDs, bisphosphonates and corticosteroids

for simultaneous, sequential or separate use in therapy.

A further preferred aspect more worthy to be mentioned of embodiment a of the present invention is a pharmaceutical product (such as, for example, a commercial package) comprising a first active ingredient which is at least one compound selected from list C,

or a salt, solvate or solvate of a salt of this compound; and

a second active ingredient selected from the group consisting of NSAIDs, COX-2 inhibitors, NO-NSAIDs, bisphosphonates and corticosteroids;

together with instructions for simultaneous, sequential or separate use, e.g. to treat or prevent gastrointestinal diseases caused by said second active ingredient and/or to treat or prevent diseases which can be treated or prevented by said second active ingredient.

A further preferred aspect more worthy to be mentioned of embodiment a of the present invention is a kit comprising a first active ingredient which is at least one compound selected from list C, or a salt, solvate or solvate of a salt of this compound; and

a second active ingredient selected from the group consisting of NSAIDs, COX-2 inhibitors, NO-NSAIDs, bisphosphonates and corticosteroids;

optionally together with instructions for simultaneous, sequential or separate use in therapy, e.g. to treat or prevent gastrointestinal diseases caused by said second active ingredient and/or to treat or prevent diseases which can be treated or prevented by said second active ingredient.

More precisely, yet a preferred aspect more worthy to be mentioned of embodiment a of the present invention is a pharmaceutical composition comprising a first active ingredient which is selected from list C,

or a salt, solvate or solvate of a salt of this compound; and

a second active ingredient which is

a NSAID, a COX-2 inhibitor, a NO-NSAID, a bisphosphonate or a corticosteroid, or, in particular,

a NSAID, a COX-2 inhibitor or a bisphosphonate, or, in a first embodimental subaspect according to this invention,

a NSAID, such as e.g. one of those NSAIDs mentioned specifically or generically above, or, in a second embodimental subaspect according to this invention,

a COX-2 inhibitor, such as e.g. one of those COX-2 inhibitors mentioned specifically or generically above, or, in a third embodimental subaspect according to this invention,

a bisphosphonate, such as e.g. one of those bisphosphonates mentioned specifically or generically above, or, in fourth embodimental subaspect according to this invention,

a corticosteroid, such as e.g. one of those corticosteroids mentioned specifically or generically above, for simultaneous, sequential or separate use in therapy in any order.

A more preferred aspect of embodiment a of the present invention is a pharmaceutical composition comprising a first active ingredient which is a compound selected from list C,

or a salt, solvate or solvate of a salt of this compound; and

a second active ingredient which is

a NSAID, such as e.g. one of those NSAIDs mentioned specifically or generically above, or, in a more detailed subaspect according to this invention,

a NSAID selected from the group consisting of

ACETYLSALICYLIC ACID, DICLOFENAC, DIFLUNISAL, ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID and TOLMETIN, or a pharmaceutically acceptable derivative thereof,

or, in a particular detailed subaspect according to this invention,

a NSAID selected from the group consisting of

DICLOFENAC, IBUPROFEN, INDOMETHACIN, NAPROXEN and PIROXICAM, or a pharmaceutically acceptable derivative thereof,

or, in a more particular detailed subaspect according to this invention,

DICLOFENAC, or a pharmaceutically acceptable derivative thereof,

for simultaneous, sequential or separates use in therapy.

A further more preferred aspect of embodiment a of the present invention is a pharmaceutical composition comprising a first active ingredient which is a compound selected from list C,

or a salt solvate or solvate of a salt of this compound; and

a second active ingredient which is

a COX-2 inhibitor, such as e.g. one of those COX-2 inhibitors mentioned specifically or generically above,

or, in a more detailed subaspect according to this invention,

a COX-2 inhibitor selected from the group consisting of

ETORICOXIB, CELECOXIB, ROFECOXIB, PARECOXIB, VALDECOXIB, LUMIRACOXIB, TILMACOXIB, CIMICOXIB, NIMESULIDE, FLOSULIDE, DUP-697, FK-3311, NS-398, L-745337, GR-253035, SC-58236, LAS-33815, CS ABT-963, GW-401381 and those COX-2 inhibitors disclosed in WO 02096427, WO 02096886 or WO 02096885, or a pharmaceutically acceptable derivative thereof.

or, in a particular detailed subaspect according to this invention,

a COX-2 inhibitor selected from the group consisting of

ETORICOXIB, CELECOXIB, ROFECOXIB, PARECOXIB, VALDECOXIB, LUMIRACOXIB, TILMACOXIB and CIMICOXIB, or a pharmaceutically acceptable derivative thereof,

for simultaneous, sequential or separate use in therapy.

A further more preferred aspect of embodiment a of the present invention is a pharmaceutical composition comprising a first active ingredient which is a compound selected from list C,

or a salt solvate or solvate of a salt of this compound; and

a second active ingredient which is

a bisphosphonate, such as e.g. one of those bisphosphonates mentioned exemplarily above, or, in a more detailed subaspect according to this invention,

a bisphosphonate selected from the group consisting of

ALENDRONIC ACID, RISEDRONIC ACID, TILUDRONIC ACID, IBANDRONIC ACID, ZOLEDRONIC ACID, CLODRONIC ACID, INCADRONIC ACID, OLPADRONIC ACID, MINODRONIC ACID, PAMIDRONIC ACID and ETIDRONIC ACID,

or a pharmaceutically acceptable derivative thereof,

for simultaneous, sequential or separate use in therapy.

A further more preferred aspect of embodiment a of the present invention is a pharmaceutical composition comprising a first active ingredient which is a compound selected from list C,

or a salt, solvate or solvate of a salt of this compound; and

a second active ingredient which is

a corticosteroid, such as e.g. one of those corticosteroids mentioned exemplarily above,

or, in a more detailed subaspect according to this invention,

a corticosteroid selected from the group consisting of those corticosteroids mentioned in embodiment 4 or a pharmaceutically acceptable derivative thereof,

or, in a particular detailed subaspect according to this invention,

a corticosteroid selected from the group consisting of those corticosteroids mentioned in embodiment 4′ or a pharmaceutically acceptable derivative thereof,

or, in a more particular detailed subaspect according to this invention,

a corticosteroid selected from the group consisting of BETAMETHASONE, DEXAMETHASONE, FLUOCORTOLONE, METHYLPREDNISOLONE, PREDNISOLONE, PREDNISONE, HYDROCORTISONE, BUDESONIDE and TRIAMCINOLONE ACETONIDE,

or a pharmaceutically acceptable derivative thereof,

for simultaneous, sequential or separate use in therapy.

Another more preferred aspect of embodiment a of the present invention is a pharmaceutical composition comprising a first active ingredient which is a compound selected from the list C,

or a salt solvate or solvate of a salt of this compound and

a second active ingredient which is selected from the group consisting of

ACETYLSALICYLIC ACID, DICLOFENAC, DIFLUNISAL, ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID, TOLMETIN, ETORICOXIB, CELECOXIB, ROFECOXIB, PARECOXIB, VALDECOXIB, LUMIRACOXIB, TILMACOXIB, CIMICOXIB, NIMESULIDE, FLOSULIDE, DUP-697, FK-3311, NS-398, L-745337, GR-253035, SC-8238, LAS 33815, CS-502, ABT-963, GW-406381, and

ALENDRONIC ACID, RISEDRONIC ACID, TILUDRONIC ACID, IBANDRONIC ACID, ZOLEDRONIC ACID, CLODRONIC ACID, INCADRONIC ACID, OLPADRONIC ACID, MINODRONIC ACID, PAMIDRONIC ACID, ETIDRONIC ACID, and

BETAMETHASONE, DEXAMETHASONE, FLUOCORTOLONE, METHYLPREDNISOLONE, PREDNISOLONE, PREDNISONE, HYDROCORTISONE, BUDESONIDE and TRIAMCINOLONE ACETONIDE,

or a pharmaceutically acceptable derivative thereof,

for simultaneous, sequential or separate use in therapy in any order.

More detailed, yet another more preferred aspect of embodiment a of the present invention is a pharmaceutical composition comprising a first active ingredient which is a compound selected from list C, or a salt, solvate or solvate of a salt of this compound; and

a second active ingredient which is selected from the group consisting of

ACETYLSALICYLIC ACID, DICLOFENAC, DIFLUNISAL, ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID, TOLMETIN, ETORICOXIB, CELECOXIB, ROFECOXIB, PARECOXIB, VALDECOXIB, LUMIRACOXIB, TILMACOXIB, CIMICOXIB, and

ALENDRONIC ACID, RISEDRONIC ACID, TILUDRONIC ACID, IBANDRONIC ACID, ZOLEDRONIC ACID, CLODRONIC ACID, INCADRONIC ACID, OLPADRONIC ACID, MINODRONIC ACID, PAMIDRONIC ACID, ETIDRONIC ACID, and

BETAMETHASONE, DEXAMETHASONE, FLUOCORTOLONE, METHYLPREDNISOLONE, PREDNISOLONE, PREDNISONE, HYDROCORTISONE, BUDESONIDE and TRIAMCINOLONE ACETONIDE,

or a pharmaceutically acceptable derivative thereof,

for simultaneous, sequential or separate use in therapy in any order.

Still more detailed, yet another more preferred aspect of embodiment a of the present invention is a pharmaceutical composition comprising a first active ingredient which is a compound selected from list C,

or a salt, solvate or solvate of a salt of this compound; and

a second active ingredient which is selected from the group consisting of

ACETYLSALICYLIC ACID, DICLOFENAC, DIFLUNISAL, ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID, TOLMETIN, ETORICOXIB, CELECOXIB, ROFECOXIB, PARECOXIB, VALDECOXIB, LUMIRACOXIB, TILMACOXIB, CIMICOXIB, and

ALENDRONIC ACID, RISEDRONIC ACID, TILUDRONIC ACID, IBANDRONIC ACID, ZOLEDRONIC ACID, CLODRONIC ACID, INCADRONIC ACID, OLPADRONIC ACID, MINODRONIC ACID, PAMIDRONIC ACID and ETIDRONIC ACID,

or a pharmaceutically acceptable derivative thereof,

for simultaneous, sequential or separate use in therapy in any order.

Yet still more detailed, a yet another more preferred aspect of embodiment a of the present invention is a pharmaceutical composition comprising a first active ingredient which is a compound selected from list C,

or a salt solvate or solvate of the salt of a compound; and

a second active ingredient which is selected from the group consisting of

ACETYLSALICYLIC ACID, DICLOFENAC, DIFLUNISAL, ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID, TOLMETIN, ETORICOXIB, CELECOXIB, ROFECOXIB, PARECOXIB, VALDECOXIB, LUMIRACOXIB, TILMACOXIB and CIMICOXIB,

or a pharmaceutically acceptable derivative thereof,

for simultaneous, sequential or separate use in therapy in any order.

Each in particular preferred individual aspects of embodiment a of the present invention refer to respective pharmaceutical compositions being based on the specific disclosure of this invention, that each and every one of the tricyclic imidazo[1,2-a]pyridine compound mentioned expressis verbis in list C as compounds 1 to 17, or a salt, solvate or solvate of a salt thereof, can be individually, specifically and independently used as first active ingredient in respective embodimental pharmaceutical compositions according to the present invention comprising said specific first active ingredient and a second active ingredient which is

a NSAID, a COX-2 inhibitor, a NO-NSAID, a bisphosphonate or a corticosteroid, or, in particular,

a NSAID, a COX-2 inhibitor or a bisphosphonate, or, in a first embodiment subaspect according to this invention,

a NSAID, such as e.g. one of those NSAIDs mentioned specifically or generically above, or, in a second embodimental subaspect according to this invention,

a COX-2 inhibitor, such as e.g. one of those COX-2 inhibitors mentioned specifically or generically above, or, in a third embodimental subaspect according to this invention,

a bisphosphonate, such as e.g. one of those bisphosphonates mentioned specifically or generically above, or, in a fourth embodimental subaspect according to this invention,

a corticosteroid, such as e.g. one of those corticosteroids mentioned specifically or generically above, or, in a more detailed embodimental subaspect according to this invention,

an agent selected from the group consisting of

ACETYLSALICYLIC ACID, DICLOFENAC, DIFLUNISAL, ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID, TOLMETIN, ETORICOXIB, CELECOXIB, ROFECOXIB, PARECOXIB, VALDECOXIB, LUMIRACOXIB, TILMACOXIB, CIMICOXIB, NIMESULIDE, FLOSULIDE, DUP-697, FK-3311, NS-398, L-745337, GR-253035, SC 58236, LAS-33815, CS-502, ABT-963, GW-406381, and

ALENDRONIC ACID, RISEDRONIC ACID, TILUDRONIC ACID, IBANDRONIC ACID, ZOLEDRONIC ACID, CLODRONIC ACID, INCADRONIC ACID, OLPADRONIC ACID, MINODRONIC ACID, PAMIDRONIC ACID, ETIDRONIC ACID, and

BETAMETHASONE, DEXAMETHASONE, FLUOCORTOLONE, METHYLPREDNISOLONE, PREDNISOLONE, PREDNISONE, HYDROCORTISONE, BUDESONIDE and TRIAMCINOLONE ACETONIDE,

or a pharmaceutically acceptable derivative thereof,

or, in a still more detailed embodimental subaspect according to this invention,

an agent selected from the group consisting of

ACETYLSALICYLIC ACID, DICLOFENAC, DIFLUNISAL, ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID, TOLMETIN, ETORICOXIB, CELECOXIB, ROFECOXIB, PARECOXIB, VALDECOXIB, LUMIRACOXIB, TILMACOXIB, CIMICOXIB, ALENDRONIC ACID, RISEDRONIC ACID, TILUDRONIC ACID, IBANDRONIC ACID, ZOLEDRONIC ACID, CLODRONIC ACID, INCADRONIC ACID, OLPADRONIC ACID, MINODRONIC ACID, PAMIDRONIC ACID and ETIDRONIC ACID,

or a pharmaceutically acceptable derivative thereof,

for simultaneous, sequential or separate use in therapy.

An in particular preferred aspect more worthy to be mentioned of embodiment a of the present invention is a pharmaceutical composition comprising a first active ingredient which is
(7R,8R,9R)-8-hydroxy-7-(2-methoxyethoxy)-2,3-dimethyl-9-phenyl-7,8,9,10-tetrahydro-imidazo[1,2-h]-1,7]naphthyridine,
   or a salt, solvate or solvate of a salt of this compound; and
   a second active ingredient which is
   a NSAID, a COX-2 inhibitor, a NO-NSAID, a bisphosphonate or a corticosteroid, or, in particular,
   a NSAID, a COX-2 inhibitor or a bisphosphate, or, in a first embodimental subaspect according to this invention,
   a NSAID, such as e.g. one of those NSAIDs mentioned specifically or generically above, or, in a second embodimental subaspect according to this invention,
   a COX-2 inhibitor, such as e.g. one of those COX-2 inhibitors mentioned specifically or generically above, or, in a third embodimental subaspect according to this invention,
   a bisphosphonate, such as e.g. one of those bisphosphonates mentioned specifically or generically above, or, in a fourth embodimental subaspect according to this invention.
   a corticosteroid, such as e.g. one of those corticosteroids mentioned specifically or generically above,
   or, in a more detailed embodimental subaspect according to this invention,
   an agent selected from the group consisting of
   ACETYLSALICYLIC ACID, DICLOFENAC, DIFLUNISAL, ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID, TOLMETIN, ETORICOXIB, CELECOXIB, ROFECOXIB, PARECOXIB, VALDECOXIB, LUMIRACOXIB, TILMACOXIB, CIMICOXIB, NIMESULIDE, FLOSULIDE, DUP-697, FK-3311, NS-398, L-745337, GR-253035, SC-8236, LAS-33815, CS-502, ABT-883, GW-406381, and
   ALENDRONIC ACID, RISEDRONIC ACID, TILUDRONIC ACID, IBANDRONIC ACID, ZOLEDRONIC ACID, CLODRONIC ACID, INCADRONIC ACID, OLPADRONIC ACID, MINODRONIC ACID, PAMIDRONIC ACID, ETIDRONIC ACID, and
   BETAMETHASONE, DEXAMETHASONE, FLUOCORTOLONE, METHYLPREDNISOLONE, PREDNISOLONE, PREDNISONE, HYDROCORTISONE, BUDESONIDE and TRIAMCINOLONE ACETONIDE,
   or a pharmaceutically acceptable derivative thereof,
   or, in a still more detailed embodimental subaspect according to this invention,
   an agent selected from the group consisting of
   ACETYLSALICYLIC ACID, DICLOFENAC, DIFLUNISAL, ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID, TOLMETIN, ETORICOXIB, CELECOXIB, ROFECOXIB, PARECOXIB, VALDECOXIB, LUMIRACOXIB, TILMACOXIB, CIMICOXIB, ALENDRONIC ACID, RISEDRONIC ACID, TILUDRONIC ACID, IBANDRONIC ACID, ZOLEDRONIC ACID, CLODRONIC ACID, INCADRONIC ACID, OLPADRONIC ACID, MINODRONIC ACID, PAMIDRONIC ACID and ETIDRONIC ACID,
   or a pharmaceutically acceptable derivative thereof,
   for simultaneous, sequential or separate use in therapy.

A yet further in particular preferred aspect more worthy to be mentioned of embodiment a of the present invention is a pharmaceutical product (such as, for example, a commercial package) comprising a first active ingredient which is
(7R,8R,9R)-8-hydroxy-7-(2-methoxyethoxy)-2,3-dimethyl-9-phenyl-7,8,9,10-tetrahydro-imidazo[1,2-h]-[1,7]naphthyridine.
   or a salt, solvate or solvate of a salt of this compound; and
   a second active ingredient which is
   a NSAID, a COX-2 inhibitor, a NO-NSAID, a bisphosphonate or a corticosteroid, or, in particular,
   a NSAID, a COX-2 inhibitor or a bisphosphonate, or, in a first subaspect,
   a NSAID, such as e.g. one of those NSAIDs mentioned above, or, in a second subaspect,
   a COX-2 inhibitor, such as e.g. one of those COX-2 inhibitors mentioned above, or, in a third subaspect,
   a bisphosphonate, such as e.g. one of those bisphosphonates mentioned above, or, in a fourth subaspect,
   a corticosteroid, such as e.g. one of those corticosteroids mentioned above,
   or, in a more detailed subaspect,
   an agent selected from the group consisting of
   ACETYLSALICYLIC ACID, DICLOFENAC, DIFLUNISAL. ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID, TOLMETIN, ETORICOXIB, CELECOXIB, ROFECOXIB, PARECOXIB, VALDECOXIB, LUMIRACOXIB, TILMACOXIB, CIMICOXIB, NIMESULIDE, FLOSULIDE, DUP-697, FK-3311, NS-398, L-745337, GR-253035, SC-58236, LAS-33815, CS-502, ABT-963, GW-406381, and
   ALENDRONIC ACID, RISEDRONIC ACID, TILUDRONIC ACID, IBANDRONIC ACID, ZOLEDRONIC ACID, CLODRONIC ACID, INCADRONIC ACID, OLPADRONIC ACID, MINODRONIC ACID, PAMIDRONIC ACID, ETIDRONIC ACID, and
   BETAMETHASONE, DEXAMETHASONE, FLUOCORTOLONE, METHYLPREDNISOLONE, PREDNISOLONE, PREDNISONE, HYDROCORTISONE, BUDESONIDE and TRIAMCINOLONE ACETONIDE,
   or a pharmaceutically acceptable derivative thereof,
   or, in a still more detailed subaspect,
   an agent selected from the group consisting of
   ACETYLSALICYLIC ACID, DICLOFENAC, DIFLUNISAL, ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID, TOLMETIN, ETORICOXIB, CELECOXIB, ROFECOXIB, PARECOXIB, VALDECOXIB, LUMIRACOXIB, TILMACOXIB, CIMICOXIB, ALENDRONIC ACID, RISEDRONIC ACID, TILUDRONIC ACID, IBANDRONIC ACID, ZOLEDRONIC ACID, CLODRONIC ACID, INCADRONIC ACID, OLPADRONIC ACID, MINODRONIC ACID, PAMIDRONIC ACID and ETIDRONIC ACID,
   or a pharmaceutically acceptable derivative thereof,
   together with instructions for simultaneous, sequential or separate use, e.g. to treat or prevent gastro intestinal diseases caused by said second active ingredient and/or to treat or prevent diseases which can be treated or prevented by said second active ingredient.

A still further in particular preferred aspect more worthy to be mentioned of embodiment a of the present invention is a kit comprising a first active ingredient which is
(7R,8R,9R)-8-hydroxy-7-(2-methoxyethoxy)-2,3-dimethyl-9-phenyl-7,8,9,10-tetrahydro-imidazo[1,2-h]-[1,7]naphthyridine,
   or a salt, solvate or solvate of a salt of this compound; and
   a second active ingredient which is
   a NSAID, a COX-2 inhibitor, a NO-NSAID, a bisphosphonate or a corticosteroid, or, in particular,
   a NSAID, a COX-2 inhibitor or a bisphosphonate, or, in a first subaspect,
   a NSAID, such as e.g. one of those NSAIDs mentioned above, or, in a second subaspect,
   a COX-2 inhibitor, such as e.g. one of those COX-2 inhibitors mentioned above, or, in a third subaspect,
   a bisphosphonate, such as e.g. one of those bisphosphonates mentioned above, or, in a fourth subaspect,
   a corticosteroid, such as e.g. one of those corticosteroids mentioned above, or, in a more detailed subaspect,
   an agent selected from the group consisting of
   ACETYLSALICYLIC ACID, DICLOFENAC, DIFLUNISAL, ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID, TOLMETIN, ETORICOXIB, CELECOXIB, ROFECOXIB, PARECOXIB, VALDECOXIB, LUMIRACOXIB, TILMACOXIB, CIMICOXIB, NIMESULIDE, FLOSULIDE, DUP-697, FK-3311, NS-398, L-745337, GR-253035, SC-58236, LAS-33815, CS-502, ABT-963, GW-406381, and
   ALENDRONIC ACID, RISEDRONIC ACID, TILUDRONIC ACID, IBANDRONIC ACID, ZOLEDRONIC ACID, CLODRONIC ACID, INCADRONIC ACID, OLPADRONIC ACID, MINODRONIC ACID, PAMIDRONIC ACID, ETIDRONIC ACID, and
   BETAMETHASONE, DEXAMETHASONE, FLUOCORTOLONE, METHYLPREDNISOLONE, PREDNISOLONE, PREDNISONE, HYDROCORTISONE, BUDESONIDE and TRIAMCINOLONE ACETONIDE,
   or a pharmaceutically acceptable derivative thereof,
   or, in a still more detailed subaspect,
   an agent selected from the group consisting of
   ACETYLSALICYLIC ACID, DICLOFENAC, DIFLUNISAL. ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID, TOLMETIN, ETORICOXIB, CELECOXIB, ROFECOXIB, PARECOXIB, VALDECOXIB, LUMIRACOXIB, TILMACOXIB, CIMICOXIB, ALENDRONIC ACID, RISEDRONIC ACID, TILUDRONIC ACID, IBANDRONIC ACID, ZOLEDRONIC ACID, CLODRONIC ACID, INCADRONIC ACID, OLPADRONIC ACID, MINODRONIC ACID, PAMIDRONIC ACID and ETIDRONIC ACID,
   or a pharmaceutically acceptable derivative thereof,
   optionally together with instructions for simultaneous, sequential or separate use, e.g. to treat or prevent gastrointestinal diseases caused by said second active ingredient and/or to treat or prevent diseases which can be treated or prevented by said second active ingredient.

Among the abovementioned aspects these are to be emphasized, in which (7R,8R,9R)-8-hydroxy-7-(2-methoxyethoxy)-2,3-dimethyl-9-phenyl-7,8,9,10-tetrahydro-imidazo[1,2-h][1,7]naphthyridine [INN: Soraprazan] or a salt, solvate or solvate of the salt of this compound is mentioned specifically as first active ingredient; and these are particularly to be emphasized, in which (7R,8R,9R)-8-hydroxy-7-(2-methoxyethoxy)-2,3-dimethyl-9-phenyl-7,8,9,10-tetrahydro-imidazo[1,2-h][1,7]napthyridine or a salt, solvate or solvate of the salt of this compound is mentioned solely as first active ingredient.

An in more particular preferred aspect of the present invention is a pharmaceutical composition comprising a first active ingredient which is
(7R,8R,9R)-8-hydroxy-7-(2-methoxyethoxy)-2,3-dimethyl-9-phenyl-7,8,9,10-tetrahydro-imidazo[1,2-h]-[1,7]naphthyridine, or a salt, solvate or solvate of a salt of this compound; and
   a second active ingredient which is DICLOFENAC, or a pharmaceutically acceptable derivative thereof,
   for simultaneous, sequential or separate use in therapy.

Additionally, another preferred aspect according to embodiment a of the present invention is also the use of a compound selected from list C,

or a salt, solvate or solvate of the salt of this compound,

in the manufacture of a pharmaceutical composition for the prevention or treatment of medicament caused gastrointestinal diseases or medicament associated gastrointestinal disorders, particularly those mentioned in this invention.

Yet another preferred aspect according to aspect a of the present invention is the use of a compound selected from list C,

or a salt, solvate or solvate of the salt of this compound,

in the manufacture of a pharmaceutical composition comprising an active ingredient (ingredient b) which is

a NSAID, a COX-2 inhibitor, a NO-NSAID, a bisphosphonate or a corticosteroid, or, in particular,

a NSAID, a COX-2 inhibitor or a bisphosphonate, or, in more particular,

a NSAID or a COX-2 inhibitor, or, in still more particular,

a NSAID, or, in a subaspect according to this invention,

a NSAID, such as e.g. one of those NSAIDs mentioned above, or, in a second subaspect according to this invention,

a COX-2 inhibitor, such as e.g. one of those COX-2 inhibitors mentioned above, or, in a third subaspect according to this invention,

a bisphosphonate, such as e.g. one of those bisphosphonates mentioned above, or in a fourth subaspect according to this invention,

a corticosteroid, such as e.g. one of those corticosteroids mentioned above,

or, in a more detailed subaspect according to this invention,

an agent selected from the group consisting of

ACETYLSALICYLIC ACID, DICLOFENAC, DIFLUNISAL, ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID, TOLMETIN, ETORICOXIB, CELECOXIB, ROFECOXIB, PARECOXIB, VALDECOXIB, LUMIRACOXIB, TILMACOXIB, CIMICOXIB, ALENDRONIC ACID, RISEDRONIC ACID, TILUDRONIC ACID, IBANDRONIC ACID, ZOLEDRONIC ACID, CLODRONIC ACID, INCADRONIC ACID, OLPADRONIC ACID, MINODRONIC ACID, PAMIDRONIC ACID and ETIDRONIC ACID,

or a pharmaceutically acceptable derivative thereof,

for the prevention or treatment of gastrointestinal diseases or disorders caused by or associated with said ingredient b, and/or

for the prevention or treatment of diseases or disorders which can be treated or prevented by said ingredient b.

Yet additionally, a particularly preferred aspect of the present invention is the use of (7R,8R,9R)-8-hydroxy-7-(2-methoxyethoxy)-2,3-dimethyl-9-phenyl-7,8,9,10-tetrahydro-[1,2-h][1,7]naphthyridine or a salt, solvate or solvate of the salt of this compound in the manufacture of pharmaceutical compositions for the prevention of medicament caused gastrointestinal diseases, particularly medicament induced gastric ulcer.

Furthermore according to embodiment a or, particularly, embodiment b of this invention, the following aspects are also to be mentioned:

An aspect of embodiment a or b of the present invention to be mentioned is a pharmaceutical composition comprising, in admixture, a first active ingredient, which is at least one tricyclic imidazo[1,2-a]pyridine compound selected from abovementioned list A or C (according to embodiment a), or list B (according 1 embodiment b) or a salt, solvate or solvate of the salt of this compound, and a second active ingredient which is selected from a group consisting of NMDs, COX-2 inhibitors, NO-NSAIDs and bisphosphonates, in particular a NSAID selected from the group consisting of those NSAIDs mentioned in embodiment 1 above, or a NO-NSAID selected from the group consisting of those a NO-NSAIDs mentioned above, a COX-2 inventor selected from the group consisting of those COX-2 inhibitors mentioned in embodiment 2 above, or a N isphosphonate selected from the group consisting of those bisphosphonates mentioned in embodiment 3′ above, or a pharmaceutical acceptable derivative of these compounds.

In more particular a NSAID or a COX-2 inhibitor or a NO-NSAID or a bisphosphonate selected from the group consisting of ACETYLSALICYLIC ACID, DICLOFENAC, DIFLUNISAL, ETODOLAC, FENOPROFEN, FLOCTAFENINE, FLURBIPROFEN, IBUPROFEN, INDOMETHAN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID, TOLMETIN, CELEBREX (CELECOXIB), VIOX (ROFECOXIB), ALENDRONATE, RISEDRONATE, TILUDRONATE, IBANDRONATE, ZOLEDRONATE, ETIDRONATE and the pharmaceutically acceptable derivatives of these compounds.

A further aspect of embodiment a or b of the present invention to be mentioned is a kit or pharmaceutical product comprising a preparation of a first active ingredient, which is at least one of said selected tricyclic imidazo[1,2-a]pyridine compounds, a preparation of a second active ingredient which is a NSAID or a COX-2 inhibitor or a NO-NSAID or a bisphosphonate selected from the group consisting of CETYLSALCYLIC ACID, DICLOFENAC, DIFLUNISAL, ETODOLAC, FENOPROFEN, FLOCTAFENINE, LURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID, TOLMETIN, CELEBREX (CELECOXIB), VIOXX (ROFECOXIB), ALENDRONATE, RISEDRONATE, TILUDRONATE, IBANDRONATE, ZOLEDRONATE, ETIDRONATE and the pharmaceutically acceptable derivatives of these compounds, and instructions for simultaneous, sequential or separate administration of the preparations to a present in need thereof, e.g. to prevent medicament induced gastric ulcer in said patient A further aspect of embodiment a or b of the present invention to be mentioned is a pharmaceutical product comprising, in combination, a preparation of a first active ingredient, which is at least one tricyclimidazo[1,2a]pyridine compound selected from above mentioned list A or C, or list B or a salt, solvate or solvate of the salt of this compound, and a preparation of a second acheive ingredient, which is a NSAID or a COX-2 inhibitor or a NO-NSAID or a bisphosphonate, for simultaneous, sequential or separate use in therapy. e.g. to prevent medicament induced gastric ulcer in a mammal.

A further aspect of embodiment a or b of the present invention to be mentioned is a kit comprising a preparation of a first active ingredient which is at least one tricyclic imidazo[1,2-a]pyridine compound selected from above mentioned list A or C, or list B or a salt, solvate or solvate of the salt of this compound, a preparation of a second active ingredient, which is selected from a group consisting of NSAIDs, COX-2 inhibitors, NO-NSAIDs and bisphosphonates, and instructions for simultaneous, sequential or separate administration of the preparations to a patient in need thereof.

A further aspect of embodiment a or b of the present invention to be mentioned is the use of a tricyclic imidazo[1,2-a]pyridine compound selected from abovementioned list A or C, or list B and of the salts, solvates and solvates of the salts of these compounds in the manufacture of pharmaceutical compositions for the prevention of medicament caused gastrointestinal diseases, particularly, medicament induced gastric ulcer, and/or in the manufacture of pharmaceutical compositions for the prevention of medicament associated gastrointestinal disorders.

An aspect of embodiment a or b of the present invention more worthy to be mentioned is a pharmaceutical composition comprising, in admixture, a first active ingredient, which is either (7R,8R,9R)-8-hydroxy-7-(2-methoxyethoxy)-2,3-dimethyl-9-phenyl-7,8,9,10-tetrahydroimidazo[1,2-h][1,7]naphthyridine or a salt, solvate or solvate of the salt of this compound, and a second active ingredient, which is selected from a group consisting of NSAIDs, COX-2 inhibitors, NO-NSAIDs and bisphosphonates.

Yet an aspect of embodiment a or b of the present invention more worthy to be mentioned is a pharmaceutical product comprising, in combination, a preparation of a first active ingredient, which is elther (7R,8R,9R)-8-hydroxy-7-(2-methoxyethoxy)-2,3-dimethyl-9-phenyl-7,8,9,10-tetrahydroimidazo[1,2-h][1,7]naphthyridine or a salt, solvate or solvate of the salt of this compound, and a preparation of a second active ingredient, which is a NSAID or a COX-2 inhibitor or a NO-NSAID or a bisphosphonate, for simultaneous, sequential or separate use in therapy, e.g. to prevent medicament induced gastric ulcer in a mammal.

Yet an aspect of embodiment a or b of the present invention more worthy to be mentioned is a kit comprising a preparation of a first active ingredient, which is ether (7R,8R,9R)-8-hydroxy-7-(2-methoxyethoxy)-2,3-dimethyl-9-phenyl-7,8,9,10-tetrahydro-imidazo[1,2-h[1,7]naphthyridine or a salt, solvate or solvate of the salt of this compound, a preparation of a second active ingredient, which is selected from a group consisting of NSAIDs, COX-2 inhibitors, NO-NSAIDs and bisphosphonates, and instructions for simultaneous, sequential or separate administration of the preparations to a patient in need thereof.

In detail, an aspect of embodiment a or b of the present invention more worthy to be mentioned is a pharmaceutical composition comprising, in admixture, a first active ingredient, which is (7R,8R,9R)-8-hydroxy-7-(2-methoxyethoxy)-2,3-dimethyl-9-phenyl-7,8,9,10-tetrahydro-imidazo[1,2-h][1,7]naphthyridine or its salt its solvate or the solvate of its salt;

and a second active ingredient, which is a NSAID or a COX-2 inhibitor a NO-NSAID or a bisphosphonate, in particular

a NSAID selected from the group consisting of those NSAIDs mentioned in embodiment 1 above, or

a NO-NSAID selected from the group consisting of those NO-NSAIDs mentioned above,

a COX-2 inhibitor selected from the group consisting of those COX-2 inhibitors mentioned in embodiment 2′ above, or

a bisphosphonate selected from the group consisting of those bisphosphonates mentioned in embodiment 3′ above, or

a pharmaceutically acceptable derivative of these compounds.

In more detail, an aspect of embodiment a or b of the present invention more worthy to be mentioned is a pharmaceutical composition comprising, in admixture, a first active ingredient, which is either (7R,8R,9R)-8-hydroxy-7-(2-methoxyethoxy)-2,3-dimethyl-9-phenyl-7,8,9,10-tetrahydro-imidazo[1,2-h][1,7]naphthyridine or a sat, solvate or solvate of the salt of this compound, and a second active ingredient, which is a NSAID or a COX-2 inhibitor or a NO-NSAID or a bisphosphonate selected from the group consisting of ACETYLSALICYLIC ACID, DICLOFENAC, DIFLUNISAL, ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID, TOLMETIN, CELEBREX (CELECOXIB), VIOXX (ROFECOXIB), ALENDRONATE, RISEDRONATE, TILUDRONATE, IBANDRONATE, ZOLEDRONATE, ETIDRONATE and the pharmaceutically acceptable derivatives of these compounds.

Yet in more detail, an aspect of embodiment a or b of the present invention more worthy to be mentioned is a pharmaceutical product comprising, in combination, a preparation of a first active ingredient which is either (7R,8R,9R)-8-hydroxy-7-(2-methoxyethoxy)-2,3-dimethyl-9-phenyl-7,8,9,10-tetrahydro-imidazo[1,2-h][1,7]naphthyridine or a salt, solvate or solvate of the salt of this compound, and a preparation of a second active ingredient which is a NSAID or a COX-2 inhibitor or a NO-NSAID or a bisphosphonate selected from the group consisting of ACETYLSALICYLIC ACID, DICLOFENAC, DIFLUNISAL, ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID, TOLMETIN, CELEBREX (CELECOXIB), VIOXX (ROFECOXIB), ALENDRONATE, RISEDRONATE, TILUDRONATE, IBANDRONATE, ZOLEDRONATE, ETIDRONATE and the pharmaceutically acceptable derivatives of these compounds, for simultaneous, sequential or separate use in therapy, e.g. to prevent-medicament induced gastric ulcer in a mammal.

Yet in more detail, an aspect of embodiment a or b of the present invention more worthy to be mentioned is a it comprising a preparation of a first active ingredient, which is either (7R,8R,9R)-8-hydroxy-7-(2-methoxyethoxy)-2,3-dimethyl-9-phenyl-7,8,9,10-tetrahydro-imidazo[1,2-h ][1,7]naphthyridine or a salt, solvate or solvate of the salt of this compound, a preparation of a second active ingredient, which is a NSAID or a COX-2 inhibitor or a NO-NSAID or a bisphosphonate selected from the group consisting of ACETYLSALICYLIC ACID, DICLOFENAC, DIFLUNISAL, ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID, TOLMETIN, CELEBREX (CELECOXIB), VIOXX (ROFECOXIB), ALENDRONATE, RISEDRONATE, TILUDRONATE, IBANDRONATE, ZOLEDRONATE, ETIDRONATE and the pharmaceutically acceptable derivatives of these compounds, and instructions for simultaneous, sequential or separate administration of the preparations to a patient in need thereof.

An aspect of embodiment a or b of the present invention to be especially worthy to be mentioned is a pharmaceutical composition comprising, in admixture, a first active ingredient, which is either (7R,8R,9R)-8-hydroxy-7-(2-methoxyethoxy)-2,3-dimethyl-9-phenyl-7,8,9,10-tetrahydro-imidazo[1,2-h][1,7]naphthylidine or a salt solvate or solvate of the salt of this compound, and a second active ingredient which is a NSAID or a COX-2 inhibitor or a NO-NSAID selected from the group consisting of ACETYLSALICYLIC ACID, DICLOFENAC, DIFLUNISAL, ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID, TOLMETIN, CELEBREX (CELECOXIB), VIOXX (ROFECOXIB) and the pharmaceutically acceptable derivatives of these compounds.

Yet an aspect of embodiment a or b of the present invention to be especially worthy to be mentioned is a pharmaceutical product comprising, in combination, a preparation of a first active ingredient, which is either (7R,8R,9R)-8-hydroxy-7-(2-methoxyethoxy)-2,3-dimethyl-9-phenyl-7,8,9,10-tetrahydro imidazo[1,2-h][1,7]naphthyridine or a salt, solvate or solvate of the salt of this compound, and a preparation of a second active ingredient, which is a NSAID or a COX-2 inhibitor or a NO-NSAID selected from the group consisting of ACETYLSALICYLIC ACID, DICLOFENAC, DIFLUNISAL, ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID, TOLMETIN, CELEBREX (CELECOXIB), VIOXX (ROFECOXIB) and the pharmaceutically acceptable derivatives of these compounds, for simultaneous, sequential or separate use in therapy, e.g. to prevent medicament induced gastric ulcer in a mammal.

Yet an aspect of embodiment a or b of the present invention to be especially worthy to be mentioned is a kit comprising a preparation of a first active ingredient, which is either (7R,8R,9R)-8-hydroxy-7-(2-methoxyethoxy)-2,3-dimethyl-9-phenyl-7,8,9,10-tetrahydro-imidazo[1,2-h][1,7]naphthyridine or a salt, solvate or solvate of the salt of this compound, a preparation of a second active ingredient, which is a NSAID or a COX-2 inhibitor or a NO-NSAID selected from the group consisting of ACETYLSALICYLIC ACID, DICLOFENAC, DIFLUNISAL, ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID, TOLMETIN, CELEBREX (CELECOXIB), VIOXX (ROFECOXIB) and the pharmaceutically acceptable derivatives of these compounds, and instructions for simultaneous, sequential or separate administration of the preparations to a patient in need thereof.

An aspect of embodiment a or b of the present invention to be more especially worthy to be mentioned is a pharmaceutical composition comprising, in admixture, a first active ingredient, which is (7R,8R,9R)-8-hydroxy-7-(2-methoxyethoxy)-2,3-dimethyl-9-phenyl-7,8,9,10-tetrahydro-imidazo[1,2-h][1,7]naphthyridine or its salt, its solvate or the solvate of its salt;

and a second active ingredient, which is DICLOFENAC or a pharmaceutically acceptable derivative of this compound.

Within the scope of this invention, “inflammatory diseases” which may be mentioned are gastrointestinal inflammatory diseases such as, for example, inflammatory bowel disease, Crohn's disease, irritable bowel syndrome, gastroesophageal reflux disease (GERD) and ulcerative colitis, or non-gastrointestinal inflammatory diseases, in particular arthritis, including but not limited to rheumatoid arthritis, osteoarthritis, systemic lupus erythematosus and juvenile arthritis; or asthma, bronchitis and skin related disorders such as psoriasis, eczema, burns and dermatitis.

“Inflammation associated disorders” which may be mentioned are, for example, pain (both chronic and acute) migraine, fever and headaches.

Furthermore, the person skilled in the art knows on the base of his/her expert knowledge which diseases, disorders or conditions can be treated, ameliorated or prevented by NSAIDs, COX-2 inhibitors, NO-NSAIDs, bisphosphonates or corticosteroids. Illustratively, as examples may be mentioned in this connection by way of example, without being restricted thereto, inflammatory, rheumatic or pain diseases.

According to the present invention, agents selected from the group consisting of NSAIDs, COX-2 inhibitors, NO-NSAIDs, bisphosphonates and corticosteroids can be combined beneficially with agents selected from the group consisting of certain tricyclic imidazo[1,2-pyridine compounds mentioned in the description of this invention to enhance or to improve safety and tolerability of the monotherapy, i.e. the monotherapy using only said agents selected from the group consisting of NSAIDs, COX-2 inhibitor, NO-NSAIDs, bisphosphonates and corticosteroids unpartnered with said tricyclic imidazo[1,2-a]pyridine compounds, by redcucing the risk of adverse effects, such as medicament-associated gastrointestinal disorders or medicament-caused gastrointestinal diseases, associated conventionally with the monotherapy.

In this context, the skilled person knows therefore on the base of his/her expert knowledge and/or on the base of the disclosure of the present invention which diseases, disorders or conditions conventionally treated, ameliorated or prevented monotherapeutically with NSAIDs, COX-2 inhibitors, NO-NSAIDs, bisphosphonates or corticosteroids can be now beneficially treated, ameliorated or prevented co-therapeutically, i.e. with the combination therapy according to the present invention.

In more detail, the combination therapy according to this invention can be applied to treat diseases, disorders or condition which can originally be treated, ameliorated or prevented by NSAIDs and/or COX-2 inhibitors, such as, for example, inflammatory diseases (in particular all kind of arthritis including rheumatoid arthritis or degenerative joint diseases including osteoarthritis) or inflammation associated disorders, and/or particularly symptoms caused by arthritis, such as inflammation, swelling, stiffness and joint pain, or other kinds of pain or painful conditions, such as e.g. gout attacks, bursitis, tendonitis, touthache, migraine, lower back and neck pain, myositis, sprains, strains or other injuries, or symptoms associated with influenza or other viral infections or common cold.

As further diseases, disorders or conditions, which can be treated, ameliorated or prevented by NSAIDs and/or, particularly, COX-2 inhibitors within the combination therapy according to this invention, can be mentioned, without being restricted thereto, neuropathic pains, (inflammatory) liver disease, stroke, epilepsy, dysmenorrhoea, ophthalmic diseases, cognitive disorders such as dementia, particularly degenerative dementia (such e.g. Alzheimer's disease) or, in more particular, cellular and neoplastic transformation and metastatic tumour growth, such e.g. certain cancerous diseases, for example colonic cancer and prostate cancer, or cancer associated with overexpression of HER-2/neu (e.g. breast cancer), or adenomatous colorectal polyps (and to reduce herewith the risk of developing colon cancer), or other conditions mediated by COX-2 (such as, e.g. conditions mediated by COX-2 overexpression during carcinogenesis).

As diseases, disorders or conditions, which can be treated, ameliorated or prevented by bisphosphonates within the combination therapy according to this invention, can be mentioned, without being restricted thereto, disorders associated with abnormal bone resorption such as, for example, osteoporosis, multiple myeloma or metastatic bone diseases (e.g. prostata, lung or breast cancer related), or tumor-induced hypercalcemia.

Oral corticosteroids can be used, for example, to treat autoimmune and inflammatory diseases, including asthma, bursitis, Crohn's disease, tendinitis, ulcerative colitis, rheumatoid arthritis, and lupus, and skin conditions, such as eczema and psoriasis. They can also be used to reduce inflammation associated with severe allergic reactions and to prevent organ rejection following transplant surgery.

Furthermore, the present invention provides also a teaching to broaden the primary therapeutic use of NSAIDs, COX-2 inhibitors, NO-NSAIDs, bisphosphonates or corticosteroids originally restricted due to risk of medicament caused gastrointestinal diseases. It is to be understood, that this broadened therapeutic use is also encompassed within the scope of this invention.

A further aspect of this invention is the combination of the abovementioned (pharmaceutical)compositions, pharmaceutical products, formulations, combinations, commercial packages or kits according to the invention with pharmaceuticals which inhibit add secretion, such as, for example. H₂ blockers (e.g. cimetidine, ranitidine), H⁺/K⁺ ATPase inhibitors (e.g. omeprazole, pantoprazole), or furthermore with so-called peripheral anticholinergics (e.g. pirenzepine, telenzepine) and also with gastrin antagonists with the aim of increasing the main action in an additive or superadditive sense and/or eliminating or decreasing the side effects.

Within the meaning of this invention the terms “use”, “administration”, “coadministration” or “administering” refer preferably to oral application. However in some cases, parenterate (e.g. intravenious), rectal or percutaneous application can be also advantageous.

The dosage of the active compounds is in a customary order of magnitude comparable with the monodosage, whereby, due to the additive and/or superadditive synergism of the single effects, the relevant doses of the active compounds in the combined dosage can be reduced compared to norm, or whereby—while maintaining the customary doses of the single components—a surprisingly higher and prolonged effect is obtained.

The person skilled in the art is are on the base of his expert knowledge of the total daily dosage of the NSAIDs, the COX-2 inhibitors, the NO-NSAIDs, the bisphosphonates or the corticosteroids which are comprised in the abovementioned pharmaceutical) compositions, pharmaceutical products, formulations, combinations, preparations, commercial packages or kits according to this invention. Said total daily dosage can vary within a wide range. For example, in the case of Diclofenac the daily doses are in a range from 100-2000 μg/kg.

In general, it has proven advantageous in human medicine to administer said selected tricyclic imidazo[1,2-a]pyridine compounds in the case of oral administration in a daily dose from approximately 0.01 to approximately 20, preferably 0.05 to 5, in particular 0.1 to 1.5, mg/kg of body weight, if appropriate in the form of several, preferably 1 to 4, individual doses to achieve the desired result in the case of parenteral treatment, similar or (in particular in the case of intravenous administration of the active compounds), as a rule, lower doses can be used.

The optimal dose and manner of administration of the active compounds necessary in each case can easily be determined by any person skilled in the art on the basis of his/her expert knowledge.

The person skilled in the art is familiar, on the basis of his/her knowledge, with carriers, diluents, adjuvants, auxiliaries or excipients which are suitable for the desired pharmaceutical compositions, formulations and/or preparations. Beside solvents, gel-forming agents, suppository bases, tablet auxiliaries and other active compounds, it is possible to use, for example, antioxidants, dispersants, emulsifiers, antifoam, flavor corrigents, preservatives, solubilizers, colorants or, in particular, permeation promoters and complexing agents (e.g. cyclodextrines).

In medicines, the active compounds are preferably employed in the form of tablets, coated tablets, capsules, suppositories, patches, emulsions, suspensions, gels or solutions, the active compound content advantageously being between 0.1 and 95%. Thus, for example with regard to the desired mode and site of action, the person skilled in the art can develop, on the basis of his/her knowledge, by appropriate choice of the excipients and the auxiliaries different galenic forms precisely tailored to the active ingredient(s) (such as, for example, retard forms or gastric add resistant foams).

A medicament or a pharmaceutical composition according to this invention can refer to a composition comprising both the said tricyclic imidazo[1,2-a]pyridine compound and the other active ingredient in a fixed combination (fixed unit dosage form), or a medicament pack comprising the two active ingredients as discrete separate dosage forms. In case of a medicament pack comprising the two active ingredients, the active ingredients are preferably packed into blister cards which are suited for improving compliance.

Each blister card preferably contains the medicaments to be taken on one day of treatment. If the medicaments are to be taken at different times of day, the medicaments can be disposed in different sections on the blister card according to the different ranges of times of day at which the medicaments are to be taken (for example morning and evening or morning, midday and evening). The blister cavities for the medicaments to be taken together at a particular time of day are accommodated in the respective range of times of day. The various times of day are, of course, also put on the blister in a clearly visible way. It is also possible, of course, for example to indicate a period in which the medicaments are to be taken, for example stating the times.

The daily sections may represent one line of the blister card, and the times of day are then identified in chronological sequence in this column.

Medicaments which must be taken together at a particular time of day are placed together at the appropriate time on the blister card, preferably a narrow distance apart, allowing them to be pushed out of the blister easily, and having the effect that removal of the dosage form from the blister is not forgotten.

All patents and patent applications referred to in this invention are herein incorporated by reference into the specification in their entirety for all purposes.

It is to be understood that the invention covers all combinations of single characteristics, aspects or embodiments of the invention as described herein.

Having described the invention in detail and by reference to the embodiments or aspects thereof, the scope of the present invention is not limited only to those described characteristics, embodiments or aspects. As will be apparent to persons skilled in the art, modifications, analogies, variations, derivations, homologisations and adaptations to the above-described invention can be made on the base of art-known knowledge and/or on the base of the disclosure (e.g. the explicite, implicit or inherent disclosure) of the present invention without departing from the spirit and scope of this invention.

The following examples serve to illustrate the invention in greater detail without restricting it. It will be readily apparent to those of ordinary skill in the art that the operating conditions, materials, procedural steps and other parameters of the invention described herein may be further modified or substituted in various ways without departing from the spirit and the scope of this invention.

### EXAMPLES

These experiments were done according to the procedure described in principle by Shay et al. Gastroenterology 1945, 5, 43-61, modified by Okabe et al. Jp. J. Pharmacol. 1974, 24, 383-371.

Rats were deprived of food 24 hours prior to the experiment with free access to water. After a midline abdominal incision under short isoflurane (Abbott no. B506) anaesthesia, the pylorus was ligated and the test substance or—regarding the control group—the vehicle (physiological saline) were given intraduodenally in 2.5 ml/kg body weight. The abdomen was dosed and 100 mg/kg of acetylsalicylic acid (ASA (Merck no. 85); suspended in 10 ml/kg of 1% Na-carboxymethylcellulose C1000P (Hoechst no. E0842965) solution] were administered orally, 4 hours after ligation, the stomach was carefully excised under isoflurane anaesthesia (keeping the esophagus dosed with a vessel forceps), opened along the greater curvature, and the gastric contents were removed. The animals were then sacrificed by atlas dislocation. The mucosa was flushed with saline and the stomach pinned on a styropor plate. The length and width of each gastric lesion was determined with a stereo-microscope using a 10-fold magnification. Each lesson was classified using the following score system
(Length+width)/2=point
no lesion=0
0.1-1.4 mm=1
1.5-2.4 mm=2
2.5-3.4 mm=3
3.5-4.4 mm=4
4.5-5.4 mm=5
≧5.5 mm=6

The sum of all points per animal represents the individual lesion index.

Conditions under which the animals were kept

Groups of 4 female rats per cage (Macrolon cage M III) were kept at about 22° C. and a relative humidity of 50-60%. They were fed ad libitum with NAFAG feed No. 9439 (NAFAG AG, CH-9200 Gossau, Switzerland) and had free access to water. Food was withdrawn 24 h before start of the experiment

Exemplary substance preparation:

(7R,8R,9R)-8-Hydroxy-7-(2-methoxyethoxy)-2,3-dimethyl-9-phenyl-7,8,9,10-tetrahydro-imidazo[1,2-h]-[1,7]naphthyridine was dissolved in DMSO and 0.1 N HCl immediately before the start of the experiment. The solution was further diluted with physiological saline and adminstered to the animals in a constant volume of 2.5 ml/kg body weight.

Table C shows the influence of exemplary compounds according to the invention given intraduodenally on gastric lesion 4 hour after pylorus ligation and oral administration of 100 mg/kg acetylsalicylic acid in the rat.

TABLE C

| | | | Reduction of lesion |
|---|---|---|---|
| Example | Compound according to the present | Dose administered | index versus control |
| number | invention | in μmol/kg | in % |
| 1 | (7R,8R,9R)-8-hydroxy-7-(2- | 3.0 | 100 |
| | methoxyethoxy)-2,3-dimethyl-9-phenyl- | | |
| | 7,8,9,10-tetrahydro-imi-dazo[1,2- | | |
| | h][1,7]naphthyridine | | |
| 2 | (7R, 8R, 9R)-2,3-dimethyl-8-hydroxy-7- | 1.0 | 100 |
| | methoxy-9-phenyl-7,8,9,10- | | |
| | tetrahydroimidazo[1,2-h][1,7]naph- | | |
| | thyridine | | |
| 3 | (7S, 8R, 9R)-2,3-dimethyl-8-hydroxy-7- | 10.0 | 100 |
| | methoxy-9-phenyl-7,8,9,10- | | |
| | tetrahydroimidazo[1,2-h][1,7]naph- | | |
| | thyridine | | |
| 4 | (7R, 8R, 9R)-2,3-dimethyl-7-ethoxy-8- | 1.0 | 100 |
| | hydroxy-9-phenyl-7,8,9,10- | | |
| | tetrahydroimidazo[1,2-h][1,7]naph- | | |
| | thyridine | | |
| 5 | (7S, 8R, 9R)-2,3-dimethyl-7-ethoxy-8- | 1.0 | 100 |
| | hydroxy-9-phenyl-7,8,9,10- | | |
| | tetrahydroimidazo[1,2-h][1,7]naph- | | |
| | thyridine | | |
| 6 | (7R, 8R, 9R)-2,3-dimethyl-8-hydroxy-7- | 1.0 | 100 |
| | (2-methoxyethoxy)-9-phenyl-7,8,9,10- | | |
| | tetrahydroimidazo[1,2- | | |
| | h][1,7]naphthyridine | | |
| 7 | (7R,8R,9R)-8-acetoxy-7-(2- | 1.0 | 100 |
| | methoxyethoxy)-2,3-dimethyl-9-phenyl- | | |
| | 7,8,9,10-tetrahydroimidazo[1,2- | | |
| | h][1,7]naphthyridine | | |
| 8 | (7R,8R,9R)-8-benzoyloxy-7-(2- | 1.0 | 100 |
| | methoxyethoxy)-2,3-dimethyl-9-phenyl- | | |
| | 7,8,9,10-tetrahydroimidazo[1,2- | | |
| | h][1,7]naphthyridine | | |
| 9 | (7R,8R,9R)-8-methoxycarbonyloxy-7-(2- | 1.0 | 100 |
| | methoxyethoxy)-2,3-dimethyl-9-phenyl- | | |
| | 7,8,9,10-tetrahydroimidazo[1,2- | | |
| | h][1,7]naphthyridine | | |
| 10 | (7R,8R,9R)-7-(2-methoxyethoxy)-2,3- | 1.0 | 100 |
| | dimethyl-8-(N,N-dimethylaminomethyl- | | |
| | carbonyloxy)-9-phenyl-7,8,9,10- | | |
| | tetrahydroimidazo[1,2- | | |
| | h][1,7]naphthyridine | | |
| 11 | (7R,8S,9R)-2,3,8-trimethyl-7,8- | 1.0 | 100 |
| | dihydroxy-9-phenyl-7,8,9,10- | | |
| | tetrahydroimidazo[1,2-h][1,7]naph- | | |
| | thyridine | | |
| 12 | (7R,8R,9R)-2,3,7-trimethyl-7,8- | 1.0 | 100 |
| | dihydroxy-9-phenyl-7,8,9,10- | | |
| | tetrahydroimidazo[1,2-h][1,7]naph- | | |
| | thyridine | | |
| 13 | (7R,8R,9R)-2,3-dimethyl-8-hydroxy-7- | 1.0 | 100 |
| | (2-methoxyethoxy)-9-phenyl-7H-8,9- | | |
| | dihydropyrano[2,3-c]imidazo[1,2- | | |
| | a]pyridine | | |
| 14 | (7R,8R,9R)-2,3-dimethyl-8-hydroxy-7- | 1.0 | 100 |
| | ethoxy-9-phenyl-7H-8,9- | | |
| | dihydropyrano[2,3-c]imidazo[1,2- | | |
| | a]pyridine | | |
| 15 | (7R,8R,9R)-8-hydroxy-2-methyl-7-(2- | 6.0 | 100 |
| | methoxyethoxy)-9-phenyl-7,8,9,10-tetra- | | |
| | hydroimidazo[1,2-h][1,7]naphthyridine | | |
| 16 | (7R,8R,9R)-3-chloro-8-hydroxy-7-(2- | 6.0 | 100 |
| | methoxyethoxy)-2-methyl-9-phenyl- | | |
| | 7.8.9.10-tetrahydroimidazo[1.2- | | |
| | h][1.7]naphthyridine | | |
| 17 | (7R,8R,9R)-3-chloro-8-hydroxy-7-(2- | 1.0 | 100 |
| | methoxyethoxy)-2-methyl-9-phenyl-7H- | | |
| | 8,9-dihydro-pyrano[2,3-c]imidazo[1,2- | | |
| | a]pyridine | | |

1. A pharmaceutical composition comprising a first active ingredient, which is a tricyclic imidazo[1,2-a]pyridine compound selected from the group consisting of the tricyclic imidazo[1,2-a]pyridine compounds which are specifically disclosed and/or individualized and/or claimed in the patent applications WO 9842707, WO 0017200, WO 0026217, WO 0063211, WO 0172756, WO 0172755, WO 0172757, WO 0234749, WO 03014120, WO 03014123, WO 03016310 and WO 03091253, and which are not substituted by a hydroxy-1-4C-alkyl radical bonded on the imidazo ring; or is a compound selected from the group consisting of
(7S,8R,9R)-2,3-dimethyl-7,8-dihydroxy-9-phenyl-7,8,9,10-tetrahydroimidazo[1,2-h][1,7]naphthyridine,
(7S,8R,9R)-7,8-isopropylidenedioxy-2,3-dimethyl-9-phenyl-7,8,9,10-tetrahydroimidazo[1,2-h][1,7]naphthyridine,
7,8-dihydroxy-9-phenyl-2,3-dimethyl-7H-8,9-dihydropyrano[2,3-c]imidazo[1,2-a]pyridine,
(7R,8R,9R)-2,3-dimethyl-8-hydroxy-7-methoxy-9-phenyl-7,8,9,10-tetrahydroimidazo[1,2-h][1,7]naphthyridine,
(7S,8S,9S)-2,3-dimethyl-8-hydroxy-7-methoxy-9-phenyl-7,8,9,10-tetrahydroimidazo[1,2-h][1,7]naphthyridine,
(7S,8R,9R)-2,3-dimethyl-8-hydroxy-7-methoxy-9-phenyl-7,8,9,10-tetrahydroimidazo[1,2-h][1,7]naphthyridine,
(7R,8S,9S)-2,3-dimethyl-8-hydroxy-7-methoxy-9-phenyl-7,8,9,10-tetrahydroimidazo[1,2-h][1,7]naphthyridine,
(7R,8R,9R)-2,3-dimethyl-7-ethoxy-8-hydroxy-9-phenyl-7,8,9,10-tetrahydroimidazo[1,2-h][1,7]naphthyridine,
(7S,8R,9R)-2,3-dimethyl-7-ethoxy-8-hydroxy-9-phenyl-7,8,9,10-tetrahydroimidazo[1,2-h][1,7]naphthyridine,
(7R,8R,9R)-2,3-dimethyl-8-hydroxy-7-(2-methoxyethoxy)-9-phenyl-7,8,9,10-tetrahydroimidazo[1,2-h][1,7]naphthyridine,
(7S,8S,9S)-2,3-dimethyl-8-hydroxy-7-(2-methoxyethoxy)-9-phenyl-7,8,9,10-tetrahydroimidazo[1,2-h][1,7]naphthyridine,
(7S,8R,9R)-2,3-dimethyl-8-hydroxy-7-(2-methoxyethoxy)-9-phenyl-7,8,9,10-tetrahydroimidazo[1,2-h][1,7]naphthyridine,
(7R,8S,9S)-2,3-dimethyl-8-hydroxy-7-(2-methoxyethoxy)-9-phenyl-7,8,9,10-tetrahydroimidazo[1,2-h][1,7]naphthyridine,
(7S,8R,9R)-2,3-dimethyl-8-hydroxy-9-phenyl-7-(2-propoxy)-7,8,9,10-tetrahydroimidazo[1,2-h][1,7]naphthyridine,
(7R,8R,9R)-2,3-dimethyl-7,8-dimethoxy-9-phenyl-7,8,9,10-tetrahydroimidazo[1,2-h][1,7]naphthyridine,
(7R,8R,9R)-2,3-dimethyl-8-hydroxy-7-(2-methylthioethyloxy)-9-phenyl-7,8,9,10-tetrahydroimidazo[1,2-h][1,7]-naphthyridine,
(7S,8R,9R)-2,3-dimethyl-8-hydroxy-7-(2-methylthioethyloxy)-9-phenyl-7,8,9,10-tetrahydroimidazo[1,2-h][1,7]-naphthyridine,
(7R,8R,9R)-2,3-dimethyl-8-hydroxy-7-(2-methylsulphinylethoxy)-9-phenyl-7,8,9,10-tetrahydroimidazo-[1,2-h][1,7]naphthyridine,
(7S,8R,9R)-2,3-dimethyl-8-hydroxy-7-(2-methylsulphinylethoxy)-9-phenyl-7,8,9,10-tetrahydroimidazo-[1,2-h][1,7]naphthyridine,
(7R,8R,9R)-2,3-dimethyl-8-hydroxy-7-(ethylthio)-9-phenyl-7,8,9,10-tetrahydroimidazo[1,2-h][1,7]naphthyridine,
(7S,8R,9R)-2,3-dimethyl-8-hydroxy-7-(ethylthio)-9-phenyl-7,8,9,10-tetrahydroimidazo[1,2-h][1,7]naphthyridine,
(7R,8R,9R)-2,3-dimethyl-8-hydroxy-7-(2,2,2-trifluoroethoxy)-9-phenyl-7,8,9,10-tetrahydroimidazo-[1,2-h][1,7]naphthyridine,
(7S,8R,9R)-2,3-dimethyl-8-hydroxy-7-(2,2,2-trifluoroethoxy)-9-phenyl-7,8,9,10-tetrahydroimidazo-[1,2-h][1,7]naphthyridine,
(7S,8R,9R)-8-acetoxy-7-(2-methoxyethoxy)-2,3-dimethyl-9-phenyl-7,8,9,10-tetrahydroimidazo[1,2-h][1,7]naphthyridine,
(7R,8R,9R)-8-acetoxy-7-(2-methoxyethoxy)-2,3-dimethyl-9-phenyl-7,8,9,10-tetrahydroimidazo[1,2-h][1,7]naphthyridine,
(7R,8R,9R)-8-acetoxy-7-methoxy-2,3-dimethyl-9-phenyl-7,8,9,10-tetrahydroimidazo[1,2-h][1,7]naphthyridine,
(7R,8R,9R)-8-acetoxy-7-ethoxy-2,3-dimethyl-9-phenyl-7,8,9,10-tetrahydroimidazo[1,2-h][1,7]naphthyridine,
(7R,8R,9R)-7-(2-methoxyethoxy)-2,3-dimethyl-9-phenyl-8-propionyloxy-7,8,9,10-tetrahydroimidazo[1,2-h]-[1,7]naphthyridine,
(7R,8R,9R)-8-benzoyloxy-7-(2-methoxyethoxy)-2,3-dimethyl-9-phenyl-7,8,9,10-tetrahydroimidazo[1,2-h][1,7]naphthyridine,
(7S,8R,9R)-8-benzoyloxy-7-(2-methoxyethoxy)-2,3-dimethyl-9-phenyl-7,8,9,10-tetrahydroimidazo[1,2-h][1,7]naphthyridine,
(7R,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,
(7S,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,
(7R,8R,9R)-8-benzoyloxy-7-methoxy-2,3-dimethyl-9-phenyl-7,8,9,10-tetrahydroimidazo[1,2-h][1,7]naphthyridine,
(7S,8R,9R)-8-benzoyloxy-7-methoxy-2,3-dimethyl-9-phenyl-7,8,9,10-tetrahydroimidazo[1,2-h][1,7]naphthyridine,
(7R,8R,9R)-7-(2-methoxyethoxy)-2,3-dimethyl-8-(4-nitrobenzoyloxy)-9-phenyl-7,8,9,10-tetrahydroimidazo[1,2-h][1,7]naphthyridine,
(7S,8R,9R)-7-(2-methoxyethoxy)-2,3-dimethyl-8-(4-nitrobenzoyloxy)-9-phenyl-7,8,9,10-tetrahydroimidazo[1,2-h][1,7]naphthyridine,
(7S,8R,9R)-7-(2-methoxyethoxy)-2,3-dimethyl-8-(3-nitrobenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(3-nitrobenzoyloxy)-9-phenyl-7,8,9,10-tetrahydroimidazo[1,2-h][1,7]naphthyridine,
(7S,8R,9R)-7-methoxy-2,3-dimethyl-8-(3-nitrobenzoyloxy)-9-phenyl-7,8,9,10-tetrahydroimidazo[1,2-h][1,7]naphthyridine,
(7R,8R,9R)-7-methoxy-2,3-dimethyl-8-(3-nitrobenzoyloxy)-9-phenyl-7,8,9,10-tetrahydroimidazo[1,2-h][1,7]naphthyridine,
(7S,8R,9R)-7-(2-methoxyethoxy)-2,3-dimethyl-8-(4-methoxybenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(4-methoxybenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,
(7S,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,
(7S,8R,9R)-7-(2-methoxyethoxy)-8-(N,N-diethylaminocarbonyloxy)-2,3-dimethyl-9-phenyl-7,8,9,10-tetrahydroimidazo[1,2-h][1,7]naphthyridine,
(7R,8R,9R)-7-(2-methoxyethoxy)-8-(N,N-diethylaminocarbonyloxy)-2,3-dimethyl-9-phenyl-7,8,9,10-tetrahydroimidazo[1,2-h][1,7]naphthyridine,
(7R,8R,9R)-8-ethylaminocarbonyloxy-7-(2-methoxyethoxy)-2,3-dimethyl-9-phenyl-7,8,9,10-tetrahydroimidazo[1,2-h][1,7]naphthyridine,
(7R,8R,9R)-8-benzoyloxy-2,3-dimethyl-7-(2-methoxyethoxy)-9-phenyl-7H-8,9-dihydropyrano[2,3-c]imidazo[1,2-a]pyridine,
(7S,8R,9R)-8-benzoyloxy-2,3-dimethyl-7-(2-methoxyethoxy)-9-phenyl-7H-8,9-dihydropyrano[2,3-c]imidazo[1,2-a]pyridine,
(7R,8R,9R)-8-[4-(methoxycarbonyl)-benzoyloxy]-2,3-dimethyl-7-(2-methoxyethoxy)-9-phenyl-7H-8,9-dihydropyrano[2,3-c]imidazo[1,2-a]pyridine,
(7S,8R,9R)-8-[4-(methoxycarbonyl)-benzoyloxy]-2,3-dimethyl-7-(2-methoxyethoxy)-9-phenyl-7H-8,9-dihydropyrano[2,3-c]imidazo[1,2-a]pyridine,
(7S,8R,9R)-2,3-dimethyl-7-methoxy-8-methoxyacetyloxy-9-phenyl-7,8,9,10-tetrahydroimidazo[1,2-h][1,7]naphthyridine,
(7R,8R,9R)-8-(N,N-diethylaminocarbonyloxy)-2,3-dimethyl-7-methoxy-9-phenyl-7,8,9,10-tetrahydroimidazo[1,2-h][1,7]naphthyridine,
(7S,8R,9R)-8-(N,N-diethylaminocarbonyloxy)-2,3-dimethyl-7-methoxy-9-phenyl-7,8,9,10-tetrahydroimidazo[1,2-h][1,7]naphthyridine,
(7R,8R,9R)-7-methoxy-8-methoxycarbonyloxy-2,3-dimethyl-9-phenyl-7,8,9,10-tetrahydroimidazo[1,2-h][1,7]naphthyridine,
(7S,8R,9R)-7-methoxy-8-methoxycarbonyloxy-2,3-dimethyl-9-phenyl-7,8,9,10-tetrahydroimidazo[1,2-h][1,7]naphthyridine,
(7R,8R,9R)-2,3-dimethyl-8-formyloxy-7-methoxy-9-phenyl-7,8,9,10-tetrahydroimidazo[1,2-h][1,7]naphthyridine,
(7S,8R,9R)-2,3-dimethyl-8-formyloxy-7-methoxy-9-phenyl-7,8,9,10-tetrahydroimidazo[1,2-h][1,7]naphthyridine,
(7R,8R,9R)-8-benzoyloxy-2,3-dimethyl-7-methoxy-9-phenyl-7,8,9,10-tetrahydroimidazo[1,2-h][1,7]naphthyridine,
(7R,8S,9R)-2,3,8-trimethyl-7,8-dihydroxy-9-phenyl-7,8,9,10-tetrahydroimidazo[1,2-h][1,7]naphthyridine,
(7S,8S,9R)-2,3-dimethyl-8-benzyl-7,8-dihydroxy-9-phenyl-7,8,9,10-tetrahydroimidazo[1,2-h][1,7]naphthyridine,
(7R,8S,9R)-2,3,8-trimethyl-7,8-0,0-isopropylidene-9-phenyl-7,8,9,10-tetrahydroimidazo[1,2-h][1,7]naphthyridine,
(7S,8S,9R)-2,3,8-trimethyl-7-(2-methoxyethoxy)-8-hydroxy-9-phenyl-7,8,9,10-tetrahydroimidazo[1,2-h][1,7]naphthyridine,
(7S,8S,9R)-2,3,8-trimethyl-7-methoxy-8-hydroxy-9-phenyl-7,8,9,10-tetrahydroimidazo[1,2-h][1,7]naphthyridine,
(7R,8R,9R)-2,3,7-trimethyl-7,8-dihydroxy-9-phenyl-7,8,9,10-tetrahydroimidazo[1,2-h][1,7]naphthyridine,
(7R,8R,9R)-2,3,7-trimethyl-7,8-[1,3]dioxolo-9-phenyl-7,8,9,10-tetrahydroimidazo[1,2-h][1,7]naphthyridine,
(8S,9R)-2,3-dimethyl-8-hydroxy-7-methylidene-9-phenyl-7,8,9,10-tetrahydroimidazo[1,2-h][1,7]naphthyridine,
(7S,8R,9R)-2,3,7-trimethyl-7,8-dihydroxy-9-phenyl-7H-8,9-dihydropyrano[2,3-c]imidazo[1,2-a]pyridine,
(7R,8R,9R)-2,3,7-trimethyl-7,8-dihydroxy-9-phenyl-7H-8,9-dihydropyrano[2,3-c]imidazo[1,2-a]pyridine,
(7S,8R,9R)-2,3-dimethyl-7,8-dihydroxy-7,9-diphenyl-7H-8,9-dihydropyrano[2,3-c]imidazo[1,2-a]pyridine,
(7S,8R,9R)-2,3-dimethyl-7-(2′,2′-dimethylvinyl)-7,8-dihydroxy-9-phenyl-7H-8,9-dihydropyrano[2,3-c]imidazo[1,2-a]pyridine,
(7R,8R,9R)-2,3-dimethyl-7,8-O-isopropylidene-9-phenyl-7-vinyl-7H-8,9-dihydropyrano[2,3-c]imidazo[1,2-a]pyridine,
(7R,8R,9R)-2,3-dimethyl-8-hydroxy-7-(2-methoxyethoxy)-9-phenyl-7H-8,9-dihydropyrano[2,3-c]imidazo[1,2-a]pyridine,
(7S,8R,9R)-2,3-dimethyl-8-hydroxy-7-(2-methoxyethoxy)-9-phenyl-7H-8,9-dihydropyrano[2,3-c]imidazo[1,2-a]pyridine,
(7R,8R,9R)-2,3-dimethyl-8-hydroxy-7-ethoxy-9-phenyl-7H-8,9-dihydropyrano[2,3-c]imidazo[1,2-a]pyridine,
(7S,8R,9R)-2,3-dimethyl-8-hydroxy-7-ethoxy-9-phenyl-7H-8,9-dihydropyrano[2,3-c]imidazo[1,2-a]pyridine,
(7R,8R,9R)-2,3-dimethyl-8-hydroxy-7-(2-methoxypropoxy)-9-phenyl-7H-8,9-dihydropyrano[2,3-c]imidazo[1,2-a]pyridine,
(7S,8R,9R)-2,3-dimethyl-8-hydroxy-7-(2-methoxypropoxy)-9-phenyl-7H-8,9-dihydropyrano[2,3-c]imidazo[1,2-a]pyridine,
(7R,8R,9R)-2,3-dimethyl-8-hydroxy-7-(2-propoxy)-9-phenyl-7H-8,9-dihydropyrano[2,3-c]imidazo[1,2-a]pyridine,
(7S,8R,9R)-2,3-dimethyl-8-hydroxy-7-(2-propoxy)-9-phenyl-7H-8,9-dihydropyrano[2,3-c]imidazo[1,2-a]pyridine,
(7R,8R,9R)-2,3-dimethyl-8-hydroxy-7-butoxy-9-phenyl-7H-8,9-dihydropyrano[2,3-c]imidazo[1,2-a]pyridine,
(7S,8R,9R)-2,3-dimethyl-8-hydroxy-7-butoxy-9-phenyl-7H-8,9-dihydropyrano[2,3-c]imidazo[1,2-a]pyridine,
(7S,8R,9R)-7,8-dihydroxy-6-methoxymethyl-2,3-dimethyl-9-phenyl-7,8,9,10-tetrahydroimidazo[1,2-h][1,7]naphthyridine,
(7R,8R,9R)-7,8-dihydroxy-6-methoxymethyl-2,3-dimethyl-9-phenyl-7,8,9,10-tetrahydroimidazo[1,2-h][1,7]naphthyridine,
(7S,8R,9R)-8-hydroxy-7-methoxy-6-methoxymethyl-2,3-dimethyl-9-phenyl-7,8,9,10-tetrahydroimidazo[1,2-h][1,7]naphthyridine,
(7R,8R,9R)-8-hydroxy-7-methoxy-6-methoxymethyl-2,3-dimethyl-9-phenyl-7,8,9,10-tetrahydroimidazo[1,2-h][1,7]naphthyridine,
(7R,8R,9R)-8-hydroxy-7-(2-methoxyethoxy)-6-methoxymethyl-2,3-dimethyl-9-phenyl-7,8,9,10-tetrahydroimidazo[1,2-h][1,7]naphthyridine,
(7S,8R,9R)-8-hydroxy-7-(2-methoxyethoxy)-6-methoxymethyl-2,3-dimethyl-9-phenyl-7,8,9,10-tetrahydroimidazo[1,2-h][1,7]naphthyridine,
(7R,8R,9R)-8-hydroxy-7-ethoxy-6-methoxymethyl-2,3-dimethyl-9-phenyl-7,8,9,10-tetrahydro-imidazo[1,2-h][1,7]naphthyridine,
(7S,8R,9R)-8-hydroxy-7-ethoxy-6-methoxymethyl-2,3-dimethyl-9-phenyl-7,8,9,10-tetrahydro-imidazo[1,2-h]-[1,7]naphthyridine,
7,8-dihydroxy-2,3-dimethyl-9-(3-thienyl)-7,8,9,10-tetrahydroimidazo[1,2-h][1,7]naphthyridine,
7-hydroxy-2,3-dimethyl-9-(3-thienyl)-7,8,9,10-tetrahydroimidazo[1,2-h][1,7]naphthyridine,
9-(3-furyl)-7-hydroxy-2,3-dimethyl-7,8,9,10-tetrahydroimidazo[1,2-h][1,7]naphthyridine,
(7R,8R,9R)-8-hydroxy-7-[2-(2-methoxyethoxy)ethoxy]-2,3-dimethyl-9-phenyl-7,8,9,10-tetrahydroimidazo[1,2-h][1,7]naphthyridine,
(7S,8R,9R)-8-hydroxy-7-[2-(2-methoxyethoxy)ethoxy]-2,3-dimethyl-9-phenyl-7,8,9,10-tetrahydroimidazo[1,2-h][1,7]naphthyridine,
(7R,8R,9R)-7,8-dihydroxy-2-methyl-9-phenyl-7,8,9,10-tetrahydroimidazo[1,2-h][1,7]naphthyridine,
(7S,8R,9R)-8-hydroxy-2-methyl-7-(2-methoxyethoxy)-9-phenyl-7,8,9,10-tetrahydroimidazo[1,2-h][1,7]naphthyridine,
(7R,8R,9R)-8-hydroxy-2-methyl-7-(2-methoxyethoxy)-9-phenyl-7,8,9,10-tetrahydroimidazo[1,2-h][1,7]naphthyridine,
(7R,8R,9R)-3-bromo-8-hydroxy-7-(2-methoxyethoxy)-2-methyl-9-phenyl-7, 8.9, 10-tetrahydroimidazo-[1,2-h][1,7]naphthyridine,
(7R,8R,9R)-3-chloro-8-hydroxy-7-(2-methoxyethoxy)-2-methyl-9-phenyl-7,8,9,10-tetrahydroimidazo-[1,2-h][1,7]naphthyridine,
(7R,8R,9R)-3-bromo-7-hydroxy-8-(2-methoxyethoxy)-2-methyl-9-phenyl-7,8,9,10-tetrahydroimidazo-[1,2-h][1,7]naphthyridine,
(7R,8R,9R)-3-chloro-8-hydroxy-7-(2-methoxyethoxy)-2-methyl-9-phenyl-7H-8,9-dihydro-pyrano[2,3-c]imidazo[1,2-a]pyridine,
(7R,8R,9R)-8-hydroxy-7-(2-methoxyethoxy)-2-methyl-9-phenyl-7H-8,9-dihydro-pyrano[2,3-c]imidazo[1,2-a]pyridine,
(7R,8R,9R)-7,8-dihydroxy-2-methyl-9-phenyl-7H-8,9-dihydropyrano[2,3-c]imidazo[1,2-a]pyridine,
(7S,8R,9R)-7,8-dihydroxy-2-methyl-9-phenyl-7,8,9,10-tetrahydroimidazo[1,2-h][1,7]naphthyridine,
(7R,8R,9R)-8-hydroxy-7-methoxy-2-methyl-9-phenyl-7,8,9,10-tetrahydroimidazo[1,2-h][1,7]naphthyridine,
(7S,8R,9R)-8-hydroxy-7-methoxy-2-methyl-9-phenyl-7,8,9,10-tetrahydroimidazo[1,2-h][1,7]naphthyridine,
(7R,8R,9R)-2,3-dimethyl-8-hydroxy-7-(2-hydroxyethoxy)-9-phenyl-7,8,9,10-tetrahydroimidazo[1,2-h][1,7]naphthyridine,
(7R,8R,9R)-3,9-diphenyl-8-hydroxy-7-(2-methoxyethoxy)-2-methyl-7,8,9,10-tetrahydroimidazo[1,2-h][1,7]naphthyridine,
(7R,8R,9R)-7,8-dihydroxy-2-methoxymethyl-3-methyl-9-phenyl-7,8,9,10-tetrahydroimidazo[1,2-h][1,7]naphthyridine,
(7S,8R,9R)-8-hydroxy-7-(2-methoxyethoxy)-2-methoxymethyl-3-methyl-9-phenyl-7,8,9,10-tetrahydroimidazo[1,2-h][1,7]naphthyridine,
(7R,8R,9R)-8-hydroxy-7-(2-methoxyethoxy)-2-methoxymethyl-3-methyl-9-phenyl-7,8,9,10-tetrahydroimidazo[1,2-h][1,7]naphthyridine,
(7S,8R,9R)-7-ethoxy-8-hydroxy-2-methoxymethyl-3-methyl-9-phenyl-7,8,9,10-tetrahydroimidazo[1,2-h][1,7]naphthyridine,
(7R,8R,9R)-7-ethoxy-8-hydroxy-2-methoxymethyl-3-methyl-9-phenyl-7,8,9,10-tetrahydroimidazo[1,2-h][1,7]naphthyridine,
(7R,8S,9R)-10-acetyl-8-hydroxy-2,3-dimethyl-7-(4-morpholino)-7,8,9,10-tetrahydroimidazo[1,2-h][1,7]naphthyridine,
(7R,8S,9R)-8-hydroxy-2,3-dimethyl-7-(4-morpholino)-7,8,9,10-tetrahydroimidazo[1,2-h][1,7]naphthyridine,
(7R,8S,9R)-10-acetyl-8-hydroxy-2,3-dimethyl-7-methylamino-7,8,9,10-tetrahydro-imidazo[1,2-h][1,7]naphthyridine,
(7R,8S,9R)-8-hydroxy-2,3-dimethyl-7-methylamino-7,8,9,10-tetrahydroimidazo[1,2-h][1,7]naphthyridine,
(7R,8S,9R)-10-acetyl-8-hydroxy-2,3-dimethyl-7-(1-pyrrolidino)-7,8,9,10-tetrahydroimidazo[1,2-h][1,7]naphthyridine,
(7R,8S,9R)-8-hydroxy-2,3-dimethyl-7-(1-pyrrolidino)-7,8,9,10-tetrahydroimidazo[1,2-h][1,7]naphthyridine,
(7R,8S,9R)-10-acetyl-7-benzylamino-8-hydroxy-2,3-dimethyl-7,8,9,10-tetrahydro-imidazo[1,2-h][1,7]naphthyridine,
(7R,8S,9R)-7-benzylamino-8-hydroxy-2,3-dimethyl-7,8,9,10-tetrahydroimidazo[1,2-h][1,7]naphthyridine,
(7R,8S,9R)-10-acetyl-8-hydroxy-7-(2-methoxyethylamino)-2,3-dimethyl-7,8,9,10-tetrahydroimidazo[1,2-h][1,7]naphthyridine,
(7R,8S,9R)-8-hydroxy-7-(2-methoxyethylamino)-2,3-dimethyl-7,8,9,10-tetrahydroimidazo[1,2-h][1,7]naphthyridine,
(7R,8S,9R)-10-acetyl-7-(dimethylamino)-8-hydroxy-2,3-dimethyl-7,8,9,10-tetrahydro-imidazo[1,2-h][1,7]naphthyridine,
(7R,8S,9R)-8-hydroxy-7-(dimethylamino)-2,3-dimethyl-7,8,9,10-tetrahydroimidazo[1,2-h][1,7]naphthyridine,
(7S,8S,9R)-8-hydroxy-2,3,7-trimethyl-7,8,9,10-tetrahydroimidazo[1,2-h][1,7]naphthyridine,
(7S,8S,9R)-7-cyanomethyl-8-hydroxy-2,3-dimethyl-7,8,9,10-tetrahydroimidazo[1,2-h][1,7]naphthyridine,
(7S,8S,9R)-8-hydroxy-2,3-dimethyl-7-propyl-7,8,9,10-tetrahydroimidazo[1,2-h][1,7]naphthyridine,
(7R,8S,9R)-8-hydroxy-2,3-dimethyl-7-(3-methoxypropyl)-7,8,9,10-tetrahydroimidazo-[1,2-h][1,7]naphthyridine,
2,3-dimethyl-9-phenyl-7H-8,9-dihydro-pyrano-[2,3-c]-N-(diethyl)imidazo[1,2-a]pyridine-6-carboxamide,
ethyl 2,3-dimethyl-9-phenyl-7H-8,9-dihydro-pyrano[2,3-c]-imidazo[1,2-a]pyridine-6-carboxylate,
2,3-dimethyl-9-phenyl-7H-8,9-dihydro-pyrano[2,3-c]-imidazo[1,2-a]pyridine-6-(N,N-dimethyl)carbamide,
(7R,8R,9R)-2,3-dimethyl-7(2-methoxyethoxy)-9-phenyl-8-(5-nitrooxy-valeryloxy)-7,8,9,10-tetrahydro-imidazo[1,2-h][1,7]naphthyridine,
(7R,8R,9R)-2,3-dimethyl-7(2-methoxyethoxy)-9-phenyl-8-(4-nitrooxy-butyryloxy)-7,8,9,10-tetrahydro-imidazo[1,2-h][1,7]naphthyridine,
(7R,8R,9R)-2,3-dimethyl-7-(2-methoxyethoxy)-9-phenyl-8-(5-nitro-oxy-valeryloxy)-7H-8,9-dihydro-pyrano[2,3-c]imdazo[1,2-a]pyridine,
(7R,8R,9R)-2,3-dimethyl-7-(2-methoxyethoxy)-9-phenyl-8-(6-nitro-oxy-2-oxa-capryloxy)-7,8,9,10-tetrahydro-imidazo[1,2-h][1,7]naphthyridine,
(7R,8R,9R)-2,3-dimethyl-7-(2-methoxyethoxy)-9-phenyl-8-(4-nitro-oxymethyl-benzoyloxy)-7,8,9,10-tetrahydroimidazo[1,2-h][1,7]naphthyridine,
and the salts, hydrates, solvates, hydrates of the salts and solvates of the salts of these compounds;
and a second active ingredient, which is selected from the group consisting of NSAIDs, COX-2 inhibitors, NO-NSAIDs, bisphosphonates and corticosteroids.

2. A pharmaceutical composition according to claim 1 comprising a first active ingredient, which is a tricyclic imidazo[1,2-a]pyridine compound selected from the group consisting of
(7S,8R,9R)-2,3-dimethyl-7,8-dihydroxy-9-phenyl-7,8,9,10-tetrahydroimidazo[1,2-h][1,7]naphthyridine,
(7S,8R,9R)-7,8-isopropylidenedioxy-2,3-dimethyl-9-phenyl-7,8,9,10-tetrahydroimidazo[1,2-h][1,7]naphthyridine,
7,8-dihydroxy-9-phenyl-2,3-dimethyl-7H-8,9-dihydropyrano[2,3-c]imidazo[1,2-a]pyridine,
(7R,8R,9R)-2,3-dimethyl-8-hydroxy-7-methoxy-9-phenyl-7,8,9,10-tetrahydroimidazo[1,2-h][1,7]naphthyridine,
(7S,8S,9S)-2,3-dimethyl-8-hydroxy-7-methoxy-9-phenyl-7,8,9,10-tetrahydroimidazo[1,2-h][1,7]naphthyridine,
(7S,8R,9R)-2,3-dimethyl-8-hydroxy-7-methoxy-9-phenyl-7,8,9,10-tetrahydroimidazo[1,2-h][1,7]naphthyridine,
(7R,8S,9S)-2,3-dimethyl-8-hydroxy-7-methoxy-9-phenyl-7,8,9,10-tetrahydroimidazo[1,2-h][1,7]naphthyridine,
(7R,8R,9R)-2,3-dimethyl-7-ethoxy-8-hydroxy-9-phenyl-7,8,9,10-tetrahydroimidazo[1,2-h][1,7]naphthyridine,
(7S,8R,9R)-2,3-dimethyl-7-ethoxy-8-hydroxy-9-phenyl-7,8,9,10-tetrahydroimidazo[1,2-h][1,7]naphthyridine,
(7R,8R,9R)-2,3-dimethyl-8-hydroxy-7-(2-methoxyethoxy)-9-phenyl-7,8,9,10-tetrahydroimidazo[1,2-h][1,7]naphthyridine,
(7S,8S,9S)-2,3-dimethyl-8-hydroxy-7-(2-methoxyethoxy)-9-phenyl-7,8,9,10-tetrahydroimidazo[1,2-h][1,7]naphthyridine,
(7S,8R,9R)-2,3-dimethyl-8-hydroxy-7-(2-methoxyethoxy)-9-phenyl-7,8,9,10-tetrahydroimidazo[1,2-h][1,7]naphthyridine,
(7R,8S,9S)-2,3-dimethyl-8-hydroxy-7-(2-methoxyethoxy)-9-phenyl-7,8,9,10-tetrahydroimidazo[1,2-h][1,7]naphthyridine,
(7S,8R,9R)-2,3-dimethyl-8-hydroxy-9-phenyl-7-(2-propoxy)-7,8,9,10-tetrahydroimidazo[1,2-h][1,7]naphthyridine,
(7R,8R,9R)-2,3-dimethyl-7,8-dimethoxy-9-phenyl-7,8,9,10-tetrahydroimidazo[1,2-h][1,7]naphthyridine,
(7R,8R,9R)-2,3-dimethyl-8-hydroxy-7-(2-methylthioethyloxy)-9-phenyl-7,8,9,10-tetrahydroimidazo[1,2-h][1,7]-naphthyridine,
(7S,8R,9R)-2,3-dimethyl-8-hydroxy-7-(2-methylthioethyloxy)-9-phenyl-7,8,9,10-tetrahydroimidazo[1,2-h][1,7]-naphthyridine,
(7R,8R,9R)-2,3-dimethyl-8-hydroxy-7-(2-methylsulphinylethoxy)-9-phenyl-7,8,9,10-tetrahydroimidazo-[1,2-h][1,7]naphthyridine,
(7S,8R,9R)-2,3-dimethyl-8-hydroxy-7-(2-methylsulphinylethoxy)-9-phenyl-7,8,9,10-tetrahydroimidazo-[1,2-h][1,7]naphthyridine,
(7R,8R,9R)-2,3-dimethyl-8-hydroxy-7-(ethylthio)-9-phenyl-7,8,9,10-tetrahydroimidazo[1,2-h][1,7]naphthyridine,
(7S,8R,9R)-2,3-dimethyl-8-hydroxy-7-(ethylthio)-9-phenyl-7,8,9,10-tetrahydroimidazo[1,2-h][1,7]naphthyridine,
(7R,8R,9R)-2,3-dimethyl-8-hydroxy-7-(2,2,2-trifluoroethoxy)-9-phenyl-7,8,9,10-tetrahydroimidazo-[1,2-h][1,7]naphthyridine,
(7S,8R,9R)-2,3-dimethyl-8-hydroxy-7-(2,2,2-trifluoroethoxy)-9-phenyl-7,8,9,10-tetrahydroimidazo-[1,2-h][1,7]naphthyridine,
(7S,8R,9R)-8-acetoxy-7-(2-methoxyethoxy)-2,3-dimethyl-9-phenyl-7,8,9,10-tetrahydroimidazo[1,2-h][1,7]naphthyridine,
(7R,8R,9R)-8-acetoxy-7-(2-methoxyethoxy)-2,3-dimethyl-9-phenyl-7,8,9,10-tetrahydroimidazo[1,2-h][1,7]naphthyridine,
(7R,8R,9R)-8-acetoxy-7-methoxy-2,3-dimethyl-9-phenyl-7,8,9,10-tetrahydroimidazo[1,2-h][1,7]naphthyridine,
(7R,8R,9R)-8-acetoxy-7-ethoxy-2,3-dimethyl-9-phenyl-7,8,9,10-tetrahydroimidazo[1,2-h][1,7]naphthyridine,
(7R,8R,9R)-7-(2-methoxyethoxy)-2,3-dimethyl-9-phenyl-8-propionyloxy-7,8,9,10-tetrahydroimidazo[1,2-h]-[1,7]naphthyridine,
(7R,8R,9R)-8-benzoyloxy-7-(2-methoxyethoxy)-2,3-dimethyl-9-phenyl-7,8,9,10-tetrahydroimidazo[1,2-h][1,7]naphthyridine,
(7S,8R,9R)-8-benzoyloxy-7-(2-methoxyethoxy)-2,3-dimethyl-9-phenyl-7,8,9,10-tetrahydroimidazo[1,2-h][1,7]naphthyridine,
(7R,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,
(7S,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,
(7R,8R,9R)-8-benzoyloxy-7-methoxy-2,3-dimethyl-9-phenyl-7,8,9,10-tetrahydroimidazo[1,2-h][1,7]naphthyridine,
(7S,8R,9R)-8-benzoyloxy-7-methoxy-2,3-dimethyl-9-phenyl-7,8,9,10-tetrahydroimidazo[1,2-h][1,7]naphthyridine,
(7R,8R,9R)-7-(2-methoxyethoxy)-2,3-dimethyl-8-(4-nitrobenzoyloxy)-9-phenyl-7,8,9,10-tetrahydroimidazo[1,2-h][1,7]naphthyridine,
(7S,8R,9R)-7-(2-methoxyethoxy)-2,3-dimethyl-8-(4-nitrobenzoyloxy)-9-phenyl-7,8,9,10-tetrahydroimidazo[1,2-h][1,7]naphthyridine,
(7S,8R,9R)-7-(2-methoxyethoxy)-2,3-dimethyl-8-(3-nitrobenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(3-nitrobenzoyloxy)-9-phenyl-7,8,9,10-tetrahydroimidazo[1,2-h][1,7]naphthyridine,
(7S,8R,9R)-7-methoxy-2,3-dimethyl-8-(3-nitrobenzoyloxy)-9-phenyl-7,8,9,10-tetrahydroimidazo[1,2-h][1,7]naphthyridine,
(7R,8R,9R)-7-methoxy-2,3-dimethyl-8-(3-nitrobenzoyloxy)-9-phenyl-7,8,9,10-tetrahydroimidazo[1,2-h][1,7]naphthyridine,
(7S,8R,9R)-7-(2-methoxyethoxy)-2,3-dimethyl-8-(4-methoxybenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(4-methoxybenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,
(7S,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,
(7S,8R,9R)-7-(2-methoxyethoxy)-8-(N,N-diethylaminocarbonyloxy)-2,3-dimethyl-9-phenyl-7,8,9,10-tetrahydroimidazo[1,2-h][1,7]naphthyridine,
(7R,8R,9R)-7-(2-methoxyethoxy)-8-(N,N-diethylaminocarbonyloxy)-2,3-dimethyl-9-phenyl-7,8,9,10-tetrahydroimidazo[1,2-h][1,7]naphthyridine,
(7R,8R,9R)-8-ethylaminocarbonyloxy-7-(2-methoxyethoxy)-2,3-dimethyl-9-phenyl-7,8,9,10-tetrahydroimidazo[1,2-h][1,7]naphthyridine,
(7R,8R,9R)-8-benzoyloxy-2,3-dimethyl-7-(2-methoxyethoxy)-9-phenyl-7H-8,9-dihydropyrano[2,3-c]imidazo[1,2-a]pyridine,
(7S,8R,9R)-8-benzoyloxy-2,3-dimethyl-7-(2-methoxyethoxy)-9-phenyl-7H-8,9-dihydropyrano[2,3-c]imidazo[1,2-a]pyridine,
(7R,8R,9R)-8-[4-(methoxycarbonyl)-benzoyloxy]-2,3-dimethyl-7-(2-methoxyethoxy)-9-phenyl-7H-8,9-dihydropyrano[2,3-c]imidazo[1,2-a]pyridine,
(7S,8R,9R)-8-[4-(methoxycarbonyl)-benzoyloxy]-2,3-dimethyl-7-(2-methoxyethoxy)-9-phenyl-7H-8,9-dihydropyrano[2,3-c]imidazo[1,2-a]pyridine,
(7S,8R,9R)-2,3-dimethyl-7-methoxy-8-methoxyacetyloxy-9-phenyl-7,8,9,10-tetrahydroimidazo[1,2-h][1,7]naphthyridine,
(7R,8R,9R)-8-(N,N-diethylaminocarbonyloxy)-2,3-dimethyl-7-methoxy-9-phenyl-7,8,9,10-tetrahydroimidazo[1,2-h][1,7]naphthyridine,
(7S,8R,9R)-8-(N,N-diethylaminocarbonyloxy)-2,3-dimethyl-7-methoxy-9-phenyl-7,8,9,10-tetrahydroimidazo[1,2-h][1,7]naphthyridine,
(7R,8R,9R)-7-methoxy-8-methoxycarbonyloxy-2,3-dimethyl-9-phenyl-7,8,9,10-tetrahydroimidazo[1,2-h][1,7]naphthyridine,
(7S,8R,9R)-7-methoxy-8-methoxycarbonyloxy-2,3-dimethyl-9-phenyl-7,8,9,10-tetrahydroimidazo[1,2-h][1,7]naphthyridine,
(7R,8R,9R)-2,3-dimethyl-8-formyloxy-7-methoxy-9-phenyl-7,8,9,10-tetrahydroimidazo[1,2-h][1,7]naphthyridine,
(7S,8R,9R)-2,3-dimethyl-8-formyloxy-7-methoxy-9-phenyl-7,8,9,10-tetrahydroimidazo[1,2-h][1,7]naphthyridine,
(7R,8R,9R)-8-benzoyloxy-2,3-dimethyl-7-methoxy-9-phenyl-7,8,9,10-tetrahydroimidazo[1,2-h][1,7]naphthyridine,
(7R,8S,9R)-2,3,8-trimethyl-7,8-dihydroxy-9-phenyl-7,8,9,10-tetrahydroimidazo[1,2-h][1,7]naphthyridine,
(7S,8S,9R)-2,3-dimethyl-8-benzyl-7,8-dihydroxy-9-phenyl-7,8,9,10-tetrahydroimidazo[1,2-h][1,7]naphthyridine,
(7R,8S,9R)-2,3,8-trimethyl-7,8-0,0-isopropylidene-9-phenyl-7,8,9,10-tetrahydroimidazo[1,2-h][1,7]naphthyridine,
(7S,8S,9R)-2,3,8-trimethyl-7-(2-methoxyethoxy)-8-hydroxy-9-phenyl-7,8,9,10-tetrahydroimidazo[1,2-h][1,7]naphthyridine,
(7S,8S,9R)-2,3,8-trimethyl-7-methoxy-8-hydroxy-9-phenyl-7,8,9,10-tetrahydroimidazo[1,2-h][1,7]naphthyridine,
(7R,8R,9R)-2,3,7-trimethyl-7,8-dihydroxy-9-phenyl-7,8,9,10-tetrahydroimidazo[1,2-h][1,7]naphthyridine,
(7R,8R,9R)-2,3,7-trimethyl-7,8-[1,3]dioxolo-9-phenyl-7,8,9,10-tetrahydroimidazo[1,2-h][1,7]naphthyridine,
(8S,9R)-2,3-dimethyl-8-hydroxy-7-methylidene-9-phenyl-7,8,9,10-tetrahydroimidazo[1,2-h][1,7]naphthyridine,
(7S,8R,9R)-2,3,7-trimethyl-7,8-dihydroxy-9-phenyl-7H-8,9-dihydropyrano[2,3-c]imidazo[1,2-a]pyridine,
(7R,8R,9R)-2,3,7-trimethyl-7,8-dihydroxy-9-phenyl-7H-8,9-dihydropyrano[2,3-c]imidazo[1,2-a]pyridine,
(7S,8R,9R)-2,3-dimethyl-7,8-dihydroxy-7,9-diphenyl-7H-8,9-dihydropyrano[2,3-c]imidazo[1,2-a]pyridine,
(7S,8R,9R)-2,3-dimethyl-7-(2′,2′-dimethylvinyl)-7,8-dihydroxy-9-phenyl-7H-8,9-dihydropyrano[2,3-c]imidazo[1,2-a]pyridine,
(7R,8R,9R)-2,3-dimethyl-7,8-O-isopropylidene-9-phenyl-7-vinyl-7H-8,9-dihydropyrano[2,3-c]imidazo[1,2-a]pyridine,
(7R,8R,9R)-2,3-dimethyl-8-hydroxy-7-(2-methoxyethoxy)-9-phenyl-7H-8,9-dihydropyrano[2,3-c]imidazo[1,2-a]pyridine,
(7S,8R,9R)-2,3-dimethyl-8-hydroxy-7-(2-methoxyethoxy)-9-phenyl-7H-8,9-dihydropyrano[2,3-c]imidazo[1,2-a]pyridine,
(7R,8R,9R)-2,3-dimethyl-8-hydroxy-7-ethoxy-9-phenyl-7H-8,9-dihydropyrano[2,3-c]imidazo[1,2-a]pyridine,
(7S,8R,9R)-2,3-dimethyl-8-hydroxy-7-ethoxy-9-phenyl-7H-8,9-dihydropyrano[2,3-c]imidazo[1,2-a]pyridine,
(7R,8R,9R)-2,3-dimethyl-8-hydroxy-7-(2-methoxypropoxy)-9-phenyl-7H-8,9-dihydropyrano[2,3-c]imidazo[1,2-a]pyridine,
(7S,8R,9R)-2,3-dimethyl-8-hydroxy-7-(2-methoxypropoxy)-9-phenyl-7H-8,9-dihydropyrano[2,3-c]imidazo[1,2-a]pyridine,
(7R,8R,9R)-2,3-dimethyl-8-hydroxy-7-(2-propoxy)-9-phenyl-7H-8,9-dihydropyrano[2,3-c]imidazo[1,2-a]pyridine,
(7S,8R,9R)-2,3-dimethyl-8-hydroxy-7-(2-propoxy)-9-phenyl-7H-8,9-dihydropyrano[2,3-c]imidazo[1,2-a]pyridine,
(7R,8R,9R)-2,3-dimethyl-8-hydroxy-7-butoxy-9-phenyl-7H-8,9-dihydropyrano[2,3-c]imidazo[1,2-a]pyridine,
(7S,8R,9R)-2,3-dimethyl-8-hydroxy-7-butoxy-9-phenyl-7H-8,9-dihydropyrano[2,3-c]imidazo[1,2-a]pyridine,
(7S,8R,9R)-7,8-dihydroxy-6-methoxymethyl-2,3-dimethyl-9-phenyl-7,8,9,10-tetrahydroimidazo[1,2-h][1,7]naphthyridine,
(7R,8R,9R)-7,8-dihydroxy-6-methoxymethyl-2,3-dimethyl-9-phenyl-7,8,9,10-tetrahydroimidazo[1,2-h][1,7]naphthyridine,
(7S,8R,9R)-8-hydroxy-7-methoxy-6-methoxymethyl-2,3-dimethyl-9-phenyl-7,8,9,10-tetrahydroimidazo[1,2-h][1,7]naphthyridine,
(7R,8R,9R)-8-hydroxy-7-methoxy-6-methoxymethyl-2,3-dimethyl-9-phenyl-7,8,9,10-tetrahydroimidazo[1,2-h][1,7]naphthyridine,
(7R,8R,9R)-8-hydroxy-7-(2-methoxyethoxy)-6-methoxymethyl-2,3-dimethyl-9-phenyl-7,8,9,10-tetrahydroimidazo[1,2-h][1,7]naphthyridine,
(7S,8R,9R)-8-hydroxy-7-(2-methoxyethoxy)-6-methoxymethyl-2,3-dimethyl-9-phenyl-7,8,9,10-tetrahydroimidazo[1,2-h][1,7]naphthyridine,
(7R,8R,9R)-8-hydroxy-7-ethoxy-6-methoxymethyl-2,3-dimethyl-9-phenyl-7,8,9,10-tetrahydro-imidazo[1,2-h][1,7]naphthyridine,
(7S,8R,9R)-8-hydroxy-7-ethoxy-6-methoxymethyl-2,3-dimethyl-9-phenyl-7,8,9,10-tetrahydro-imidazo[1,2-h]-[1,7]naphthyridine,
7,8-dihydroxy-2,3-dimethyl-9-(3-thienyl)-7,8,9,10-tetrahydroimidazo[1,2-h][1,7]naphthyridine,
7-hydroxy-2,3-dimethyl-9-(3-thienyl)-7,8,9,10-tetrahydroimidazo[1,2-h][1,7]naphthyridine,
9-(3-furyl)-7-hydroxy-2,3-dimethyl-7,8,9,10-tetrahydroimidazo[1,2-h][1,7]naphthyridine,
(7R,8R,9R)-8-hydroxy-7-[2-(2-methoxyethoxy)ethoxy]-2,3-dimethyl-9-phenyl-7,8,9,10-tetrahydroimidazo[1,2-h][1,7]naphthyridine,
(7S,8R,9R)-8-hydroxy-7-[2-(2-methoxyethoxy)ethoxy]-2,3-dimethyl-9-phenyl-7,8,9,10-tetrahydroimidazo[1,2-h)[1,7]naphthyridine,
(7R,8R,9R)-7,8-dihydroxy-2-methyl-9-phenyl-7,8,9,10-tetrahydroimidazo[1,2-h][1,7]naphthyridine,
(7S,8R,9R)-8-hydroxy-2-methyl-7-(2-methoxyethoxy)-9-phenyl-7,8,9,10-tetrahydroimidazo[1,2-h][1,7]naphthyridine,
(7R,8R,9R)-8-hydroxy-2-methyl-7-(2-methoxyethoxy)-9-phenyl-7,8,9,10-tetrahydroimidazo[1,2-h][1,7]naphthyridine,
(7R,8R,9R)-3-bromo-8-hydroxy-7-(2-methoxyethoxy)-2-methyl-9-phenyl-7,8,9,10-tetrahydroimidazo-[1,2-h][1,7]naphthyridine,
(7R,8R,9R)-3-chloro-8-hydroxy-7-(2-methoxyethoxy)-2-methyl-9-phenyl-7,8,9,10-tetrahydroimidazo-[1,2-h][1,7]naphthyridine,
(7R,8R,9R)-3-bromo-7-hydroxy-8-(2-methoxyethoxy)-2-methyl-9-phenyl-7,8,9,10-tetrahydroimidazo-[1,2-h][1,7]naphthyridine,
(7R,8R,9R)-3-chloro-8-hydroxy-7-(2-methoxyethoxy)-2-methyl-9-phenyl-7H-8,9-dihydro-pyrano[2,3-c]imidazo[1,2-a]pyridine,
(7R,8R,9R)-8-hydroxy-7-(2-methoxyethoxy)-2-methyl-9-phenyl-7H-8,9-dihydro-pyrano[2,3-c]imidazo[1,2-a]pyridine,
(7R,8R,9R)-7,8-dihydroxy-2-methyl-9-phenyl-7H-8,9-dihydropyrano[2,3-c]imidazo[1,2-a]pyridine,
(7S,8R,9R)-7,8-dihydroxy-2-methyl-9-phenyl-7,8,9,10-tetrahydroimidazo[1,2-h][1,7]naphthyridine,
(7R,8R,9R)-8-hydroxy-7-methoxy-2-methyl-9-phenyl-7,8,9,10-tetrahydroimidazo[1,2-h][1,7]naphthyridine,
(7S,8R,9R)-8-hydroxy-7-methoxy-2-methyl-9-phenyl-7,8,9,10-tetrahydroimidazo[1,2-h][1,7]naphthyridine,
(7R,8R,9R)-2,3-dimethyl-8-hydroxy-7-(2-hydroxyethoxy)-9-phenyl-7,8,9,10-tetrahydroimidazo[1,2-h][1,7]naphthyridine,
(7R,8R,9R)-3,9-diphenyl-8-hydroxy-7-(2-methoxyethoxy)-2-methyl-7,8,9,10-tetrahydroimidazo[1,2-h][1,7]naphthyridine,
(7R,8R,9R)-7,8-dihydroxy-2-methoxymethyl-3-methyl-9-phenyl-7,8,9,10-tetrahydroimidazo[1,2-h][1,7]naphthyridine,
(7S,8R,9R)-8-hydroxy-7-(2-methoxyethoxy)-2-methoxymethyl-3-methyl-9-phenyl-7,8,9,10-tetrahydroimidazo[1,2-h][1,7]naphthyridine,
(7R,8R,9R)-8-hydroxy-7-(2-methoxyethoxy)-2-methoxymethyl-3-methyl-9-phenyl-7,8,9,10-tetrahydroimidazo[1,2-h][1,7]naphthyridine,
(7S,8R,9R)-7-ethoxy-8-hydroxy-2-methoxymethyl-3-methyl-9-phenyl-7,8,9,10-tetrahydroimidazo[1,2-h][1,7]naphthyridine,
(7R,8R,9R)-7-ethoxy-8-hydroxy-2-methoxymethyl-3-methyl-9-phenyl-7,8,9,10-tetrahydroimidazo[1,2-h][1,7]naphthyridine,
(7R,8S,9R)-10-acetyl-8-hydroxy-2,3-dimethyl-7-(4-morpholino)-7,8,9,10-tetrahydroimidazo[1,2-h][1,7]naphthyridine,
(7R,8S,9R)-8-hydroxy-2,3-dimethyl-7-(4-morpholino)-7,8,9,10-tetrahydroimidazo[1,2-h][1,7]naphthyridine,
(7R,8S,9R)-10-acetyl-8-hydroxy-2,3-dimethyl-7-methylamino-7,8,9,10-tetrahydro-imidazo[1,2-h][1,7]naphthyridine,
(7R,8S,9R)-8-hydroxy-2,3-dimethyl-7-methylamino-7,8,9,10-tetrahydroimidazo[1,2-h][1,7]naphthyridine,
(7R,8S,9R)-10-acetyl-8-hydroxy-2,3-dimethyl-7-(1-pyrrolidino)-7,8,9,10-tetrahydroimidazo[1,2-h][1,7]naphthyridine,
(7R,8S,9R)-8-hydroxy-2,3-dimethyl-7-(1-pyrrolidino)-7,8,9,10-tetrahydroimidazo[1,2-h][1,7]naphthyridine,
(7R,8S,9R)-10-acetyl-7-benzylamino-8-hydroxy-2,3-dimethyl-7,8,9,10-tetrahydro-imidazo[1,2-h][1,7]naphthyridine,
(7R,8S,9R)-7-benzylamino-8-hydroxy-2,3-dimethyl-7,8,9,10-tetrahydroimidazo[1,2-h][1,7]naphthyridine,
(7R,8S,9R)-10-acetyl-8-hydroxy-7-(2-methoxyethylamino)-2,3-dimethyl-7,8,9,10-tetrahydroimidazo[1,2-h][1,7]naphthyridine,
(7R,8S,9R)-8-hydroxy-7-(2-methoxyethylamino)-2,3-dimethyl-7,8,9,10-tetrahydroimidazo[1,2-h][1,7]naphthyridine,
(7R,8S,9R)-10-acetyl-7-(dimethylamino)-8-hydroxy-2,3-dimethyl-7,8,9,10-tetrahydro-imidazo[1,2-h][1,7]naphthyridine,
(7R,8S,9R)-8-hydroxy-7-(dimethylamino)-2,3-dimethyl-7,8,9,10-tetrahydroimidazo[1,2-h][1,7]naphthyridine,
(7S,8S,9R)-8-hydroxy-2,3,7-trimethyl-7,8,9,10-tetrahydroimidazo[1,2-h][1,7]naphthyridine,
(7S,8S,9R)-7-cyanomethyl-8-hydroxy-2,3-dimethyl-7,8,9,10-tetrahydroimidazo[1,2h][1,7]naphthyridine,
(7S,8S,9R)-8-hydroxy-2,3-dimethyl-7-propyl-7,8,9,10-tetrahydroimidazo[1,2-h][1,7]naphthyridine,
(7R,8S,9R)-8-hydroxy-2,3-dimethyl-7-(3-methoxypropyl)-7,8,9,10-tetrahydroimidazo-[1,2-h][1,7]naphthyridine,
2,3-dimethyl-9-phenyl-7H-8,9-dihydro-pyrano-[2,3-c]-N-(diethyl)imidazo[1,2-a]pyridine-6-carboxamide,
ethyl 2,3-dimethyl-9-phenyl-7H-8,9-dihydro-pyrano[2,3-c]-imidazo[1,2-a]pyridine-6-carboxylate,
2,3-dimethyl-9-phenyl-7H-8,9-dihydro-pyrano[2,3-c]-imidazo[1,2-a]pyridine-6-(N,N-dimethyl)carbamide,
(7R,8R,9R)-2,3-dimethyl-7(2-methoxyethoxy)-9-phenyl-8-(5-nitrooxy-valeryloxy)-7,8,9,10-tetrahydro-imidazo[1,2-h][1,7]naphthyridine,
(7R,8R,9R)-2,3-dimethyl-7(2-methoxyethoxy)-9-phenyl-8-(4-nitrooxy-butyryloxy)-7,8,9,10-tetrahydro-imidazo[1,2-h][1,7]naphthyridine,
(7R,8R,9R)-2,3-dimethyl-7-(2-methoxyethoxy)-9-phenyl-8-(5-nitro-oxy-valeryloxy)-7H-8,9-dihydro-pyrano[2,3-c]imdazo[1,2-a]pyridine,
(7R,8R,9R)-2,3-dimethyl-7-(2-methoxyethoxy)-9-phenyl-8-(6-nitro-oxy-2-oxa-capryloxy)-7,8,9,10-tetrahydro-imidazo[1,2-h][1,7]naphthyridine,
(7R,8R,9R)-2,3-dimethyl-7-(2-methoxyethoxy)-9-phenyl-8-(4-nitro-oxymethyl-benzoyloxy)-7,8,9,10-tetrahydroimidazo[1,2-h][1,7]naphthyridine,
and the salts, hydrates, solvates, hydrates of the salts and solvates of the salts of these compounds; and a second active ingredient, which is selected from the group consisting of NSAIDs, COX-2 inhibitors, NO-NSAIDs, bisphosphonates and corticosteroids,
for simultaneous, sequential or separate use in therapy in any order.

3. A pharmaceutical composition according to claim 1 wherein the first active ingredient is a compound selected from the group consisting of
(7R,8R,9R)-8-hydroxy-7-(2-methoxyethoxy)-2,3-dimethyl-9-phenyl-7,8,9,10-tetrahydro-imidazo[1,2-h][1,7]naphthyridine,
(7R,8R,9R)-2,3-dimethyl-8-hydroxy-7-methoxy-9-phenyl-7,8,9,10-tetrahydroimidazo[1,2-h][1,7]naphthyridine,
(7S,8R,9R)-2,3-dimethyl-8-hydroxy-7-methoxy-9-phenyl-7,8,9,10-tetrahydroimidazo[1,2-h][1,7]naphthyridine,
(7R,8R,9R)-2,3-dimethyl-7-ethoxy-8-hydroxy-9-phenyl-7,8,9,10-tetrahydroimidazo[1,2-h][1,7]naphthyridine,
(7S,8R,9R)-2,3-dimethyl-7-ethoxy-8-hydroxy-9-phenyl-7,8,9,10-tetrahydroimidazo[1,2-h][1,7]naphthyridine,
(7R,8R,9R)-2,3-dimethyl-8-hydroxy-7-(2-methoxyethoxy)-9-phenyl-7,8,9,10-tetrahydroimidazo[1,2-h][1,7]naphthyridine,
(7R,8R,9R)-8-acetoxy-7-(2-methoxyethoxy)-2,3-dimethyl-9-phenyl-7,8,9,10-tetrahydroimidazo[1,2-h][1,7]naphthyridine,
(7R,8R,9R)-8-benzoyloxy-7-(2-methoxyethoxy)-2,3-dimethyl-9-phenyl-7,8,9,10-tetrahydroimidazo[1,2-h][1,7]naphthyridine,
(7R,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,
(7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,
(7R,8S,9R)-2,3,8-trimethyl-7,8-dihydroxy-9-phenyl-7,8,9,10-tetrahydroimidazo[1,2-h][1,7]naphthyridine,
(7R,8R,9R)-2,3,7-trimethyl-7,8-dihydroxy-9-phenyl-7,8,9,10-tetrahydroimidazo[1,2-h][1,7]naphthyridine,
(7R,8R,9R)-2,3-dimethyl-8-hydroxy-7-(2-methoxyethoxy)-9-phenyl-7H-8,9-dihydropyrano[2,3-c]imidazo[1,2-a]pyridine,
(7R,8R,9R)-2,3-dimethyl-8-hydroxy-7-ethoxy-9-phenyl-7H-8,9-dihydropyrano[2,3-c]imidazo[1,2-a]pyridine,
(7R,8R,9R)-8-hydroxy-2-methyl-7-(2-methoxyethoxy)-9-phenyl-7,8,9,10-tetrahydroimidazo[1,2-h][1,7]naphthyridine,
(7R,8R,9R)-3-chloro-8-hydroxy-7-(2-methoxyethoxy)-2-methyl-9-phenyl-7,8,9,10-tetrahydroimidazo[1,2-h][1,7]naphthyridine,
(7R,8R,9R)-3-chloro-8-hydroxy-7-(2-methoxyethoxy)-2-methyl-9-phenyl-7H-8,9-dihydro-pyrano[2,3-c]imidazo[1,2-a]pyridine
and the salts, hydrates, solvates, hydrates of the salts and solvates of the salts of these compounds.

4. A pharmaceutical composition according to claim 1 comprising a first active ingredient, which is a tricyclic imidazo[1,2-a]pyridine compound selected from a group consisting of the tricyclic imidazo[1,2-a]pyridine compounds which are specifically disclosed and/or individualized and/or claimed in the patent applications WO 9842707, WO 0017200, WO 0026217, WO 0063211, WO 0172756, WO 0172755, WO 0172757 and WO 0234749, and which are not substituted by a hydroxy-1-4C-alkyl radical bonded on the imidazo ring;
or is a compound selected from the group consisting of
(7S,8R,9R)-2,3-dimethyl-7,8-dihydroxy-9-phenyl-7,8,9,10-tetrahydroimidazo[1,2-h][1,7]naphthyridine,
(7S,8R,9R)-7,8-isopropylidenedioxy-2,3-dimethyl-9-phenyl-7,8,9,10-tetrahydroimidazo[1,2-h][1,7]naphthyridine,
7,8-dihydroxy-9-phenyl-2,3-dimethyl-7H-8,9-dihydropyrano[2,3-c]imidazo[1,2-a]pyridine,
(7R,8R,9R)-2,3-dimethyl-8-hydroxy-7-methoxy-9-phenyl-7,8,9,10-tetrahydroimidazo[1,2-h][1,7]naphthyridine,
(7S,8S,9S)-2,3-dimethyl-8-hydroxy-7-methoxy-9-phenyl-7,8,9,10-tetrahydroimidazo[1,2-h][1,7]naphthyridine,
(7S,8R,9R)-2,3-dimethyl-8-hydroxy-7-methoxy-9-phenyl-7,8,9,10-tetrahydroimidazo[1,2-h][1,7]naphthyridine,
(7R,8S,9S)-2,3-dimethyl-8-hydroxy-7-methoxy-9-phenyl-7,8,9,10-tetrahydroimidazo[1,2-h][1,7]naphthyridine,
(7R,8R,9R)-2,3-dimethyl-7-ethoxy-8-hydroxy-9-phenyl-7,8,9,10-tetrahydroimidazo[1,2-h][1,7]naphthyridine,
(7S,8R,9R)-2,3-dimethyl-7-ethoxy-8-hydroxy-9-phenyl-7,8,9,10-tetrahydroimidazo[1,2-h][1,7]naphthyridine,
(7R,8R,9R)-2,3-dimethyl-8-hydroxy-7-(2-methoxyethoxy)-9-phenyl-7,8,9,10-tetrahydroimidazo[1,2-h][1,7]naphthyridine,
(7S,8S,9S)-2,3-dimethyl-8-hydroxy-7-(2-methoxyethoxy)-9-phenyl-7,8,9,10-tetrahydroimidazo[1,2-h][1,7]naphthyridine,
(7S,8R,9R)-2,3-dimethyl-8-hydroxy-7-(2-methoxyethoxy)-9-phenyl-7,8,9,10-tetrahydroimidazo[1,2-h][1,7]naphthyridine,
(7R,8S,9S)-2,3-dimethyl-8-hydroxy-7-(2-methoxyethoxy)-9-phenyl-7,8,9,10-tetrahydroimidazo[1,2-h][1,7]naphthyridine,
(7S,8R,9R)-2,3-dimethyl-8-hydroxy-9-phenyl-7-(2-propoxy)-7,8,9,10-tetrahydroimidazo[1,2-h][1,7]naphthyridine,
(7R,8R,9R)-2,3-dimethyl-7,8-dimethoxy-9-phenyl-7,8,9,10-tetrahydroimidazo[1,2-h][1,7]naphthyridine,
(7R,8R,9R)-2,3-dimethyl-8-hydroxy-7-(2-methylthioethyloxy)-9-phenyl-7,8,9,10-tetrahydroimidazo[1,2-h]-[1,7]naphthyridine,
(7S,8R,9R)-2,3-dimethyl-8-hydroxy-7-(2-methylthioethyloxy)-9-phenyl-7,8,9,10-tetrahydroimidazo[1,2-h][1,7]-naphthyridine,
(7R,8R,9R)-2,3-dimethyl-8-hydroxy-7-(2-methylsulphinylethoxy)-9-phenyl-7,8,9,10-tetrahydroimidazo-[1,2-h][1,7]naphthyridine,
(7S,8R,9R)-2,3-dimethyl-8-hydroxy-7-(2-methylsulphinylethoxy)-9-phenyl-7,8,9,10-tetrahydroimidazo-[1,2-h][1,7]naphthyridine,
(7R,8R,9R)-2,3-dimethyl-8-hydroxy-7-(ethylthio)-9-phenyl-7,8,9,10-tetrahydroimidazo[1,2-h][1,7]naphthyridine,
(7S,8R,9R)-2,3-dimethyl-8-hydroxy-7-(ethylthio)-9-phenyl-7,8,9,10-tetrahydroimidazo[1,2-h][1,7]naphthyridine,
(7R,8R,9R)-2,3-dimethyl-8-hydroxy-7-(2,2,2-trifluoroethoxy)-9-phenyl-7,8,9,10-tetrahydroimidazo-[1,2-h][1,7]naphthyridine,
(7S,8R,9R)-2,3-dimethyl-8-hydroxy-7-(2,2,2-trifluoroethoxy)-9-phenyl-7,8,9,10-tetrahydroimidazo-[1,2-h][1,7]naphthyridine,
(7S,8R,9R)-8-acetoxy-7-(2-methoxyethoxy)-2,3-dimethyl-9-phenyl-7,8,9,10-tetrahydroimidazo[1,2-h][1,7]naphthyridine,
(7R,8R,9R)-8-acetoxy-7-(2-methoxyethoxy)-2,3-dimethyl-9-phenyl-7,8,9,10-tetrahydroimidazo[1,2-h][1,7]naphthyridine,
(7R,8R,9R)-8-acetoxy-7-methoxy-2,3-dimethyl-9-phenyl-7,8,9,10-tetrahydroimidazo[1,2-h][1,7]naphthyridine,
(7R,8R,9R)-8-acetoxy-7-ethoxy-2,3-dimethyl-9-phenyl-7,8,9,10-tetrahydroimidazo[1,2-h][1,7]naphthyridine,
(7R,8R,9R)-7-(2-methoxyethoxy)-2,3-dimethyl-9-phenyl-8-propionyloxy-7,8,9,10-tetrahydroimidazo[1,2-h]-[1,7]naphthyridine,
(7R,8R,9R)-8-benzoyloxy-7-(2-methoxyethoxy)-2,3-dimethyl-9-phenyl-7,8,9,10-tetrahydroimidazo[1,2-h][1,7]naphthyridine,
(7S,8R,9R)-8-benzoyloxy-7-(2-methoxyethoxy)-2,3-dimethyl-9-phenyl-7,8,9,10-tetrahydroimidazo[1,2-h][1,7]naphthyridine,
(7R,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,
(7S,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,
(7R,8R,9R)-8-benzoyloxy-7-methoxy-2,3-dimethyl-9-phenyl-7,8,9,10-tetrahydroimidazo[1,2-h][1,7]naphthyridine,
(7S,8R,9R)-8-benzoyloxy-7-methoxy-2,3-dimethyl-9-phenyl-7,8,9,10-tetrahydroimidazo[1,2-h][1,7]naphthyridine,
(7R,8R,9R)-7-(2-methoxyethoxy)-2,3-dimethyl-8-(4-nitrobenzoyloxy)-9-phenyl-7,8,9,10-tetrahydroimidazo[1,2-h][1,7]naphthyridine,
(7S,8R,9R)-7-(2-methoxyethoxy)-2,3-dimethyl-8-(4-nitrobenzoyloxy)-9-phenyl-7,8,9,10-tetrahydroimidazo[1,2-h][1,7]naphthyridine,
(7S,8R,9R)-7-(2-methoxyethoxy)-2,3-dimethyl-8-(3-nitrobenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(3-nitrobenzoyloxy)-9-phenyl-7,8,9,10-tetrahydroimidazo[1,2-h][1,7]naphthyridine,
(7S,8R,9R)-7-methoxy-2,3-dimethyl-8-(3-nitrobenzoyloxy)-9-phenyl-7,8,9,10-tetrahydroimidazo[1,2-h][1,7]naphthyridine,
(7R,8R,9R)-7-methoxy-2,3-dimethyl-8-(3-nitrobenzoyloxy)-9-phenyl-7,8,9,10-tetrahydroimidazo[1,2-h][1,7]naphthyridine,
(7S,8R,9R)-7-(2-methoxyethoxy)-2,3-dimethyl-8-(4-methoxybenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(4-methoxybenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,
(7S,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,
(7S,8R,9R)-7-(2-methoxyethoxy)-8-(N,N-diethylaminocarbonyloxy)-2,3-dimethyl-9-phenyl-7,8,9,10-tetrahydroimidazo[1,2-h][1,7]naphthyridine,
(7R,8R,9R)-7-(2-methoxyethoxy)-8-(N,N-diethylaminocarbonyloxy)-2,3-dimethyl-9-phenyl-7,8,9,10-tetrahydroimidazo[1,2-h][1,7]naphthyridine,
(7R,8R,9R)-8-ethylaminocarbonyloxy-7-(2-methoxyethoxy)-2,3-dimethyl-9-phenyl-7,8,9,10-tetrahydroimidazo[1,2-h][1,7]naphthyridine,
(7R,8R,9R)-8-benzoyloxy-2,3-dimethyl-7-(2-methoxyethoxy)-9-phenyl-7H-8,9-dihydropyrano[2,3-c]imidazo[1,2-a]pyridine,
(7S,8R,9R)-8-benzoyloxy-2,3-dimethyl-7-(2-methoxyethoxy)-9-phenyl-7H-8,9-dihydropyrano[2,3-c]imidazo[1,2-a]pyridine,
(7R,8R,9R)-8-[4-(methoxycarbonyl)-benzoyloxy]-2,3-dimethyl-7-(2-methoxyethoxy)-9-phenyl-7H-8,9-dihydropyrano[2,3-c]imidazo[1,2-a]pyridine,
(7S,8R,9R)-8-[4—(methoxycarbonyl)-benzoyloxy]-2,3-dimethyl-7-(2-methoxyethoxy)-9-phenyl-7H-8,9-dihydropyrano[2,3-c]imidazo[1,2-a]pyridine,
(7S,8R,9R)-2,3-dimethyl-7-methoxy-8-methoxyacetyloxy-9-phenyl-7,8,9,10-tetrahydroimidazo[1,2-h][1,7]naphthyridine,
(7R,8R,9R)-8-(N,N-diethylaminocarbonyloxy)-2,3-dimethyl-7-methoxy-9-phenyl-7,8,9,10-tetrahydroimidazo[1,2-h][1,7]naphthyridine,
(7S,8R,9R)-8-(N,N-diethylaminocarbonyloxy)-2,3-dimethyl-7-methoxy-9-phenyl-7,8,9,10-tetrahydroimidazo[1,2-h][1,7]naphthyridine,
(7R,8R,9R)-7-methoxy-8-methoxycarbonyloxy-2,3-dimethyl-9-phenyl-7,8,9,10-tetrahydroimidazo[1,2-h][1,7]naphthyridine,
(7S,8R,9R)-7-methoxy-8-methoxycarbonyloxy-2,3-dimethyl-9-phenyl-7,8,9,10-tetrahydroimidazo[1,2-h][1,7]naphthyridine,
(7R,8R,9R)-2,3-dimethyl-8-formyloxy-7-methoxy-9-phenyl-7,8,9,10-tetrahydroimidazo[1,2-h][1,7]naphthyridine,
(7S,8R,9R)-2,3-dimethyl-8-formyloxy-7-methoxy-9-phenyl-7,8,9,10-tetrahydroimidazo[1,2-h][1,7]naphthyridine,
(7R,8R,9R)-8-benzoyloxy-2,3-dimethyl-7-methoxy-9-phenyl-7,8,9,10-tetrahydroimidazo[1,2-h][1,7]naphthyridine,
(7R,8S,9R)-2,3,8-trimethyl-7,8-dihydroxy-9-phenyl-7,8,9,10-tetrahydroimidazo[1,2-h][1,7]naphthyridine,
(7S,8S,9R)-2,3-dimethyl-8-benzyl-7,8-dihydroxy-9-phenyl-7,8,9,10-tetrahydroimidazo[1,2-h][1,7]naphthyridine,
(7R,8S,9R)-2,3,8-trimethyl-7,8-0,0-isopropylidene-9-phenyl-7,8,9,10-tetrahydroimidazo[1,2-h][1,7]naphthyridine,
(7S,8S,9R)-2,3,8-trimethyl-7-(2-methoxyethoxy)-8-hydroxy-9-phenyl-7,8,9,10-tetrahydroimidazo[1,2-h][1,7]naphthyridine,
(7S,8S,9R)-2,3,8-trimethyl-7-methoxy-8-hydroxy-9-phenyl-7,8,9,10-tetrahydroimidazo[1,2-h][1,7]naphthyridine,
(7R,8R,9R)-2,3,7-trimethyl-7,8-dihydroxy-9-phenyl-7,8,9,10-tetrahydroimidazo[1,2-h][1,7]naphthyridine,
(7R,8R,9R)-2,3,7-trimethyl-7,8-[1,3]dioxolo-9-phenyl-7,8,9,10-tetrahydroimidazo[1,2-h][1,7]naphthyridine, (8S,9R)-2,3-dimethyl-8-hydroxy-7-methylidene-9-phenyl-7,8,9,10-tetrahydroimidazo[1,2-h][1,7]naphthyridine,
(7S,8R,9R)-2,3,7-trimethyl-7,8-dihydroxy-9-phenyl-7H-8,9-dihydropyrano[2,3-c]imidazo[1,2-a]pyridine,
(7R,8R,9R)-2,3,7-trimethyl-7,8-dihydroxy-9-phenyl-7H-8,9-dihydropyrano[2,3-c]imidazo[1,2-a]pyridine,
(7S,8R,9R)-2,3-dimethyl-7,8-dihydroxy-7,9-diphenyl-7H-8,9-dihydropyrano[2,3-c]imidazo[1,2-a]pyridine,
(7S,8R,9R)-2,3-dimethyl-7-(2′,2′-dimethylvinyl)-7,8-dihydroxy-9-phenyl-7H-8,9-dihydropyrano[2,3-c]imidazo[1,2-a]pyridine,
(7R,8R,9R)-2,3-dimethyl-7,8-O-isopropylidene-9-phenyl-7-vinyl-7H-8,9-dihydropyrano[2,3-c]imidazo[1,2-a]pyridine,
(7R,8R,9R)-2,3-dimethyl-8-hydroxy-7-(2-methoxyethoxy)-9-phenyl-7H-8,9-dihydropyrano[2,3-c]imidazo[1,2-a]pyridine,
(7S,8R,9R)-2,3-dimethyl-8-hydroxy-7-(2-methoxyethoxy)-9-phenyl-7H-8,9-dihydropyrano[2,3-c]imidazo[1,2-a]pyridine,
(7R,8R,9R)-2,3-dimethyl-8-hydroxy-7-ethoxy-9-phenyl-7H-8,9-dihydropyrano[2,3-c]imidazo[1,2-a]pyridine,
(7S,8R,9R)-2,3-dimethyl-8-hydroxy-7-ethoxy-9-phenyl-7H-8,9-dihydropyrano[2,3-c]imidazo[1,2-a]pyridine,
(7R,8R,9R)-2,3-dimethyl-8-hydroxy-7-(2-methoxypropoxy)-9-phenyl-7H-8,9-dihydropyrano[2,3-c]imidazo[1,2-a]pyridine,
(7S,8R,9R)-2,3-dimethyl-8-hydroxy-7-(2-methoxypropoxy)-9-phenyl-7H-8,9-dihydropyrano[2,3-c]imidazo[1,2-a]pyridine,
(7R,8R,9R)-2,3-dimethyl-8-hydroxy-7-(2-propoxy)-9-phenyl-7H-8,9-dihydropyrano[2,3-c]imidazo[1,2-a]pyridine,
(7S,8R,9R)-2,3-dimethyl-8-hydroxy-7-(2-propoxy)-9-phenyl-7H-8,9-dihydropyrano[2,3-c]imidazo[1,2-a]pyridine,
(7R,8R,9R)-2,3-dimethyl-8-hydroxy-7-butoxy-9-phenyl-7H-8,9-dihydropyrano[2,3-c]imidazo[1,2-a]pyridine,
(7S,8R,9R)-2,3-dimethyl-8-hydroxy-7-butoxy-9-phenyl-7H-8,9-dihydropyrano[2,3-c]imidazo[1,2-a]pyridine,
(7S,8R,9R)-7,8-dihydroxy-6-methoxymethyl-2,3-dimethyl-9-phenyl-7,8,9,10-tetrahydroimidazo[1,2-h][1,7]naphthyridine,
(7R,8R,9R)-7,8-dihydroxy-6-methoxymethyl-2,3-dimethyl-9-phenyl-7,8,9,10-tetrahydroimidazo[1,2-h][1,7]naphthyridine,
(7S,8R,9R)-8-hydroxy-7-methoxy-6-methoxymethyl-2,3-dimethyl-9-phenyl-7,8,9,10-tetrahydroimidazo[1,2-h][1,7]naphthyridine,
(7R,8R,9R)-8-hydroxy-7-methoxy-6-methoxymethyl-2,3-dimethyl-9-phenyl-7,8,9,10-tetrahydroimidazo[1,2-h][1,7]naphthyridine,
(7R,8R,9R)-8-hydroxy-7-(2-methoxyethoxy)-6-methoxymethyl-2,3-dimethyl-9-phenyl-7,8,9,10-tetrahydroimidazo[1,2-h][1,7]naphthyridine,
(7S,8R,9R)-8-hydroxy-7-(2-methoxyethoxy)-6-methoxymethyl-2,3-dimethyl-9-phenyl-7,8,9,10-tetrahydroimidazo[1,2-h][1,7]naphthyridine,
(7R,8R,9R)-8-hydroxy-7-ethoxy-6-methoxymethyl-2,3-dimethyl-9-phenyl-7,8,9,10-tetrahydro-imidazo[1,2-h][1,7]naphthyridine,
(7S,8R,9R)-8-hydroxy-7-ethoxy-6-methoxymethyl-2,3-dimethyl-9-phenyl-7,8,9,10-tetrahydro-imidazo[1,2-h][1,7]naphthyridine,
7,8-dihydroxy-2,3-dimethyl-9-(3-thienyl)-7,8,9,10-tetrahydroimidazo[1,2-h][1,7]naphthyridine,
7-hydroxy-2,3-dimethyl-9-(3-thienyl)-7,8,9,10-tetrahydroimidazo[1,2-h][1,7]naphthyridine,
9-(3-furyl)-7-hydroxy-2,3-dimethyl-7,8,9,10-tetrahydroimidazo[1,2-h][1,7]naphthyridine,
(7R,8R,9R)-8-hydroxy-7-[2-(2-methoxyethoxy)ethoxy]-2,3-dimethyl-9-phenyl-7,8,9,10-tetrahydroimidazo[1,2-h][1,7]naphthyridine,
(7S,8R,9R)-8-hydroxy-7-[2-(2-methoxyethoxy)ethoxy]-2,3-dimethyl-9-phenyl-7,8,9,10-tetrahydroimidazo[1,2-h][1,7]naphthyridine,
(7R,8R,9R)-7,8-dihydroxy-2-methyl-9-phenyl-7,8,9,10-tetrahydroimidazo[1,2-h][1,7]naphthyridine,
(7S,8R,9R)-8-hydroxy-2-methyl-7-(2-methoxyethoxy)-9-phenyl-7,8,9,10-tetrahydroimidazo[1,2-h][1,7]naphthyridine,
(7R,8R,9R)-8-hydroxy-2-methyl-7-(2-methoxyethoxy)-9-phenyl-7,8,9,10-tetrahydroimidazo[1,2-h][1,7]naphthyridine,
(7R,8R,9R)-3-bromo-8-hydroxy-7-(2-methoxyethoxy)-2-methyl-9-phenyl-7,8,9,10-tetrahydroimidazo[1,2-h][1,7]naphthyridine,
(7R,8R,9R)-3-chloro-8-hydroxy-7-(2-methoxyethoxy)-2-methyl-9-phenyl-7,8,9,10-tetrahydroimidazo[1,2-h][1,7]naphthyridine,
(7R,8R,9R)-3-bromo-7-hydroxy-8-(2-methoxyethoxy)-2-methyl-9-phenyl-7,8,9,10-tetrahydroimidazo[1,2-h][1,7]naphthyridine,
(7R,8R,9R)-3-chloro-8-hydroxy-7-(2-methoxyethoxy)-2-methyl-9-phenyl-7H-8,9-dihydro-pyrano[2,3-c]imidazo[1,2-a]pyridine,
(7R,8R,9R)-8-hydroxy-7-(2-methoxyethoxy)-2-methyl-9-phenyl-7H-8,9-dihydro-pyrano[2,3-c]imidazo[1,2-a]pyridine,
(7R,8R,9R)-7,8-dihydroxy-2-methyl-9-phenyl-7H-8,9-dihydropyrano[2,3-c]imidazo[1,2-a]pyridine,
(7S,8R,9R)-7,8-dihydroxy-2-methyl-9-phenyl-7,8,9,10-tetrahydroimidazo[1,2-h][1,7]naphthyridine,
(7R,8R,9R)-8-hydroxy-7-methoxy-2-methyl-9-phenyl-7,8,9,10-tetrahydroimidazo[1,2-h][1,7]naphthyridine,
(7S,8R,9R)-8-hydroxy-7-methoxy-2-methyl-9-phenyl-7,8,9,10-tetrahydroimidazo[1,2-h][1,7]naphthyridine,
(7R,8R,9R)-2,3-dimethyl-8-hydroxy-7-(2-hydroxyethoxy)-9-phenyl-7,8,9,10-tetrahydroimidazo[1,2-h][1,7]naphthyridine,
(7R,8R,9R)-3,9-diphenyl-8-hydroxy-7-(2-methoxyethoxy)-2-methyl-7,8,9,10-tetrahydroimidazo[1,2-h][1,7]naphthyridine,
(7R,8R,9R)-7,8-dihydroxy-2-methoxymethyl-3-methyl-9-phenyl-7,8,9,10-tetrahydroimidazo[1,2-h][1,7]naphthyridine,
(7S,8R,9R)-8-hydroxy-7-(2-methoxyethoxy)-2-methoxymethyl-3-methyl-9-phenyl-7,8,9,10-tetrahydroimidazo[1,2-h][1,7]naphthyridine,
(7R,8R,9R)-8-hydroxy-7-(2-methoxyethoxy)-2-methoxymethyl-3-methyl-9-phenyl-7,8,9,10-tetrahydroimidazo[1,2-h][1,7]naphthyridine,
(7S,8R,9R)-7-ethoxy-8-hydroxy-2-methoxymethyl-3-methyl-9-phenyl-7,8,9,10-tetrahydroimidazo[1,2-h][1,7]naphthyridine,
(7R,8R,9R)-7-ethoxy-8-hydroxy-2-methoxymethyl-3-methyl-9-phenyl-7,8,9,10-tetrahydroimidazo[1,2-h][1,7]naphthyridine;
and the salts, hydrates, solvates, hydrates of the salts and solvates of the salts of these compounds;
and a second active ingredient, which is selected from the group consisting of NSAIDs, COX-2 inhibitors, NO-NSAIDs, bisphosphonates and corticosteroids.

5. A pharmaceutical composition according to claim 1, wherein the first active ingredient is (7R,8R,9R)-8-hydroxy-7-(2-methoxyethoxy)-2,3-dimethyl-9-phenyl-7,8,9,10-tetrahydroimidazo[1,2-h][1,7]naphthyridine or a salt, hydrate, solvate, hydrate of a salt or solvate of a salt of this compound.

6. A pharmaceutical composition according to claim 1, wherein the second active ingredient is a NSAID selected from the group consisting of glycolic acid [o-(2,6-dichloroanilino)phenyl]acetate(ester) [INN: ACECLOFENAC]; 1-(4-chlorobenzoyl)-5-methoxy-2-methyl-1H-indole-3-acetic acid carboxymethyl ester [INN: ACEMETACIN]; 2-(acetyloxy)benzoic acid [ACETYLSALICYLIC ACID], 2-methoxyphenyl-alpha-methyl-4-(isobutyl)phenylacetate [Research Code: AF-2259], (4-allyloxy-3-chlorophenyl)acetic acid [INN: ALCLOFENAC], p-[(2-methylallyl)amino]hydratropic acid [INN: ALMINOPROFEN], 2-amino-3-benzoylphenylacetic acid [INN: AMFENAC], (plus/minus)-4-(1-hydroxyethoxy)-2-methyl-N-2-pyridyl-2H-1,2-benzothiazine-3-carboxamide ethylcarbonate (ester), 1,1-dioxide [INN: AMPIROXICAM], 2-methoxyphenyl-1-methyl-5-(p-methylbenzoyl)pyrrol-2-acetamido-acetate [INN: AMTOLMETINGUACIL], (plus/minus)-2,3-dihydro-5-(4-methoxybenzoyl)-1H pyrrolizine-1-carboxylic acid [INN: ANIROLAC], 2-[4-(alpha,alpha,alpha-trifluoro-m-tolyl)-1-piperazinyl]ethyl-N-(7-trifluoromethyl-4-quinolyl)anthranilate [INN: ANTRAFENINE], 5-(dimethylamino)-9-methyl-2-propyl-1H-pyrazolo[1,2-a][1,2,4]benzo-triazine-1,3(2H)-dione [INN: AZAPROPAZONE], 4-acetamidophenyl salicylate acetate [INN: BENORILATE], 2-(8-methyl-10,11-dihydro-11-oxodibenz[b,f]oxepin-2-yl)propionic acid [INN: BERMOPROFEN], 2-[(1-benzyl-1H-indazol-3-yl)methoxy]-2-methylpropionic acid [INN: BINDARIT], [2-amino-3-(p-bromobenzoyl)phenyl]acetic acid [INN: BROMFENAC], 3-(3-chloro-4-cyclohexylbenzoyl)propionic acid [INN: BUCLOXIC ACID], 5-butyl-1-cyclohexylbarbituric acid [INN: BUCOLOM], 4-butoxy-N-hydroxybenzeneacetamide [INN: BUFEXAMAC], butylmalonic acid mono-(1,2-diphenylhydrazide) [INN: BUMADIZONE], alpha-ethyl-4-(2-methylpropyl)benzeneacetic acid [INN: BUTIBUFEN], 2-(4-biphenylyl)butyric acid, trans-4-phenylcyclohexylamine salt (1:1) [INN: BUTIXIRATE], 2-(acetyloxy)-benzoic acid, calcium salt, compound with urea (1:1) [INN: CARBASALATE CALCIUM], (plus/minus)-6-chloro-alpha-methylcarbazole-2-acetic acid [INN: CARPROFEN], 1-cinnamoyl-5-methoxy-2-methylindole-3-acetic acid [INN: CINMETACIN], N-(2-pyridyl)-2-methyl-4-cinnamoyloxy-2H-1,2-benzothiazine-3-carboxamide-1,1-dioxide [INN: CINNOXICAM], 6-chloro-5-cyclohexyl-1-indancarboxylic acid [INN: CLIDANAC], 2-[4-(p-chlorophenyl)benzyloxy]-2-methylpropionic acid [INN: CLOBUZARIT], 5-methoxy-2-methyl-3-indolylacetohydroxamic acid [INN: DEBOXAMET], (S)-(+)-p-isobutylhydratropic acid [INN: DEXIBUPROFEN], (+)-(S)-m-benzoylhydratropic acid [INN: DEXKETOPROFEN], 2-[(2,6-di-chlorophenyl)amino]benzeneacetic acid [INN: DICLOFENAC], 2′,4′-Difluoro-4-hydroxy-3-biphenylcarboxylic acid [INN: DIFLUNISAL], 4-(2,6-dichloroanilino)-3-thiopheneacetic acid [INN: ELTENAC], N-beta-phenethyl-anthranilic acid [INN: ENFENAMIC ACID] salicylic acid acetate, ester with beta-hydroxy p-acetophenetidide [INN: ETERSALATE], 1,8-diethyl-1,3,4,9-tetrahydropyrano[3,4-b]indole-1-acetic acid [INN: ETODOLAC], 2-[[3-(trifluoromethyl)phenyl]amino]benzoic acid 2-(2-hydroxyethoxy)-ethyl ester [INN: ETOFENAMATE], p-chlorobenzoic acid, ester with 4-butyl-4-(hydroxymethyl)-1,2-diphenyl-3,5-pyrazolidinedione [INN: FECLOBUZONE], 4-biphenylacetic acid [INN: FELBINAC], 3-(4-biphenylylcarbonyl)propionic acid [INN: FENBUFEN], [o-(2,4-dichlorophenoxy)phenyl]acetic acid [INN: FENCLOFENAC], (plus/minus)-m-phenoxyhydratropic acid [INN: FENOPROFEN], 4-(p-chlorophenyl)-2-phenyl-5-thiazoleacetic acid [INN: FENTIAZAC], (plus/minus)-alpha-[[(2-hydroxy-1,1-dimethylethyl)amino]methyl]-benzyl alcohol [INN: FEPRADINOL], 4-(2′,4′-difluorobiphenylyl)-4-oxo-2-methylbutanoic acid [INN: FLOBUFEN], 2,3-dihydroxypropyl N-[8-(trifluoromethyl)-4-quinoly]anthranilate [INN: FLOCTAFENINE], N-(alpha,alpha,alpha-trifluoro-m-tolyl)-anthranilic acid [INN: FLUFENAMIC ACID], (plus)-2-(p-fluorophenyl)-alpha-methyl-5-benzoxazoleacetic acid [INN: FLUNOXAPROFEN], 2-fluoro-alpha-methyl-4-biphenylacetic acid [INN: FLURBIPROFEN], (plus/minus)-2-(2-fluoro-4-biphenylyl)propionic acid 1 (acetoxy)ethyl ester [INN: FLURBIPROFEN AXETIL], 2-ethyl-2,3-dihydro-5-benzofuranacetic acid [INN: FUROFENAC], furoyl)phenyl]propionic acid [INN: FURPROFEN], 2-[2-[1-(p-chlorobenzoyl)-5-methoxy-2-methylindol-3-yl]acetamido]-2-deoxy-D-glucose [INN: GLUCAMETACIN], 2-(2-fluorobiphenyl-4-yl)propionic acid 4-nitrooxybutylester [Research Code: HCT-1026], (p-isobutylphenyl)acetic acid [INN: IBUFENAC], alpha-p-isobutylphenylpropionic acid [INN: IBUPROFEN], methyl 4-(3-thienyl)phenyl-alpha-methylacetate [Research Code: IDPH-8261], (plus/minus)-2-[p-(1-oxo-2-isoindolinyl)phenyl]butyric acid [INN: INDOBUFEN], 1-(4-chlorobenzoyl)-5-methoxy-2-methyl-1H-indole-3-acetic acid [INN: INDOMETACIN], 1-(4-chlorobenzoyl)-5-methoxy-2-methyl-1H-indole-3-acetic acid, 3,7,11-trimethyl-2,6,10-dodecatrienyl ester [INN: INDOMETACIN FARNESIL], p-(1-oxo-2-isoindolinyl)hydratropic acid [INN: INDOPROFEN], 2-(10-methoxy-4H-benzo[4,5]cyclohepta[1,2-b]thiophen-4-ylidene)-acetic acid [Research Code: IX-207-887], m-benzoylhydratropic acid [INN: KETOPROFEN], (DL)-5-benzoyl-3H-1,2-dihydropyrrolo[1,2-a]pyrrole-1-carboxylic acid [INN: KETOROLAC], 2,3-dihydro-5-hydroxy-6-[2-(hydroxymethyl)cinnamyl]benzofuran [Research Code: L-651896], N-(2-carboxyphenyl)-4-chloroanthranilic acid [INN: LOBENZARIT], 3-(p-chlorophenyl)-1-phenylpyrazole-4-acetic acid [INN: LONAZOLAC], 6-chloro-4-hydroxy-2-methyl-N-2-pyridyl-2H-thieno[2,3-e]-1,2-thiazine-3-carboxamide 1,1-dioxide [INN: LORNOXICAM], 2-[4-(2-oxocyclopentan-1-ylmethyl)phenyl]-propionate [INN: LOXOPROFEN], 2(R)-[4-(3-methyl-2-thienyl)phenyl]propionic acid [Research Code: M-5010], N-(2,3-xylyl)anthranilic acid [INN: MEFENAMIC ACID], 4-hydroxy-2-methyl-N-(5-methyl-2-thiazolyl)-2H-1,2-benzothiazine-3-carboxamide 1,1-dioxide [INN: MELOXICAM], 5-aminosalicylic acid [INN: MESALAZINE], (2,2-dimethyl-6-(4-chlorophenyl)-7-phenyl-2,3-dihydro-1H-pyrrolizin-5-yl)-acetic acid [Research Code: ML-3000], 3,4-bis(4-methoxyphenyl)-5-isoxazoleacetic acid [INN: MOFEZOLAC], 4-(6-methoxy-2-naphthyl)-2-butanone [INN: NABUMETONE], (plus)-6-methoxy-alpha-methyl-2-naphthalineacetic acid [INN: NAPROXEN], 2-[3-(trifluoromethyl)anilino]nicotinic acid [INN: NIFLUMIC ACID], 5,5′-azodisalicylic acid [INN: OLSALAZINE], 4,5-diphenyl-2-oxazolepropionic acid [INN: OXAPROZIN], alpha-methyl-4-[(2-oxocyclohexylidene)methyl]benzene acetic acid [INN: PELUBIPROFEN], 4-butyl-1,2-diphenyl-3,5-pyrazolidinedione [INN: PHENYLBUTAZONE], 2-(p-isobutylphenyl)propionic acid 2-pyridyl-methyl ester [INN: PIMEPROFEN], 4-(p-chlorophenyl)-1-(p-fluorophenyl)pyrazole-3-acetic acid [INN: PIRAZOLAC], 4-hydroxy-2-methyl-N-2-pyridyl-2H-1,2-benzothiadiazin-3-carboxamide 1,1-dioxide [INN: PIROXICAM], 3-chloro-4-(3-pyrrolin-1-yl)hydratropic acid [INN: PIRPROFEN], 2-[5H-(1)benzopyrano]2,3-b]pyridin-7-yl]propionic acid [INN: PRANOPROFEN], 2,6-di-tert-butyl-4-(2′-thenoyl)phenol [INN: PRIFELONE], alpha-cyano-1-methyl-beta-oxopyrrole-2-propionanilide [INN: PRINOMIDE], 3-[4-(2-hydroxyethyl)-1-piperazinyl]-propyl-D,L-4-benzamido-N,N-dipropylglutaramat 1-(p-chlorobenzoyl)-5-methoxy-2-methyl-indole-3-acetate (ester) [INN: PROGLUMETACIN], 7-methyl-1-(1-methylethyl)-4-phenyl-2(1H)quinazolinone [INN: PROQUAZONE], 7-methoxy-alpha,10-dimethylphenothiazine-2-acetic acid [INN: PROTIZINIC ACID], 2-[[2-(p-chlorophenyl)-4-methyl-5-oxazolyl]methoxy]-2-methylpropionic acid [INN: ROMAZARIT], o-hydroxybenzamide [SALICYLAMIDE], 2-hydroxybenzoic acid [SALICYLIC ACID], N-acetyl-L-cysteine salicylate (ester), acetate (ester) [INN: SALMISTEINE], N-acetyl-L-cysteine salicylate (ester) [INN: SALNACEDIN], 2-hydroxybenzoic acid 2-carboxyphenyl ester [INN: SALSALATE], 4-[1-(2-fluorobiphenyl-4-yl)ethyl]-N-methylthiazole-2-amine [Research Code: SM-8849], (Z)-5-fluoro-2-methyl-1-[p-(methylsulfinyl)benzylidene]indene-3-acetic acid [INN: SULINDAC], p-2-thenoylhydratropic acid [INN: SUPROFEN] 2-(4-(3-methyl-2-butenyl)phenyl)propionic acid [Research Code: TA-60], phthalidyl 2-(alpha,alpha,alpha-trifluoro-m-toluidino)nicotinate [INN: TALNIFLUMATE], (Z)-5-chloro-3-(2-thenoyl)-2-oxoindole-1-carboxamide [INN: TENIDAP], 2-thiophenecarboxylic acid, ester with salicylic acid [INN: TENOSAL], 4-hydroxy-2-methyl-N-2-pyridyl-2H-thieno[2,3-e]-1,2-thiazine-3-carboxamide [INN: TENOXICAM], 5-(4-chlorophenyl)-N-hydroxy-1-(4-methoxyphenyl)-N-methyl-1H-pyrazole-3-propionamide [INN: TEPOXALIN], alpha-(5-benzoyl-2-thienyl)propionic acid [INN: TIAPROFENIC ACID], 5-chloro-3-[4-(2-hydroxyethyl)-1-piperazinyl]carbonylmethyl-2-benzo-thiazolin one [INN: TIARAMIDE], 2-(2-methyl-5H-[l]benzopyrano[2,3-b]pyridin-7-yl)-propionic acid N,N-dimethylcarbamoylmethyl ester [INN: TILNOPROFEN ARBAMEL], 1-Cyclohexyl-2-(2-methyl-4-quinolyl)-3-(2-thiazolyl)guanidine [INN: TIMEGADINE), 2-amino-6-benzyl-4,5,6,7-tetrahydrothieno[2,3-c]pyridine [INN: TINORIDINE], N-(3-chloro-o-tolyl)anthranilic acid [INN: TOLFENAMIC ACID], 1-methyl-5-(4-methylbenzoyl)-1H-pyrrole-2-acetic acid [INN: TOLMETIN], hydroxybis[alpha-methyl-4-(2-methylpropyl)benzene acetato-O]-aluminium [Research Code: U-18573-G], N-(3-trifluoromethylphenyl)-anthranilic acid n-butyl ester [INN: UFENAMATE], 2-[4-[3-(hydroxyimino)cyclohexyl]phenyl}propionic acid [INN: XIMOPROFEN], 2-(10,11-dihydro-10-oxo-dibenz[b,f]thiepin-2-yl-propionic acid [INN: ZALTOPROFEN] and 2-[4-(2-thiazolyloxy)phenyl]-propionic acid [INN: ZOLIPROFEN]; or a NO-NSAID selected from the group consisting of those NO-NSAIDs which are disclosed in WO 96/32946, WO 96/35416, WO 96/38136, WO 96/39409, WO 00/50037, U.S. Pat. No. 6,057,347, WO 94/04484, WO 94/12463, WO 95/09831, WO 95/30641, WO 97/31654, WO 99/44595, WO 99/45004 and WO 01/45703; or a COX-2 inhibitor selected from the group consisting of 5-chloro-6′-methyl-3-[4-(methylsulfonyl)phenyl]-2,3′-bipyridine [INN: ETORICOXIB], 4-[5-(4-methylphenyl)-3-(trifluoromethyl)-1H-pyrazol-1-yl]benzene-sulfonamide [INN: CELECOXIB], 4-[p-(methylsulfonyl)phenyl]-3-phenyl-2(5H)-furanone [INN: ROFECOXIB], N-[[p-(5-methyl-3-phenyl-4-isoxazolyl)phenyl]sulfonyl]propion-amide [INN: PARECOXIB], p-(5-methyl-3-phenyl-4-isoxazolyl)benzenesulfonamide [INN: VALDECOXIB], 2-[2-(2-chloro-6-fluorophenylamino)-5-methylphenyl]acetic acid [INN: LUMIRACOXIB], 4-(4-cyclohexyl-2-methyloxazol-5-yl)-2-fluorobenzene-sulfonamide [INN: TILMACOXIB], 4-[4-chloro-5-(3-fluoro-4-methoxyphenyl)-1H-imidazol-1-yl]benzene-sulfonamide [INN: CIMICOXIB], 4′-nitro-2′-phenoxymethanesulfonanilide [INN: NIMESULIDE], 6-(2,4-difluorophenoxy)-5-methylsulfonylamino-1-indanone [INN: FLOSULIDE], 5-bromo-2-(4-fluoro-phenyl)-3-(4-methanesulfonyl-phenyl)-thiophene [DUP-697], 4-acetyl-2-(2,4-difluorophenoxy)methanesulfonanilide [FK-3311], N-[2-(cyclohexyloxy)-4-nitrophenyl]methanesulfonamide [NS-398], 5-methanesulfonamido-6-(2,4-difluorophenylthio)-1-indanone [L-745337], 8-acetyl-3-(4-fluorophenyl)-2-[4-(methanesulfonyl)phenyl]imidazo[1,2-a]-pyridine [GR-253035], 4-[5-(4-chlorophenyl)-3-(trifluoromethyl)pyrazol-1-yl]benzenesulfon-amide [SC-58236], 4-(2,3-dihydro-2-oxo-3-phenyl-4-oxazolyl)-benzenesulfonamide [LAS-33815], CS-502, 2-(3,4-difluorophenyl)-4-(3-hydroxy-3-methylbutoxy)-5-[4-(methyl-sulfonyl)phenyl]-3(2H)-pyridazinone [ABT-963], GW-406381, and those COX-2 inhibitors disclosed in WO 02096427, WO 02096886 and WO 02096885; or
a bisphosphonate selected from the group consisting of ALENDRONIC ACID, RISEDRONIC ACID, TILUDRONIC ACID, IBANDRONIC ACID, ZOLEDRONIC ACID, CLODRONIC ACID, INCADRONIC ACID, OLPADRONIC ACID, MINODRONIC ACID, PAMIDRONIC ACID and ETIDRONIC ACID; or a corticosteroid selected from the group consisting of HYDROCORTISONE, PREDNISONE, PREDNISOLONE, METHYLPREDNISOLONE, TRIAMCINOLONE ACETONIDE, AMCINONIDE, CLOBETASONE, CLOBETASOL, DEFLAZACORT, DESONIDE, CLOPREDNOL, DEXAMETHASONE, DIFLORASONE, DIFLUCORTOLONE, DIFLUPREDNATE, FLUDROXYCORTIDE, FLUDROCORTISONE, FLUMETASONE, TIXOCORTOL PIVALATE, FLUOCORTIN BUTYL, CLOCORTOLONE, FLUOCINOLONE ACETONIDE, FLUOCORTOLONE, FLUOROMETHOLONE, FLUPREDNIDENE, FLUPREDNISOLONE, BETAMETHASONE, HALCINONIDE, BUDESONIDE, HALOMETASONE, RIMEXOLONE, PARAMETHASONE, PREDNYLIDENE, LOTEPREDNOL ETABONATE and PREDNICARBATE;
or a pharmaceutically acceptable derivative of any of these second ingredients.

7. A pharmaceutical composition according to claim 1, wherein the second active ingredient is selected from the group consisting of glycolic acid [o-(2,6-dichloroanilino)phenyl]acetate(ester) [INN: ACECLOFENAC]; 1-(4-chlorobenzoyl)-5-methoxy-2-methyl-1H-indole-3-acetic acid carboxymethyl ester (INN: ACEMETACIN]; 2-(acetyloxy)benzoic acid [ACETYLSALICYLIC ACID], 2-methoxyphenyl-alpha-methyl-4-(isobutyl)phenylacetate [Research Code: AF-2259], (4-allyloxy-3-chlorophenyl)acetic acid [INN: ALCLOFENAC], p-[(2-methylallyl)amino]hydratropic acid [INN: ALMINOPROFEN], 2-amino-3-benzoylphenylacetic acid [INN: AMFENAC], (plus/minus)-4-(1-hydroxyethoxy)-2-methyl-N-2-pyridyl-2H-1,2-benzothiazine-3-carboxamide ethylcarbonate (ester), 1,1-dioxide [INN: AMPIROXICAM], 2-methoxyphenyl-1-methyl-5-(p-methylbenzoyl)pyrrol-2-acetamido-acetate [INN: AMTOLMETINGUACIL], (plus/minus)-2,3-dihydro-5-(4-methoxybenzoyl)-1H pyrrolizine-1-carboxylic acid [INN: ANIROLAC], 2-[4-(alpha,alpha,alpha-trifluoro-m-tolyl)-1-piperazinyl]ethyl-N-(7-trifluoromethyl-4-quinolyl)anthranilate [INN: ANTRAFENINE], 5-(dimethylamino)-9-methyl-2-propyl-1H-pyrazolo[1,2-a][1,2,4]benzo-triazine-1,3(2H)-dione [INN: AZAPROPAZONE], 4-acetamidophenyl salicylate acetate [INN: BENORILATE], 2-(8-methyl-10,11-dihydro-11-oxodibenz[b,f]oxepin-2-yl)propionic acid [INN: BERMOPROFEN], 2-[(1-benzyl-1H-indazol-3-yl)methoxy]-2-methylpropionic acid [INN: BINDARIT], [2-amino-3-(p-bromobenzoyl)phenyl]acetic acid [INN: BROMFENAC], 3-(3-chloro-4-cyclohexylbenzoyl)propionic acid [INN: BUCLOXIC ACID], 5-butyl-1-cyclohexylbarbituric acid [INN: BUCOLOM], 4-butoxy-N-hydroxybenzeneacetamide [INN: BUFEXAMAC], butylmalonic acid mono-(1,2-diphenylhydrazide) [INN: BUMADIZONE], alpha-ethyl-4-(2-methylpropyl)benzeneacetic acid [INN: BUTIBUFEN], 2-(4-biphenylyl)butyric acid, trans-4-phenylcyclohexylamine salt (1:1) [INN: BUTIXIRATE], 2-(acetyloxy)-benzoic acid, calcium salt, compound with urea (1:1) [INN: CARBASALATE CALCIUM], (plus/minus)-6-chloro-alpha-methylcarbazole-2-acetic acid [INN: CARPROFEN], 1-cinnamoyl-5-methoxy-2-methylindole-3-acetic acid [INN: CINMETACIN], N-(2-pyridyl)-2-methyl-4-cinnamoyloxy-2H-1,2-benzothiazine-3-carboxamide-1,1-dioxide [INN: CINNOXICAM], 6-chloro-5-cyclohexyl-1-indancarboxylic acid [INN: CLIDANAC], 2-[4-(p-chlorophenyl)benzyloxy]-2-methylpropionic acid [INN: CLOBUZARIT], 5-methoxy-2-methyl-3-indolylacetohydroxamic acid [INN: DEBOXAMET], (S)-(+)-p-isobutylhydratropic acid [INN: DEXIBUPROFEN], (+)-(S)-m-benzoylhydratropic acid [INN: DEXKETOPROFEN], 2-[(2,6-di-chlorophenyl)amino]benzeneacetic acid [INN: DICLOFENAC], 2′,4′-Difluoro-4-hydroxy-3-biphenylcarboxylic acid [INN: DIFLUNISAL], 4-(2,6-dichloroanilino)-3-thiopheneacetic acid [INN: ELTENAC], N-beta-phenethyl-anthranilic acid [INN: ENFENAMIC ACID] salicylic acid acetate, ester with beta-hydroxy p-acetophenetidide [INN: ETERSALATE], 1,8-diethyl-1,3,4,9-tetrahydropyrano[3,4-b]indole-1-acetic acid [INN: ETODOLAC], 2-[[3-(trifluoromethyl)phenyl]amino]benzoic acid 2-(2-hydroxyethoxy)-ethyl ester [INN: ETOFENAMATE], p-chlorobenzoic acid, ester with 4-butyl-4-(hydroxymethyl)-1,2-diphenyl-3,5-pyrazolidinedione [INN: FECLOBUZONE], 4-biphenylacetic acid [INN: FELBINAC], 3-(4-biphenylylcarbonyl)propionic acid [INN: FENBUFEN], [o-(2,4-dichlorophenoxy)phenyl]acetic acid [INN: FENCLOFENAC], (plus/minus)-m-phenoxyhydratropic acid [INN: FENOPROFEN], 4-(p-chlorophenyl)-2-phenyl-5-thiazoleacetic acid [INN: FENTIAZAC], (plus/minus)-alpha-[[(2-hydroxy-1,1-dimethylethyl)amino]methyl]-benzyl alcohol [INN: FEPRADINOL], 4-(2′,4′-difluorobiphenylyl)-4-oxo-2-methylbutanoic acid [INN: FLOBUFEN], 2,3-dihydroxypropyl N-[8-(trifluoromethyl)-4-quinoly]anthranilate [INN: FLOCTAFENINE], N-(alpha,alpha,alpha-trifluoro-m-tolyl)-anthranilic acid [INN: FLUFENAMIC ACID], (plus)-2-(p-fluorophenyl)-alpha-methyl-5-benzoxazoleacetic acid [INN: FLUNOXAPROFEN], 2-fluoro-alpha-methyl-4-biphenylacetic acid [INN: FLURBIPROFEN], (plus/minus)-2-(2-fluoro-4-biphenylyl)propionic acid 1 (acetoxy)ethyl ester [INN: FLURBIPROFEN AXETIL], 2-ethyl-2,3-dihydro-5-benzofuranacetic acid [INN: FUROFENAC], 2-[4-(2′-furoyl)phenyl]propionic acid [INN: FURPROFEN], 2-[2-[1-(p-chlorobenzoyl)-5-methoxy-2-methylindol-3-yl]acetamido]-2-deoxy-D-glucose [INN: GLUCAMETACIN], 2-(2-fluorobiphenyl-4-yl)propionic acid 4-nitrooxybutylester [Research Code: HCT-1026], (p-isobutylphenyl)acetic acid [INN: IBUFENAC], alpha-p-isobutylphenylpropionic acid [INN: IBUPROFEN], methyl 4-(3-thienyl)phenyl-alpha-methylacetate [Research Code: IDPH-8261], (plus/minus)-2-[p-(1-oxo-2-isoindolinyl)phenyl]butyric acid [INN: INDOBUFEN], 1-(4-chlorobenzoyl)-5-methoxy-2-methyl-1H-indole-3-acetic acid [INN: INDOMETACIN], 1-(4-chlorobenzoyl)-5-methoxy-2-methyl-1H-indole-3-acetic acid, 3,7,11-trimethyl-2,6,10-dodecatrienyl ester [INN: INDOMETACIN FARNESIL], p-(1-oxo-2-isoindolinyl)hydratropic acid [INN: INDOPROFEN], 2-(10-methoxy-4H-benzo[4,5]cyclohepta[1,2-b]thiophen-4-ylidene)-acetic acid [Research Code: IX-207-887], m-benzoylhydratropic acid [INN: KETOPROFEN], (DL)-5-benzoyl-3H-1,2-dihydropyrrolo[1,2-a]pyrrole-1-carboxylic acid [INN: KETOROLAC], 2,3-dihydro-5-hydroxy-6-[2-(hydroxy-methyl)cinnamyl]benzofuran [Research Code: L-651896], N-(2-carboxyphenyl)-4-chloroanthranilic acid [INN: LOBENZARIT], 3-(p-chlorophenyl)-1-phenylpyrazole-4-acetic acid [INN: LONAZOLAC], 6-chloro-4-hydroxy-2-methyl-N-2-pyridyl-2H-thieno[2,3-e]-1,2-thiazine-3-carboxamide 1,1-dioxide [INN: LORNOXICAM], 2-[4-(2-oxocyclopentan-1-ylmethyl)phenyl]-propionate [INN: LOXOPROFEN], 2(R)-[4-(3-methyl-2-thienyl)phenyl]propionic acid [Research Code: M-5010], N-(2,3-xylyl)anthranilic acid [INN: MEFENAMIC ACID], 4-hydroxy-2-methyl-N-(5-methyl-2-thiazolyl)-2H-1,2-benzothiazine-3-carboxamide 1,1-dioxide [INN: MELOXICAM], 5-aminosalicylic acid [INN: MESALAZINE], (2,2-dimethyl-6-(4-chlorophenyl)-7-phenyl-2,3-dihydro-1H-pyrrolizin-5-yl)-acetic acid [Research Code: ML-3000], 3,4-bis(4-methoxyphenyl)-5-isoxazoleacetic acid [INN: MOFEZOLAC], 4-(6-methoxy-2-naphthyl)-2-butanone [INN: NABUMETONE], (plus)-6-methoxy-alpha-methyl-2-naphthalineacetic acid [INN: NAPROXEN], 2-[3-(trifluoromethyl)anilino]nicotinic acid [INN: NIFLUMIC ACID], 5,5′-azodisalicylic acid [INN: OLSALAZINE], 4,5-diphenyl-2-oxazolepropionic acid [INN: OXAPROZIN], alpha-methyl-4-[(2-oxocyclohexylidene)methyl]benzene acetic acid [INN: PELUBIPROFEN], 4-butyl-1,2-diphenyl-3,5-pyrazolidinedione [INN: PHENYLBUTAZONE], 2-(p-isobutylphenyl)propionic acid 2-pyridyl-methyl ester [INN: PIMEPROFEN], 4-(p-chlorophenyl)-1-(p-fluorophenyl)pyrazole-3-acetic acid [INN: PIRAZOLAC], 4-hydroxy-2-methyl-N-2-pyridyl-2H-1,2-benzothiadiazin-3-carboxamide 1,1-dioxide [INN: PIROXICAM], 3-chloro-4-(3-pyrrolin-1-yl)hydratropic acid [INN: PIRPROFEN], 2-[5H-(1)benzopyrano]2,3-b]pyridin-7-yl]propionic acid [INN: PRANOPROFEN], 2,6-di-tert-butyl-4-(2′-thenoyl)phenol [INN: PRIFELONE], alpha-cyano-1-methyl-beta-oxopyrrole-2-propionanilide [INN: PRINOMIDE], 3-[4-(2-hydroxyethyl)-1-piperazinyl]-propyl-D,L-4-benzamido-N,N-dipropylglutaramat 1-(p-chlorobenzoyl)-5-methoxy-2-methyl-indole-3-acetate (ester) [INN: PROGLUMETACIN], 7-methyl-1-(1-methylethyl)-4-phenyl-2(1H)quinazolinone [INN: PROQUAZONE], 7-methoxy-alpha,10-dimethylphenothiazine-2-acetic acid [INN: PROTIZINIC ACID], 2-[[2-(p-chlorophenyl)-4-methyl-5-oxazolyl]methoxy]-2-methylpropionic acid [INN: ROMAZARIT], o-hydroxybenzamide [SALICYLAMIDE], 2-hydroxybenzoic acid [SALICYLIC ACID], N-acetyl-L-cysteine salicylate (ester), acetate (ester) [INN: SALMISTEINE], N-acetyl-L-cysteine salicylate (ester) [INN: SALNACEDIN], 2-hydroxybenzoic acid 2-carboxyphenyl ester [INN: SALSALATE], 4-[1-(2-fluorobiphenyl-4-yl)ethyl]-N-methylthiazole-2-amine [Research Code: SM-8849], (Z)-5-fluoro-2-methyl-1-[p-(methylsulfinyl)benzylidene]indene-3-acetic acid [INN: SULINDAC], p-2-thenoylhydratropic acid [INN: SUPROFEN] 2-(4-(3-methyl-2-butenyl)phenyl)propionic acid [Research Code: TA-60], phthalidyl 2-(alpha,alpha,alpha-trifluoro-m-toluidino)nicotinate [INN: TALNIFLUMATE], (Z)-5-chloro-3-(2-thenoyl)-2-oxoindole-1-carboxamide [INN: TENIDAP], 2-thiophenecarboxylic acid, ester with salicylic acid [INN: TENOSAL], 4-hydroxy-2-methyl-N-2-pyridyl-2H-thieno[2,3-e]-1,2-thiazine-3-carboxamide [INN: TENOXICAM], 5-(4-chlorophenyl)-N-hydroxy-1-(4-methoxyphenyl)-N-methyl-1H-pyrazole-3-propionamide [INN: TEPOXALIN], alpha-(5-benzoyl-2-thienyl)propionic acid [INN: TIAPROFENIC ACID], 5-chloro-3-[4-(2-hydroxyethyl)-1-piperazinyl]carbonylmethyl-2-benzo-thiazolin one [INN: TIARAMIDE], 2-(2-methyl-5H-[1]benzopyrano[2,3-b]pyridin-7-yl)-propionic acid N,N-dimethylcarbamoylmethyl ester [INN: TILNOPROFEN ARBAMEL], 1-Cyclohexyl-2-(2-methyl-4-quinolyl)-3-(2-thiazolyl)guanidine [INN: TIMEGADINE], 2-amino-6-benzyl-4,5,6,7-tetrahydrothieno[2,3-c]pyridine [INN: TINORIDINE], N-(3-chloro-o-tolyl)anthranilic acid [INN: TOLFENAMIC ACID], 1-methyl-5-(4-methylbenzoyl)-1H-pyrrole-2-acetic acid [INN: TOLMETIN], hydroxybis[alpha-methyl-4-(2-methylpropyl)benzene acetato-O]-aluminium [Research Code: U-18573-G], N-(3-trifluoromethylphenyl)-anthranilic acid n-butyl ester [INN: UFENAMATE], 2-[4-[3-(hydroxyimino)cyclohexyl]phenyl]propionic acid [INN: XIMOPROFEN], 2-(10,11-dihydro-10-oxo-dibenz[b,f]thiepin-2-yl-propionic acid [INN: ZALTOPROFEN], 2-[4-(2-thiazolyloxy)phenyl]-propionic acid [INN: ZOLIPROFEN], 5-chloro-6′-methyl-3-[4-(methylsulfonyl)phenyl]-2,3′-bipyridine [INN: ETORICOXIB], 4-[5-(4-methylphenyl)-3-(trifluoromethyl)-1H-pyrazol-1-yl]benzene-sulfonamide [INN: CELECOXIB], 4-[p-(methylsulfonyl)phenyl]-3-phenyl-2(5H)-furanone [INN: ROFECOXIB], N-[[p-(5-methyl-3-phenyl-4-isoxazolyl)phenyl]sulfonyl]propion-amide [INN: PARECOXIB], p-(5-methyl-3-phenyl-4-isoxazolyl)benzenesulfonamide [INN: VALDECOXIB], 2-[2-(2-chloro-6-fluorophenylamino)-5-methylphenyl]acetic acid [INN: LUMIRACOXIB], 4-(4-cyclohexyl-2-methyloxazol-5-yl)-2-fluorobenzene-sulfonamide [INN: TILMACOXIB], 4-[4-chloro-5-(3-fluoro-4-methoxyphenyl)-1H-imidazol-1-yl]benzene-sulfonamide [INN: CIMICOXIB], 4′-nitro-2′-phenoxymethanesulfonanilide [INN: NIMESULIDE], 6-(2,4-difluorophenoxy)-5-methylsulfonylamino-1-indanone [INN: FLOSULIDE], 5-bromo-2-(4-fluoro-phenyl)-3-(4-methanesulfonyl-phenyl)-thiophene [DUP-697], 4-acetyl-2-(2,4-difluorophenoxy)methanesulfonanilide [FK-3311], N-[2-(cyclohexyloxy)-4-nitrophenyl]methanesulfonamide [NS-398], 5-methanesulfonamido-6-(2,4-difluorophenylthio)-1-indanone [L-745337], 8-acetyl-3-(4-fluorophenyl)-2-[4-(methanesulfonyl)phenyl]imidazo[1,2-a]-pyridine [GR-253035], 4-[5-(4-chlorophenyl)-3-(trifluoromethyl)pyrazol-1-yl]benzenesulfon-amide [SC-58236], 4-(2,3-dihydro-2-oxo-3-phenyl-4-oxazolyl)-benzenesulfonamide [LAS-33815], CS-502, 2-(3,4-difluorophenyl)-4-(3-hydroxy-3-methylbutoxy)-5-[4-(methylsulfonyl)phenyl]-3(2H)-pyridazinone [ABT-963], GW-406381, ALENDRONIC ACID, RISEDRONIC ACID, TILUDRONIC ACID, IBANDRONIC ACID, ZOLEDRONIC ACID, CLODRONIC ACID, INCADRONIC ACID, OLPADRONIC ACID, MINODRONIC ACID, PAMIDRONIC ACID, ETIDRONIC ACID, BETAMETHASONE, DEXAMETHASONE, FLUOCORTOLONE, METHYLPREDNISOLONE, PREDNISOLONE, PREDNISONE, HYDROCORTISONE, BUDESONIDE, TRIAMCINOLONE ACETONIDE; and the pharmaceutically acceptable derivatives of any of these second active ingredients.

8. A pharmaceutical composition according to claim 1, wherein the second active ingredient is a NSAID selected from the group consisting of glycolic acid [o-(2,6-dichloroanilino)phenyl]acetate(ester) [INN: ACECLOFENAC]; 1-(4-chlorobenzoyl)-5-methoxy-2-methyl-1H-indole-3-acetic acid carboxymethyl ester [INN: ACEMETACIN]; 2-(acetyloxy)benzoic acid [ACETYLSALICYLIC ACID], 2-methoxyphenyl-alpha-methyl-4-(isobutyl)phenylacetate [Research Code: AF-2259], (4-allyloxy-3-chlorophenyl)acetic acid [INN: ALCLOFENAC], p-[(2-methylallyl)amino]hydratropic acid [INN: ALMINOPROFEN], 2-amino-3-benzoylphenylacetic acid [INN: AMFENAC], (plus/minus)-4-(1-hydroxyethoxy)-2-methyl-N-2-pyridyl-2H-1,2-benzothiazine-3-carboxamide ethylcarbonate (ester), 1,1-dioxide [INN: AMPIROXICAM], 2-methoxyphenyl-1-methyl-5-(p-methylbenzoyl)pyrrol-2-acetamido-acetate [INN: AMTOLMETINGUACIL], (plus/minus)-2,3-dihydro-5-(4-methoxybenzoyl)-1H pyrrolizine-1-carboxylic acid [INN: ANIROLAC], 2-[4-(alpha,alpha,alpha-trifluoro-m-tolyl)-1-piperazinyl]ethyl-N-(7-trifluoromethyl-4-quinolyl)anthranilate [INN: ANTRAFENINE], 5-(dimethylamino)-9-methyl-2-propyl-1H-pyrazolo[1,2-a][1,2,4]benzo-triazine-1,3(2H)-dione [INN: AZAPROPAZONE], 4-acetamidophenyl salicylate acetate [INN: BENORILATE], 2-(8-methyl-10,11-dihydro-11-oxodibenz[b,f]oxepin-2-yl)propionic acid [INN: BERMOPROFEN], 2-[(1-benzyl-1H-indazol-3-yl)methoxy]-2-methylpropionic acid [INN: BINDARIT], [2-amino-3-(p-bromobenzoyl)phenyl]acetic acid [INN: BROMFENAC], 3-(3-chloro-4-cyclohexylbenzoyl)propionic acid [INN: BUCLOXIC ACID], 5-butyl-1-cyclohexylbarbituric acid [INN: BUCOLOM], 4-butoxy-N-hydroxybenzeneacetamide [INN: BUFEXAMAC], butylmalonic acid mono(1,2-diphenylhydrazide) [INN: BUMADIZONE], alpha-ethyl-4-(2-methylpropyl)benzeneacetic acid [INN: BUTIBUFEN], 2-(4-biphenylyl)butyric acid, trans-4-phenylcyclohexylamine salt (1:1) [INN: BUTIXIRATE], 2-(acetyloxy)-benzoic acid, calcium salt, compound with urea (1:1) [INN: CARBASALATE CALCIUM], (plus/minus)-6-chloro-alpha-methylcarbazole-2-acetic acid [INN: CARPROFEN], 1-cinnamoyl-5-methoxy-2-methylindole-3-acetic acid [INN: CINMETACIN], N-(2-pyridyl)-2-methyl-4-cinnamoyloxy-2H-1,2-benzothiazine-3-carboxamide-1,1-dioxide [INN: CINNOXICAM], 6-chloro-5-cyclohexyl-1-indancarboxylic acid [INN: CLIDANAC], 2-[4-(p-chlorophenyl)benzyloxy]-2-methylpropionic acid [INN: CLOBUZARIT], 5-methoxy-2-methyl-3-indolylacetohydroxamic acid [INN: DEBOXAMET], (S)-(+)-p-isobutylhydratropic acid [INN: DEXIBUPROFEN], (+)-(S)-m-benzoylhydratropic acid [INN: DEXKETOPROFEN], 2-[(2,6-dichlorophenyl)amino]benzeneacetic acid [INN: DICLOFENAC], 2′,4′-Difluoro-4-hydroxy-3-biphenylcarboxylic acid [INN: DIFLUNISAL], 4-(2,6-dichloroanilino)-3-thiopheneacetic acid [INN: ELTENAC], N-beta-phenethyl-anthranilic acid [INN: ENFENAMIC ACID] salicylic acid acetate, ester with beta-hydroxy p-acetophenetidide [INN: ETERSALATE], 1,8-diethyl-1,3,4,9-tetrahydropyrano[3,4-b]indole-1-acetic acid [INN: ETODOLAC], 2-[[3-(trifluoromethyl)phenyl]amino]benzoic acid 2-(2-hydroxyethoxy)-ethyl ester [INN: ETOFENAMATE], p-chlorobenzoic acid, ester with 4-butyl-4-(hydroxymethyl)-1,2-diphenyl-3,5-pyrazolidinedione [INN: FECLOBUZONE], 4-biphenylacetic acid [INN: FELBINAC], 3-(4-biphenylylcarbonyl)propionic acid [INN: FENBUFEN], [o-(2,4-dichlorophenoxy)phenyl]acetic acid [INN: FENCLOFENAC], (plus/minus)-m-phenoxyhydratropic acid [INN: FENOPROFEN], 4-(p-chlorophenyl)-2-phenyl-5-thiazoleacetic acid [INN: FENTIAZAC], (plus/minus)-alpha-[[(2-hydroxy-1,1-dimethylethyl)amino]methyl]-benzyl alcohol [INN: FEPRADINOL], 4-(2′,4′-difluorobiphenylyl)-4-oxo-2-methylbutanoic acid [INN: FLOBUFEN], 2,3-dihydroxypropyl N-[8-(trifluoromethyl)-4-quinoly]anthranilate [INN: FLOCTAFENINE], N-(alpha,alpha,alpha-trifluoro-m-tolyl)anthranilic acid [INN: FLUFENAMIC ACID], (plus)-2-(p-fluorophenyl)-alpha-methyl-5-benzoxazoleacetic acid [INN: FLUNOXAPROFEN], 2-fluoro-alpha-methyl-4-biphenylacetic acid [INN: FLURBIPROFEN], (plus/minus)-2-(2-fluoro-4-biphenylyl)propionic acid 1 (acetoxy)ethyl ester [INN: FLURBIPROFEN AXETIL], 2-ethyl-2,3-dihydro-5-benzofuranacetic acid [INN: FUROFENAC], 2-[4-(2′-furoyl)phenyl]propionic acid [INN: FURPROFEN], 2-[2-[1-(p-chlorobenzoyl)-5-methoxy-2-methylindol-3-yl]acetamido]-2-deoxy-D-glucose [INN: GLUCAMETACIN], 2-(2-fluorobiphenyl-4-yl)propionic acid 4-nitrooxybutylester [Research Code: HCT-1026], (p-isobutylphenyl)acetic acid [INN: IBUFENAC], alpha-p-isobutylphenylpropionic acid [INN: IBUPROFEN], methyl 4-(3-thienyl)phenyl-alpha-methylacetate [Research Code: IDPH-8261], (plus/minus)-2-[p-(1-oxo-2-isoindolinyl)phenyl]butyric acid [INN: INDOBUFEN], 1-(4-chlorobenzoyl)-5-methoxy-2-methyl-1H-indole-3-acetic acid [INN: INDOMETACIN], 1-(4-chlorobenzoyl)-5-methoxy-2-methyl-1H-indole-3-acetic acid, 3,7,11-trimethyl-2,6,10-dodecatrienyl ester [INN: INDOMETACIN FARNESIL], p-(1-oxo-2-isoindolinyl)hydratropic acid [INN: INDOPROFEN], 2-(10-methoxy-4H-benzo[4,5]cyclohepta[1,2-b]thiophen-4-ylidene)-acetic acid [Research Code: IX-207-887], m-benzoylhydratropic acid [INN: KETOPROFEN], (DL)-5-benzoyl-3H-1,2-dihydropyrrolo[1,2-a]pyrrole-1-carboxylic acid [INN: KETOROLAC], 2,3-dihydro-5-hydroxy-6-[2-(hydroxymethyl)cinnamyl]benzofuran [Research Code: L-651896], N-(2-carboxyphenyl)-4-chloroanthranilic acid [INN: LOBENZARIT], 3-(p-chlorophenyl)-1-phenylpyrazole-4-acetic acid [INN: LONAZOLAC], 6-chloro-4-hydroxy-2-methyl-N-2-pyridyl-2H-thieno[2,3-e]-1,2-thiazine-3-carboxamide 1,1-dioxide [INN: LORNOXICAM], 2-[4-(2-oxocyclopentan-1-ylmethyl)phenyl]-propionate [INN: LOXOPROFEN], 2(R)-[4-(3-methyl-2-thienyl)phenyl]propionic acid [Research Code: M-5010], N-(2,3-xylyl)anthranilic acid [INN: MEFENAMIC ACID], 4-hydroxy-2-methyl-N-(5-methyl-2-thiazolyl)-2H-1,2-benzothiazine-3-carboxamide 1,1-dioxide [INN: MELOXICAM], 5-aminosalicylic acid [INN: MESALAZINE], (2,2-dimethyl-6-(4-chlorophenyl)-7-phenyl-2,3-dihydro-1H-pyrrolizin-5-yl)-acetic acid [Research Code: ML-3000], 3,4-bis(4-methoxyphenyl)-5-isoxazoleacetic acid [INN: MOFEZOLAC], 4-(6-methoxy-2-naphthyl)-2-butanone [INN: NABUMETONE], (plus)-6-methoxy-alpha-methyl-2-naphthalineacetic acid [INN: NAPROXEN], 2-[3-(trifluoromethyl)anilino]nicotinic acid [INN: NIFLUMIC ACID], 5,5′-azodisalicylic acid [INN: OLSALAZINE], 4,5-diphenyl-2-oxazolepropionic acid [INN: OXAPROZIN], alpha-methyl-4-[(2-oxocyclohexylidene)methyl]benzene acetic acid [INN: PELUBIPROFEN], 4-butyl-1,2-diphenyl-3,5-pyrazolidinedione [INN: PHENYLBUTAZONE], 2-(p-isobutylphenyl)propionic acid 2-pyridyl-methyl ester [INN: PIMEPROFEN], 4-(p-chlorophenyl)-1-(p-fluorophenyl)pyrazole-3-acetic acid [INN: PIRAZOLAC], 4-hydroxy-2-methyl-N-2-pyridyl-2H-1,2-benzothiadiazin-3-carboxamide 1,1-dioxide [INN: PIROXICAM], 3-chloro-4-(3-pyrrolin-1-yl)hydratropic acid [INN: PIRPROFEN], 2-[5H-(1)benzopyrano]2,3-b]pyridin-7-yl]propionic acid [INN: PRANOPROFEN], 2,6-di-tert-butyl-4-(2′-thenoyl)phenol [INN: PRIFELONE], alpha-cyano-1-methyl-beta-oxopyrrole-2-propionanilide [INN: PRINOMIDE], 3-[4-(2-hydroxyethyl)-1-piperazinyl]-propyl-D,L-4-benzamido-N,N-dipropylglutaramat 1-(p-chlorobenzoyl)-5-methoxy-2-methylindole-3-acetate (ester) [INN: PROGLUMETACIN], 7-methyl-1-(1-methylethyl)-4-phenyl-2(1H)quinazolinone [INN: PROQUAZONE], 7-methoxy-alpha,10-dimethylphenothiazine-2-acetic acid [INN: PROTIZINIC] ACID], 2-[[2-(p-chlorophenyl)-4-methyl-5-oxazolyl]methoxy]-2-methylpropionic acid [INN: ROMAZARIT], o-hydroxybenzamide [SALICYLAMIDE], 2-hydroxybenzoic acid [SALICYLIC ACID], N-acetyl-L-cysteine salicylate (ester), acetate (ester) [INN: SALMISTEINE], N-acetyl-L-cysteine salicylate (ester) [INN: SALNACEDIN], 2-hydroxybenzoic acid 2-carboxyphenyl ester [INN: SALSALATE], 4-[1-(2-fluorobiphenyl-4-yl)ethyl]-N-methylthiazole-2-amine [Research Code: SM-8849], (Z)-5-fluoro-2-methyl-1-[p-(methylsulfinyl)benzylidene]indene-3-acetic acid [INN: SULINDAC], p-2-thenoylhydratropic acid [INN: SUPROFEN] 2-(4-(3-methyl-2-butenyl)phenyl)propionic acid [Research Code: TA-60], phthalidyl 2-(alpha,alpha,alpha-trifluoro-m-toluidino)nicotinate [INN: TALNIFLUMATE], (Z)-5-chloro-3-(2-thenoyl)-2-oxoindole-1-carboxamide [INN: TENIDAP], 2-thiophenecarboxylic acid, ester with salicylic acid [INN: TENOSAL], 4-hydroxy-2-methyl-N-2-pyridyl-2H-thieno[2,3-e]-1,2-thiazine-3-carboxamide [INN: TENOXICAM], 5-(4-chlorophenyl)-N-hydroxy-1-(4-methoxyphenyl)-N-methyl-1H-pyrazole-3-propionamide [INN: TEPOXALIN], alpha-(5-benzoyl-2-thienyl)propionic acid [INN: TIAPROFENIC ACID], 5-chloro-3-[4-(2-hydroxyethyl)-1-piperazinyl]carbonylmethyl-2-benzo-thiazolin one [INN: TIARAMIDE], 2-(2-methyl-5H-[l]benzopyrano[2,3-b]pyridin-7-yl)-propionic acid N,N-dimethylcarbamoylmethyl ester [INN: TILNOPROFEN ARBAMEL], 1-Cyclohexyl-2-(2-methyl-4-quinolyl)-3-(2-thiazolyl)guanidine [INN: TIMEGADINE], 2-amino-6-benzyl-4,5,6,7-tetrahydrothieno[2,3-c]pyridine [INN: TINORIDINE], N-(3-chloro-o-tolyl)anthranilic acid [INN: TOLFENAMIC ACID], 1-methyl-5-(4-methylbenzoyl)-1H-pyrrole-2-acetic acid [INN: TOLMETIN], hydroxybis[alpha-methyl-4-(2-methylpropyl)benzene acetato-O]-aluminium [Research Code: U-18573-G], N-(3-trifluoromethylphenyl)-anthranilic acid n-butyl ester [INN: UFENAMATE], 2-[4-[3-(hydroxyimino)cyclohexyl]phenyl]propionic acid [INN: XIMOPROFEN], 2-(10,11-dihydro-10-oxo-dibenz[b,f]thiepin-2-yl-propionic acid [INN: ZALTOPROFEN] and 2-[4-(2-thiazolyloxy)phenyl]-propionic acid [INN: ZOLIPROFEN]; or a NO-NSAID selected from the group consisting of those NO-NSAIDs which are disclosed in WO 96/32946, WO 96/35416, WO 96/38136, WO 96/39409, WO 00/50037, U.S. Pat. No. 6,057,347, WO 94/04484, WO 94/12463, WO 95/09831, WO 95/30641, WO 97/31654, WO 99/44595, WO 99/45004 and WO 01/45703; or a COX-2 inhibitor selected from the group consisting of CELEBREX (CELECOXIB) and VIOXX (ROFECOXIB); or a bisphosphonate selected from the group consisting of ALENDRONATE, RISEDRONATE, TILUDRONATE, IBANDRONATE, ZOLEDRONATE and ETIDRONATE;
or a pharmaceutically acceptable derivative of these compounds.

9. A pharmaceutical composition according to claim 1, wherein the second active ingredient is selected from the group consisting of ACETYLSALICYLIC ACID, DICLOFENAC, DIFLUNISAL, ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID, TOLMETIN, ETORICOXIB, CELECOXIB, ROFECOXIB, PARECOXIB, VALDECOXIB, LUMIRACOXIB, TILMACOXIB, CIMICOXIB, NIMESULIDE, FLOSULIDE, DUP-697, FK-3311, NS-398, L-745337, GR-253035, SC-58236, LAS-33815, CS-502, ABT-963, GW-406381, ALENDRONIC ACID, RISEDRONIC ACID, TILUDRONIC ACID, IBANDRONIC ACID, ZOLEDRONIC ACID, CLODRONIC ACID, INCADRONIC ACID, OLPADRONIC ACID, MINODRONIC ACID, PAMIDRONIC ACID, ETIDRONIC ACID, BETAMETHASONE, DEXAMETHASONE, FLUOCORTOLONE, METHYLPREDNISOLONE, PREDNISOLONE, PREDNISONE, HYDROCORTISONE, BUDESONIDE, TRIAMCINOLONE ACETONIDE,
and the pharmaceutically acceptable derivatives thereof.

10. A pharmaceutical composition according to claim 1, wherein the second active ingredient is selected from the group consisting of ACETYLSALICYLIC ACID, DICLOFENAC, DIFLUNISAL, ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID, TOLMETIN, ETORICOXIB, CELECOXIB, ROFECOXIB, PARECOXIB, VALDECOXIB, LUMIRACOXIB, TILMACOXIB, CIMICOXIB, ALENDRONIC ACID, RISEDRONIC ACID, TILUDRONIC ACID, IBANDRONIC ACID, ZOLEDRONIC ACID, CLODRONIC ACID, INCADRONIC ACID, OLPADRONIC ACID, MINODRONIC ACID, PAMIDRONIC ACID, ETIDRONIC ACID, and the pharmaceutically acceptable derivatives of any of these second ingredients.

11. A pharmaceutical composition according to claim 1, wherein the second active ingredient is a NSAID or a COX-2 inhibitor or a bisphosphonate selected from the group consisting of ACETYLSALICYLIC ACID, DICLOFENAC, DIFLUNISAL, ETODOLAC, FENOPROFEN, FLOCTAFENINE, FLURBIPROFEN, IBUPROFEN, INDOMETHACIN, KETOPROFEN, MECLOFENAMATE, MEFENAMIC ACID, MELOXICAM, NABUMETONE, NAPROXEN, OXAPROZIN, PHENYLBUTAZONE, PIROXICAM, SULINDAC, TENOXICAM, TIAPROFENIC ACID, TOLMETIN, CELEBREX (CELECOXIB), VIOXX (ROFECOXIB), ALENDRONATE, RISEDRONATE, TILUDRONATE, IBANDRONATE, ZOLEDRONATE, ETIDRONATE and the pharmaceutically acceptable derivatives of these compounds.

12. A pharmaceutical composition according to claim 1, wherein the second active ingredient is selected from the group consisting of DICLOFENAC, IBUPROFEN, INDOMETHACIN, NAPROXEN, PIROXICAM, ETORICOXIB, CELECOXIB, ROFECOXIB, PARECOXIB, VALDECOXIB, LUMIRACOXIB, TILMACOXIB, CIMICOXIB, NIMESULIDE, FLOSULIDE, DUP-697, FK-3311, NS-398, L-745337, GR-253035, SC-58236, LAS-33815, CS-502, ABT-963, GW-406381 and the pharmaceutically acceptable derivatives thereof.

13. A pharmaceutical composition according to claim 1, wherein the second active ingredient is DICLOFENAC or a pharmaceutically acceptable derivative of this compound.

14.-15. (canceled)

16. A kit comprising a dosage unit of an agent as defined as a first active ingredient in claim 1 and a dosage unit of an agent as defined as a second active ingredient selected from the group consisting of a NSAID selected from the group consisting of glycolic acid [o-(2,6-dichloroanilino)phenyl]acetate(ester) [INN: ACECLOFENAC]; 1-(4-chlorobenzoyl)-5-methoxy-2-methyl-1H-indole-3-acetic acid carboxymethyl ester [INN: ACEMETACIN]; 2-(acetyloxy)benzoic acid [ACETYLSALICYLIC ACID], 2-methoxyphenyl-alpha-methyl-4-(isobutyl)phenylacetate [Research Code: AF-2259], (4-allyloxy-3-chlorophenyl)acetic acid [INN: ALCLOFENAC], p-[(2-methylallyl)amino]hydratropic acid [INN: ALMINOPROFEN], 2-amino-3-benzoylphenylacetic acid [INN: AMFENAC], (plus/minus)-4-(1-hydroxyethoxy)-2-methyl-N-2-pyridyl-2H-1,2-benzothiazine-3-carboxamide ethylcarbonate (ester), 1,1-dioxide [INN: AMPIROXICAM], 2-methoxyphenyl-1-methyl-5-(p-methylbenzoyl)pyrrol-2-acetamido-acetate [INN: AMTOLMETINGUACIL], (plus/minus)-2,3-dihydro-5-(4-methoxybenzoyl)-1H pyrrolizine-1-carboxylic acid [INN: ANIROLAC], 2-[4-(alpha,alpha,alpha-trifluoro-m-tolyl)-1-piperazinyl]ethyl-N-(7-trifluoromethyl-4-quinolyl)anthranilate [INN: ANTRAFENINE], 5-(dimethylamino)-9-methyl-2-propyl-1H-pyrazolo[1,2-a][1,2,4]benzo-triazine-1,3(2H)-dione [INN: AZAPROPAZONE], 4-acetamidophenyl salicylate acetate [INN: BENORILATE], 2-(8-methyl-10,11-dihydro-11-oxodibenz[b,f]oxepin-2-yl)propionic acid [INN: BERMOPROFEN], 2-[(1-benzyl-1H-indazol-3-yl)methoxy]-2-methylpropionic acid [INN: BINDARIT], [2-amino-3-(p-bromobenzoyl)phenyl]acetic acid [INN: BROMFENAC], 3-(3-chloro-4-cyclohexylbenzoyl)propionic acid [INN: BUCLOXIC ACID], 5-butyl-1-cyclohexylbarbituric acid [INN: BUCOLOM], 4-butoxy-N-hydroxybenzeneacetamide [INN: BUFEXAMAC], butylmalonic acid mono-(1,2-diphenylhydrazide) [INN: BUMADIZONE], alpha-ethyl-4-(2-methylpropyl)benzeneacetic acid [INN: BUTIBUFEN], 2-(4-biphenylyl)butyric acid, trans-4-phenylcyclohexylamine salt (1:1) [INN: BUTIXIRATE], 2-(acetyloxy)-benzoic acid, calcium salt, compound with urea (1:1) [INN: CARBASALATE CALCIUM], (plus/minus)-6-chloro-alpha-methylcarbazole-2-acetic acid [INN: CARPROFEN], 1-cinnamoyl-5-methoxy-2-methylindole-3-acetic acid [INN: CINMETACIN], N-(2-pyridyl)-2-methyl-4-cinnamoyloxy-2H-1,2-benzothiazine-3-carboxamide-1,1-dioxide [INN: CINNOXICAM], 6-chloro-5-cyclohexyl-1-indancarboxylic acid [INN: CLIDANAC], 2-[4-(p-chlorophenyl)benzyloxy]-2-methylpropionic acid [INN: CLOBUZARIT], 5-methoxy-2-methyl-3-indolylacetohydroxamic acid [INN: DEBOXAMET], (S)-(+)-p-isobutylhydratropic acid [INN: DEXIBUPROFEN], (+)-(S)-m-benzoylhydratropic acid [INN: DEXKETOPROFEN], 2-[(2,6-dichlorophenyl)amino]benzeneacetic acid [INN: DICLOFENAC], 2′,4′-Difluoro-4-hydroxy-3-biphenylcarboxylic acid [INN: DIFLUNISAL], 4-(2,6-dichloroanilino)-3-thiopheneacetic acid [INN: ELTENAC], N-beta-phenethyl-anthranilic acid [INN: ENFENAMIC ACID] salicylic acid acetate, ester with beta-hydroxy p-acetophenetidide [INN: ETERSALATE], 1,8-diethyl-1,3,4,9-tetrahydropyrano[3,4-b]indole-1-acetic acid [INN: ETODOLAC], 2-[[3-(trifluoromethyl)phenyl]amino]benzoic acid 2-(2-hydroxyethoxy)-ethyl ester [INN: ETOFENAMATE], p-chlorobenzoic acid, ester with 4-butyl-4-(hydroxymethyl)-1,2-diphenyl-3,5-pyrazolidinedione [INN: FECLOBUZONE], 4-biphenylacetic acid [INN: FELBINAC], 3-(4-biphenylylcarbonyl)propionic acid [INN: FENBUFEN], [o-(2,4-dichlorophenoxy)phenyl]acetic acid [INN: FENCLOFENAC], (plus/minus)-m-phenoxyhydratropic acid [INN: FENOPROFEN], 4-(p-chlorophenyl)-2-phenyl-5-thiazoleacetic acid [INN: FENTIAZAC], (plus/minus)-alpha-[[(2-hydroxy-1,1-dimethylethyl)amino]methyl]-benzyl alcohol [INN: FEPRADINOL], 4-(2′,4′-difluorobiphenylyl)-4-oxo-2-methylbutanoic acid [INN: FLOBUFEN], 2,3-dihydroxypropyl N-[8-(trifluoromethyl)-4-quinoly]anthranilate [INN: FLOCTAFENINE], N-(alpha,alpha,alpha-trifluoro-m-tolyl)-anthranilic acid [INN: FLUFENAMIC ACID], (plus)-2-(p-fluorophenyl)-alpha-methyl-5-benzoxazoleacetic acid [INN: FLUNOXAPROFEN], 2-fluoro-alpha-methyl-4-biphenylacetic acid [INN: FLURBIPROFEN], (plus/minus)-2-(2-fluoro-4-biphenylyl)propionic acid 1 (acetoxy)ethyl ester [INN: FLURBIPROFEN AXETIL], 2-ethyl-2,3-dihydro-5-benzofuranacetic acid [INN: FUROFENAC], 2-[4-(2′-furoyl)phenyl]propionic acid [INN: FURPROFEN], 2-[2-[1-(p-chlorobenzoyl)-5-methoxy-2-methylindol-3-yl]acetamido]-2-deoxy-D-glucose [INN: GLUCAMETACIN], 2-(2-fluorobiphenyl-4-yl)propionic acid 4-nitrooxybutylester [Research Code: HCT-1026], (p-isobutylphenyl)acetic acid [INN: IBUFENAC], alpha-p-isobutylphenylpropionic acid [INN: IBUPROFEN], methyl 4-(3-thienyl)phenyl-alpha-methylacetate [Research Code: IDPH-8261], (plus/minus)-2-[p-(1-oxo-2-isoindolinyl)phenyl]butyric acid [INN: INDOBUFEN], 1-(4-chlorobenzoyl)-5-methoxy-2-methyl-1H-indole-3-acetic acid [INN: INDOMETACIN], 1-(4-chlorobenzoyl)-5-methoxy-2-methyl-1H-indole-3-acetic acid, 3,7,11-trimethyl-2,6,10-dodecatrienyl ester [INN: INDOMETACIN FARNESIL], p-(1-oxo-2-isoindolinyl)hydratropic acid [INN: INDOPROFEN], 2-(10-methoxy-4H-benzo[4,5]cyclohepta[1,2-b]thiophen-4-ylidene)-acetic acid [Research Code: IX-207-887], m-benzoylhydratropic acid [INN: KETOPROFEN], (DL)-5-benzoyl-3H-1,2-dihydropyrrolo[1,2-a]pyrrole-1-carboxylic acid [INN: KETOROLAC], 2,3-dihydro-5-hydroxy-6-[2-(hydroxy-methyl)cinnamyl]benzofuran [Research Code: L-651896], N-(2-carboxyphenyl)-4-chloroanthranilic acid [INN: LOBENZARIT], 3-(p-chlorophenyl)-1-phenylpyrazole-4-acetic acid [INN: LONAZOLAC], 6-chloro-4-hydroxy-2-methyl-N-2-pyridyl-2H-thieno[2,3-e]-1,2-thiazine-3-carboxamide 1,1-dioxide [INN: LORNOXICAM], 2-[4-(2-oxocyclopentan-1-ylmethyl)phenyl]-propionate [INN: LOXOPROFEN], 2(R)-[4-(3-methyl-2-thienyl)phenyl]propionic acid [Research Code: M-5010], N-(2,3-xylyl)anthranilic acid [INN: MEFENAMIC ACID], 4-hydroxy-2-methyl-N-(5-methyl-2-thiazolyl)-2H-1,2-benzothiazine-3-carboxamide 1,1-dioxide [INN: MELOXICAM], 5-aminosalicylic acid [INN: MESALAZINE], (2,2-dimethyl-6-(4-chlorophenyl)-7-phenyl-2,3-dihydro-1H-pyrrolizin-5-yl)-acetic acid [Research Code: ML-3000], 3,4-bis(4-methoxyphenyl)-5-isoxazoleacetic acid [INN: MOFEZOLAC], 4-(6-methoxy-2-naphthyl)-2-butanone [INN: NABUMETONE], (plus)-6-methoxy-alpha-methyl-2-naphthalineacetic acid [INN: NAPROXEN], 2-[3-(trifluoromethyl)anilino]nicotinic acid [INN: NIFLUMIC ACID], 5,5′-azodisalicylic acid [INN: OLSALAZINE], 4,5-diphenyl-2-oxazolepropionic acid [INN: OXAPROZIN], alpha-methyl-4-[(2-oxocyclohexylidene)methyl]benzene acetic acid [INN: PELUBIPROFEN], 4-butyl-1,2-diphenyl-3,5-pyrazolidinedione [INN: PHENYLBUTAZONE], 2-(p-isobutylphenyl)propionic acid 2-pyridyl-methyl ester [INN: PIMEPROFEN], 4-(p-chlorophenyl)-1-(p-fluorophenyl)pyrazole-3-acetic acid [INN: PIRAZOLAC], 4-hydroxy-2-methyl-N-2-pyridyl-2H-1,2-benzothiadiazin-3-carboxamide 1,1-dioxide [INN: PIROXICAM], 3-chloro-4-(3-pyrrolin-1-yl)hydratropic acid [INN: PIRPROFEN], 2-[5H-(1)benzopyrano]2,3-b]pyridin-7-yl]propionic acid [INN: PRANOPROFEN], 2,6-di-tert-butyl-4-(2′-thenoyl)phenol [INN: PRIFELONE], alpha-cyano-1-methyl-beta-oxopyrrole-2-propionanilide [INN: PRINOMIDE], 3-[4-(2-hydroxyethyl)-1-piperazinyl]-propyl-D,L-4-benzamido-N,N-dipropylglutaramat 1-(p-chlorobenzoyl)-5-methoxy-2-methyl-indole-3-acetate (ester) [INN: PROGLUMETACIN], 7-methyl-1-(1-methylethyl)-4-phenyl-2(1H)quinazolinone [INN: PROQUAZONE], 7-methoxy-alpha,10-dimethylphenothiazine-2-acetic acid [INN: PROTIZINIC ACID], 2-[[2-(p-chlorophenyl)-4-methyl-5-oxazolyl]methoxy]-2-methylpropionic acid [INN: ROMAZARIT], o-hydroxybenzamide [SALICYLAMIDE], 2-hydroxybenzoic acid [SALICYLIC ACID], N-acetyl-L-cysteine salicylate (ester), acetate (ester) [INN: SALMISTEINE], N-acetyl-L-cysteine salicylate (ester) [INN: SALNACEDIN], 2-hydroxybenzoic acid 2-carboxyphenyl ester [INN: SALSALATE], 4-[1-(2-fluorobiphenyl-4-yl)ethyl]-N-methylthiazole-2-amine [Research Code: SM-8849], (Z)-5-fluoro-2-methyl-1-[p-(methylsulfinyl)benzylidene]indene-3-acetic acid [INN: SULINDAC], p-2-thenoylhydratropic acid [INN: SUPROFEN] 2-(4-(3-methyl-2-butenyl)phenyl)propionic acid [Research Code: TA-60], phthalidyl 2-(alpha,alpha,alpha-trifluoro-m-toluidino)nicotinate [INN: TALNIFLUMATE], (Z)-5-chloro-3-(2-thenoyl)-2-oxoindole-1-carboxamide [INN: TENIDAP], 2-thiophenecarboxylic acid, ester with salicylic acid [INN: TENOSAL], 4-hydroxy-2-methyl-N-2-pyridyl-2H-thieno[2,3-e]-1,2-thiazine-3-carboxamide [INN: TENOXICAM], 5-(4-chlorophenyl)-N-hydroxy-1-(4-methoxyphenyl)-N-methyl-1H-pyrazole-3-propionamide [INN: TEPOXALIN], alpha-(5-benzoyl-2-thienyl)propionic acid [INN: TIAPROFENIC ACID], 5-chloro-3-[4-(2-hydroxyethyl)-1-piperazinyl]carbonylmethyl-2-benzo-thiazolin one [INN: TIARAMIDE], 2-(2-methyl-5H-[l]benzopyrano[2,3-b]pyridin-7-yl)-propionic acid N,N-dimethylcarbamoylmethyl ester [INN: TILNOPROFEN ARBAMEL], l-Cyclohexyl-2-(2-methyl-4-quinolyl)-3-(2-thiazolyl)guanidine [INN: TIMEGADINE], 2-amino-6-benzyl-4,5,6,7-tetrahydrothieno[2,3-c]pyridine [INN: TINORIDINE], N-(3-chloro-o-tolyl)anthranilic acid [INN: TOLFENAMIC ACID], 1-methyl-S-(4-methylbenzoyl)-1H-pyrrole-2-acetic acid [INN: TOLMETIN], hydroxybis[alpha-methyl-4-(2-methylpropyl)benzene acetato-O]-aluminium [Research Code: U-18573-G], N-(3-trifluoromethylphenyl)-anthranilic acid n-butyl ester [INN: UFENAMATE], 2-[4-[3-(hydroxyimino)cyclohexyl]phenyl]propionic acid [INN: XIMOPROFEN], 2-(10,11-dihydro-10-oxo-dibenz[b,f]thiepin-2-yl-propionic acid [INN: ZALTOPROFEN] and 2-[4-(2-thiazolyloxy)phenyl]-propionic acid [INN: ZOLIPROFEN]; a NO-NSAID selected from the group consisting of those NO-NSAIDs which are disclosed in WO 96/32946, WO 96/35416, WO 96/38136, WO 96/39409, WO 00/50037, U.S. Pat. No. 6,057,347, WO 94/04484, WO 94/12463, WO 95/09831, WO 95/30641, WO 97/31654, WO 99/44595, WO 99/45004 and WO 01/45703; a COX-2 inhibitor selected from the group consisting of 5-chloro-6′-methyl-3-[4-(methylsulfonyl)phenyl]-2,3′-bipyridine [INN: ETORICOXIB], 4-[5-(4-methylphenyl)-3-(trifluoromethyl)-1H-pyrazol-1-yl]benzene-sulfonamide [INN: CELECOXIB], 4-[p-(methylsulfonyl)phenyl]-3-phenyl-2(5H)-furanone [INN: ROFECOXIB], N-[[p-(5-methyl-3-phenyl-4-isoxazolyl)phenyl]sulfonyl]propion-amide [INN: PARECOXIB], p-(5-methyl-3-phenyl-4-isoxazolyl)benzenesulfonamide [INN: VALDECOXIB], 2-[2-(2-chloro-6-fluorophenylamino)-5-methylphenyl]acetic acid [INN: LUMIRACOXIB], 4-(4-cyclohexyl-2-methyloxazol-5-yl)-2-fluorobenzene-sulfonamide [INN: TILMACOXIB], 4-[4-chloro-5-(3-fluoro-4-methoxyphenyl)-1H-imidazol-1-yl]benzene-sulfonamide [INN: CIMICOXIB], 4′-nitro-2′-phenoxymethanesulfonanilide [INN: NIMESULIDE], 6-(2,4-difluorophenoxy)-5-methylsulfonylamino-1-indanone [INN: FLOSULIDE], 5-bromo-2-(4-fluoro-phenyl)-3-(4-methanesulfonyl-phenyl)-thiophene [DUP-697], 4-acetyl-2-(2,4-difluorophenoxy)methanesulfonanilide [FK-3311], N-[2-(cyclohexyloxy)-4-nitrophenyl]methanesulfonamide [NS-398], 5-methanesulfonamido-6-(2,4-difluorophenylthio)-1-indanone [L-745337], 8-acetyl-3-(4-fluorophenyl)-2-[4-(methanesulfonyl)phenyl]imidazo[1,2-a]-pyridine [GR-253035], 4-[5-(4-chlorophenyl)-3-(trifluoromethyl)pyrazol-1-yl]benzenesulfon-amide [SC-58236], 4-(2,3-dihydro-2-oxo-3-phenyl-4-oxazolyl)-benzenesulfonamide [LAS-33815], CS-502, 2-(3,4-difluorophenyl)-4-(3-hydroxy-3-methylbutoxy)-5-[4-(methyl-sulfonyl)phenyl]-3(2H)-pyridazinone [ABT-963], GW-406381, and those COX-2 inhibitors disclosed in WO 02096427, WO 02096886 and WO 02096885; a bisphosphonate selected from the group consisting of ALENDRONIC ACID, RISEDRONIC ACID, TILUDRONIC ACID, IBANDRONIC ACID, ZOLEDRONIC ACID, CLODRONIC ACID, INCADRONIC ACID, OLPADRONIC ACID, MINODRONIC ACID, PAMIDRONIC ACID and ETIDRONIC ACID; a corticosteroid selected from the group consisting of HYDROCORTISONE, PREDNISONE, PREDNISOLONE, METHYLPREDNISOLONE, TRIAMCINOLONE ACETONIDE, AMCINONIDE, CLOBETASONE, CLOBETASOL, DEFLAZACORT, DESONIDE, CLOPREDNOL, DEXAMETHASONE, DIFLORASONE, DIFLUCORTOLONE, DIFLUPREDNATE, FLUDROXYCORTIDE, FLUDROCORTISONE, FLUMETASONE, TIXOCORTOL PIVALATE, FLUOCORTIN BUTYL, CLOCORTOLONE, FLUOCINOLONE ACETONIDE, FLUOCORTOLONE, FLUOROMETHOLONE, FLUPREDNIDENE, FLUPREDNISOLONE, BETAMETHASONE, HALCINONIDE, BUDESONIDE, HALOMETASONE, RIMEXOLONE, PARAMETHASONE, PREDNYLIDENE, LOTEPREDNOL ETABONATE and PREDNICARBATE; and pharmaceutically acceptable derivatives of any of these second active ingredients optionally together with instructions for simultaneous, sequential or separate use in therapy.

17. (canceled)

18. A pharmaceutical formulation comprising a composition according to claim 1, together with a pharmaceutically acceptable carrier or diluent.

19. A pharmaceutical composition according to claim 1 in unit dosage form comprising said first and second active ingredients in admixture for simultaneous oral administration.

20. A method for treating or preventing a disease or disorder which can be treated or prevented by an agent selected from a group consisting of NSAIDs, COX-2 inhibitors, NO-NSAIDs, bisphosphonates and corticosteroids while reducing the risk of gastrointestinal diseases caused by said agent or reducing the risk of gastrointestinal disorders associated with said agent in a human patient in need of such treatment or prevention and at risk of gastrointestinal diseases caused by said agent or gastrointestinal disorders associated with said agent in a patient, comprising administering to said patient said agent in an amount effective to treat or to prevent said diseases or disorders, which can be treated or prevented by said agent, simultaneously, separately or sequentially with a tricyclic imidazo[1,2-a]pyridine compound as defined as first active ingredient in claim 1 or a salt, hydrate, solvate, hydrate of a salt or solvate of a salt thereof in an amount effective to reduce the risk of gastrointestinal diseases caused by said agent or gastrointestinal disorders associated with said agent.

21. A method for treating or preventing a medicament caused gastrointestinal disease or a medicament associated gastrointestinal disorder comprising administering to a patient in need thereof a therapeutically effective amount of a tricyclic imidazo[1,2-a]pyridine compound as defined as first active ingredient in claim 1 or a salt, hydrate, solvate, hydrate of a salt or solvate of a salt thereof simultaneously, separately or sequentially with a therapeutically effective amount of a NSAID, a COX-2 inhibitor, a NO-NSAID, a bisphosphonate or a corticosteroid.

22. A commercial package comprising a NSAID, a COX-2 inhibitor, a NO-NSAID, a bisphosphonate or a corticosteroid as active agent together with instructions for simultaneous, sequential or separate use with any of the tricyclic imidazo[1,2-a]pyridine compounds as defined as first active ingredient in claim 1 or a salt, hydrate, solvate, hydrate of a salt or solvate of a salt thereof.

23. A commercial package comprising one of the tricyclic imidazo[1,2-a]pyridine compounds as defined as first active ingredient in claim 1, or a salt, hydrate, solvate, hydrate of a salt or solvate of a salt thereof, as active agent together with instructions for simultaneous, sequential or separate use with a NSAID, a COX-2 inhibitor, a NO-NSAID, a bisphosphonate or a corticosteroid.

24. The kit according to claim 16, wherein said therapy is selected from the group consisting of treating or preventing gastrointestinal diseases caused by NSAIDs, COX-2 inhibitors, NO-NSAIDs, bisphosphonates or corticosteroids, and treating or preventing diseases which can be treated or prevented by said NSAIDs, COX-2 inhibitors, NO-NSAIDs, bisphosphonates or corticosteroids.

25. The method according to claim 20, wherein said agent is selected from the group consisting of a NSAID selected from the group consisting of glycolic acid [o-(2,6-dichloroanilino)phenyl]acetate(ester) [INN: ACECLOFENAC]; 1-(4-chlorobenzoyl)-5-methoxy-2-methyl-1H-indole-3-acetic acid carboxymethyl ester [INN: ACEMETACIN]; 2-(acetyloxy)benzoic acid [ACETYLSALICYLIC ACID], 2-methoxyphenyl-alpha-methyl-4-(isobutyl)phenylacetate [Research Code: AF-2259], (4-allyloxy-3-chlorophenyl)acetic acid [INN: ALCLOFENAC], p-[(2-methylallyl)amino]hydratropic acid [INN: ALMINOPROFEN], 2-amino-3-benzoylphenylacetic acid [INN: AMFENAC], (plus/minus)-4-(1-hydroxyethoxy)-2-methyl-N-2-pyridyl-2H-1,2-benzothiazine-3-carboxamide ethylcarbonate (ester), 1,1-dioxide [INN: AMPIROXICAM], 2-methoxyphenyl-1-methyl-5-(p-methylbenzoyl)pyrrol-2-acetamido-acetate [INN: AMTOLMETINGUACIL], (plus/minus)-2,3-dihydro-5-(4-methoxybenzoyl)-1H pyrrolizine-1-carboxylic acid [INN: ANIROLAC], 2-[4-(alpha,alpha,alpha-trifluoro-m-tolyl)-1-piperazinyl]ethyl-N-(7-trifluoromethyl-4-quinolyl)anthranilate [INN: ANTRAFENINE], 5-(dimethylamino)-9-methyl-2-propyl-1H-pyrazolo[1,2-a][1,2,4]benzo-triazine-1,3(2H)-dione [INN: AZAPROPAZONE], 4-acetamidophenyl salicylate acetate [INN: BENORILATE], 2-(8-methyl-10,11-dihydro-11-oxodibenz[b,f]oxepin-2-yl)propionic acid [INN: BERMOPROFEN], 2-[(1-benzyl-1H-indazol-3-yl)methoxy]-2-methylpropionic acid [INN: BINDARIT], [2-amino-3-(p-bromobenzoyl)phenyl]acetic acid [INN: BROMFENAC], 3-(3-chloro-4-cyclohexylbenzoyl)propionic acid [INN: BUCLOXIC ACID], 5-butyl-1-cyclohexylbarbituric acid [INN: BUCOLOM], 4-butoxy-N-hydroxybenzeneacetamide [INN: BUFEXAMAC], butylmalonic acid mono-(1,2-diphenylhydrazide) [INN: BUMADIZONE], alpha-ethyl-4-(2-methylpropyl)benzeneacetic acid [INN: BUTIBUFEN], 2-(4-biphenylyl)butyric acid, trans-4-phenylcyclohexylamine salt (1:1) [INN: BUTIXIRATE], 2-(acetyloxy)-benzoic acid, calcium salt, compound with urea (1:1) [INN: CARBASALATE CALCIUM], (plus/minus)-6-chloro-alpha-methylcarbazole-2-acetic acid [INN: CARPROFEN], 1-cinnamoyl-5-methoxy-2-methylindole-3-acetic acid [INN: CINMETACIN], N-(2-pyridyl)-2-methyl-4-cinnamoyloxy-2H-1,2-benzothiazine-3-carboxamide-1,1-dioxide [INN: CINNOXICAM], 6-chloro-5-cyclohexyl-1-indancarboxylic acid [INN: CLIDANAC], 2-[4-(p-chlorophenyl)benzyloxy]-2-methylpropionic acid [INN: CLOBUZARIT], 5-methoxy-2-methyl-3-indolylacetohydroxamic acid [INN: DEBOXAMET], (S)-(+)-p-isobutylhydratropic acid [INN: DEXIBUPROFEN], (+)-(S)-m-benzoylhydratropic acid [INN: DEXKETOPROFEN], 2-[(2,6-dichlorophenyl)amino]benzeneacetic acid [INN: DICLOFENAC], 2′,4′-Difluoro-4-hydroxy-3-biphenylcarboxylic acid [INN: DIFLUNISAL], 4-(2,6-dichloroanilino)-3-thiopheneacetic acid [INN: ELTENAC], N-beta-phenethyl-anthranilic acid [INN: ENFENAMIC ACID] salicylic acid acetate, ester with beta-hydroxy p-acetophenetidide [INN: ETERSALATE], 1,8-diethyl-1,3,4,9-tetrahydropyrano[3,4-b]indole-1-acetic acid [INN: ETODOLAC], 2-[[3-(trifluoromethyl)phenyl]amino]benzoic acid 2-(2-hydroxyethoxy)-ethyl ester [INN: ETOFENAMATE], p-chlorobenzoic acid, ester with 4-butyl-4-(hydroxymethyl)-1,2-diphenyl-3,5-pyrazolidinedione [INN: FECLOBUZONE], 4-biphenylacetic acid [INN: FELBINAC], 3-(4-biphenylylcarbonyl)propionic acid [INN: FENBUFEN], [o-(2,4-dichlorophenoxy)phenyl]acetic acid [INN: FENCLOFENAC], (plus/minus)-m-phenoxyhydratropic acid [INN: FENOPROFEN], 4-(p-chlorophenyl)-2-phenyl-5-thiazoleacetic acid [INN: FENTIAZAC], (plus/minus)-alpha-[[(2-hydroxy-1,1-dimethylethyl)amino]methyl]-benzyl alcohol [INN: FEPRADINOL], 4-(2′,4′-difluorobiphenylyl)-4-oxo-2-methylbutanoic acid [INN: FLOBUFEN], 2,3-dihydroxypropyl N-[8-(trifluoromethyl)-4-quinoly]anthranilate [INN: FLOCTAFENINE], N-(alpha,alpha,alpha-trifluoro-m-tolyl)-anthranilic acid [INN: FLUFENAMIC ACID], (plus)-2-(p-fluorophenyl)-alpha-methyl-5-benzoxazoleacetic acid [INN: FLUNOXAPROFEN], 2-fluoro-alpha-methyl-4-biphenylacetic acid [INN: FLURBIPROFEN], (plus/minus)-2-(2-fluoro-4-biphenylyl)propionic acid 1 (acetoxy)ethyl ester [INN: FLURBIPROFEN AXETIL], 2-ethyl-2,3-dihydro-5-benzofuranacetic acid [INN: FUROFENAC], 2-[4-(2′-furoyl)phenyl]propionic acid [INN: FURPROFEN], 2-[2-[1-(p-chlorobenzoyl)-5-methoxy-2-methylindol-3-yl]acetamido]-2-deoxy-D-glucose [INN: GLUCAMETACIN], 2-(2-fluorobiphenyl-4-yl)propionic acid 4-nitrooxybutylester [Research Code: HCT-1026], (p-isobutylphenyl)acetic acid [INN: IBUFENAC], alpha-p-isobutylphenylpropionic acid [INN: IBUPROFEN], methyl 4-(3-thienyl)phenyl-alpha-methylacetate [Research Code: IDPH-8261], (plus/minus)-2-[p-(1-oxo-2-isoindolinyl)phenyl]butyric acid [INN: INDOBUFEN], 1-(4-chlorobenzoyl)-5-methoxy-2-methyl-1H-indole-3-acetic acid [INN: INDOMETACIN], 1-(4-chlorobenzoyl)-5-methoxy-2-methyl-1H-indole-3-acetic acid, 3,7,11-trimethyl-2,6,10-dodecatrienyl ester [INN: INDOMETACIN FARNESIL], p-(1-oxo-2-isoindolinyl)hydratropic acid [INN: INDOPROFEN], 2-(10-methoxy-4H-benzo[4,5]cyclohepta[1,2-b]thiophen-4-ylidene)-acetic acid [Research Code: IX-207-887], m-benzoylhydratropic acid [INN: KETOPROFEN], (DL)-5-benzoyl-3H-1,2-dihydropyrrolo[1,2-a]pyrrole-1-carboxylic acid [INN: KETOROLAC], 2,3-dihydro-5-hydroxy-6-[2-(hydroxymethyl)-cinnamyl]benzofuran [Research Code: L-651896], N-(2-carboxyphenyl)-4-chloroanthranilic acid [INN: LOBENZARIT], 3-(p-chlorophenyl)-1-phenylpyrazole-4-acetic acid [INN: LONAZOLAC], 6-chloro-4-hydroxy-2-methyl-N-2-pyridyl-2H-thieno[2,3-e]-1,2-thiazine-3-carboxamide 1,1-dioxide [INN: LORNOXICAM], 2-[4-(2-oxocyclopentan-1-ylmethyl)phenyl]-propionate [INN: LOXOPROFEN], 2(R)-[4-(3-methyl-2-thienyl)phenyl]propionic acid [Research Code: M-5010], N-(2,3-xylyl)anthranilic acid [INN: MEFENAMIC ACID], 4-hydroxy-2-methyl-N-(5-methyl-2-thiazolyl)-2H-1,2-benzothiazine-3-carboxamide 1,1-dioxide [INN: MELOXICAM], 5-aminosalicylic acid [INN: MESALAZINE], (2,2-dimethyl-6-(4-chlorophenyl)-7-phenyl-2,3-dihydro-1H-pyrrolizin-5-yl)-acetic acid [Research Code: ML-3000], 3,4-bis(4-methoxyphenyl)-5-isoxazoleacetic acid [INN: MOFEZOLAC], 4-(6-methoxy-2-naphthyl)-2-butanone [INN: NABUMETONE], (plus)-6-methoxy-alpha-methyl-2-naphthalineacetic acid [INN: NAPROXEN], 2-[3-(trifluoromethyl)anilino]nicotinic acid [INN: NIFLUMIC ACID], 5,5′-azodisalicylic acid [INN: OLSALAZINE], 4,5-diphenyl-2-oxazolepropionic acid [INN: OXAPROZIN], alpha-methyl-4-[(2-oxocyclohexylidene)methyl]benzene acetic acid [INN: PELUBIPROFEN], 4-butyl-1,2-diphenyl-3,5-pyrazolidinedione [INN: PHENYLBUTAZONE], 2-(p-isobutylphenyl)propionic acid 2-pyridyl-methyl ester [INN: PIMEPROFEN], 4-(p-chlorophenyl)-1-(p-fluorophenyl)pyrazole-3-acetic acid [INN: PIRAZOLAC], 4-hydroxy-2-methyl-N-2-pyridyl-2H-1,2-benzothiadiazin-3-carboxamide 1,1-dioxide [INN: PIROXICAM], 3-chloro-4-(3-pyrrolin-1-yl)hydratropic acid [INN: PIRPROFEN], 2-[5H-(1)benzopyrano]2,3-b]pyridin-7-yl]propionic acid [INN: PRANOPROFEN], 2,6-di-tert-butyl-4-(2′-thenoyl)phenol [INN: PRIFELONE], alpha-cyano-1-methyl-beta-oxopyrrole-2-propionanilide [INN: PRINOMIDE], 3-[4-(2-hydroxyethyl)-1-piperazinyl]-propyl-D,L-4-benzamido-N,N-dipropylglutaramat 1-(p-chlorobenzoyl)-5-methoxy-2-methylindole-3-acetate (ester) [INN: PROGLUMETACIN], 7-methyl-1-(1-methylethyl)-4-phenyl-2(1H)quinazolinone [INN: PROQUAZONE], 7-methoxy-alpha,10-dimethylphenothiazine-2-acetic acid [INN: PROTIZINIC ACID], 2-[[2-(p-chlorophenyl)-4-methyl-5-oxazolyl]methoxy]-2-methylpropionic acid [INN: ROMAZARIT], o-hydroxybenzamide [SALICYLAMIDE], 2-hydroxybenzoic acid [SALICYLIC ACID], N-acetyl-L-cysteine salicylate (ester), acetate (ester) [INN: SALMISTEINE], N-acetyl-L-cysteine salicylate (ester) [INN: SALNACEDIN], 2-hydroxybenzoic acid 2-carboxyphenyl ester [INN: SALSALATE], 4-[1-(2-fluorobiphenyl-4-yl)ethyl]-N-methylthiazole-2-amine [Research Code: SM-8849], (Z)-5-fluoro-2-methyl-1-[p-(methylsulfinyl)benzylidene]indene-3-acetic acid [INN: SULINDAC], p-2-thenoylhydratropic acid [INN: SUPROFEN] 2-(4-(3-methyl-2-butenyl)phenyl)propionic acid [Research Code: TA-60], phthalidyl 2-(alpha,alpha,alpha-trifluoro-m-toluidino)nicotinate [INN: TALNIFLUMATE], (Z)-5-chloro-3-(2-thenoyl)-2-oxoindole-1-carboxamide [INN: TENIDAP], 2-thiophenecarboxylic acid, ester with salicylic acid [INN: TENOSAL], 4-hydroxy-2-methyl-N-2-pyridyl-2H-thieno[2,3-e]-1,2-thiazine-3-carboxamide [INN: TENOXICAM], 5-(4-chlorophenyl)-N-hydroxy-1-(4-methoxyphenyl)-N-methyl-1H-pyrazole-3-propionamide [INN: TEPOXALIN], alpha-(5-benzoyl-2-thienyl)propionic acid [INN: TIAPROFENIC ACID], 5-chloro-3-[4-(2-hydroxyethyl)-1-piperazinyl]carbonylmethyl-2-benzo-thiazolin one [INN: TIARAMIDE], 2-(2-methyl-5H-[1]benzopyrano[2,3-b]pyridin-7-yl)-propionic acid N,N-dimethylcarbamoylmethyl ester [INN: TILNOPROFEN ARBAMEL], 1-Cyclohexyl-2-(2-methyl-4-quinolyl)-3-(2-thiazolyl)guanidine [INN: TIMEGADINE], 2-amino-6-benzyl-4,5,6,7-tetrahydrothieno[2,3-c]pyridine [INN: TINORIDINE], N-(3-chloro-o-tolyl)anthranilic acid [INN: TOLFENAMIC ACID], 1-methyl-5-(4-methylbenzoyl)-1H-pyrrole-2-acetic acid [INN: TOLMETIN], hydroxybis[alpha-methyl-4-(2-methylpropyl)benzene acetato-O]-aluminium [Research Code: U-18573-G], N-(3-trifluoromethylphenyl)-anthranilic acid n-butyl ester [INN: UFENAMATE], 2-[4-[3-(hydroxyimino)cyclohexyl]phenyl]propionic acid [INN: XIMOPROFEN], 2-(10,11-dihydro-10-oxo-dibenz[b,f]thiepin-2-yl-propionic acid [INN: ZALTOPROFEN] and 2-[4-(2-thiazolyloxy)phenyl]-propionic acid [INN: ZOLIPROFEN]; or a NO-NSAID selected from the group consisting of those NO-NSAIDs which are disclosed in WO 96/32946, WO 96/35416, WO 96/38136, WO 96/39409, WO 00/50037, U.S. Pat. No. 6,057,347, WO 94/04484, WO 94/12463, WO 95/09831, WO 95/30641, WO 97/31654, WO 99/44595, WO 99/45004 and WO 01/45703; or a COX-2 inhibitor selected from the group consisting of 5-chloro-6′-methyl-3-[4-(methylsulfonyl)phenyl]-2,3′-bipyridine [INN: ETORICOXIB], 4-[5-(4-methylphenyl)-3-(trifluoromethyl)-1H-pyrazol-1-yl]benzene-sulfonamide [INN: CELECOXIB], 4-[p-(methylsulfonyl)phenyl]-3-phenyl-2(5H)-furanone [INN: ROFECOXIB], N-[[p-(5-methyl-3-phenyl-4-isoxazolyl)phenyl]sulfonyl]propion-amide [INN: PARECOXIB], p-(5-methyl-3-phenyl-4-isoxazolyl)benzenesulfonamide [INN: VALDECOXIB], 2-[2-(2-chloro-6-fluorophenylamino)-5-methylphenyl]acetic acid [INN: LUMIRACOXIB], 4-(4-cyclohexyl-2-methyloxazol-5-yl)-2-fluorobenzene-sulfonamide [INN: TILMACOXIB], 4-[4-chloro-5-(3-fluoro-4-methoxyphenyl)-1H-imidazol-1-yl]benzene-sulfonamide [INN: CIMICOXIB], 4′-nitro-2′-phenoxymethanesulfonanilide [INN: NIMESULIDE], 6-(2,4-difluorophenoxy)-5-methylsulfonylamino-1-indanone [INN: FLOSULIDE], 5-bromo-2-(4-fluoro-phenyl)-3-(4-methanesulfonyl-phenyl)-thiophene [DUP-697], 4-acetyl-2-(2,4-difluorophenoxy)methanesulfonanilide [FK-3311], N-[2-(cyclohexyloxy)-4-nitrophenyl]methanesulfonamide [NS-398], 5-methanesulfonamido-6-(2,4-difluorophenylthio)-1-indanone [L-745337], 8-acetyl-3-(4-fluorophenyl)-2-[4-(methanesulfonyl)phenyl]imidazo[1,2-a]-pyridine [GR-253035], 4-[5-(4-chlorophenyl)-3-(trifluoromethyl)pyrazol-1-yl]benzenesulfon-amide [SC-58236], 4-(2,3-dihydro-2-oxo-3-phenyl-4-oxazolyl)-benzenesulfonamide [LAS-33815], CS-502, 2-(3,4-difluorophenyl)-4-(3-hydroxy-3-methylbutoxy)-5-[4-(methyl-sulfonyl)phenyl]-3(2H)-pyridazinone [ABT-963], GW-406381, and those COX-2 inhibitors disclosed in WO 02096427, WO 02096886 and WO 02096885; or a bisphosphonate selected from the group consisting of ALENDRONIC ACID, RISEDRONIC ACID, TILUDRONIC ACID, IBANDRONIC ACID, ZOLEDRONIC ACID, CLODRONIC ACID, INCADRONIC ACID, OLPADRONIC ACID, MINODRONIC ACID, PAMIDRONIC ACID and ETIDRONIC ACID; or a corticosteroid selected from the group consisting of HYDROCORTISONE, PREDNISONE, PREDNISOLONE, METHYLPREDNISOLONE, TRIAMCINOLONE ACETONIDE, AMCINONIDE, CLOBETASONE, CLOBETASOL, DEFLAZACORT, DESONIDE, CLOPREDNOL, DEXAMETHASONE, DIFLORASONE, DIFLUCORTOLONE, DIFLUPREDNATE, FLUDROXYCORTIDE, FLUDROCORTISONE, FLUMETASONE, TIXOCORTOL PIVALATE, FLUOCORTIN BUTYL, CLOCORTOLONE, FLUOCINOLONE ACETONIDE, FLUOCORTOLONE, FLUOROMETHOLONE, FLUPREDNIDENE, FLUPREDNISOLONE, BETAMETHASONE, HALCINONIDE, BUDESONIDE, HALOMETASONE, RIMEXOLONE, PARAMETHASONE, PREDNYLIDENE, LOTEPREDNOL ETABONATE and PREDNICARBATE;
or a pharmaceutically acceptable derivative of any of these second ingredients.

26. The method according to claim 21, wherein said NSAID is selected from the group consisting of glycolic acid [o-(2,6-dichloroanilino)phenyl]acetate(ester) [INN: ACECLOFENAC]; 1-(4-chlorobenzoyl)-5-methoxy-2-methyl-1H-indole-3-acetic acid carboxymethyl ester [INN: ACEMETACIN]; 2-(acetyloxy)benzoic acid [ACETYLSALICYLIC ACID], 2-methoxyphenyl-alpha-methyl-4-(isobutyl)phenylacetate [Research Code: AF-2259], (4-allyloxy-3-chlorophenyl)acetic acid [INN: ALCLOFENAC], p-[(2-methylallyl)amino]hydratropic acid [INN: ALMINOPROFEN], 2-amino-3-benzoylphenylacetic acid [INN: AMFENAC], (plus/minus)-4-(1-hydroxyethoxy)-2-methyl-N-2-pyridyl-2H-1,2-benzothiazine-3-carboxamide ethylcarbonate (ester), 1,1-dioxide [INN: AMPIROXICAM], 2-methoxyphenyl-1-methyl-5-(p-methylbenzoyl)pyrrol-2-acetamido-acetate [INN: AMTOLMETINGUACIL], (plus/minus)-2,3-dihydro-5-(4-methoxybenzoyl)-1H pyrrolizine-1-carboxylic acid [INN: ANIROLAC], 2-[4-(alpha,alpha,alpha-trifluoro-m-tolyl)-1-piperazinyl]ethyl-N-(7-trifluoromethyl-4-quinolyl)anthranilate [INN: ANTRAFENINE], 5-(dimethylamino)-9-methyl-2-propyl-1H-pyrazolo[1,2-a][1,2,4]benzo-triazine-1,3(2H)-dione [INN: AZAPROPAZONE], 4-acetamidophenyl salicylate acetate [INN: BENORILATE], 2-(8-methyl-10,11-dihydro-11-oxodibenz[b,f]oxepin-2-yl)propionic acid [INN: BERMOPROFEN], 2-[(1-benzyl-1H-indazol-3-yl)methoxy]-2-methylpropionic acid [INN: BINDARIT], [2-amino-3-(p-bromobenzoyl)phenyl]acetic acid [INN: BROMFENAC], 3-(3-chloro-4-cyclohexylbenzoyl)propionic acid [INN: BUCLOXIC ACID], 5-butyl-1-cyclohexylbarbituric acid [INN: BUCOLOM], 4-butoxy-N-hydroxybenzeneacetamide [INN: BUFEXAMAC], butylmalonic acid mono-(1,2-diphenylhydrazide) [INN: BUMADIZONE], alpha-ethyl-4-(2-methylpropyl)benzeneacetic acid [INN: BUTIBUFEN], 2-(4-biphenylyl)butyric acid, trans-4-phenylcyclohexylamine salt (1:1) [INN: BUTIXIRATE], 2-(acetyloxy)-benzoic acid, calcium salt, compound with urea (1:1) [INN: CARBASALATE CALCIUM], (plus/minus)-6-chloro-alpha-methylcarbazole-2-acetic acid [INN: CARPROFEN], 1-cinnamoyl-5-methoxy-2-methylindole-3-acetic acid [INN: CINMETACIN], N-(2-pyridyl)-2-methyl-4-cinnamoyloxy-2H-1,2-benzothiazine-3-carboxamide-11,1-dioxide [INN: CINNOXICAM], 6-chloro-5-cyclohexyl-1-indancarboxylic acid [INN: CLIDANAC], 2-[4-(p-chlorophenyl)benzyloxy]-2-methylpropionic acid [INN: CLOBUZARIT], 5-methoxy-2-methyl-3-indolylacetohydroxamic acid [INN: DEBOXAMET], (S)-(+)-p-isobutylhydratropic acid [INN: DEXIBUPROFEN], (+)-(S)-m-benzoylhydratropic acid [INN: DEXKETOPROFEN], 2-[(2,6-dichlorophenyl)amino]benzeneacetic acid [INN: DICLOFENAC], 2′,4′-Difluoro-4-hydroxy-3-biphenylcarboxylic acid [INN: DIFLUNISAL], 4-(2,6-dichloroanilino)-3-thiopheneacetic acid [INN: ELTENAC], N-beta-phenethyl-anthranilic acid [INN: ENFENAMIC ACID] salicylic acid acetate, ester with beta-hydroxy p-acetophenetidide [INN: ETERSALATE], 1,8-diethyl-1,3,4,9-tetrahydropyrano[3,4-b]indole-1-acetic acid [INN: ETODOLAC], 2-[[3-(trifluoromethyl)phenyl]amino]benzoic acid 2-(2-hydroxyethoxy)-ethyl ester [INN: ETOFENAMATE], p-chlorobenzoic acid, ester with 4-butyl-4-(hydroxymethyl)-1,2-diphenyl-3,5-pyrazolidinedione [INN: FECLOBUZONE), 4-biphenylacetic acid [INN: FELBINAC], 3-(4-biphenylylcarbonyl)propionic acid [INN: FENBUFEN], [o-(2,4-dichlorophenoxy)phenyl]acetic acid [INN: FENCLOFENAC], (plus/minus)-m-phenoxyhydratropic acid [INN: FENOPROFEN], 4-(p-chlorophenyl)-2-phenyl-5-thiazoleacetic acid [INN: FENTIAZAC], (plus/minus)-alpha-[[(2-hydroxy-1,1-dimethylethyl)amino]methyl]-benzyl alcohol [INN: FEPRADINOL], 4-(2′,4′-difluorobiphenylyl)-4-oxo-2-methylbutanoic acid [INN: FLOBUFEN], 2,3-dihydroxypropyl N-[8-(trifluoromethyl)-4-quinoly]anthranilate [INN: FLOCTAFENINE], N-(alpha,alpha,alpha-trifluoro-m-tolyl)-anthranilic acid [INN: FLUFENAMIC ACID], (plus)-2-(p-fluorophenyl)-alpha-methyl-5-benzoxazoleacetic acid [INN: FLUNOXAPROFEN], 2-fluoro-alpha-methyl-4-biphenylacetic acid [INN: FLURBIPROFEN], (plus/minus)-2-(2-fluoro-4-biphenylyl)propionic acid 1 (acetoxy)ethyl ester [INN: FLURBIPROFEN AXETIL], 2-ethyl-2,3-dihydro-5-benzofuranacetic acid [INN: FUROFENAC], 2-[4-(2′-furoyl)phenyl]propionic acid [INN: FURPROFEN], 2-[2-[1-(p-chlorobenzoyl)-5-methoxy-2-methylindol-3-yl]acetamido]-2-deoxy-D-glucose [INN: GLUCAMETACIN], 2-(2-fluorobiphenyl-4-yl)propionic acid 4-nitrooxybutylester [Research Code: HCT-1026], (p-isobutylphenyl)acetic acid [INN: IBUFENAC], alpha-p-isobutylphenylpropionic acid [INN: IBUPROFEN], methyl 4-(3-thienyl)phenyl-alpha-methylacetate [Research Code: IDPH-8261], (plus/minus)-2-[p-(1-oxo-2-isoindolinyl)phenyl]butyric acid [INN: INDOBUFEN], 1-(4-chlorobenzoyl)-5-methoxy-2-methyl-1H-indole-3-acetic acid [INN: INDOMETACIN], 1-(4-chlorobenzoyl)-5-methoxy-2-methyl-1H-indole-3-acetic acid, 3,7,11-trimethyl-2,6,10-dodecatrienyl ester [INN: INDOMETACIN FARNESIL], p-(1-oxo-2-isoindolinyl)hydratropic acid [INN: INDOPROFEN], 2-(10-methoxy-4H-benzo[4,5]cyclohepta[1,2-b]thiophen-4-ylidene)-acetic acid [Research Code: IX-207-887], m-benzoylhydratropic acid [INN: KETOPROFEN], (DL)-5-benzoyl-3H-1,2-dihydropyrrolo[1,2-a]pyrrole-1-carboxylic acid [INN: KETOROLAC], 2,3-dihydro-5-hydroxy-6-[2-(hydroxy-methyl)cinnamyl]benzofuran [Research Code: L-651896], N-(2-carboxyphenyl)-4-chloroanthranilic acid [INN: LOBENZARIT], 3-(p-chlorophenyl)-1-phenylpyrazole-4-acetic acid [INN: LONAZOLAC], 6-chloro-4-hydroxy-2-methyl-N-2-pyridyl-2H-thieno[2,3-e]-1,2-thiazine-3-carboxamide 1,1-dioxide [INN: LORNOXICAM], 2-[4-(2-oxocyclopentan-1-ylmethyl)phenyl]-propionate [INN: LOXOPROFEN], 2(R)-[4-(3-methyl-2-thienyl)phenyl]propionic acid [Research Code: M-5010], N-(2,3-xylyl)anthranilic acid [INN: MEFENAMIC ACID], 4-hydroxy-2-methyl-N-(5-methyl-2-thiazolyl)-2H-1,2-benzothiazine-3-carboxamide 1,1-dioxide [INN: MELOXICAM], 5-aminosalicylic acid [INN: MESALAZINE], (2,2-dimethyl-6-(4-chlorophenyl)-7-phenyl-2,3-dihydro-1H-pyrrolizin-5-yl)-acetic acid [Research Code: ML-3000], 3,4-bis(4-methoxyphenyl)-5-isoxazoleacetic acid [INN: MOFEZOLAC], 4-(6-methoxy-2-naphthyl)-2-butanone [INN: NABUMETONE], (plus)-6-methoxy-alpha-methyl-2-naphthalineacetic acid [INN: NAPROXEN], 2-[3-(trifluoromethyl)anilino]nicotinic acid [INN: NIFLUMIC ACID], 5,5′-azodisalicylic acid [INN: OLSALAZINE], 4,5-diphenyl-2-oxazolepropionic acid [INN: OXAPROZIN], alpha-methyl-4-[(2-oxocyclohexylidene)methyl]benzene acetic acid [INN: PELUBIPROFEN], 4-butyl-1,2-diphenyl-3,5-pyrazolidinedione [INN: PHENYLBUTAZONE], 2-(p-isobutylphenyl)propionic acid 2-pyridyl-methyl ester [INN: PIMEPROFEN], 4-(p-chlorophenyl)-1-(p-fluorophenyl)pyrazole-3-acetic acid [INN: PIRAZOLAC], 4-hydroxy-2-methyl-N-2-pyridyl-2H-1,2-benzothiadiazin-3-carboxamide 1,1-dioxide [INN: PIROXICAM], 3-chloro-4-(3-pyrrolin-1-yl)hydratropic acid [INN: PIRPROFEN], 2-[5H-(1)benzopyrano]2,3-b]pyridin-7-yl]propionic acid [INN: PRANOPROFEN], 2,6-di-tert-butyl-4-(2′-thenoyl)phenol [INN: PRIFELONE], alpha-cyano-1-methyl-beta-oxopyrrole-2-propionanilide [INN: PRINOMIDE], 3-[4-(2-hydroxyethyl)-1-piperazinyl]-propyl-D,L-4-benzamido-N,N-dipropylglutaramat 1-(p-chlorobenzoyl)-5-methoxy-2-methylindole-3-acetate (ester) [INN: PROGLUMETACIN], 7-methyl-1-(1-methylethyl)-4-phenyl-2(1H)quinazolinone [INN: PROQUAZONE], 7-methoxy-alpha,10-dimethylphenothiazine-2-acetic acid [INN: PROTIZINIC ACID], 2-[[2-(p-chlorophenyl)-4-methyl-5-oxazolyl]methoxy]-2-methylpropionic acid [INN: ROMAZARIT], o-hydroxybenzamide [SALICYLAMIDE], 2-hydroxybenzoic acid [SALICYLIC ACID], N-acetyl-L-cysteine salicylate (ester), acetate (ester) [INN: SALMISTEINE], N-acetyl-L-cysteine salicylate (ester) [INN: SALNACEDIN], 2-hydroxybenzoic acid 2-carboxyphenyl ester [INN: SALSALATE], 4-[1-(2-fluorobiphenyl-4-yl)ethyl]-N-methylthiazole-2-amine [Research Code: SM-8849], (Z)-5-fluoro-2-methyl-1-[p-(methylsulfinyl)benzylidene]indene-3-acetic acid [INN: SULINDAC], p-2-thenoylhydratropic acid [INN: SUPROFEN] 2-(4-(3-methyl-2-butenyl)phenyl)propionic acid [Research Code: TA-60], phthalidyl 2-(alpha,alpha,alpha-trifluoro-m-toluidino)nicotinate [INN: TALNIFLUMATE], (Z)-5-chloro-3-(2-thenoyl)-2-oxoindole-1-carboxamide [INN: TENIDAP], 2-thiophenecarboxylic acid, ester with salicylic acid [INN: TENOSAL], 4-hydroxy-2-methyl-N-2-pyridyl-2H-thieno[2,3-e]-1,2-thiazine-3-carboxamide [INN: TENOXICAM], 5-(4-chlorophenyl)-N-hydroxy-1-(4-methoxyphenyl)-N-methyl-1H-pyrazole-3-propionamide [INN: TEPOXALIN], alpha-(5-benzoyl-2-thienyl)propionic acid [INN: TIAPROFENIC ACID], 5-chloro-3-[4-(2-hydroxyethyl)-1-piperazinyl]carbonylmethyl-2-benzo-thiazolin one [INN: TIARAMIDE], 2-(2-methyl-5H-[l]benzopyrano[2,3-b]pyridin-7-yl)-propionic acid N,N-dimethylcarbamoylmethyl ester [INN: TILNOPROFEN ARBAMEL], 1-Cyclohexyl-2-(2-methyl-4-quinolyl)-3-(2-thiazolyl)guanidine [INN: TIMEGADINE], 2-amino-6-benzyl-4,5,6,7-tetrahydrothieno[2,3-c]pyridine [INN: TINORIDINE], N-(3-chloro-o-tolyl)anthranilic acid [INN: TOLFENAMIC ACID], 1-methyl-5-(4-methylbenzoyl)-1H-pyrrole-2-acetic acid [INN: TOLMETIN], hydroxybis[alpha-methyl-4-(2-methylpropyl)benzene acetato-O]-aluminium [Research Code: U-18573-G], N-(3-trifluoromethylphenyl)-anthranilic acid n-butyl ester [INN: UFENAMATE], 2-[4-[3-(hydroxyimino)cyclohexyl]phenyl]propionic acid [INN: XIMOPROFEN], 2-(10,11-dihydro-10-oxo-dibenz[b,f]thiepin-2-yl-propionic acid [INN: ZALTOPROFEN] and 2-[4-(2-thiazolyloxy)phenyl]-propionic acid [INN: ZOLIPROFEN]; wherein said NO-NSAID is selected from the group consisting of those NO-NSAIDs which are disclosed in WO 96/32946, WO 96/35416, WO 96/38136, WO 96/39409, WO 00/50037, U.S. Pat. No. 6,057,347, WO 94/04484, WO 94/12463, WO 95/09831, WO 95/30641, WO 97/31654, WO 99/44595, WO 99/45004 and WO 01/45703; wherein said COX-2 inhibitor is selected from the group consisting of 5-chloro-6′-methyl-3-[4-(methylsulfonyl)phenyl]-2,3′-bipyridine [INN: ETORICOXIB], 4-[5-(4-methylphenyl)-3-(trifluoromethyl)-1H-pyrazol-1-yl]benzene-sulfonamide [INN: CELECOXIB], 4-[p-(methylsulfonyl)phenyl]-3-phenyl-2(5H)-furanone [INN: ROFECOXIB], N-[[p-(5-methyl-3-phenyl-4-isoxazolyl)phenyl]sulfonyl]propion-amide [INN: PARECOXIB], p-(5-methyl-3-phenyl-4-isoxazolyl)benzenesulfonamide [INN: VALDECOXIB], 2-[2-(2-chloro-6-fluorophenylamino)-5-methylphenyl]acetic acid [INN: LUMIRACOXIB], 4-(4-cyclohexyl-2-methyloxazol-5-yl)-2-fluorobenzene-sulfonamide [INN: TILMACOXIB], 4-[4-chloro-5-(3-fluoro-4-methoxyphenyl)-1H-imidazol-1-yl]benzene-sulfonamide [INN: CIMICOXIB], 4′-nitro-2′-phenoxymethanesulfonanilide [INN: NIMESULIDE], 6-(2,4-difluorophenoxy)-5-methylsulfonylamino-1-indanone [INN: FLOSULIDE], 5-bromo-2-(4-fluoro-phenyl)-3-(4-methanesulfonyl-phenyl)-thiophene [DUP-697], 4-acetyl-2-(2,4-difluorophenoxy)methanesulfonanilide [FK-3311], N-[2-(cyclohexyloxy)-4-nitrophenyl]methanesulfonamide [NS-398], 5-methanesulfonamido-6-(2,4-difluorophenylthio)-1-indanone [L-745337], 8-acetyl-3-(4-fluorophenyl)-2-[4-(methanesulfonyl)phenyl]imidazo[1,2-a]-pyridine [GR-253035], 4-[5-(4-chlorophenyl)-3-(trifluoromethyl)pyrazol-1-yl]benzenesulfon-amide [SC-58236], 4-(2,3-dihydro-2-oxo-3-phenyl-4-oxazolyl)-benzenesulfonamide [LAS-33815], CS-502, 2-(3,4-difluorophenyl)-4-(3-hydroxy-3-methylbutoxy)-5-[4-(methyl-sulfonyl)phenyl]-3(2H)-pyridazinone [ABT-963], GW-406381, and those COX-2 inhibitors disclosed in WO 02096427, WO 02096886 and WO 02096885; wherein said bisphosphonate is selected from the group consisting of ALENDRONIC ACID, RISEDRONIC ACID, TILUDRONIC ACID, IBANDRONIC ACID, ZOLEDRONIC ACID, CLODRONIC ACID, INCADRONIC ACID, OLPADRONIC ACID, MINODRONIC ACID, PAMIDRONIC ACID and ETIDRONIC ACID; wherein said corticosteroid is selected from the group consisting of HYDROCORTISONE, PREDNISONE, PREDNISOLONE, METHYLPREDNISOLONE, TRIAMCINOLONE ACETONIDE, AMCINONIDE, CLOBETASONE, CLOBETASOL, DEFLAZACORT, DESONIDE, CLOPREDNOL, DEXAMETHASONE, DIFLORASONE, DIFLUCORTOLONE, DIFLUPREDNATE, FLUDROXYCORTIDE, FLUDROCORTISONE, FLUMETASONE, TIXOCORTOL PIVALATE, FLUOCORTIN BUTYL, CLOCORTOLONE, FLUOCINOLONE ACETONIDE, FLUOCORTOLONE, FLUOROMETHOLONE, FLUPREDNIDENE, FLUPREDNISOLONE, BETAMETHASONE, HALCINONIDE, BUDESONIDE, HALOMETASONE, RIMEXOLONE, PARAMETHASONE, PREDNYLIDENE, LOTEPREDNOL ETABONATE and PREDNICARBATE; and pharmaceutically acceptable derivatives of any of these compounds.

27. The commercial package according to claim 22, wherein said NSAID is selected from the group consisting of glycolic acid [o-(2,6-dichloroanilino)phenyl]acetate(ester) [INN: ACECLOFENAC]; 1-(4-chlorobenzoyl)-5-methoxy-2-methyl-1H-indole-3-acetic acid carboxymethyl ester [INN: ACEMETACIN]; 2-(acetyloxy)benzoic acid [ACETYLSALICYLIC ACID], 2-methoxyphenyl-alpha-methyl-4-(isobutyl)phenylacetate [Research Code: AF-2259], (4-allyloxy-3-chlorophenyl)acetic acid [INN: ALCLOFENAC], p-[(2-methylallyl)amino]hydratropic acid [INN: ALMINOPROFEN], 2-amino-3-benzoylphenylacetic acid [INN: AMFENAC], (plus/minus)-4-(1-hydroxyethoxy)-2-methyl-N-2-pyridyl-2H-1,2-benzothiazine-3-carboxamide ethylcarbonate (ester), 1,1-dioxide [INN: AMPIROXICAM], 2-methoxyphenyl-1-methyl-5-(p-methylbenzoyl)pyrrol-2-acetamido-acetate [INN: AMTOLMETINGUACIL], (plus/minus)-2,3-dihydro-5-(4-methoxybenzoyl)-1H pyrrolizine-1-carboxylic acid [INN: ANIROLAC], 2-[4-(alpha,alpha,alpha-trifluoro-m-tolyl)-1-piperazinyl]ethyl-N-(7-trifluoromethyl-4-quinolyl)anthranilate [INN: ANTRAFENINE], 5-(dimethylamino)-9-methyl-2-propyl-1H-pyrazolo[1,2-a][1,2,4]benzo-triazine-1,3(2H)-dione [INN: AZAPROPAZONE], 4-acetamidophenyl salicylate acetate [INN: BENORILATE], 2-(8-methyl-10,11-dihydro-11-oxodibenz[b,f]oxepin-2-yl)propionic acid [INN: BERMOPROFEN], 2-[(1-benzyl-1H-indazol-3-yl)methoxy]-2-methylpropionic acid [INN: BINDARIT], [2-amino-3-(p-bromobenzoyl)phenyl]acetic acid [INN: BROMFENAC], 3-(3-chloro-4-cyclohexylbenzoyl)propionic acid [INN: BUCLOXIC ACID], 5-butyl-1-cyclohexylbarbituric acid [INN: BUCOLOM], 4-butoxy-N-hydroxybenzeneacetamide [INN: BUFEXAMAC], butylmalonic acid mono-(1,2-diphenylhydrazide) [INN: BUMADIZONE], alpha-ethyl-4-(2-methylpropyl)benzeneacetic acid [INN: BUTIBUFEN], 2-(4-biphenylyl)butyric acid, trans-4-phenylcyclohexylamine salt (1:1) [INN: BUTIXIRATE], 2-(acetyloxy)-benzoic acid, calcium salt, compound with urea (1:1) [INN: CARBASALATE CALCIUM], (plus/minus)-6-chloro-alpha-methylcarbazole-2-acetic acid [INN: CARPROFEN], 1-cinnamoyl-5-methoxy-2-methylindole-3-acetic acid [INN: CINMETACIN], N-(2-pyridyl)-2-methyl-4-cinnamoyloxy-2H-1,2-benzothiazine-3-carboxamide-1,1-dioxide [INN: CINNOXICAM], 6-chloro-5-cyclohexyl-1-indancarboxylic acid [INN: CLIDANAC], 2-[4-(p-chlorophenyl)benzyloxy]-2-methylpropionic acid [INN: CLOBUZARIT], 5-methoxy-2-methyl-3-indolylacetohydroxamic acid [INN: DEBOXAMET], (S)-(+)-p-isobutylhydratropic acid [INN: DEXIBUPROFEN], (+)-(S)-m-benzoylhydratropic acid [INN: DEXKETOPROFEN], 2-[(2,6-dichlorophenyl)amino]benzeneacetic acid [INN: DICLOFENAC], 2′,4′-Difluoro-4-hydroxy-3-biphenylcarboxylic acid [INN: DIFLUNISAL], 4-(2,6-dichloroanilino)-3-thiopheneacetic acid [INN: ELTENAC], N-beta-phenethyl-anthranilic acid [INN: ENFENAMIC ACID] salicylic acid acetate, ester with beta-hydroxy p-acetophenetidide [INN: ETERSALATE], 1,8-diethyl-1,3,4,9-tetrahydropyrano[3,4-b]indole-1-acetic acid [INN: ETODOLAC], 2-[[3-(trifluoromethyl)phenyl]amino]benzoic acid 2-(2-hydroxyethoxy)-ethyl ester [INN: ETOFENAMATE], p-chlorobenzoic acid, ester with 4-butyl-4-(hydroxymethyl)-1,2-diphenyl-3,5-pyrazolidinedione [INN: FECLOBUZONE], 4-biphenylacetic acid [INN: FELBINAC], 3-(4-biphenylylcarbonyl)propionic acid [INN: FENBUFEN], [o-(2,4-dichlorophenoxy)phenyl]acetic acid [INN: FENCLOFENAC], (plus/minus)-m-phenoxyhydratropic acid [INN: FENOPROFEN], 4-(p-chlorophenyl)-2-phenyl-5-thiazoleacetic acid [INN: FENTIAZAC], (plus/minus)-alpha-[[(2-hydroxy-1,1-dimethylethyl)amino]methyl]-benzyl alcohol [INN: FEPRADINOL], 4-(2′,4′-difluorobiphenylyl)-4-oxo-2-methylbutanoic acid [INN: FLOBUFEN), 2,3-dihydroxypropyl N-[8-(trifluoromethyl)-4-quinoly]anthranilate [INN: FLOCTAFENINE], N-(alpha,alpha,alpha-trifluoro-m-tolyl)-anthranilic acid [INN: FLUFENAMIC ACID], (plus)-2-(p-fluorophenyl)-alpha-methyl-5-benzoxazoleacetic acid [INN: FLUNOXAPROFEN], 2-fluoro-alpha-methyl-4-biphenylacetic acid [INN: FLURBIPROFEN], (plus/minus)-2-(2-fluoro-4-biphenylyl)propionic acid 1 (acetoxy)ethyl ester [INN: FLURBIPROFEN AXETIL], 2-ethyl-2,3-dihydro-5-benzofuranacetic acid [INN: FUROFENAC], 2-[4-(2′-furoyl)phenyl]propionic acid [INN: FURPROFEN], 2-[2-[1-(p-chlorobenzoyl)-5-methoxy-2-methylindol-3-yl]acetamido]-2-deoxy-D-glucose [INN: GLUCAMETACIN], 2-(2-fluorobiphenyl-4-yl)propionic acid 4-nitrooxybutylester [Research Code: HCT-1026], (p-isobutylphenyl)acetic acid [INN: IBUFENAC], alpha-p-isobutylphenylpropionic acid [INN: IBUPROFEN], methyl 4-(3-thienyl)phenyl-alpha-methylacetate [Research Code: IDPH-8261], (plus/minus)-2-[p-(1-oxo-2-isoindolinyl)phenyl]butyric acid [INN: INDOBUFEN], 1-(4-chlorobenzoyl)-5-methoxy-2-methyl-1H-indole-3-acetic acid [INN: INDOMETACIN], 1-(4-chlorobenzoyl)-5-methoxy-2-methyl-1H-indole-3-acetic acid, 3,7,11-trimethyl-2,6,10-dodecatrienyl ester [INN: INDOMETACIN FARNESIL], p-(1-oxo-2-isoindolinyl)hydratropic acid [INN: INDOPROFEN], 2-(10-methoxy-4H-benzo[4,5]cyclohepta[1,2-b]thiophen-4-ylidene)-acetic acid [Research Code: IX-207-887], m-benzoylhydratropic acid [INN: KETOPROFEN], (DL)-5-benzoyl-3H-1,2-dihydropyrrolo[1,2-a]pyrrole-1-carboxylic acid [INN: KETOROLAC], 2,3-dihydro-5-hydroxy-6-[2-(hydroxymethyl)cinnamyl]benzofuran [Research Code: L-651896], N-(2-carboxyphenyl)-4-chloroanthranilic acid [INN: LOBENZARIT], 3-(p-chlorophenyl)-1-phenylpyrazole-4-acetic acid [INN: LONAZOLAC], 6-chloro-4-hydroxy-2-methyl-N-2-pyridyl-2H-thieno[2,3-e]-1,2-thiazine-3-carboxamide 1,1-dioxide [INN: LORNOXICAM], 2-[4-(2-oxocyclopentan-1-ylmethyl)phenyl]-propionate [INN: LOXOPROFEN], 2(R)-[4-(3-methyl-2-thienyl)phenyl]propionic acid [Research Code: M-5010], N-(2,3-xylyl)anthranilic acid [INN: MEFENAMIC ACID], 4-hydroxy-2-methyl-N-(5-methyl-2-thiazolyl)-2H-1,2-benzothiazine-3-carboxamide 1,1-dioxide [INN: MELOXICAM], 5-aminosalicylic acid [INN: MESALAZINE], (2,2-dimethyl-6-(4-chlorophenyl)-7-phenyl-2,3-dihydro-1H-pyrrolizin-5-yl)-acetic acid [Research Code: ML-3000], 3,4-bis(4-methoxyphenyl)-5-isoxazoleacetic acid [INN: MOFEZOLAC], 4-(6-methoxy-2-naphthyl)-2-butanone [INN: NABUMETONE], (plus)-6-methoxy-alpha-methyl-2-naphthalineacetic acid [INN: NAPROXEN], 2-[3-(trifluoromethyl)anilino]nicotinic acid [INN: NIFLUMIC ACID], 5,5′-azodisalicylic acid [INN: OLSALAZINE], 4,5-diphenyl-2-oxazolepropionic acid [INN: OXAPROZIN], alpha-methyl-4-[(2-oxocyclohexylidene)methyl]benzene acetic acid [INN: PELUBIPROFEN], 4-butyl-1,2-diphenyl-3,5-pyrazolidinedione [INN: PHENYLBUTAZONE], 2-(p-isobutylphenyl)propionic acid 2-pyridyl-methyl ester [INN: PIMEPROFEN], 4-(p-chlorophenyl)-1-(p-fluorophenyl)pyrazole-3-acetic acid [INN: PIRAZOLAC], 4-hydroxy-2-methyl-N-2-pyridyl-2H-1,2-benzothiadiazin-3-carboxamide 1,1-dioxide [INN: PIROXICAM], 3-chloro-4-(3-pyrrolin-1-yl)hydratropic acid [INN: PIRPROFEN], 2-[5H-(1)benzopyrano]2,3-b]pyridin-7-yl]propionic acid [INN: PRANOPROFEN], 2,6-di-tert-butyl-4-(2′-thenoyl)phenol [INN: PRIFELONE], alpha-cyano-1-methyl-beta-oxopyrrole-2-propionanilide [INN: PRINOMIDE], 3-[4-(2-hydroxyethyl)-1-piperazinyl]-propyl-D,L-4-benzamido-N,N-dipropylglutaramat 1-(p-chlorobenzoyl)-5-methoxy-2-methylindole-3-acetate (ester) [INN: PROGLUMETACIN], 7-methyl-1-(1-methylethyl)-4-phenyl-2(1H)quinazolinone [INN: PROQUAZONE], 7-methoxy-alpha,10-dimethylphenothiazine-2-acetic acid [INN: PROTIZINIC ACID], 2-[[2-(p-chlorophenyl)-4-methyl-5-oxazolyl]methoxy]-2-methylpropionic acid [INN: ROMAZARIT], o-hydroxybenzamide [SALICYLAMIDE], 2-hydroxybenzoic acid [SALICYLIC ACID], N-acetyl-L-cysteine salicylate (ester), acetate (ester) [INN: SALMISTEINE], N-acetyl-L-cysteine salicylate (ester) [INN: SALNACEDIN], 2-hydroxybenzoic acid 2-carboxyphenyl ester [INN: SALSALATE], 4-[1-(2-fluorobiphenyl-4-yl)ethyl]-N-methylthiazole-2-amine [Research Code: SM-8849], (Z)-5-fluoro-2-methyl-1-[p-(methylsulfinyl)benzylidene]indene-3-acetic acid [INN: SULINDAC], p-2-thenoylhydratropic acid [INN: SUPROFEN] 2-(4-(3-methyl-2-butenyl)phenyl)propionic acid [Research Code: TA-60], phthalidyl 2-(alpha,alpha,alpha-trifluoro-m-toluidino)nicotinate [INN: TALNIFLUMATE], (Z)-5-chloro-3-(2-thenoyl)-2-oxoindole-1-carboxamide [INN: TENIDAP], 2-thiophenecarboxylic acid, ester with salicylic acid [INN: TENOSAL], 4-hydroxy-2-methyl-N-2-pyridyl-2H-thieno[2,3-e]-1,2-thiazine-3-carboxamide [INN: TENOXICAM], 5-(4-chlorophenyl)-N-hydroxy-1-(4-methoxyphenyl)-N-methyl-1H-pyrazole-3-propionamide [INN: TEPOXALIN], alpha-(5-benzoyl-2-thienyl)propionic acid [INN: TIAPROFENIC ACID], 5-chloro-3-[4-(2-hydroxyethyl)-1-piperazinyl]carbonylmethyl-2-benzo-thiazolin one [INN: TIARAMIDE], 2-(2-methyl-5H-[1]benzopyrano[2,3-b]pyridin-7-yl)-propionic acid N,N-dimethylcarbamoylmethyl ester [INN: TILNOPROFEN ARBAMEL], 1-Cyclohexyl-2-(2-methyl-4-quinolyl)-3-(2-thiazolyl)guanidine [INN: TIMEGADINE], 2-amino-6-benzyl-4,5,6,7-tetrahydrothieno[2,3-c]pyridine [INN: TINORIDINE], N-(3-chloro-o-tolyl)anthranilic acid [INN: TOLFENAMIC ACID], 1-methyl-5-(4-methylbenzoyl)-1H-pyrrole-2-acetic acid [INN: TOLMETIN], hydroxybis[alpha-methyl-4-(2-methylpropyl)benzene acetato-O]-aluminium [Research Code: U-18573-G], N-(3-trifluoromethylphenyl)-anthranilic acid n-butyl ester [INN: UFENAMATE], 2-[4-[3-(hydroxyimino)cyclohexyl]phenyl]propionic acid [INN: XIMOPROFEN], 2-(10,11-dihydro-10-oxo-dibenz[b,f]thiepin-2-yl-propionic acid [INN: ZALTOPROFEN] and 2-[4-(2-thiazolyloxy)phenyl]-propionic acid [INN: ZOLIPROFEN]; wherein said NO-NSAID is selected from the group consisting of those NO-NSAIDs which are disclosed in WO 96/32946, WO 96/35416, WO 96/38136, WO 96/39409, WO 00/50037, U.S. Pat. No. 6,057,347, WO 94/04484, WO 94/12463, WO 95/09831, WO 95/30641, WO 97/31654, WO 99/44595, WO 99/45004 and WO 01/45703; wherein said COX-2 inhibitor is selected from the group consisting of 5-chloro-6′-methyl-3-[4-(methylsulfonyl)phenyl]-2,3′-bipyridine [INN: ETORICOXIB], 4-[5-(4-methylphenyl)-3-(trifluoromethyl)-1H-pyrazol-1-yl]benzene-sulfonamide [INN: CELECOXIB], 4-[p-(methylsulfonyl)phenyl]-3-phenyl-2(5H)-furanone [INN: ROFECOXIB], N-[[p-(5-methyl-3-phenyl-4-isoxazolyl)phenyl]sulfonyl]propion-amide [INN: PARECOXIB], p-(5-methyl-3-phenyl-4-isoxazolyl)benzenesulfonamide [INN: VALDECOXIB], 2-[2-(2-chloro-6-fluorophenylamino)-5-methylphenyl]acetic acid [INN: LUMIRACOXIB], 4-(4-cyclohexyl-2-methyloxazol-5-yl)-2-fluorobenzene-sulfonamide [INN: TILMACOXIB], 4-[4-chloro-5-(3-fluoro-4-methoxyphenyl)-1H-imidazol-1-yl]benzene-sulfonamide [INN: CIMICOXIB], 4′-nitro-2′-phenoxymethanesulfonanilide [INN: NIMESULIDE], 6-(2,4-difluorophenoxy)-5-methylsulfonylamino-1-indanone [INN: FLOSULIDE], 5-bromo-2-(4-fluoro-phenyl)-3-(4-methanesulfonyl-phenyl)-thiophene [DUP-697], 4-acetyl-2-(2,4-difluorophenoxy)methanesulfonanilide [FK-3311], N-[2-(cyclohexyloxy)-4-nitrophenyl]methanesulfonamide [NS-398], 5-methanesulfonamido-6-(2,4-difluorophenylthio)-1-indanone [L-745337], 8-acetyl-3-(4-fluorophenyl)-2-[4-(methanesulfonyl)phenyl]imidazo[1,2-a]-pyridine [GR-253035], 4-[5-(4-chlorophenyl)-3-(trifluoromethyl)pyrazol-1-yl]benzenesulfon-amide [SC-58236], 4-(2,3-dihydro-2-oxo-3-phenyl-4-oxazolyl)-benzenesulfonamide [LAS-33815], CS-502, 2-(3,4-difluorophenyl)-4-(3-hydroxy-3-methylbutoxy)-5-[4-(methyl-sulfonyl)phenyl]-3(2H)-pyridazinone [ABT-963], GW-406381, and those COX-2 inhibitors disclosed in WO 02096427, WO 02096886 and WO 02096885; wherein said bisphosphonate is selected from the group consisting of ALENDRONIC ACID, RISEDRONIC ACID, TILUDRONIC ACID, IBANDRONIC ACID, ZOLEDRONIC ACID, CLODRONIC ACID, INCADRONIC ACID, OLPADRONIC ACID, MINODRONIC ACID, PAMIDRONIC ACID and ETIDRONIC ACID; wherein said corticosteroid is selected from the group consisting of HYDROCORTISONE, PREDNISONE, PREDNISOLONE, METHYLPREDNISOLONE, TRIAMCINOLONE ACETONIDE, AMCINONIDE, CLOBETASONE, CLOBETASOL, DEFLAZACORT, DESONIDE, CLOPREDNOL, DEXAMETHASONE, DIFLORASONE, DIFLUCORTOLONE, DIFLUPREDNATE, FLUDROXYCORTIDE, FLUDROCORTISONE, FLUMETASONE, TIXOCORTOL PIVALATE, FLUOCORTIN BUTYL, CLOCORTOLONE, FLUOCINOLONE ACETONIDE, FLUOCORTOLONE, FLUOROMETHOLONE, FLUPREDNIDENE, FLUPREDNISOLONE, BETAMETHASONE, HALCINONIDE, BUDESONIDE, HALOMETASONE, RIMEXOLONE, PARAMETHASONE, PREDNYLIDENE, LOTEPREDNOL ETABONATE and PREDNICARBATE; and pharmaceutically acceptable derivatives of any of these compounds.

28. A commercial package according to claim 23, wherein said NSAID is selected from the group consisting of glycolic acid [o-(2,6-dichloroanilino)phenyl]acetate(ester) [INN: ACECLOFENAC]; 1-(4-chlorobenzoyl)-5-methoxy-2-methyl-1H-indole-3-acetic acid carboxymethyl ester [INN: ACEMETACIN]; 2-(acetyloxy)benzoic acid [ACETYLSALICYLIC ACID], 2-methoxyphenyl-alpha-methyl-4-(isobutyl)phenylacetate [Research Code: AF-2259], (4-allyloxy-3-chlorophenyl)acetic acid [INN: ALCLOFENAC], p-[(2-methylallyl)amino]hydratropic acid [INN: ALMINOPROFEN], 2-amino-3-benzoylphenylacetic acid [INN: AMFENAC], (plus/minus)-4-(1-hydroxyethoxy)-2-methyl-N-2-pyridyl-2H-1,2-benzothiazine-3-carboxamide ethylcarbonate (ester), 1,1-dioxide [INN: AMPIROXICAM], 2-methoxyphenyl-1-methyl-5-(p-methylbenzoyl)pyrrol-2-acetamido-acetate [INN: AMTOLMETINGUACIL], (plus/minus)-2,3-dihydro-5-(4-methoxybenzoyl)-1H pyrrolizine-1-carboxylic acid [INN: ANIROLAC], 2-[4-(alpha,alpha,alpha-trifluoro-m-tolyl)-1-piperazinyl]ethyl-N-(7-trifluoromethyl-4-quinolyl)anthranilate [INN: ANTRAFENINE], 5-(dimethylamino)-9-methyl-2-propyl-1H-pyrazolo[1,2-a][1,2,4]benzo-triazine-1,3(2H)-dione [INN: AZAPROPAZONE], 4-acetamidophenyl salicylate acetate [INN: BENORILATE], 2-(8-methyl-10,11-dihydro-11-oxodibenz[b,f]oxepin-2-yl)propionic acid [INN: BERMOPROFEN], 2-[(1-benzyl-1H-indazol-3-yl)methoxy]-2-methylpropionic acid [INN: BINDARIT], [2-amino-3-(p-bromobenzoyl)phenyl]acetic acid [INN: BROMFENAC], 3-(3-chloro-4-cyclohexylbenzoyl)propionic acid [INN: BUCLOXIC ACID], 5-butyl-1-cyclohexylbarbituric acid [INN: BUCOLOM], 4-butoxy-N-hydroxybenzeneacetamide [INN: BUFEXAMAC], butylmalonic acid mono-(1,2-diphenylhydrazide) [INN: BUMADIZONE], alpha-ethyl-4-(2-methylpropyl)benzeneacetic acid [INN: BUTIBUFEN], 2-(4-biphenylyl)butyric acid, trans-4-phenylcyclohexylamine salt (1:1) [INN: BUTIXIRATE], 2-(acetyloxy)-benzoic acid, calcium salt, compound with urea (1:1) [INN: CARBASALATE CALCIUM], (plus/minus)-6-chloro-alpha-methylcarbazole-2-acetic acid [INN: CARPROFEN], 1-cinnamoyl-5-methoxy-2-methylindole-3-acetic acid [INN: CINMETACIN], N-(2-pyridyl)-2-methyl-4-cinnamoyloxy-2H-1,2-benzothiazine-3-carboxamide-1,1-dioxide [INN: CINNOXICAM], 6-chloro-5-cyclohexyl-1-indancarboxylic acid [INN: CLIDANAC], 2-[4-(p-chlorophenyl)benzyloxy]-2-methylpropionic acid [INN: CLOBUZARIT], 5-methoxy-2-methyl-3-indolylacetohydroxamic acid [INN: DEBOXAMET], (S)-(+)-p-isobutylhydratropic acid [INN: DEXIBUPROFEN], (+)-(S)-m-benzoylhydratropic acid [INN: DEXKETOPROFEN], 2-[(2,6-dichlorophenyl)amino]benzeneacetic acid [INN: DICLOFENAC], 2′,4′-Difluoro-4-hydroxy-3-biphenylcarboxylic acid [INN: DIFLUNISAL], 4-(2,6-dichloroanilino)-3-thiopheneacetic acid [INN: ELTENAC], N-beta-phenethyl-anthranilic acid [INN: ENFENAMIC ACID] salicylic acid acetate, ester with beta-hydroxy p-acetophenetidide [INN: ETERSALATE], 1,8-diethyl-1,3,4,9-tetrahydropyrano[3,4-b]indole-1-acetic acid [INN: ETODOLAC], 2-[[3-(trifluoromethyl)phenyl]amino]benzoic acid 2-(2-hydroxyethoxy)-ethyl ester [INN: ETOFENAMATE], p-chlorobenzoic acid, ester with 4-butyl-4-(hydroxymethyl)-1,2-diphenyl-3,5-pyrazolidinedione [INN: FECLOBUZONE], 4-biphenylacetic acid [INN: FELBINAC], 3-(4-biphenylylcarbonyl)propionic acid [INN: FENBUFEN], [o-(2,4-dichlorophenoxy)phenyl]acetic acid [INN: FENCLOFENAC], (plus/minus)-m-phenoxyhydratropic acid [INN: FENOPROFEN], 4-(p-chlorophenyl)-2-phenyl-5-thiazoleacetic acid [INN: FENTIAZAC], (plus/minus)-alpha-[[(2-hydroxy-1,1-dimethylethyl)amino]methyl]-benzyl alcohol [INN: FEPRADINOL], 4-(2′,4′-difluorobiphenylyl)-4-oxo-2-methylbutanoic acid [INN: FLOBUFEN], 2,3-dihydroxypropyl N-[8-(trifluoromethyl)-4-quinoly]anthranilate [INN: FLOCTAFENINE], N-(alpha,alpha,alpha-trifluoro-m-tolyl)-anthranilic acid [INN: FLUFENAMIC ACID], (plus)-2-(p-fluorophenyl)-alpha-methyl-5-benzoxazoleacetic acid [INN: FLUNOXAPROFEN], 2-fluoro-alpha-methyl-4-biphenylacetic acid [INN: FLURBIPROFEN], (plus/minus)-2-(2-fluoro-4-biphenylyl)propionic acid 1 (acetoxy)ethyl ester [INN: FLURBIPROFEN AXETIL], 2-ethyl-2,3-dihydro-5-benzofuranacetic acid [INN: FUROFENAC], 2-[4-(2′-furoyl)phenyl]propionic acid [INN: FURPROFEN], 2-[2-[1-(p-chlorobenzoyl)-5-methoxy-2-methylindol-3-yl]acetamido]-2-deoxy-D-glucose [INN: GLUCAMETACIN], 2-(2-fluorobiphenyl-4-yl)propionic acid 4-nitrooxybutylester [Research Code: HCT-1026], (p-isobutylphenyl)acetic acid [INN: IBUFENAC], alpha-p-isobutylphenylpropionic acid [INN: IBUPROFEN], methyl 4-(3-thienyl)phenyl-alpha-methylacetate [Research Code: IDPH-8261], (plus/minus)-2-[p-(1-oxo-2-isoindolinyl)phenyl]butyric acid [INN: INDOBUFEN], 1-(4-chlorobenzoyl)-5-methoxy-2-methyl-1H-indole-3-acetic acid [INN: INDOMETACIN], 1-(4-chlorobenzoyl)-5-methoxy-2-methyl-1H-indole-3-acetic acid, 3,7,11-trimethyl-2,6,10-dodecatrienyl ester [INN: INDOMETACIN FARNESIL], p-(1-oxo-2-isoindolinyl)hydratropic acid [INN: INDOPROFEN], 2-(10-methoxy-4H-benzo[4,5]cyclohepta[1,2-b]thiophen-4-ylidene)-acetic acid [Research Code: IX-207-887], m-benzoylhydratropic acid [INN: KETOPROFEN], (DL)-5-benzoyl-3H-1,2-dihydropyrrolo[1,2-a]pyrrole-1-carboxylic acid [INN: KETOROLAC], 2,3-dihydro-5-hydroxy-6-[2-(hydroxymethyl)cinnamyl]benzofuran [Research Code: L-651896], N-(2-carboxyphenyl)-4-chloroanthranilic acid [INN: LOBENZARIT], 3-(p-chlorophenyl)-1-phenylpyrazole-4-acetic acid [INN: LONAZOLAC], 6-chloro-4-hydroxy-2-methyl-N-2-pyridyl-2H-thieno[2,3-e]-1,2-thiazine-3-carboxamide 1,1-dioxide [INN: LORNOXICAM], 2-[4-(2-oxocyclopentan-1-ylmethyl)phenyl]-propionate [INN: LOXOPROFEN], 2(R)-[4-(3-methyl-2-thienyl)phenyl]propionic acid [Research Code: M-5010], N-(2,3-xylyl)anthranilic acid [INN: MEFENAMIC ACID], 4-hydroxy-2-methyl-N-(5-methyl-2-thiazolyl)-2H-1,2-benzothiazine-3-carboxamide 1,1-dioxide [INN: MELOXICAM], 5-aminosalicylic acid [INN: MESALAZINE], (2,2-dimethyl-6-(4-chlorophenyl)-7-phenyl-2,3-dihydro-1H-pyrrolizin-5-yl)-acetic acid [Research Code: ML-3000], 3,4-bis(4-methoxyphenyl)-5-isoxazoleacetic acid [INN: MOFEZOLAC], 4-(6-methoxy-2-naphthyl)-2-butanone [INN: NABUMETONE], (plus)-6-methoxy-alpha-methyl-2-naphthalineacetic acid [INN: NAPROXEN], 2-[3-(trifluoromethyl)anilino]nicotinic acid [INN: NIFLUMIC ACID], 5,51-azodisalicylic acid [INN: OLSALAZINE], 4,5-diphenyl-2-oxazolepropionic acid [INN: OXAPROZIN], alpha-methyl-4-[(2-oxocyclohexylidene)methyl]benzene acetic acid [INN: PELUBIPROFEN], 4-butyl-1,2-diphenyl-3,5-pyrazolidinedione [INN: PHENYLBUTAZONE], 2-(p-isobutylphenyl)propionic acid 2-pyridyl-methyl ester [INN: PIMEPROFEN], 4-(p-chlorophenyl)-1-(p-fluorophenyl)pyrazole-3-acetic acid [INN: PIRAZOLAC], 4-hydroxy-2-methyl-N-2-pyridyl-2H-1,2-benzothiadiazin-3-carboxamide 1,1-dioxide [INN: PIROXICAM], 3-chloro-4-(3-pyrrolin-1-yl)hydratropic acid [INN: PIRPROFEN], 2-[5H-(1)benzopyrano]2,3-b]pyridin-7-yl]propionic acid [INN: PRANOPROFEN], 2,6-di-tert-butyl-4-(2′-thenoyl)phenol [INN: PRIFELONE], alpha-cyano-1-methyl-beta-oxopyrrole-2-propionanilide [INN: PRINOMIDE], 3-[4-(2-hydroxyethyl)-1-piperazinyl]-propyl-D,L-4-benzamido-N,N-dipropylglutaramat 1-(p-chlorobenzoyl)-5-methoxy-2-methylindole-3-acetate (ester) [INN: PROGLUMETACIN], 7-methyl-1-(1-methylethyl)-4-phenyl-2(1H)quinazolinone [INN: PROQUAZONE], 7-methoxy-alpha,10-dimethylphenothiazine-2-acetic acid [INN: PROTIZINIC ACID], 2-[[2-(p-chlorophenyl)-4-methyl-5-oxazolyl]methoxy]-2-methylpropionic acid [INN: ROMAZARIT], o-hydroxybenzamide [SALICYLAMIDE], 2-hydroxybenzoic acid [SALICYLIC ACID], N-acetyl-L-cysteine salicylate (ester), acetate (ester) [INN: SALMISTEINE], N-acetyl-L-cysteine salicylate (ester) [INN: SALNACEDIN], 2-hydroxybenzoic acid 2-carboxyphenyl ester [INN: SALSALATE], 4-[1-(2-fluorobiphenyl-4-yl)ethyl]-N-methylthiazole-2-amine [Research Code: SM-8849], (Z)-5-fluoro-2-methyl-1-[p-(methylsulfinyl)benzylidene]indene-3-acetic acid [INN: SULINDAC], p-2-thenoylhydratropic acid [INN: SUPROFEN] 2-(4-(3-methyl-2-butenyl)phenyl)propionic acid [Research Code: TA-60], phthalidyl 2-(alpha,alpha,alpha-trifluoro-m-toluidino)nicotinate [INN: TALNIFLUMATE], (Z)-5-chloro-3-(2-thenoyl)-2-oxoindole-1-carboxamide [INN: TENIDAP], 2-thiophenecarboxylic acid, ester with salicylic acid [INN: TENOSAL], 4-hydroxy-2-methyl-N-2-pyridyl-2H-thieno[2,3-e]-1,2-thiazine-3-carboxamide [INN: TENOXICAM], 5-(4-chlorophenyl)-N-hydroxy-1-(4-methoxyphenyl)-N-methyl-1H-pyrazole-3-propionamide [INN: TEPOXALIN], alpha-(5-benzoyl-2-thienyl)propionic acid [INN: TIAPROFENIC ACID], 5-chloro-3-[4-(2-hydroxyethyl)-1-piperazinyl]carbonylmethyl-2-benzo-thiazolin one [INN: TIARAMIDE], 2-(2-methyl-5H-[I]benzopyrano[2,3-b]pyridin-7-yl)-propionic acid N,N-dimethylcarbamoylmethyl ester [INN: TILNOPROFEN ARBAMEL], 1-Cyclohexyl-2-(2-methyl-4-quinolyl)-3-(2-thiazolyl)guanidine [INN: TIMEGADINE], 2-amino-6-benzyl-4,5,6,7-tetrahydrothieno[2,3-c]pyridine [INN: TINORIDINE], N-(3-chloro-o-tolyl)anthranilic acid [INN: TOLFENAMIC ACID], 1-methyl-5-(4-methylbenzoyl)-1H-pyrrole-2-acetic acid [INN: TOLMETIN], hydroxybis[alpha-methyl-4-(2-methylpropyl)benzene acetato-O]-aluminium [Research Code: U-18573-G], N-(3-trifluoromethylphenyl)-anthranilic acid n-butyl ester [INN: UFENAMATE], 2-[4-[3-(hydroxyimino)cyclohexyl]phenyl]propionic acid [INN: XIMOPROFEN], 2-(10,11-dihydro-10-oxo-dibenz[b,f]thiepin-2-yl-propionic acid [INN: ZALTOPROFEN] and 2-[4-(2-thiazolyloxy)phenyl]-propionic acid [INN: ZOLIPROFEN]; wherein said NO-NSAID is selected from the group consisting of those NO-NSAIDs which are disclosed in WO 96/32946, WO 96/35416, WO 96/38136, WO 96/39409, WO 00/50037, U.S. Pat. No. 6,057,347, WO 94/04484, WO 94/12463, WO 95/09831, WO 95/30641, WO 97/31654, WO 99/44595, WO 99/45004 and WO 01/45703; wherein said COX-2 inhibitor is selected from the group consisting of 5-chloro-6′-methyl-3-[4-(methylsulfonyl)phenyl]-2,3′-bipyridine [INN: ETORICOXIB], 4-[5-(4-methylphenyl)-3-(trifluoromethyl)-1H-pyrazol-1-yl]benzene-sulfonamide [INN: CELECOXIB], 4-[p-(methylsulfonyl)phenyl]-3-phenyl-2(5H)-furanone [INN: ROFECOXIB], N-[[p-(5-methyl-3-phenyl-4-isoxazolyl)phenyl]sulfonyl]propion-amide [INN: PARECOXIB], p-(5-methyl-3-phenyl-4-isoxazolyl)benzenesulfonamide [INN: VALDECOXIB], 2-[2-(2-chloro-6-fluorophenylamino)-5-methylphenyl]acetic acid [INN: LUMIRACOXIB], 4-(4-cyclohexyl-2-methyloxazol-5-yl)-2-fluorobenzene-sulfonamide [INN: TILMACOXIB], 4-[4-chloro-5-(3-fluoro-4-methoxyphenyl)-1H-imidazol-1-yl]benzene-sulfonamide [INN: CIMICOXIB], 4′-nitro-2′-phenoxymethanesulfonanilide [INN: NIMESULIDE], 6-(2,4-difluorophenoxy)-5-methylsulfonylamino-1-indanone [INN: FLOSULIDE], 5-bromo-2-(4-fluoro-phenyl)-3-(4-methanesulfonyl-phenyl)-thiophene [DUP-697], 4-acetyl-2-(2,4-difluorophenoxy)methanesulfonanilide [FK-3311], N-[2-(cyclohexyloxy)-4-nitrophenyl]methanesulfonamide [NS-398], 5-methanesulfonamido-6-(2,4-difluorophenylthio)-1-indanone [L-745337], 8-acetyl-3-(4-fluorophenyl)-2-[4-(methanesulfonyl)phenyl]imidazo[1,2-a]-pyridine [GR-253035], 4-[5-(4-chlorophenyl)-3-(trifluoromethyl)pyrazol-1-yl]benzenesulfon-amide [SC-58236], 4-(2,3-dihydro-2-oxo-3-phenyl-4-oxazolyl)-benzenesulfonamide [LAS-33815], CS-502, 2-(3,4-difluorophenyl)-4-(3-hydroxy-3-methylbutoxy)-5-[4-(methyl-sulfonyl)phenyl]-3(2H)-pyridazinone [ABT-963], GW-406381, and those COX-2 inhibitors disclosed in WO 02096427, WO 02096886 and WO 02096885; wherein said bisphosphonate is selected from the group consisting of ALENDRONIC ACID, RISEDRONIC ACID, TILUDRONIC ACID, IBANDRONIC ACID, ZOLEDRONIC ACID, CLODRONIC ACID, INCADRONIC ACID, OLPADRONIC ACID, MINODRONIC ACID, PAMIDRONIC ACID and ETIDRONIC ACID; wherein said corticosteroid is selected from the group consisting of HYDROCORTISONE, PREDNISONE, PREDNISOLONE, METHYLPREDNISOLONE, TRIAMCINOLONE ACETONIDE, AMCINONIDE, CLOBETASONE, CLOBETASOL, DEFLAZACORT, DESONIDE, CLOPREDNOL, DEXAMETHASONE, DIFLORASONE, DIFLUCORTOLONE, DIFLUPREDNATE, FLUDROXYCORTIDE, FLUDROCORTISONE, FLUMETASONE, TIXOCORTOL PIVALATE, FLUOCORTIN BUTYL, CLOCORTOLONE, FLUOCINOLONE ACETONIDE, FLUOCORTOLONE, FLUOROMETHOLONE, FLUPREDNIDENE, FLUPREDNISOLONE, BETAMETHASONE, HALCINONIDE, BUDESONIDE, HALOMETASONE, RIMEXOLONE, PARAMETHASONE, PREDNYLIDENE, LOTEPREDNOL ETABONATE and PREDNICARBATE; and pharmaceutically acceptable derivatives of any of these compounds.
